(19) United States
(12) Patent Application Publication
(10) Pub. No.: US 2024/0216605 A1
(43) Pub. Date: Jul. 4, 2024

(54) Infusion Set and Inserter Assembly Apparatuses, Systems, and Methods

(71) Applicant: DEKA Products Limited Partnership, Manchester, NH (US)

(72) Inventor: Richard J. Lanigan, Concord, NH (US)

(21) Appl. No.: 18/606,463

(22) Filed: Mar. 15, 2024

Related U.S. Application Data
(63) Continuation of application No. 17/896,395, filed on Aug. 26, 2022.
(60) Provisional application No. 63/237,259, filed on Aug. 26, 2021.

(51) Int. Cl. A61M 5/158

(52) U.S. Cl. CPC A61M 5/158; A61M 2005/1585; A61M 2005/1586

(57) ABSTRACT

An inserter assembly may comprise a casing having an open end and a closed end. The inserter assembly may further comprise a first unit at least partially within the casing movable relative to the casing. The first unit may comprise a device base. The first unit may further comprise a sharp. The first unit may further comprise a body configured to displace in tandem with the sharp at least during displacement of the sharp from a forward to a retracted state. The first unit may further comprise a bias member configured to displace the body toward the closed end and displace the sharp to the retracted state when a release latch is transitioned from an engaged to a released state. One of the casing and the body may include a projection which engenders tilting of the body as the body and sharp are displaced by the bias member.

### CROSS REFERENCE TO RELATED APPLICATIONS

The present application is a Continuation of U.S. patent application Ser. No. 17/896,395, filed on Aug. 26, 2022, now US2023/0062738A1, published Mar. 2, 2023, and entitled Infusion Set and Inserter Assembly Apparatuses, Systems, and Methods (Attorney Docket No. 00101.00338.AA906) which claims the benefit of U.S. Provisional Application Ser. No. 63/237,259 filed Aug. 26, 2021 and entitled Infusion Set and Inserter Assembly Apparatuses, Systems, and Methods (Attorney Docket No. 00101.00318.AA596), each of which being hereby incorporated herein by reference in their entireties.

### BACKGROUND

### Field of Disclosure

This application relates generally to infusion sets and inserter assemblies for infusion sets, and more particularly, to infusion sets and inserter assemblies as well as methods for the use thereof.

### Description of Related Art

Many potentially valuable medicines or compounds, including biologicals, are not orally active due to poor absorption, hepatic metabolism or other pharmacokinetic factors. Additionally, some therapeutic compounds, although they can be orally administered, are sometimes required to be taken so often that it is difficult for a patient to maintain the desired schedule. In these cases, parenteral delivery is often employed or could be employed.

Effective parenteral delivery routes of drugs, other fluid, and compounds such as subcutaneous injection, intramuscular injection, and intravenous (IV) administration include puncture of the skin with a needle or stylet. Insulin is an example of a therapeutic fluid that is self-injected by millions of diabetic patients. Users of parenterally delivered drugs may benefit from a wearable device that would automatically deliver needed drugs/compound over a period of time.

To this end, there have been efforts to design portable and wearable devices for the controlled release of therapeutics. Such devices are known to have a reservoir such as a cartridge, syringe, or bag, and to be electronically controlled. These devices suffer from a number of drawbacks including the malfunction rate. Reducing the size, weight, and cost of these devices is also an ongoing challenge. Additionally, these devices often apply to the skin and pose the challenge of frequent relocation for application.

### SUMMARY

In accordance with an example embodiment of the present disclosure an inserter assembly may comprise a casing having an open end and a closed end. The inserter assembly may further comprise a first unit at least partially within the casing. The first unit may be movable relative to the casing. The first unit may comprise a device base. The first unit may further comprise an insertion sharp displaceable between a raised state, a forward state, and a retracted state. The first unit may further comprise a body configured to displace in tandem with the insertion sharp at least during displacement of the insertion sharp from the forward state to the retracted state. The first unit may further comprise a first bias member configured to urge the body toward the closed end of the casing. The first unit may further comprise a bias member release latch having an engaged state and a released state. The first bias member may be configured to displace the body toward the closed end and displace the insertion sharp to the retracted state when the release latch is transitioned from the engaged state to the released state. One of the casing and the body may include a tipping projection which obstructs displacement of a portion of the body and engenders tilting of the body as the body and insertion sharp are displaced by the first bias member.

In some embodiments, the inserter assembly may include at least one guide. The body may be displaced along and then out of engagement with the at least one guide as the body is displaced toward the closed end by the first bias member. In some embodiments, first unit may further comprise a second bias member configured to propel the insertion sharp from the raised state to the forward state. In some embodiments, the inserter assembly may further comprise a release latch for the second bias member configured to be transitioned from an engaged state to a released state after a magnitude of displacement of the casing relative to the first unit exceeds a threshold. In some embodiments, the device base may be coupled to an adhesive. The adhesive may be configured to anchor the first unit against a patient while the casing is pulled away from the patient and the magnitude of displace of the casing relative to the first unit increases. In some embodiments, the first unit may further comprise a cannula assembly carried by the insertion sharp. In some embodiments, the cannula assembly may be configured to couple to the device base and transition the bias member release latch to the released state when the insertion sharp reaches the forward position. In some embodiments, the inserter assembly may include an interior housing within the casing. The interior housing may including a guard. At least the tip of the insertion sharp may be displaced into the guard as the insertion sharp is displaced to the retracted state. In some embodiments, the guard may double as a guide which directs displacement of the body as the body is displaced toward the closed end.

In accordance with another exemplary embodiment of the present disclosure, an inserter assembly may comprise a casing having an open end and a closed end. The inserter assembly may further comprise a first unit at least partially within the casing. The first unit may be movable relative to the casing. The first unit may comprise a device base. The first unit may further comprise a sharp holder including an insertion sharp. The sharp holder may be displaceable between a raised state, a forward state, and a retracted state. The first unit may further comprise a retractor. The sharp holder may be at least partially disposed within the retractor. The first unit may further comprise a first bias member configured to displace the retractor from an initial position to a retracted position. The first unit may further comprise a bias member release latch having an engaged state and a released state. The first bias member may be configured to displace the retractor and insertion sharp to a retracted state when the release latch is transitioned from the engaged state to the released state. One of the casing and the retractor may include a tipping projection which obstructs displacement of a portion of the sharp retractor and engenders tilting of the retractor as the retractor and insertion sharp are displaced to the retracted state.

In some embodiments, the inserter assembly may include an interior housing within the casing defining at least one retractor guide. The retractor may be displaced along and then out of the at least one guide as the retractor and insertion sharp are displaced to the retracted state. In some embodiments, the retractor and sharp holder may move together in unison for at least a portion of the displacement of the retractor and insertion sharp to the retracted state. In some embodiments, the first unit may further comprise a second bias member configured to propel the sharp holder from the raised state to the forward state. In some embodiments, the inserter assembly may further comprise a release latch for the second bias member configured to be transitioned from an engaged state to a released state after a magnitude of displacement of the casing relative to the first unit exceeds a threshold. In some embodiments, the device base may be coupled to an adhesive. The adhesive may be configured to anchor the first unit against a patient while the casing is pulled away from the patient and the magnitude of displace of the casing relative to the first unit increases. In some embodiments, the first unit may further comprise a cannula assembly carried by the insertion sharp. The cannula assembly may be configured to couple to the device base and transition the bias member release latch to the released state when the sharp holder is in the forward position. In some embodiments, the inserter assembly may include an interior housing within the casing. The interior housing may include a guard. The insertion sharp may tilt at least partially into the guard as the retractor and the insertion sharp are displaced to the retracted state. In some embodiments, the guard may double as a retractor guide which directs displacement of the retractor as the retractor is displaced to the retracted state.

In accordance with another example embodiment of the present disclosure, an inserter assembly may comprise a casing having an open end and a closed end. There may be a tipping projection disposed at the closed end. The inserter assembly may further comprise a guide body disposed within the casing and including at least one guide. The inserter assembly may further comprise a patient care assembly base. The inserter assembly may further comprise a sharp holder including an insertion sharp. The sharp holder may be displaceable between a raised state, a forward state, and a retracted state. The inserter assembly may further comprise a sharp retractor. The sharp holder may be at least partially disposed within the sharp retractor. The inserter assembly may further comprise a retraction spring configured to drive the sharp retractor from an initial position to a retracted position. The inserter assembly may further comprise a retraction spring release latch having an engaged state and a released state. The retraction spring may be configured to displace the sharp retractor and insertion sharp along a retraction stroke when the release latch is transitioned from the engaged state to the released state. The sharp retractor displaces along the at least one guide in a first portion of the retraction stroke and is unguided for a second portion of the retraction stroke. The tipping project may be configured to block displacement of a portion of the sharp retractor during at least a part of the second portion of the retraction stroke.

In some embodiments, the sharp retractor and insertion sharp may be non-parallel to an axis of the casing at a conclusion of the retraction stroke. In some embodiments, the guide body may include a guard. At least a tip of the insertion sharp may be displaced into the guard during the second portion of the retraction stroke. In some embodiments, the sharp retractor and sharp holder may move together in unison for at least a portion of the retraction stroke. In some embodiments, the patient care assembly base may be an infusion set base and the inserter assembly may further comprise a cannula assembly carried by the insertion sharp. In some embodiments, the cannula assembly may be coupled to the infusion set base when the sharp holder is displaced to the forward state. In some embodiments, the retraction spring latch may couple the patient care assembly to the sharp retractor when the retraction spring latch is in an engaged state. In some embodiments, the insertion sharp may be closer to the closed end of the casing when in the retracted state compared to the raised state.

In accordance with another example embodiment of the present disclosure, a method of placing a patient care assembly with an inserter assembly may comprise releasing a first latch of the inserter assembly and driving a spring loaded insertion sharp of the inserter assembly from a raised state to a forward state. The method may further comprise releasing a second latch of the inserter assembly and displacing a spring loaded sharp retractor and the insertion sharp through a retraction stroke to a retraction state. The method may further comprise guiding with a guide body, displacement of the sharp retractor in a first stage of the retraction stroke. The method may further comprise tilting the insertion sharp away from alignment with the axis of the inserter assembly and into a tilted state during a second stage of the retraction stroke. The method may further comprise holding, with a spring of the spring loaded sharp retractor, the insertion sharp in the tilted state.

In some embodiments, releasing the second latch may further comprise releasing at least a portion of the patient care assembly. In some embodiments, tilting the insertion sharp may comprise displacing a tip of the insertion sharp into a guard. In some embodiments, the method may further comprise adhering a portion of the patient care assembly to a biological barrier and releasing the first latch may comprise displacing a casing of the inserter assembly in a direction away from the biological barrier. In some embodiments, tilting the insertion sharp may comprise displacing a first portion of the sharp retractor into contact with a closed end of a housing of the inserter assembly and obstructing displacement of a second portion of the sharp retractor shy of the closed end of the housing.

In accordance with another example embodiment of the present disclosure, an inserter assembly for a patient care assembly may comprise an exterior housing having a closed end and an open end. The inserter assembly may further comprise a sharp holder with an insertion sharp coupled thereto disposed within the exterior housing. The inserter assembly may further comprise a latch arrangement including a ledge defined on the sharp holder and a catch defined on a body within the exterior housing. The ledge may be configured to displace along a displacement pathway to transition the latch arrangement from an engaged state and a disengaged state. The inserter assembly may further comprise a bias member held in a distorted state when the latch arrangement is in the engaged state. The bias member may be configured to urge the sharp holder from a raised state toward the open end of the exterior housing and into a forward state when the latch arrangement is transitioned to the disengaged state. The inserter assembly may further comprise a removable lock member extending through the exterior housing and a passage on the body. The lock member may block displacement of the ledge off of the catch and extend substantially perpendicular to the displacement pathway.

In some embodiments, the body may include at least one projection configured to prevent deflection of the lock member in the direction of the displacement pathway. In some embodiments, the body may include a plate having a bridge on a surface thereof. The plate and bridge together may define the passage. In some embodiments, the lock member may include a first projection having a deflectable region. The deflectable region may be substantially in a non-deflected state when the lock member is fully installed in the inserter assembly. In some embodiments, the deflectable region may include a nub. The nub may be configured to abut the wall of the passage and deflect the arm to a deflected state as the lock member is installed in the inserter assembly. In some embodiments, the deflectable region may generate at least one of a tactile and an audible cue when the deflectable region restores from the deflected state to the non-deflected state. In some embodiments, the deflectable region may be formed by a cantilevered arm. In some embodiments, the lock member may include a second projection having a deflectable region. A portion of the lock member including the first and second projections may be substantially symmetric. In some embodiments, the inserter assembly may further comprise an interior housing. The lock member may extend through the interior housing. In some embodiments, the ledge may be defined on a cantilever arm of the sharp holder.

In accordance with yet another example embodiment of the present disclosure, an infusion set base may comprise a first portion including a platform extending substantially along a plane. There may be a receptacle wall projecting from the platform. The receptacle wall may define a receptacle. The first portion may include a set of receptacle wall extensions defining a connector guide channel leading to the receptacle and including an open end opposite the receptacle. The infusion set base may further comprise a second portion on a region of the base opposite the open end of the guide channel formed by a contoured wall. The contoured wall may trend toward dropping in the direction of the plane of the platform portion as proximity to a periphery of the infusion set base increases. The infusion set base may further comprise a transition wall extending from an edge of the platform to an edge of the contoured wall. The infusion set base may further comprise a set of connector receivers disposed on the platform lateral to the receptacle wall extensions.

In some embodiments, the transition wall may extend substantially perpendicular to the plane of the platform. In some embodiments, the receptacle wall may include at least one cantilevered section having a protuberance on an unsupported end thereof. In some embodiments, the unsupported end of the at least one cantilevered section may form a portion of the cantilevered section most distal to the plane of the platform. In some embodiments, the receptacle wall extensions may each include a cantilevered section having a protuberance on respective unsupported ends thereof. In some embodiments, the unsupported ends of the cantilevered sections may form portions of the respective cantilevered sections which are most distal to the plane of the platform. In some embodiments, an open cavity may be present on an underside of the contoured wall. In some embodiments, the transition wall may include a set of passages extending through the transition wall to the cavity.

In accordance with another example embodiment of the present disclosure, an infusion assembly may comprise an infusion set base. The infusion set base may comprise a platform extending substantially along a plane. The infusion set base may further comprise a receptacle wall projecting from the platform. The receptacle wall may define a receptacle. The first portion may include a set of receptacle wall extensions defining a connector guide channel leading to the receptacle and including an open end opposite the receptacle. The infusion set base may further comprise a set of connector receivers on the platform lateral to the guide channel. The infusion assembly may further comprise a cannula subassembly configured to be retained within the receptacle. The infusion assembly may further comprise a connector coupled to a run of tubing. The connector may comprise a connector body including deflectable portions each having an underside from which a connector latch projects. Each connector latch may be configured to engage a respective connector receiver. The connector body may be a shield for the connector receivers and connector latches when the connector latches are engaged with the respective connector receivers. The connector may further comprise a flow hub having a sharp projecting therefrom.

In some embodiments, the infusion set base may further comprise a contoured wall opposite the open end of the guide channel and a transition wall extending from the platform to an edge of the contoured wall. In some embodiments, the transition wall may include a set of passages extending through the transition wall to a cavity on the underside of the contoured wall. In some embodiments, the connector may include a set of sharp flanking projections. The sharp flanking projections may extend through respective passages and partially into the cavity when the connector latches are in engagement with the connector receivers. In some embodiments, the cannula subassembly may be configured to be captured within the receptacle by at least one cantilevered latching projection. In some embodiments, the connector may include a set of sharp flanking projections extending substantially parallel to the sharp. The guide channel may define guides for the set of sharp flanking projections. In some embodiments, the connector body may cover the platform when the connector latches are engaged with the connector receivers. In some embodiments, the infusion set base may include a contoured wall opposite the open end of the guide channel. The contoured wall may be even with adjacent portions of the connector body when the connector latches are engaged with the connector receivers. In some embodiments, the deflectable portions may be configured to deflect toward an axis of the sharp. The connector latches may be disengaged from the connector receivers when the deflectable portions have been deflected toward the axis of the sharp beyond a threshold amount.

In accordance with still another example embodiment of the present disclosure an infusion assembly may comprise an infusion set having a base including a platform, a receptacle wall projecting from the platform and defining a receptacle, a connector guide leading to the receptacle including an open end opposite the receptacle, and a set of connector receivers on the platform flanking the guide. The infusion set may further include a cannula subassembly within the receptacle. The infusion assembly may further comprise a connector coupled to a run of tubing. The connector may have a connector body including deflectable portions each having an underside from which a connector latch projects. Each connector latch may be configured to engage a respective connector receiver. The connector body may cover the connector receivers when the connector latches are engaged with the respective connector receivers. The connector may have a flow hub with a sharp projecting therefrom.

In some embodiments, the deflectable portions may be configured to deflect toward an axis of the sharp. The connector latches may be disengaged from the connector receivers when the deflectable portions have been deflected toward the axis of the sharp beyond a threshold amount. In some embodiments, base may include a contoured wall on a portion of the base opposite the open end of the guide. The contoured wall may trend toward dropping in the direction of the plane of the platform portion as proximity to a periphery of the infusion set base increases. The base may further include a transition wall extending from an edge of the platform to an edge of the contoured wall. In some embodiments, the contoured wall may be even with adjacent portions of the connector body when the connector latches are engaged with the connector receivers.

In accordance with an embodiment of the present disclosure a medication delivery set may comprise a base including a receptacle. The set may further comprise a cannula subassembly configured to couple into the receptacle. The cannula subassembly may include a housing, a cannula, and at least one septum within the housing. The set may further comprise a connector disposed at a terminal end of a flow path and including a delivery sharp and at least one guide surface. The connector may be displaceable with respect to the base from a first position, through an intermediate position and to a delivery position to couple to the connector to the base. The cannula subassembly may be adjusted to a home position within the receptacle by the at least one guide surface as the connector is displaced from the first position to the intermediate position.

In some embodiments, one of the at least one septum may define a fluid receiving volume in communication with a lumen of the cannula. The septum may form a fluid tight seal within the cannula subassembly preventing flow out of the fluid receiving volume other than through the cannula. In some embodiments, an outlet of the delivery sharp may be disposed in the fluid receiving volume when the connector is in the delivery position. In some embodiments, the delivery sharp may be out of contact with the septum in the intermediate position. In some embodiments, the connector may include at least one sharp flanking projection flanking the delivery sharp. The at least one guide surface may be included on the at least one sharp flanking projection. In some embodiments, the at least one guide surface may include at least one lateral adjusting guide surface and at least one height adjusting guide surface. In some embodiments, the connector may include two sharp flanking projections disposed on opposite sides of the delivery sharp. Each of the sharp flanking projections may include at least one of the at least one guide surface. In some embodiments, each of the two sharp flanking projections may include a lateral adjusting guide surface and a height adjusting guide surface. In some embodiments, the connector may include a third sharp flanking projection extending over the sharp and including at least one of the at least one guide surface thereon. In some embodiments, the third sharp flanking projection may include at least one lateral adjusting guide surface and at least one height adjusting guide surface. In some embodiments, the at least one guide surface includes a plurality of guide surfaces which adjust the position of the cannula subassembly along two axes substantially perpendicular to one another and in a plane substantially perpendicular to a direction of elongation of the delivery sharp and base includes a positioner extending into the receptacle that inhibits displacement of the cannula subassembly along an axis substantially perpendicular to the two axes. In some embodiments, an opposing terminal end of the flow path opposite the connector may include a coupling for connection of the flow path to a fluid source. In some embodiments, the cannula may be formed integral with the housing. In some embodiments, the base may include a platform and at least one wall extending from the platform to form the receptacle. The delivery sharp may be substantially parallel to the platform when the connector is coupled to the base.

In accordance with another embodiment of the present disclosure a medication delivery set may comprise a base including a receptacle. The set may further comprise a cannula subassembly configured to couple into the receptacle. The cannula subassembly may include a housing, a cannula, and at least one septum within the housing. The set may further comprise a connector disposed at a terminal end of a flow path and including a delivery sharp and at least one guide surface. The connector may be displaceable with respect to the base from a first position, through an intermediate position and to a delivery position in which the connector is coupled to the base. The at least one guide surface may adjust the position of the cannula subassembly to a home position within the receptacle each time the connector is displaced from the first position to the intermediate position.

In some embodiments, one of the at least one septum may define a fluid receiving volume in communication with the cannula and may forms a fluid tight seal within the cannula subassembly inhibiting flow out of the fluid receiving volume other than through the cannula. In some embodiments, an outlet of the delivery sharp may be disposed in the fluid receiving volume when the connector is in the delivery position and the delivery sharp is out of contact with the septum in the intermediate position. In some embodiments, the connector may include at least one sharp flanking projection. The at least one guide surface may be included on the at least one sharp flanking projection. In some embodiments, the at least one guide surface may include at least one lateral adjusting guide surface and at least one height adjusting guide surface. In some embodiments, the at least one guide surface may include a plurality of guide surfaces which adjust the position of the cannula subassembly along two axes substantially perpendicular to one another and in a plane substantially perpendicular to a direction of elongation of the delivery sharp and the base includes a positioner extending into the receptacle which inhibits displacement of the cannula subassembly along an axis substantially perpendicular to the two axes. In some embodiments, the connector may include a plurality of sharp flanking projections each including at least one of the at least one guide surface thereon. In some embodiments, the connector may include a plurality of sharp flanking projections each including at least one lateral adjusting guide surface and at least one height adjusting guide surface of the at least one guide surface thereon. In some embodiments, at least one of the at least one guide surface may include a sloped section configured to funnel the cannula subassembly toward the home position as the connector is displaced from the first position toward the intermediate position. In some embodiments, the base may include a platform and at least one wall extending from the platform to form the receptacle. The delivery sharp may be substantially parallel to the platform when the connector is coupled to the base.

In accordance with another embodiment of the present disclosure a medication delivery set may comprise a base including a receptacle. The set may further comprise a cannula subassembly coupled into the receptacle. The cannula subassembly may include a housing, a cannula, and at least one septum within the housing. The set may further comprise a connector disposed at a terminal end of a flow path and including a delivery sharp and at least one guide surface. The at least one guide surface may adjust the position of the cannula subassembly to a home position before the delivery sharp contacts a septum of the at least one septum when the connector is displaced from a state in which the connector is uncoupled to the base to a state in which the connector is coupled to the base.

In some embodiments, one of the at least one septum may define a fluid receiving volume in communication with the cannula, and may form a fluid tight seal within the cannula subassembly inhibiting flow out of the fluid receiving volume other than through the cannula. In some embodiments, the at least one guide surface may include a plurality of guide surfaces which adjust the position of the cannula subassembly along two axes substantially perpendicular to one another and in a plane substantially perpendicular to a direction of elongation of the delivery sharp and the base includes a positioner extending into the receptacle that inhibits displacement of the cannula subassembly along an axis substantially perpendicular to the two axes. In some embodiments, the connector may include a plurality of sharp flanking projections each including at least one lateral adjusting guide surface and at least one height adjusting guide surface of the at least one guide surface thereon. In some embodiments, at least one of the at least one guide surface may include a sloped section configured to funnel the cannula subassembly toward the home position as the connector is displaced from the first position toward the intermediate position. In some embodiments, the base may include a platform and at least one wall extending from the platform to form the receptacle. The delivery sharp may be substantially parallel to the platform when the connector is coupled to the base.

In accordance with still another embodiment of the present disclosure a fluid delivery set may comprise a base having a platform portion and a receptacle defined by at least one receptacle wall extending from the platform portion. The at least one receptacle wall may include a deflector body. The set may further comprise a cannula subassembly configured to couple into the receptacle. The cannula subassembly may include a cannula, housing, and a septum disposed partially within the housing and having an exposed portion opposite the cannula accessible from the exterior of the housing. The set may further comprise a connector disposed at a terminal end of a flow path and including a delivery sharp and a sharp flanking projection. The connector may be configured to couple to the base. The deflector body may be configured to deflect the sharp flanking projection against the exposed portion when the connector is coupled to the base.

In some embodiments, the deflector body may be disposed on a portion of the receptacle wall most distal to the platform portion. In some embodiments, the cannula subassembly may further comprise a retainer clip coupled to the housing. The exposed portion of the septum may extend though an aperture in the retainer clip. In some embodiments, the sharp flanking projection may be cantilevered from a flow hub of the connector to which the delivery sharp and flow path are coupled. In some embodiments, the cannula subassembly may include a second septum disposed at least partially within the housing. In some embodiments, the housing may include at least one delivery sharp aperture. The delivery sharp may extend through the aperture and into fluid communication with a fluid introduction volume defined in the septum when the connector is coupled to the base. In some embodiments, the delivery sharp may extend substantially parallel to the platform portion when the connector is coupled to the base. In some embodiments, the base may further comprise a track configured to guide the sharp flanking projection as the connector is coupled to the base. The track may be disposed on the receptacle wall and including the deflector body.

In accordance with yet another embodiment of the present disclosure a base for an infusion set may comprise a platform portion. The base may further comprise a set of walls extending from the platform portion. The walls may partially define a receptacle. Each of the walls may include a segment oriented such that the walls are separated by a first distance proximate the platform portion and a second distance shorter than the first distance distal to the base. There may be a break in each of the walls extending from the segment to the platform portion. The base may further comprise a set of notches defined in the walls. The base may further comprise a receptacle wall span connecting each wall of the set of walls and partially defining the receptacle. The receptacle wall span may include a cantilevered section with a latch protuberance. The cantilevered section may include at least one projecting region extending from the cantilevered section and into the receptacle.

In some embodiments, the notches may be recessed into the walls at a section of the walls most distal the platform portion. In some embodiments, the walls may define a channel extending from the receptacle toward a periphery of the platform portion. In some embodiments, the latch protuberance may be in the form of a ramp which increases in thickness as distance to an unsupported end of the cantilevered section decreases. In some embodiments, the at least one projection region may include a rib which extends along the majority of the cantilevered section. In some embodiments, the at least one projection region may include a rib extending substantially parallel to the axis of extension of the cantilevered section. In some embodiments, the platform portion may be substantially planar and the segment of each of the walls may be oriented substantially parallel to the platform portion. In some embodiments, at least a portion of the notches may be tapered and the notches may be in line with the breaks in the walls. In some embodiments, the notches may each include a first tapered region and a second tapered region, the first and second tapered regions having different tapers. In some embodiments, the segment of each wall and the portion of the walls between the platform portion and the segments may form receiving tracks configured to guide portion of a tubing set connector as the tubing set connector is coupled to the base.

In accordance with another embodiment of the present disclosure a cannula subassembly for coupling to an infusion set base may comprise a cannula having an outlet at a first end and an enlarged region at a second end. The enlarged region may include a sharp guide continuous with a lumen of the cannula and an exterior surface. The cannula subassembly may further comprise a housing separate from the cannula including an end wall with a passage therethrough surrounded by a cannula seat. The housing may have a sidewall extending from the end wall with at least one delivery sharp passage therein. The cannula subassembly may further comprise a septum disposed within the housing and forming a fluid tight seal at least against the exterior surface. The cannula subassembly may further comprise a retention clip coupled to the housing and capturing the septum at least partially within the housing.

In some embodiments, the exterior surface may include a frusto-conic portion and a straight walled portion, the straight walled portion may be substantially parallel to an axis of extension of the cannula. In some embodiments, the exterior surface may be straight walled and extends substantially parallel to an axis of extension of the cannula. In some embodiments, the retention clip may include a channel therethrough. A nub portion of the septum may extend through the channel. A portion of the nub extending beyond the channel may be compressed by at least one projection which partially surrounds the channel. In some embodiments, the at least one projection which partially surrounds the channel may be part of a crenellated wall surrounding the channel. In some embodiments, the at least one projection which partially surrounds the channel may include a set of merlons disposed on opposing sides of the channel. In some embodiments, the retention clip may include a channel therethrough. A nub portion of the septum may extend through the channel and the channel may exert a compressive force against the nub in a direction normal to an axis of extension of the cannula. In some embodiments, the retention clip may include a channel therethrough. A nub portion of the septum may extend through the channel and may be compressed by the channel. The channel may have a width which is at least 50% of the width of the retention clip. In some embodiments, the enlarged region of the cannula may include a flange disposed at an end of the enlarged region most proximate the outlet. In some embodiments, the seat may be a post surrounding the passage. In some embodiments, the seat may be a receptacle recessed into the portion of the end wall surrounding the passage. In some embodiments, the septum housing may include a set of cars projecting outwardly from the sidewall. The cars may each include an expanse which projects from a main portion the car toward the outlet of the cannula. The expanse may decrease in thickness as distance to the outlet decreases. In some embodiments, each car may include a set of wedge bodies disposed on opposing faces of the cars. In some embodiments, a portion of the expanse of each car most proximate the outlet of the cannula may be configured to deform when the portion contacts the infusion set base during coupling of the infusion set base and cannula subassembly. In some embodiments, the septum housing may include a set of protuberances configured to compress the septum. In some embodiments, the cannula subassembly may further comprise at least one additional septum. In some embodiments, the septum may have at least one additional septum nested therein. In some embodiments, the sidewall may include a catch configured to engage with the infusion set base to couple the cannula subassembly to the infusion set base.

In accordance with another embodiment of the present disclosure a cannula subassembly for coupling to an infusion set base may comprise a housing including a first wall, a side wall extending from the first wall having at least one delivery sharp aperture therein, and a plurality of latch surfaces. The cannula subassembly may further comprise a cannula having an outlet at a first end and an enlarged region at a second end. The enlarged region may have an exterior surface with a flange projecting outwardly therefrom. There may be a set of latch arms spaced about the periphery of the flange. Each of the latch arms may be in engagement with one of the latch surfaces. The flange may close a second end of the housing opposite the first wall. The cannula subassembly may further comprise a septum captured between the cannula and the housing and forming a fluid tight seal against the exterior surface.

In some embodiments, each of the plurality of latch surfaces may be a face of a respective notch of a set of notches which are recessed though the first wall of the housing. In some embodiments, an interior surface of the sidewall may include a set of latch arm guides. Each of the guides may extend to one of the plurality of latch surfaces. In some embodiments, the guides may be recessed into the interior surface of the sidewall. In some embodiments, the flange may include at least one ledge member extending from the periphery of the flange. The at least one ledge member may be configured to engage a retainer included on the infusion set base. In some embodiments, the septum housing may include a recess in the sidewall adjacent at least one of the at least one ledge member. In some embodiments, the first wall may include an aperture therethrough. The septum may include a portion which extends at least partially into the aperture. In some embodiments, the housing may include a set of cars which extend outwardly from an exterior surface of the housing. In some embodiments, the ears may each include an expanse which projects from a main portion the car toward the outlet of the cannula and decreases in thickness as distance to the outlet decreases. In some embodiments, a portion of the expanse of each car most proximate the outlet of the cannula may be configured to deform when the portion contacts the infusion set base during coupling of the infusion set base and cannula subassembly. In some embodiments, each ear may include a set of wedge bodies disposed on opposing faces of the cars. In some embodiments, the septum may be disposed between and compressed by a portion of the catch arms. In some embodiments, a face of the enlarged region most proximal the cannula outlet may include a recess surrounding the portion of the cannula extending from the face. In some embodiments, at least a portion of the exterior surface may extend in a direction substantially parallel to an axis of elongation of the cannula. In some embodiments, the exterior surface surrounds a sharp guide which may be continuous with a lumen of the cannula.

In accordance with still another embodiment of the present disclosure, a cannula subassembly for coupling to an infusion set base may comprise a cannula having an outlet at a first end and an enlarged region at a second end. The enlarged region may include and exterior surface with an outwardly extending flange. The cannula subassembly may further comprise a housing separate from the cannula. The housing may include an end wall with a passage therethrough and a sidewall extending from end wall with at least one delivery sharp passage. The cannula subassembly may further comprise a first septum disposed within the housing and forming a fluid tight seal against at least a portion of the exterior surface. The cannula subassembly may further comprise a second septum disposed within the housing and in abutment with the end wall. The second septum may include an aperture. The cannula subassembly may further comprise a retention clip coupled to the housing and capturing the first septum and second septum at least partially within the housing. A portion of the cannula may extend through the aperture and passage to the exterior of the septum housing.

In some embodiments, the flange may be captured between the first and second septum. In some embodiments, the flange may be disposed in a recess formed in the wall of a cavity of the first septum. In some embodiments, one of the first septum and second septum may include a peripheral rim which seals against an outermost side surface of flange. In some embodiments, the first septum may be at least partially nested within a receptacle of the second septum and may form a fluid tight seal against the second septum. In some embodiments, the exterior surface may include a straight expanse which extends substantially parallel to the axis of elongation of the cannula. In some embodiments, the first and second septum may be constructed of different materials. In some embodiments, the septum housing may include a plurality of protuberances which extend into an interior of the septum housing. The protuberances may contact and compress at least one of the first septum and second septum. In some embodiments, the septum housing may include a plurality of cars. Each car may include a main portion having a tapered expanse extending therefrom. The expanse may decrease in thickness as proximity to the outlet increases. In some embodiments, each of the cars may include a wedge body adjacent the sidewall. In some embodiments, the second septum may include a protruding section which extends at least partially through the passage of the end wall. In some embodiments, the enlarged region may include a sharp guide continuous with a lumen of the cannula.

In accordance with yet another embodiment of the present disclosure a method for assembling a cannula subassembly may comprise providing a housing. The method may further comprise disposing a first septum inside the housing. The method may further comprise placing a cannula in the cannula subassembly such that a first portion of the cannula is inside the housing and against a surface of the first septum, the cannula extending through the first septum and the housing such that a second portion of the cannula is external to the housing. The method may further comprise seating a second septum against the first septum and forming a fluid tight seal between at least the second septum and the first portion of the cannula. The method may further comprise capturing the first septum, second septum, and a portion of the cannula within the housing by coupling a septum retainer to the septum housing.

In some embodiments, disposing the first septum inside the housing may comprise disposing a surface of the first septum against an end wall of the housing. In some embodiments, the end wall may include a passage therethrough and positioning the first septum inside the housing further may comprise aligning an aperture extending through the septum with the passage, the cannula extending through the aperture and passage. In some embodiments, placing the cannula in the cannula subassembly may comprise placing the first portion of the cannula within a cavity of the first septum. In some embodiments, seating the second septum against the first septum may comprise installing the second septum in a cavity of the first septum. In some embodiments, seating the second septum against the first septum may comprise sandwiching a flange of the first portion of the cannula between a section of the first septum and a section of the second septum. In some embodiments, forming a fluid tight seal between at least the second septum and the first portion of the cannula may comprise receiving at least a section of the first portion of the cannula within a cavity of the second septum. In some embodiments, forming a fluid tight seal between at least the second septum and the first portion of the cannula may further comprise retaining a flange of the first portion of the cannula within a recess defined in the wall of the cavity of the second septum. In some embodiments, placing the cannula in the cannula subassembly may comprise placing the first portion of the cannula within a receptacle region of the first septum surrounded by a peripheral rim of the first septum. In some embodiments, seating the second septum against the first septum may comprise displacing a peripheral rim of the second septum against the first septum and locating a flange of the first portion of the cannula with a receptacle region of the second septum surrounded by the peripheral rim. In some embodiments, seating the second septum against the first septum may comprise forming a fluid tight seal between the first septum and the second septum.

In accordance with another embodiment of the present disclosure an infusion set assembly for delivery of agent into a patient may comprise a base. The base may comprise a platform portion. The base may further comprise a set of walls extending from the platform portion. The walls may define a receptacle with a cantilevered section including a latch portion and at least one projecting region extending from the cantilevered section and into the receptacle. The base may further comprise a set of notches defined in the walls. The infusion set assembly may further comprise a cannula subassembly within the receptacle. The cannula subassembly may comprise a septum housing including a catch surface engaged with the latch portion. The septum housing may include a set of wedge bodies extending from a sidewall of the septum housing. The wedge bodies may be disposed at least partially within the notches and create a mechanical interference against tilting of the cannula subassembly within the receptacle. The at least one projecting region may be configured to create a mechanical interference against tilting of the cannula subassembly within the receptacle. The cannula subassembly may further comprise a cannula. The cannula subassembly may further comprise a septum forming a fluid tight seal against a portion of the cannula and including a fluid introduction volume in fluid communication with a lumen of the cannula.

In some embodiments, the notches may be recessed into the walls at a section of the walls most distal the platform portion. In some embodiments, the walls define a channel extending from the receptacle toward a periphery of the platform portion. In some embodiments, each of the walls may include a segment oriented such that the walls are separated by a first distance proximate the platform portion and a second distance shorter than the first distance distal to the base. There may be a break in each of the walls extending from the segment to the platform portion. In some embodiments, the platform portion may be substantially planar and the segment of each of the walls may be oriented substantially parallel to the platform portion. In some embodiments, at least a portion of the notches may be tapered and the notches may be in line with the breaks in the walls. In some embodiments, the segment of each wall and the portion of the walls between the platform portion and the segments may form receiving tracks configured to guide portion of a tubing set connector as the tubing set connector is coupled to the base. In some embodiments, the notches may each include a first tapered region and a second tapered region, the first and second tapered regions may have different tapers. In some embodiments, the latch portion may be in the form of a ramp which increases in thickness as distance to an unsupported end of the cantilevered section decreases. In some embodiments, the at least one projection region may include a rib which extends along the majority of the cantilevered section. In some embodiments, the at least one projection region may include a rib extending substantially parallel to the axis of extension of the cantilevered section. In some embodiments, the cannula subassembly may include a second septum in contact with the first septum. The cannula may extend through an orifice in the second septum.

In accordance with yet another embodiment of the present disclosure a cannula subassembly may comprise a housing. The cannula subassembly may further comprise a first septum disposed in the housing. The cannula subassembly may further comprise a cannula having a first portion disposed inside the housing and against a surface of the first septum. The cannula may have a second portion extending through the first septum and housing. The cannula subassembly may further comprise a second septum disposed against the first septum. The second septum may form a fluid tight seal at least against the first portion of the cannula. The cannula subassembly may further comprise a retainer coupled to the housing. The retainer may capture the first septum, second septum, and the first portion of the cannula within the housing.

In some embodiments, the cannula may be a discrete molded component separate from the housing. In some embodiments, the housing and retainer may be made of molded plastic and the first and second septum are made of an elastomer. In some embodiments, a fluid seal may be formed between abutting surfaces of the first and second septum. In some embodiments, the retainer may comprise a substantially planar body. The retainer may further comprise a plurality of cantilevered projections extending substantially perpendicularly from the body and disposed opposite one another. The retainer may further comprise a plurality of latch members disposed on the ends of the cantilevered projections. In some embodiments, the second septum may comprise a nub projecting through a channel defined in the retainer. The second septum may further comprise a cavity defining a fluid introduction volume. The fluid introduction volume may be in fluid communication with a lumen of the cannula. In some embodiments, the second septum may further comprise a recess in a wall of the cavity. A flange of the cannula may be disposed within the recess. In some embodiments, the housing may comprise a plurality of cars molded on the side of the housing. In some embodiments, a septum receptacle may be recessed into a central portion of a first end of the housing. In some embodiments, a cannula passage may extend from a second end of the housing opposite the first end into the receptacle. At least one catch configured to engage a portion of the retainer to couple the retainer to the housing may be included. At least one connector needle passage disposed in a sidewall of the housing may be included. In some embodiments, the first septum may be disposed in a receptacle of the housing. The receptacle may comprise a plurality of guides on an interior wall of the receptacle. In some embodiments, the first septum may be disposed in a receptacle of the housing. The receptacle may comprise a plurality of protuberances extending from the wall of the receptacle and into contact with at least the first septum. In some embodiments, the first portion of the cannula may comprise an enlarged region. The enlarged region may comprise an insertion sharp guide. The enlarged region may further comprise a septum interface region disposed surrounding the insertion sharp guide. The enlarged region may further comprise a flange extending outward from the first portion of the cannula. In some embodiments, the first portion of the cannula may include a flange. The flange may be captured in a fluid tight relationship between the first septum and the second septum. In some embodiments, the first portion of the cannula may include a flange. The flange may be disposed within a receptacle of the second septum. There may be a fluid tight seal between the flange and the second septum. In some embodiments, the second portion of the cannula may include an outlet at an end thereof.

In accordance with another embodiment of the present disclosure an inserter assembly for placing an infusion set into communication with an infusion site may comprise a housing having a closed end and an open end. The inserter assembly may further comprise a cannula subassembly. The inserter assembly may further comprise an infusion set base separate from the cannula subassembly and disposed at the open end. The inserter assembly may further comprise an actuation assembly. The inserter assembly may further comprise an adhering assembly for attaching the infusion set base to the infusion site. The adhering assembly may comprise an adhesive patch attached to the infusion set base and including a patch aperture. The adhering assembly may further comprise a liner having an enlarged region covering the adhesive patch and having a liner aperture. The liner may have a strip portion extending from the enlarged region, and tab extending from the enlarged region adjacent the strip portion. The strip portion may be coupled to the housing. There may be a slit through the enlarged region extending from a point between the tab and the strip portion and tangent to a far side of the aperture with respect to the tab.

In some embodiments, the actuation assembly may be configured to displace an insertion sharp from an initial position, through an aperture in the infusion set base to an insertion position, and retract the insertion sharp into the housing to a retracted position. The cannula subassembly may be carried by the insertion sharp and coupled into a receptacle of the infusion set base as the insertion sharp is displaced to the insertion position. In some embodiments, the inserter assembly may further comprise a removable lock member extending into the housing. The lock member may block actuation of the actuation assembly. The strip portion may extend over the lock member. In some embodiments, the lock member may include a set of arcuate members disposed on opposing sides of a main body of the lock member, the arcuate members resiliently deflectable toward the main body. In some embodiments, the adhering assembly may further comprise an adhesive body and the liner may be coupled to the housing via the adhesive body. In some embodiments, the strip portion may have a length greater than the height of the housing and an end of the strip portion opposite the enlarged region may be coupled the closed end of the housing. In some embodiments, the strip portion may be coupled to the housing at an end of the strip portion opposite the enlarged region in a manner selected from a group consisting of: via adhesive, heat staking, rf welding, sonic welding, and ultrasonic welding. In some embodiments, the adhesive patch may include a main region and a protruding region extending from the periphery of the main region. A segment of the strip portion of the liner may cover adhesive present on the protruding region. In some embodiments, the adhesive patch may have a footprint larger than a footprint of the infusion set base. In some embodiments, the slit may extend in a straight line. In some embodiments, the liner aperture may be defined by a sidewall including a first rounded span opposite a second rounded span. The first rounded span may have a tighter curvature than the second rounded span. The first and second rounded spans may be connected to one another by straight side wall spans. In some embodiments, the second rounded span may be more distal the tab than the first rounded span. In some embodiments, the shortest distances between points on the second rounded span and the periphery of the liner may be shorter than the shortest distance between the periphery of the liner and any other portion of the liner aperture. In some embodiments, the liner may be a waxed paper material. In some embodiments the liner may be a polymer material. In some embodiments, the adhesive patch may be coupled to the infusion set base in a manner selected from a group consisting of: via adhesive, heat staking, rf welding, sonic welding, and ultrasonic welding.

In accordance with another embodiment of the present disclosure an inserter assembly for placing an infusion set into communication with an infusion site may comprise a housing having a closed end and an open end. The inserter assembly may further comprise an infusion set. The inserter assembly may further comprise an actuation assembly. The inserter assembly may further comprise an adhering assembly for attaching the infusion set to the infusion site. The adhering assembly may comprise an adhesive patch attached to a portion of the infusion set and including a patch aperture. The adhering assembly may further comprise a backing having a first portion covering the adhesive patch and having a backing aperture. The backing may have a second portion, and a third portion both extending from the first portion and being adjacent one another. The second portion may be coupled to the housing. There may be a slit through the first portion extending from a point between the second and third portion and tangent to a far side of the aperture with respect to the point.

In some embodiments, the infusion set may be disassembled within the inserter assembly. In some embodiments, the actuation assembly may be configured to displace an insertion sharp from an initial position, through an aperture in a base of the infusion set to an insertion position, and retract the insertion sharp into the housing to a retracted position. A cannula subassembly of the infusion set may be carried by the insertion sharp and coupled into a receptacle of the base as the insertion sharp is displaced to the insertion position. In some embodiments, the inserter assembly may further comprise a removable lock member extending into the housing. The lock member may block actuation of the actuation assembly. At least a portion of the backing may extend over the lock member. In some embodiments, the lock member may include a main body having a pair of arcuate members on opposing sides thereof. The arcuate members may resiliently deflect toward the main body. In some embodiments, the second portion may have a length greater than a height of the housing and an end of the second portion opposite the first portion of the backing may be coupled the closed end of the housing. In some embodiments, the second portion may be coupled to the housing at an end of the second portion opposite the first portion in a manner selected from a group consisting of: via adhesive, heat staking, rf welding, sonic welding, and ultrasonic welding. Ins some embodiments, the adhesive patch may include a main region and a protruding region extending from the periphery of the main region. A segment of the second portion of the backing may cover adhesive present on the protruding region. In some embodiments, the adhesive patch may have a footprint larger than a footprint of the infusion set. In some embodiments, the slit may extend in a straight line. In some embodiments, the backing aperture may be defined by a sidewall including a first rounded span opposite a second rounded span. The first rounded span may have a tighter curvature than the second rounded span. The first and second rounded spans may be connected to one another by straight side wall spans. In some embodiments, the second rounded span may be more distal the second portion of the backing than the first rounded span. In some embodiments, the shortest distances between points on the second rounded span and a periphery of the backing may be shorter than the shortest distance between the periphery of the backing and any other portion of the backing aperture. In some embodiments, the backing may be a waxed paper material. In some embodiments, the backing may be a polymer material. In some embodiments, the adhesive patch may be coupled to the portion of the infusion set in a manner selected from a group consisting of: via adhesive, heat staking, rf welding, sonic welding, and ultrasonic welding. In some embodiments, the backing may cover a majority of the open end of the housing.

In accordance with yet another embodiment of the present disclosure an adhering assembly for attaching an infusion set to the infusion site may comprise an adhesive patch attached to a portion of the infusion set and including a patch aperture. The adhering assembly may further comprise a backing. The backing may comprise a first portion covering the adhesive patch. The backing may further comprise a second portion. The backing may further comprise a third portion. The second and third portion may both extend from the first portion and may be adjacent one another. The backing may further comprise an aperture disposed in the first portion defined by a sidewall having a first rounded span and a second rounded span connected by straight sidewall sections. The second rounded span may have a gentler curvature than the first rounded span. The backing may further comprise a slit extending across a section of the first portion from a point between the second and third portion. The slit may have a point of tangency on a far side of the first rounded span with respect to the point.

In some embodiments, the second portion of the backing may include an adhesive body coupled thereto for affixing the second portion to the housing of an inserter assembly. In some embodiments, the second portion may have a length greater than the third portion. In some embodiments, the second portion may be an elongate strip. In some embodiments, the adhesive patch may include a main region and a protruding region extending from the periphery of the main region. A segment of the second portion of the backing may cover adhesive present on the protruding region. In some embodiments, the slit may extend in a straight line. In some embodiments, the patch aperture may be aligned with and has the same shape as the backing aperture. In some embodiments, the second rounded span may be more distal the second portion of the backing than the first rounded span. In some embodiments, the shortest distances between points on the second rounded span and a periphery of the backing may be shorter than the shortest distance between the periphery of the backing and any other portion of the backing aperture. In some embodiments, force required to peel the backing from an adhesive of the adhesive patch in a region of the backing between the periphery of the backing and the second round span of the aperture may be lower than the force required to peel the backing from the adhesive at any other region of the first portion of the backing. In some embodiments, the backing may be a waxed paper material. In some embodiments, the backing may be a polymer material. In some embodiments, the adhesive patch may include an infusion set adhesive for coupling to a portion of the infusion set on a face of the adhesive patch opposite the backing.

In accordance with another example embodiment of the present disclosure an inserter assembly may comprise a housing. The inserter assembly may further comprise an infusion set. The inserter assembly may further comprise a sharp bearing body. The inserter assembly may further comprise a trigger. The inserter assembly may further comprise a drive spring held in an energy storing state by the trigger. The drive spring may urge the sharp bearing body from a raised state to a forward state when the trigger is released. The inserter assembly may further comprise a means for releasing the trigger as the inserter assembly is tugged away from the body.

In some embodiments, the inserter assembly may further comprise a means for preventing release of the trigger before skin at a selected infusion set placement site has been lifted from underlying anatomy. In some embodiments, a portion of the infusion set may include an adhesive for anchoring the portion of the infusion set to a patch of skin. In some embodiments, the infusion set may include a base and a cannula assembly. In some embodiments, the cannula subassembly may be carried by the sharp bearing body and uncoupled to the base when the sharp bearing body is in the raised state. In some embodiments, the cannula subassembly may be coupled to the base when the sharp bearing body is urged to the forward state. In some embodiments, the inserter assembly may further comprise a retraction spring configured to displace the sharp bearing body from the forward state to a retracted state when the retraction spring is freed from an energy storing state. In some embodiments, the infusion set may be configured to be decoupled from the inserter assembly when the sharp bearing body is urged to the forward state. In some embodiments, the inserter assembly may further comprise a retraction spring held in an energy storing state by a coupling between the infusion set and another portion of the inserter assembly. The coupling may be released when the sharp bearing body is urged to the forward position. The retraction spring may urge the sharp bearing body to a retracted state when freed from its energy storing state. In some embodiments, the sharp bearing body may be in a different location within the inserter assembly in the raised state compared to the retracted state.

In accordance with yet another example embodiment of the present disclosure an inserter assembly may comprise a housing. The inserter assembly may further comprise an infusion set having adhesive on a portion thereof for adhering the portion of the infusion set to skin. The inserter assembly may further comprise a sharp bearing body. The inserter assembly may further comprise a trigger. The inserter assembly may further comprise an insertion bias member held in an energy storing state by the trigger. The insertion bias member may urge the sharp bearing body from a raised state to a forward state when the trigger is released. The inserter assembly may further comprise a means for releasing the trigger after inserter assembly is withdrawn from the body and skin adhered to the portion of the infusion set has been lifted from underlying anatomy.

In some embodiments, the infusion set may include a base and a cannula assembly. In some embodiments, the cannula subassembly may be carried by the sharp bearing body and uncoupled to the base when the sharp bearing body is in the raised state. In some embodiments, the cannula subassembly may be configured to couple to the base when the sharp bearing body is urged to the forward state. In some embodiments, the inserter assembly may further comprise a retraction bias member configured to displace the sharp bearing body from the forward state to a retracted state when the retraction bias member is freed from an energy storing state. In some embodiments, the inserter assembly may further comprise a means for releasing the infusion set when the sharp bearing body is displaced to the forward state. In some embodiments, the inserter assembly may further comprise a means for retracting the sharp bearing body to a retracted state after the sharp bearing body has been urged to the forward state. In some embodiments, the sharp bearing body may be in a different location within the inserter assembly in the raised state compared to the retracted state. In some embodiments, the infusion set may include an infusion set base and a cannula subassembly and the infusion set base may include a means for coupling the cannula subassembly to the infusion set base.

In accordance with yet another exemplary embodiment of the present disclosure an inserter assembly may comprise a housing. The inserter assembly may further comprise an infusion set having adhesive on a portion thereof for adhering the portion of the infusion set to skin. The inserter assembly may further comprise a sharp bearing body. The inserter assembly may further comprise a trigger arrangement. The inserter assembly may further comprise a bias member held in an energy storing state by the trigger arrangement. The bias member may urge the sharp bearing body from a raised state to a forward state when the trigger arrangement is released. The inserter assembly may further comprise a means for preventing release of the trigger arrangement before skin adhered to the infusion set has been lifted from underlying anatomy.

In some embodiments, the inserter assembly may further comprise a means for actuating the trigger arrangement one the skin has been lifted. In some embodiments, the infusion set may include a base and a cannula assembly. In some embodiments, the cannula subassembly may be carried by the sharp bearing body and uncoupled to the base when the sharp bearing body is in the raised state. In some embodiments, the cannula subassembly may be configured to couple to the base when the sharp bearing body is urged to the forward state. In some embodiments, the inserter assembly may further comprise a retraction bias member configured to displace the sharp bearing body from the forward state to a retracted state when the retraction bias member is freed from an energy storing state. In some embodiments, the inserter assembly may further comprise a means for releasing the infusion set when the sharp bearing body is displaced to the forward state. In some embodiments, the inserter assembly may further comprise a means for retracting the sharp bearing body to a retracted state after the sharp bearing body has been urged to the forward state. In some embodiments, the sharp bearing body may be in a different location within the inserter assembly in the raised state compared to the retracted state. In some embodiments, the infusion set may include an infusion set base and a cannula subassembly and the infusion set base may include a means for coupling the cannula subassembly to the infusion set base.

In accordance with another example embodiment of the present disclosure, an inserter assembly may comprise a casing unit comprising at least one projection. The inserter assembly may further comprise a second unit. The second unit may comprise an infusion set having an adhesive on a portion of the infusion set for anchoring the infusion set to a patch of skin. The second unit may further comprise a sharp holder including an insertion sharp. The second unit may further comprise a trigger. The second unit may further comprise a drive spring held in an energy storing state by the trigger. The drive spring may urge the sharp bearing body from a raised state to a forward state when the trigger is released. The second unit may further comprise at least one resilient member. An interference between each of the at least one resilient member and an associated projection of the at least one projection may be present. The interference may block relative displacement of the casing unit and second unit when the adhesive is adhered to the patch of skin until the inserter assembly is displaced in a direction away from an infusion site and the patch of skin is lifted to a point that force exerted via elasticity of the skin reaches a threshold that deflects each of the at least one resilient member out of an interfering relationship with the associated projection of the at least one projection.

In some embodiments, the trigger may comprise a catch which engages with a ledge defined on the sharp holder. In some embodiments, the drive spring may be a compression spring. In some embodiments, each of the at least one resilient member may be a cantilevered arm. In some embodiments, each of the at least one projection may be a deflector. In some embodiments, the casing may further comprise a triggering projection. In some embodiments, the inserter assembly may further comprise a means for retracting the sharp bearing body to a retracted state after the sharp bearing body has been urged to the forward state. In some embodiments, the portion of the infusion set may be disposed within an opening in an end of the casing unit and is substantially even with the end of the casing unit when the inserter assembly is in an initial state.

In accordance with still another example embodiment of the present disclosure an inserter assembly may comprise a first unit including triggering body. The inserter assembly may further comprise a second unit including an actuation assembly including a trigger. The inserter assembly may further comprise an infusion set base releasably coupled to the second unit and having an adhesive thereon for anchoring the infusion set base to a patch of skin. When the adhesive is anchoring the infusion set base to the patch of skin and the first unit is displaced in a direction away from the patch of skin, the second unit may be restricted from displacing relative to the patch of skin. Relative motion between the first and second units may displace the triggering body into the trigger.

In some embodiments, the second unit may further comprise a resilient member aligned with a deflector included on part of the first unit. In some embodiments, the second unit may further comprise a resilient member configured to abut a portion of the first unit to inhibit relative motion of the first unit and second unit until a threshold force applied to separate the first and second unit is exceeded. In some embodiments, the portion of the first unit may be a deflector member and the resilient member may be a cantilevered arm extending from part of the second unit. In some embodiments, the threshold force may be selected such that, as the first unit is displaced in a direction away from the patch of skin, the patch of skin is lifted from underlying anatomy due to adhesion of the adhesive at least a certain distance from its resting position before the triggering body is displaced into the trigger. In some embodiments, the force threshold may be selected such that, as the first unit is displaced in a direction away from the patch of skin, the patch of skin is lifted from underlying anatomy due to adhesion of the adhesive before relative displacement of the first and second unit begins. In some embodiments, the force threshold may be selected such that, as the first unit is displaced in a direction away from the patch of skin, the patch of skin is lifted due to adhesion of the adhesive at least a certain distance from its resting position. In some embodiments, the adhesive may be configured to maintain adherence to the patch of skin and withstand force generated by the elasticity of the skin when the adhesive is in an adhering relationship with the patch of skin and the first unit is displaced away from an infusion site.

In accordance with yet another exemplary embodiment of the present disclosure an inserter assembly may comprise a first portion including triggering body. The inserter assembly may further comprise an infusion set base having an adhesive thereon for anchoring the infusion set base to a patch of skin. The inserter assembly may further comprise a second portion including an actuation assembly with a trigger. The infusion set base may be releasably coupled to the second unit. The second unit may be constrained from displacing relative to the patch of skin when the infusion set base is coupled to the second portion and the infusion set base is anchored to the skin by the adhesive. The first portion may be displaceable away from the second portion from an initial position to a triggering position. The triggering body may actuate the trigger as the first portion is displaced toward the triggering position.

In some embodiments, the second portion may further comprise a resilient member which is in abutment with a deflector of the first portion when the first portion is in the initial position. In some embodiments, the second portion may further comprise a resilient member configured to abut a portion of the first portion to inhibit relative motion of the first portion and second portion until a force threshold is exceeded. In some embodiments, the portion of the first portion may be a deflector member and the resilient member may be a cantilevered arm extending from part of the second portion. In some embodiments, the force threshold may be greater than a threshold necessary to lift the patch of skin from underlying anatomy when the infusion set base is anchored to the patch of skin via the adhesive. In some embodiments, the force threshold may be selected such that when the infusion set base is anchored to the patch of skin via the adhesive and the first unit is displaced in a direction away from the patch of skin, the patch of skin is lifted due to adhesion of the adhesive at least a certain distance from its resting position. In some embodiments, the adhesive may be configured to maintain adherence to the patch of skin and withstand force generated by the elasticity of the skin when the adhesive is in an adhering relationship with the patch of skin and the first unit is displaced away from the second portion.

In accordance with another example embodiment of the present disclosure an inserter assembly may comprise a first portion including triggering body. The inserter assembly may further comprise an infusion set base having an adhesive thereon for anchoring the infusion set base to a patch of skin. The inserter assembly may further comprise a second portion including an actuation assembly with a trigger. The infusion set base may be releasably coupled to the second unit. The second unit may be constrained from displacing relative to the patch of skin when the infusion set base is coupled to the second portion and the infusion set base is anchored to the skin by the adhesive. The first portion may be configured to displace with respect to second portion from an initial position to a triggering position once a threshold force in a direction away the second portion is applied to the first portion. The triggering body may be configured to actuate the trigger as the first portion is displaced toward the triggering position.

In some embodiments, the threshold force may be greater than a threshold necessary to lift the patch of skin from underlying anatomy when the infusion set base is anchored to the patch of skin via the adhesive. In some embodiments, the force threshold may be selected such that when the infusion set base is anchored to the patch of skin via the adhesive and the first unit is displaced in a direction away from the patch of skin, the patch of skin is lifted due to adhesion of the adhesive at least a certain distance from its resting position. In some embodiments, the adhesive may be configured to maintain adherence to the patch of skin and withstand force generated by the elasticity of the skin when the adhesive is in an adhering relationship with the patch of skin and the first unit is displaced in a direction away from the patch of skin. In some embodiments, first portion may be releasably engaged with the second portion and the first portion and second portion may be disengaged from one another when the threshold force is applied. In some embodiments, an interference may be present between the first portion and the second portion and a portion of the second portion may deflect out of an interfering relationship with the first portion when the threshold force is applied. In some embodiments, the inserter assembly may further comprise a removable lock member. The lock member may be positioned to inhibit relative displacement of the first and second portion even when the threshold force is applied. In some embodiments, the inserter assembly may further comprise a removable liner which covers the adhesive. In some embodiments, the first portion may form a casing for the inserter assembly and the infusion set base may be positioned within an opening in an end of the casing and even with the end when the inserter assembly is in an initial state. In some embodiments, the inserter assembly may further comprise a cannula subassembly. The actuation assembly may be configured to displace the cannula subassembly into engagement with the infusion set base when the trigger is actuated.

In accordance with still another example embodiment of the present disclosure an inserter assembly may comprise a first unit. The inserter assembly may further comprise a second unit. There may be an interference between the first unit and second unit which inhibits relative displacement of the first unit and second unit until more than a threshold force is applied to pull apart the first and second unit. The inserter assembly may further comprise an infusion set having an adhesive on a portion thereof for adhering the portion of the infusion set to a patch of skin. The inserter assembly may further comprise an insertion actuation assembly including a trigger. The trigger may be configured to be actuated once a magnitude of relative displacement separating the first and second unit exceeds a displacement threshold.

In some embodiments, the adhesive may be configured to maintain adherence to the patch of skin and withstand force generated by the elasticity of the skin when the adhesive is in an adhering relationship with the patch of skin and the inserter assembly is displaced away from an infusion site. In some embodiments, the threshold force may be selected to ensure that the patch of skin is lifted from underlying anatomy when the adhesive is in an adhering relationship with the patch of skin and first unit is displaced in a direction away from the patch of skin. In some embodiments, the interference may be supplied by an abutment of a resilient member of one of the first and second unit and a deflector of the other of the first and second unit. In some embodiments, the interference may be supplied by at least one cantilevered arm of the second unit which each abut an associated deflector of the first unit. In some embodiments, the first unit may be a casing for the inserter assembly. In some embodiments, the infusion set may include a base and a cannula assembly. In some embodiments, the cannula subassembly may be carried by a spring loaded sharp bearing body of the second unit. The spring loaded sharp bearing body may be propelled by a bias member from a raised state to a forward state when the trigger is actuated. In some embodiments, the infusion set base may be releasably coupled to the second unit and the cannula subassembly may be uncoupled to and spaced from the base when the sharp bearing body is in the raised state. In some embodiments, the cannula subassembly may be coupled to the base when the sharp bearing body is urged to the forward state. In some embodiments, the inserter assembly may further comprise a spring loaded sharp bearing body included as part of the second unit. The spring loaded sharp bearing body may be configured to be propelled by a bias member from a raised state to a forward state when the trigger is actuated. In some embodiments, the inserter assembly may further comprise a retraction spring configured to displace the sharp bearing body from the forward state to a retracted state when the retraction spring is freed from an energy storing state. In some embodiments, the infusion set may be releasably coupled to the second unit and may be configured to be decoupled from the second unit when the sharp bearing body is urged to the forward state. In some embodiments, the inserter assembly may further comprise a retraction spring held in an energy storing state at least by a coupling between a portion of the infusion set and the second unit. The coupling may be released when the sharp bearing body is urged to the forward position. The retraction spring may urge the sharp bearing body to a retracted state when freed from its energy storing state. In some embodiments, the sharp bearing body may be in a different location within the inserter assembly in the raised state compared to the retracted state. In some embodiments, the insertion actuation assembly may be included as part of the second unit.

In accordance with another example embodiment of the present disclosure an inserter assembly may comprise a first unit. The inserter assembly may further comprise a second unit in an interfering relationship with the first unit that blocks relative displacement of the first and second units until more than a threshold force is applied to separate the first and second units. The inserter assembly may further comprise an infusion set having an adhesive on a portion thereof configured to maintain adherence to a patch of skin and withstand force generated by the elasticity of the skin when the adhesive is in an adhering relationship with the patch of skin and the inserter assembly is displaced away from an infusion site. The inserter assembly may further comprise an insertion actuation assembly.

In some embodiments, the threshold force may be selected to ensure that the patch of skin is lifted from underlying anatomy when the adhesive is in an adhering relationship with the patch of skin and first unit is displaced in a direction away from the patch of skin. In some embodiments, the first unit may include a housing of the inserter assembly. In some embodiments, the infusion set may comprise an infusion set base and a cannula assembly. The cannula assembly may be uncoupled from and in spaced relation to the infusion set base prior to activation of the insertion actuation assembly. In some embodiments, the adhesive may be disposed on a bottom surface of the infusion set base. In some embodiments, the insertion actuation assembly may include a trigger arrangement and a spring loaded sharp bearing body. In some embodiments, the interfering relationship may be established by an abutment of a resilient member of one of the first and second unit and a deflector of the other of the first and second unit. In some embodiments, the interfering relationship may be established by at least one cantilevered arm of the second unit which each abut an associated deflector of the first unit. In some embodiments, the insertion actuation assembly may be included as part of the second unit.

In accordance with still another example embodiment of the present disclosure an inserter assembly may comprise a first unit. The inserter assembly may further comprise a second unit in an interfering relationship with the first unit that inhibits relative displacement of the first and second units until more than a threshold force is applied to separate the first and second unit. The inserter assembly may further comprise an infusion set having an adhesive on a portion thereof for adhering the portion of the infusion set to a patch of skin. The adhesive may be configured to lift the patch of skin from underlying anatomy when the adhesive is in an adhering relationship with the patch of skin and the inserter assembly is displaced away from an infusion site. The inserter assembly may further comprise an insertion actuation assembly.

In some embodiments, the infusion set may comprise an infusion set base and a cannula assembly. The cannula assembly may be uncoupled from and in spaced relation to the infusion set base prior to activation of the insertion actuation assembly. In some embodiments, the adhesive may be provided on a bottom surface of the infusion set base. In some embodiments, the first unit may include a casing for the inserter assembly and the infusion set base may be releasably coupled to the second unit and disposed at an opening in an end of the casing and even with the end of the casing when the inserter assembly is in an initial state. In some embodiments, the adhesive may be covered by a removable backing. In some embodiments, the inserter assembly may further comprise a removable lock member configured to inhibit activation of the insertion actuation assembly. In some embodiments, the inserter assembly may further comprise a removable lock member configured to prevent separation of the first and second units when the threshold force is exceeded. In some embodiments, the insertion actuation assembly may include a trigger arrangement and a spring loaded sharp bearing body. In some embodiments, the interfering relationship may be established by an abutment of at least one resilient member of one of the first and second unit and an associated deflector for each of the at least one resilient member included in the other of the first and second unit. In some embodiments, the interfering relationship may be established by at least one cantilevered arm of the second unit. Each of the at least one cantilevered arm may be abutting an associated projection of the first unit. In some embodiments, the insertion actuation assembly may be included as part of the second unit.

In accordance with another example embodiment of the present disclosure an inserter assembly may comprise a first unit. The inserter assembly may further comprise a second unit releasably engaged with the first unit. The second unit may be configured to displace in tandem with the first unit until more than a threshold force is applied to separate the first and second unit. The inserter assembly may further comprise an infusion set having an adhesive on a portion thereof for adhering the portion of the infusion set to a patch of skin. The adhesive may be configured to lift the patch of skin from underlying anatomy when the adhesive is in an adhering relationship with the patch of skin and the inserter assembly is displaced away from an infusion site. The inserter assembly may further comprise an insertion actuation assembly.

In some embodiments, the insertion actuation assembly may be included as part of the second unit. In some embodiments, the second unit may be releasably engaged with the first unit via an interference between part of the first unit and the second unit. In some embodiments, a portion of the second unit may be configured to deflect to a deflected state when the threshold force is applied. The portion of the second unit may be displaced out of an interfering relationship with the part of the first unit when in the deflected state. In some embodiments, the second unit may be releasably engaged with the first unit via a resilient member which is in abutting engagement with a deflector disposed on a portion of the first unit. In some embodiments, the resilient member may be configured to deflect to a deflected state when the threshold force is applied. The resilient member may be out of abutting engagement with the deflector when in the deflected state. In some embodiments, the second unit may be releasable engaged with the first unit via at least one cantilevered arm of the second unit, each of which being in abutting engagement with a cooperating projection on a part of the first unit for each of the at least one cantilevered arm. In some embodiments, each of the at least one cantilevered arm may be configured to deflect to a deflected state when the threshold force is applied. Each of the at least one cantilevered arm may be out of abutting engagement with the associated cooperating projection when in the deflected state. In some embodiments, the insertion actuation assembly may include a trigger arrangement and a spring loaded sharp bearing body. In some embodiments, the infusion set may comprise an infusion set base and a cannula assembly. The cannula assembly may be uncoupled from and in spaced relation to the infusion set base prior to activation of the insertion actuation assembly. In some embodiments, the adhesive may be provided on a bottom surface of the infusion set base. In some embodiments, the first unit may include a casing for the inserter assembly and the infusion set base may be releasably coupled to the second unit and disposed at an opening in an end of the casing even with the end of the casing when the inserter assembly is in an initial state. In some embodiments, the adhesive may be covered by a removable backing. In some embodiments, the inserter assembly may further comprise a removable lock member configured to inhibit activation of the insertion actuation assembly. In some embodiments, the inserter assembly may further comprise a removable lock member configured to block relative displacement of the first and second unit even when more than the threshold force is applied to separate the first and second unit.

In accordance with another exemplary embodiment of the present disclosure a method of placing an infusion set of an inserter assembly at an infusion site may comprise adhering a portion of the inserter assembly to skin at the infusion site. The method may further comprise pulling the inserter assembly away from the body to lift skin at the infusion site from underlying anatomy, via adhesion of skin to the inserter assembly. The method may further comprise triggering an actuation assembly of the inserter assembly after the skin has been lifted at least a certain distance. The method may further comprise driving an insertion sharp through the skin.

In some embodiments, the method may further comprise releasing a bias member of the actuation assembly from an energy storing state to drive the insertion sharp through the skin. In some embodiments, the method may further comprise releasing the portion of the infusion set from the inserter assembly. In some embodiments, the method may further comprise preventing triggering of the actuation assembly with a removable lock member. In some embodiments, the method may further comprise removing the lock member from the inserter assembly. In some embodiments, the method further may comprise removing an adhesive backing from the inserter assembly. In some embodiments, the method may further comprise releasing a retraction prevention latch and driving the insertion sharp in a direction away from infusion site. In some embodiments, the method may further comprise driving a cannula subassembly carried by the insertion sharp into a portion of the infusion set. In some embodiments, driving the cannula subassembly into the portion of the infusion set may comprise driving a cannula of the cannula subassembly through the skin. In some embodiments, the method may further comprise inhibiting relative movement between a casing of the inserter assembly and a second portion of the inserter assembly at least when the skin begins being lifted.

In accordance with another example embodiment of the present disclosure, a method of placing an infusion set of an inserter assembly at an infusion site may comprise coupling the inserter assembly to skin at the infusion site. The method may further comprise lifting the skin coupled to the inserter assembly from underlying anatomy at the infusion site by displacing the inserter assembly in a direction away from the infusion site. The method may further comprise triggering an actuation assembly of the inserter assembly automatically after the skin has been lifted at least a certain distance.

In some embodiments, the method may further comprise releasing a bias member of the actuation assembly from an energy storing state to drive an insertion sharp through the skin. In some embodiments, the method may further comprise assembling the infusion set by driving a cannula subassembly of the infusion set which is carried by the insertion sharp into another portion of the infusion set. In some embodiments, driving the cannula subassembly into the another portion of the infusion set may comprise driving a cannula of the cannula subassembly through the skin. In some embodiments, the method may further comprise releasing a retraction prevention latch and driving the insertion sharp in a direction away from infusion site. In some embodiments, the method may further comprise releasing a portion of the infusion set from the inserter assembly. In some embodiments, the method may further comprise removing a lock member which prevents triggering of the actuation assembly and removing an adhesive backing from the inserter assembly.

In accordance with still another example embodiment of the present disclosure a method of placing an infusion set of an inserter assembly at an infusion site may comprise anchoring, via adhesive, an infusion set base releasably coupled to the rest inserter assembly to skin at the infusion site. The method may further comprise pulling the inserter assembly away from the body and lifting the skin anchored to the infusion set base away from underlying anatomy. The method may further comprise inhibiting relative displacement of a first portion and a second portion of the inserter assembly until the skin has been displaced to a point at which the elasticity of the skin exerts more than a threshold force against the infusion set base. The method may further comprise displacing an actuator of the first portion into a trigger arrangement of the second portion. The method may further comprise driving an insertion sharp through the skin.

In some embodiments, the method may further comprise freeing a bias member from an energy storing state to drive the insertion sharp through the skin. In some embodiments, the method may further comprise decoupling the infusion set base from the rest of the inserter assembly. In some embodiments, the method may further comprise removing a lock member from the inserter assembly and removing an adhesive backing from the infusion set base. In some embodiments, the method may further comprise driving a cannula subassembly along with the insertion sharp toward the skin such that the cannula subassembly is driven into a retraction prevention latch. In some embodiments, the method may further comprise driving the insertion sharp in a direction away from the skin. In some embodiments, the method may further comprise driving a cannula subassembly carried by the insertion sharp into the infusion set base. In some embodiments, driving the cannula subassembly into the infusion set base may comprise driving a cannula of the cannula subassembly through the skin. In some embodiments, inhibiting relative displacement of a first portion and a second portion of the inserter assembly may comprise inhibiting relative movement between a casing of the inserter assembly and at least another portion of the inserter assembly at least when the skin begins being displaced. In some embodiments, the method may further comprise releasing a retraction prevention latch and driving the insertion sharp in a direction away from infusion site. In some embodiments, the method may further comprise displacing the insertion sharp to a protected position within the inserter assembly.

In accordance with another example embodiment of the present disclosure a method of triggering an inserter assembly for placing an infusion set at an infusion site may comprise establishing an interference relationship which inhibits relative displacement of a first portion of an inserter assembly and a second portion of the inserter assembly. The method may further comprise adhering a portion of the infusion set to skin at the infusion site. The portion of the infusion set may be releasably coupled to the second portion. The method may further comprise tugging the skin at the infusion site away from underlying anatomy by displacing the inserter assembly in a direction away from the infusion site. The method may further comprise deflecting at least one portion of the second portion out of the interference relationship when force exerted by the elasticity of the skin exceeds a threshold. The method may further comprise driving an actuator of the first portion into a trigger arrangement of the second portion.

In some embodiments, establishing the interference relationship may comprise placing a resilient member of the second portion into abutment with a deflector of the first portion. In some embodiments, adhering a portion of the infusion set to the skin at the infusion site may comprise adhering an infusion set base to the skin. The infusion set base may be spaced and separate from a cannula assembly of the infusion set. In some embodiments, driving the actuator of the first portion into the trigger arrangement may comprise increasing a magnitude of relative displacement between the first and second portion of the inserter assembly after deflecting the at least one portion of the second portion out of the interference relationship. In some embodiments, driving the actuator of the first portion into the trigger arrangement may comprise driving a projection of the first portion into the trigger arrangement. In some embodiments, driving the actuator of the first portion into the trigger arrangement may comprise driving the actuator of the first portion into a ledge of a spring loaded sharp bearing body. The ledge may be engaged with a catch on the second portion. In some embodiments, the method may further comprise disengaging the ledge from the catch.

In accordance with another example embodiment of the present disclosure a method of triggering an inserter assembly for placing an infusion set at an infusion site may comprise establishing a releasable engagement between a first portion and a second portion of the inserter assembly which inhibits displacement of the first portion away from a second portion. The method may further comprise adhering a portion of the infusion set to skin at the infusion site. The portion of the infusion set may be releasably coupled to the second portion. The method may further comprise pulling the skin at the infusion site away from underlying anatomy by displacing the inserter assembly in a direction away from the infusion site. The method may further comprise disengaging the first portion from the second portion when a force compelling separation of the first and second portion exceeds a threshold. The method may further comprise increasing a magnitude of relative displacement between the first and second portion of the at least until an actuator of the first portion displaces into a trigger arrangement of the second portion.

In some embodiments, establishing the releasable engagement may comprise placing a resilient member of the second portion into an interfering relationship with a deflector of the first portion. In some embodiments, adhering a portion of the infusion set to the skin at the infusion site may comprise adhering an infusion set base to the skin. The infusion set base may be spaced and separate from a cannula assembly of the infusion set. In some embodiments, driving the actuator of the first portion into the trigger arrangement may comprise driving a projection of the first portion into the trigger arrangement. In some embodiments, driving the actuator of the first portion into the trigger arrangement may comprise driving the actuator of the first portion into a ledge of a spring loaded sharp bearing body. The ledge may be engaged with a catch on the second portion. In some embodiments, the method may further comprise disengaging the ledge from the catch. In some embodiments, establishing the releasable engagement may comprise creating an interference between the first and second portions.

In accordance with still another example embodiment of the present disclosure a method of triggering an inserter assembly for placing an infusion set at an infusion site may comprise establishing a releasable engagement between a first portion and a second portion of the inserter assembly which inhibits displacement of the first portion away from a second portion. The method may further comprise adhering a portion of the inserter assembly to skin at the infusion site. The method may further comprise pulling the skin at the infusion site away from underlying anatomy by displacing the inserter assembly in a direction away from the infusion site. The method may further comprise disengaging the first portion from the second portion when a force compelling separation of the first and second portion exceeds a threshold. The method may further comprise increasing a magnitude of relative displacement between the first and second portion of the at least until an actuator of the first portion displaces into a trigger arrangement of the second portion.

In some embodiments, establishing the releasable engagement may comprise placing a resilient member of the second portion into an interfering relationship with a deflector of the first portion. In some embodiments, adhering a portion of the inserter assembly to the skin at the infusion site may comprise adhering an infusion set base to the skin, the infusion set base being spaced and separate from a cannula assembly of the infusion set. In some embodiments, driving the actuator of the first portion into the trigger arrangement may comprise driving a projection of the first portion into the trigger arrangement. In some embodiments, driving the actuator of the first portion into the trigger arrangement may comprise driving the actuator of the first portion into a ledge of a spring loaded sharp bearing body. The ledge may be engaged with a catch on the second portion. In some embodiments, the method may further comprise disengaging the ledge from the catch. In some embodiments, establishing the releasable engagement may comprise creating an interference between the first and second portions.

In accordance with another example embodiment of the present disclosure an inserter assembly may comprise a first unit. The inserter assembly may further comprise a second unit releasably engaged with the first unit. The second unit may be configured to displace in tandem with the first unit until more than a threshold force is applied to separate the first and second unit. The inserter assembly may further comprise an infusion set. The inserter assembly may further comprise an adhesive configured to lift a patch of skin from underlying anatomy when the adhesive is in an adhering relationship with the patch of skin and the inserter assembly is displaced away from an infusion site. The inserter assembly may further comprise an insertion actuation assembly.

In some embodiments, the insertion actuation assembly may be included as part of the second unit. In some embodiments, the second unit may be releasably engaged with the first unit via an interference between part of the first unit and the second unit. In some embodiments, a portion of the second unit may be configured to deflect to a deflected state when the threshold force is applied. The portion of the second unit may be displaced out of an interfering relationship with the part of the first unit when in the deflected state. In some embodiments, the second unit may be releasably engaged with the first unit via a resilient member which is in abutting engagement with a deflector disposed on a portion of the first unit. In some embodiments, the resilient member may be configured to deflect to a deflected state when the threshold force is applied. The resilient member may be out of abutting engagement with the deflector when in the deflected state. In some embodiments, the second unit may be releasable engaged with the first unit via at least one cantilevered arm of the second unit, each of which being in abutting engagement with a cooperating projection on a part of the first unit for each of the at least one cantilevered arm. In some embodiments, each of the at least one cantilevered arm may be configured to deflect to a deflected state when the threshold force is applied. Each of the at least one cantilevered arm may be out of abutting engagement with the associated cooperating projection when in the deflected state. In some embodiments, the insertion actuation assembly may include a trigger arrangement and a spring loaded sharp bearing body. In some embodiments, the infusion set may comprise an infusion set base and a cannula assembly. The cannula assembly may be uncoupled from and in spaced relation to the infusion set base prior to activation of the insertion actuation assembly. In some embodiments, the adhesive may be provided on a bottom surface of the infusion set base. In some embodiments, the first unit may include a casing for the inserter assembly and the infusion set base is releasably coupled to the second unit and disposed at an opening in an end of the casing even with the end of the casing when the inserter assembly is in an initial state. In some embodiments, the adhesive may be covered by a removable backing. In some embodiments, the removable backing includes a strip which projects from a periphery of a main portion of the removable backing, an end of the strip being coupled to another portion of the inserter assembly. In some embodiments, the inserter assembly may further comprise a removable lock member configured to inhibit activation of the insertion actuation assembly. In some embodiments, the inserter assembly may further comprise a removable lock member configured to block relative displacement of the first and second unit even when more than the threshold force is applied to separate the first and second unit.

In accordance with another embodiment of the present disclosure an inserter assembly for transcutaneously placing a cannula may comprise a casing. The inserter assembly may further comprise an infusion set including the cannula. The inserter assembly may further comprise a spring loaded sharp bearing body. The inserter assembly may further comprise an adhesive configured to adhere to a patch of skin at an infusion site and lift the skin from underlying anatomy when the casing is displaced in a direction away from the infusion site. The inserter assembly may further comprise a trigger for the spring loaded sharp bearing body actuatable between an untriggered state and a triggered state. The trigger may be configured to remain in the untriggered state at least until a force pulling the adhesive in a direction toward the infusion site reaches a threshold.

In some embodiments, a spring of the spring loaded sharp bearing body may be held in an energy storing state when the trigger is in an untriggered state. In some embodiments, the spring loaded sharp bearing body may be urged by the spring from a raised state to a forward state when the trigger is actuated to the triggered state. In some embodiments, the infusion set may be disassembled in the inserter assembly and may include an infusion set base and a cannula subassembly including the cannula. The cannula subassembly may be carried by the spring loaded sharp bearing body. In some embodiments, the casing may include a triggering projection. In some embodiments, the adhesive may be configured to restrict movement of a portion of the inserter assembly including the trigger when the adhesive is in an adhered to the skin and the casing is displaced in a direction away from the infusion site. In some embodiments, the casing may be configured to displace relative to the portion of the inserter assembly including the trigger between an initial relative position and a triggering position in which the triggering projection contacts a portion of the trigger when the adhesive is in an adhering relationship with the patch of skin. In some embodiments, the inserter assembly may further comprise a retraction spring configured to displace the spring loaded sharp bearing body from a forward state to a retracted state when the retraction spring is freed from an energy storing state. In some embodiments, the inserter assembly may further comprise a retraction spring held in an energy storing state at least by a coupling between a portion of the infusion set and a portion of the inserter assembly including the trigger. The coupling may be released when the spring loaded sharp bearing body is urged to a forward position. The retraction spring may be configured to urge the spring loaded sharp bearing body to a retracted state when freed from its energy storing state.

The details of one or more embodiments are set forth in the accompanying drawings and the description below. Other features and advantages will become apparent from the description, the drawings, and the claims.

### BRIEF DESCRIPTION OF THE DRAWINGS

These and other aspects will become more apparent from the following detailed description of the various embodiments of the present disclosure with reference to the drawings wherein:

FIG. 1A depicts an exploded view of an exemplary inserter assembly;

FIG. 1B depicts an exploded view of another exemplary inserter assembly;

FIG. 1C depicts an exploded view of another exemplary inserter assembly;

FIG. 2 depicts an exploded view of another exemplary inserter assembly;

FIG. 3 depicts an exploded view of another exemplary inserter assembly;

FIG. 4A depicts a perspective view of an exemplary infusion set base;

FIG. 4B depicts another perspective view of the example infusion set base of FIG. 4A;

FIG. 5A depicts an exploded view of an exemplary cannula subassembly;

FIG. 5B depicts a cross-sectional view of the example cannula subassembly shown in FIG. 5A;

FIG. 6 depicts a perspective view of an example infusion set and tubing connector;

FIG. 7A depicts a perspective view of another exemplary infusion set base;

FIG. 7B depicts another perspective view of the example infusion set base of FIG. 7A;

FIG. 8A depicts an exploded view of an exemplary cannula subassembly;

FIG. 8B depicts a cross-sectional view of the example cannula subassembly shown in FIG. 8A;

FIG. 9 depicts a perspective view of another example infusion set and tubing connector;

FIG. 10A depicts a perspective view of an example infusion set base;

FIG. 10B depicts another perspective view of the infusion set base of FIG. 10A;

FIG. 11A depicts a perspective view of an example infusion set base;

FIG. 11B depicts another perspective view of the infusion set base of FIG. 11A;

FIG. 12A depicts a perspective view of an example infusion set base;

FIG. 12B depicts another perspective view of the infusion set base of FIG. 12A;

FIG. 13A depicts a perspective view of an example infusion set base;

FIG. 13B depicts a bottom plan view of the infusion set base of FIG. 13A;

FIG. 14A depicts a perspective view of an example infusion set base;

FIG. 14B depicts a bottom plan view of the infusion set base of FIG. 14A;

FIGS. 15A-15F depict various views of an exemplary tubing set connector;

FIG. 16A depicts an example tubing set connector and an example cannula subassembly which is in an exaggerated askew position for illustrative purposes;

FIG. 16B depicts an example tubing set connector and an example cannula subassembly with the cannula subassembly being displaced by the tubing set connector to a home position;

FIG. 16C depicts an example tubing set connector and an example cannula subassembly with the tubing set connector puncturing a septum of the cannula subassembly such that a delivery sharp of the tubing set connector is in fluid communication with a fluid introduction volume of the cannula subassembly;

FIG. 17 depicts an example infusion set base with an example deflector;

FIGS. 18A-18B depict views of an example tubing set connector and an example infusion set assembly, the tubing set connector including an example tine;

FIG. 18C depicts a perspective view of an exemplary tubing set connector including an example tine;

FIG. 19A depicts a top down view of an example infusion set assembly and an example tubing set connector including an example clamp, the tubing set connector being decoupled from the infusion set assembly;

FIG. 19B depicts a view of an example tubing set connector having an example clamp;

FIG. 19C depicts a view of an example tubing set connector with an example clamp coupled to an example infusion set assembly;

FIG. 20A depicts a side view of an example infusion set base;

FIG. 20B depicts another side view of the example infusion set base of FIG. 20A;

FIG. 20C depicts a perspective view of the example infusion set base of FIG. 20A;

FIGS. 21A-21F depict various views of another example infusion set base;

FIGS. 22A-22C depict various views of another example infusion set base;

FIGS. 23A-23C depict various views of another example infusion set base;

FIGS. 24A-24C depict various views of another example infusion set base;

FIGS. 25A-25C depict various views of an exemplary tubing set connector;

FIG. 26A depicts a view of an exemplary infusion set base and exemplary tubing set connector;

FIG. 26B depicts a cross-sectional view of the exemplary infusion set base and exemplary tubing set connector of FIG. 26A;

FIG. 26C depicts a perspective view of the exemplary tubing set connector of FIG. 26A;

FIG. 27A depicts a perspective view of an example infusion set;

FIG. 27B depicts another perspective view of the example infusion set of FIG. 27A;

FIGS. 28A-28D depict a number of views of an exemplary cannula subassembly;

FIGS. 29A-29B depict embodiments of cannulas which may be used in a cannula subassembly;

FIGS. 30A-30G depict a number of views of another example cannula subassembly;

FIGS. 31A-31D depict a number of views of another example cannula subassembly;

FIGS. 32A-33B depict a number of views of another example cannula subassembly;

FIGS. 34A-34F depict a number of views of another exemplary cannula subassembly;

FIGS. 35A-35F depict a number of views of another example cannula subassembly;

FIGS. 36A-36E depict a number of views of yet another example cannula subassembly;

FIGS. 37A and 37B depict views of example septums which may be included in an example cannula subassembly;

FIGS. 38A-38E depict views of another example cannula subassembly;

FIGS. 39A-39E depict views of another example cannula subassembly;

FIGS. 40A-40C depict views of an example cannula and set of exemplary septums which may be included in a cannula subassembly;

FIG. 41A depicts a side view of an example inserter assembly having an exemplary lock member installed therein;

FIG. 41B depicts another side view of an example inserter assembly having an example lock member installed therein;

FIG. 42A depicts a perspective view of an example inserter assembly with a lock member and adhering assembly in place;

FIG. 42B depicts a side view of the inserter assembly of FIG. 42A;

FIG. 43 depicts a perspective view of an example infusion set base and an example adhering assembly;

FIG. 44 depicts a perspective view of an exemplary adhering assembly for a portion of an infusion set assembly;

FIG. 45 depicts a bottom plan view of the adhering assembly of FIG. 44;

FIG. 46A depicts a top down view of an example inserter assembly with its exterior housing removed;

FIG. 46B depicts a perspective view of an example inserter assembly with its exterior housing removed;

FIG. 46C depicts a detailed view of a portion of the FIG. 46B;

FIG. 47 depicts a top down view of an example lock member;

FIG. 48 depicts a perspective view of another example lock member;

FIG. 49A depicts a cross sectional view of an example inserter assembly having an example lock member installed therein;

FIG. 49B depicts a detailed view of a portion of FIG. 49A;

FIG. 50 depicts a perspective view of an example lock member;

FIG. 51 depicts a view of an example inserter assembly which has been sectioned to reveal an exemplary lock member disposed partially within the inserter assembly;

FIG. 52 depicts a sectional view of an example inserter assembly with an example lock member installed;

FIG. 53 depicts a perspective view of an example lock member;

FIGS. 54A-54B depict views of yet another example lock member which may be used to prevent triggering of an example inserter assembly;

FIG. 55 depicts a cross-sectional view of an example inserter assembly with an example lock member installed;

FIGS. 56A-56E depict views of a variety of exemplary embodiments of lock members;

FIG. 57 depicts a flowchart detailing a number of actions which may be used to actuate an inserter assembly;

FIG. 58 depicts a top down view of an example inserter assembly;

FIGS. 59A-59B depict cross-sectional views of an exemplary inserter assembly about to be applied to a user's skin;

FIG. 60 depicts a side view of an exemplary sharp holder;

FIG. 61 depicts another side view of the exemplary sharp holder shown in FIG. 60;

FIG. 62 depicts yet another side view of the example sharp holder shown in FIG. 60;

FIG. 63 depicts a perspective view of the example sharp holder shown in FIG. 60;

FIG. 64 depicts a perspective view of a sharp holder, sharp, and cannula assembly;

FIG. 65 depicts a side view of an example sharp holder;

FIG. 66 depicts another side view of the example sharp holder of FIG. 65;

FIG. 67 depicts a perspective view of the example sharp holder of FIG. 65;

FIG. 68 depicts a detailed view of the indicated region of FIG. 67;

FIG. 69 depicts a perspective view of an example retainer base;

FIG. 70A depicts a perspective view of an example sharp retractor;

FIG. 70B depicts another perspective view of the exemplary sharp retractor shown in FIG. 70A;

FIG. 70C depicts a detailed view of a portion of FIG. 70B;

FIG. 71 depicts a cross-sectional view of an example inserter assembly taken through an arm included on a sharp retractor of the inserter assembly;

FIG. 72 depicts a perspective view of an example exterior housing;

FIGS. 73A-73B depict cross-sectional views of an example inserter assembly including the exterior housing depicted in FIG. 72;

FIGS. 74A-74B depict cross-sectional views of an example inserter assembly being withdrawn away from a user after application to the skin;

FIGS. 74C-74D depict cross-sectional views of exemplary inserter assemblies including additional springs;

FIGS. 75A-75B depict cross-sectional views of an example inserter assembly being withdrawn away from a user after application to the skin;

FIGS. 76A-76B depict cross-sectional views of an example inserter assembly after the sharp of the inserter assembly has pierced the skin of the user;

FIGS. 77A-77B depict cross-sectional views of an example inserter assembly after the sharp retractor has been freed to retract;

FIGS. 78A-78B depict cross-section views of an example inserter assembly during retraction of the sharp retractor;

FIG. 78C depicts a cross sectional view of an alternative embodiment of an inserter assembly;

FIGS. 79A-79B depict cross-sectional views of an inserter assembly after inserter assembly actuation has completed;

FIGS. 79C-D depict cross-sectional views of an example inserter assembly after an inserter assembly actuation has completed;

FIG. 79E depicts a bottom plan view of an example inserter assembly after an inserter assembly actuation has completed;

FIG. 79F depicts a cross-sectional view of an example inserter assembly;

FIG. 80 depicts a perspective view of an example retainer base;

FIG. 81 depicts a bottom view of an example inserter assembly;

FIG. 82 depicts a cross-sectional view taken at the indicated cut plane of FIG. 81;

FIGS. 83A-83B depict cross-sectional views of another exemplary inserter assembly about to be applied to a user's skin;

FIG. 84A depicts a side view of an alternative embodiment of a sharp holder;

FIG. 84B depicts a perspective view of the sharp holder shown in FIG. 84A;

FIG. 84C depicts another side view of the sharp holder illustrated in FIG. 84A;

FIG. 85 depicts a perspective view of an exemplary retainer cap;

FIG. 86 depicts a cross-sectional view of an example retainer cap and sharp holder;

FIG. 87 depicts a detailed view of a portion of FIG. 86;

FIG. 88A depicts a perspective view of an exemplary sharp retractor;

FIG. 88B depicts another perspective view of the sharp retractor shown in FIG. 88A;

FIG. 89 depicts a perspective view of an example inserter assembly including a button;

FIG. 90A depicts a top down view of the inserter assembly shown in FIG. 89;

FIG. 90B depicts a cross-sectional view taken at cut plane 90B-90B of FIG. 90A;

FIG. 90C depicts a perspective three-quarter section view taken along 90C-90C of FIG. 90A;

FIG. 91 depicts a perspective view of an example exterior housing of an inserter assembly including a button;

FIG. 92 depicts a perspective view of an example inserter assembly with an exterior housing of the inserter assembly removed;

FIG. 93 depicts a perspective view of an example inserter assembly with an exterior housing of the inserter assembly removed;

FIG. 94 depicts a detailed view of the indicated region of FIG. 93;

FIG. 95 depicts a perspective view of an exemplary sled which may be included in an inserter assembly;

FIG. 96 depicts a perspective view of an example exterior housing for an inserter assembly including a deformable region;

FIG. 97 depicts a perspective view of another example exterior housing for an inserter assembly including a protuberance on an interior face thereof;

FIG. 98 depicts a flowchart detailing a number of actions which may be used to assemble an inserter assembly;

FIG. 99 depicts a side view of an example inserter assembly and an example cartridge which may be coupled to the inserter assembly;

FIG. 100A depicts an exploded view of an example cartridge;

FIG. 100B depicts another exploded view of the example cartridge shown in FIG. 100A;

FIG. 101 depicts a perspective view of an exemplary infusion set base retainer;

FIG. 102 depicts a perspective view of an example interior housing of an example cartridge;

FIG. 103 depicts a bottom plan view of an example cartridge;

FIG. 104A depicts a cross sectional view taken at cut plane 104A-104A of FIG. 103;

FIG. 104B depicts another cross sectional view taken at cut plane 104B-104B of FIG. 103;

FIG. 105A depicts an exploded view of an example inserter assembly which may be reusable;

FIG. 105B depicts another exploded view of the example inserter assembly in FIG. 105A;

FIG. 106A depicts an exploded view of another example inserter assembly which may be reusable;

FIG. 106B depicts another exploded view of the example inserter assembly in FIG. 106A;

FIG. 107 depicts a cross sectional view of an example inserter assembly;

FIG. 108 depicts a view of an example inserter assembly exploded away from an example cartridge;

FIG. 109 depicts a detailed view of the indicated region of FIG. 108;

FIG. 110 depicts a detailed view of the indicated region of FIG. 108;

FIG. 111 depicts a perspective view of an example inserter assembly with a cartridge coupled thereto;

FIG. 112 depicts a cross sectional view of an example inserter assembly;

FIG. 113 depicts a perspective view of an example lock member;

FIG. 114 depicts a perspective view of a retraction latch body;

FIG. 115 depicts a cross sectional view of an example inserter assembly;

FIG. 116 depicts a cross sectional view of an example inserter assembly;

FIG. 117 depicts a view of an example resetting body coupled to an example retraction spring retainer;

FIG. 118 depicts a top plan view of an example interior housing of an example inserter assembly;

FIG. 119 depicts a cross sectional view taken along 119-119 of FIG. 118;

FIG. 120 depicts a cross sectional view of an example inserter assembly;

FIG. 121 depicts a cross sectional view of an example inserter assembly;

FIG. 122 depicts a top plan view of an example inserter assembly;

FIG. 123 depicts a cross sectional view taken at 123-123 of FIG. 122;

FIG. 124 depicts a cross sectional perspective view of an example inserter assembly;

FIG. 125 depicts a detailed view of the indicated region of FIG. 124;

FIG. 126 depicts a perspective view of an example interior housing of an example inserter assembly;

FIG. 127 depicts a cross sectional perspective view of an example inserter assembly;

FIG. 128 depicts a cross sectional view of a retainer cap of an example inserter assembly;

FIG. 129 depicts a detailed view of the indicated region of FIG. 128;

FIG. 130 depicts a cross sectional view of an example inserter assembly;

FIG. 131 depicts a cross sectional view of an example inserter assembly;

FIG. 132 depicts a cross sectional view of an example inserter assembly;

FIG. 133 depicts a cross sectional view of an example inserter assembly;

FIG. 134A depicts a cross sectional view of an example inserter assembly;

FIG. 134B depicts a cross sectional view of an example inserter assembly;

FIG. 135 depicts a cross sectional view of an example inserter assembly;

FIG. 136 depicts a perspective view of a cartridge coupled to an inserter assembly with an outer housing of the cartridge removed;

FIG. 137 depicts a perspective view of an example tubing connector;

FIG. 138A depicts a bottom up view of the tubing connector shown in FIG. 137;

FIG. 138B depicts a detailed view of a portion of FIG. 138A;

FIG. 139 depicts a perspective view of a tubing connector in place on a fixture;

FIG. 140 depicts a bottom up view of the fixture shown in FIG. 139;

FIG. 141 depicts a perspective view of a tubing connector aligned for installation into a fixture;

FIG. 142 depicts a cross-sectional view of a fixture with a tubing connector installed thereon and a sharp being introduced into the tubing connector;

FIG. 143 depicts a cross-sectional view of a fixture with a tubing connector installed thereon and a sharp in place and rotationally clocked to a prescribed position within the tubing connector; and

FIG. 144 depicts a flowchart detailing a number of example actions which may be used to assembly of tubing connector.

### DETAILED DESCRIPTION

In various embodiments, an infusion set may be used in conjunction with an infusion device, system, and related method as well as used in conjunction with an inserter assembly. In various embodiments, example infusion sets may be configured to be inserted into the subcutaneous layer of a user's skin and be fluidly connected to a fluid source. In various embodiments, example infusion sets may be fluidly connected to a length of tubing and/or to an infusion device. Infusion devices include any infusion pump and may include, but are not limited to, the various infusion devices described in U.S. patent application Ser. No. 15/434,906, Filed Feb. 16, 2017 and entitled Infusion Set and Inserter Assembly (Attorney Docket No. U64), now U.S. Pat. No. 10,792,419 issued Oct. 6, 2020, U.S. patent application Ser. No. 13/788,260, filed Mar. 7, 2013 and entitled Infusion Pump Assembly, now U.S. Publication No. US-2014-0107579, published Apr. 17, 2014 (Attorney Docket No. K40); U.S. Pat. No. 8,491,570, issued Jul. 23, 2013 and entitled Infusion Pump Assembly (Attorney Docket No. G75); U.S. Pat. No. 8,414,522, issued Apr. 9, 2013 and entitled Fluid Delivery Systems and Methods (Attorney Docket No. E70); U.S. Pat. No. 8,262,616, issued Sep. 11, 2012 and entitled Infusion Pump Assembly (Attorney Docket No. F51); and U.S. Pat. No. 7,306,578, issued Dec. 11, 2007 and entitled Loading Mechanism for Infusion Pump (Attorney Docket No. C54); all of which are hereby incorporated herein by reference in their entireties.

Various embodiments are described and shown herein. Each embodiment of each element of each device may be used in any other device embodiment. Each embodiment of the inserter assembly may be used with any embodiment of an infusion set.

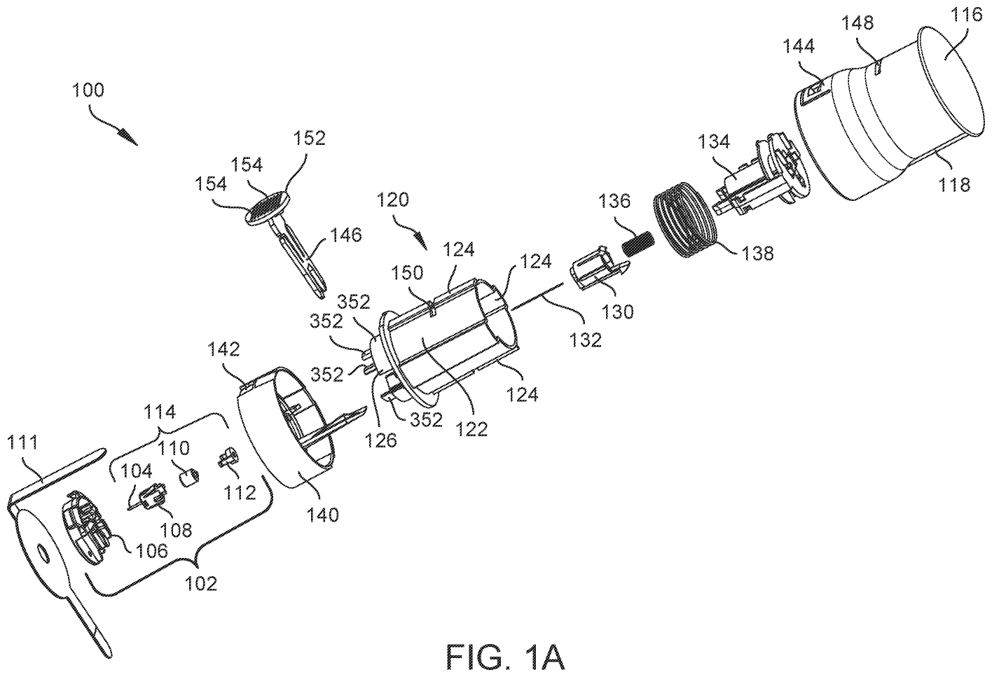

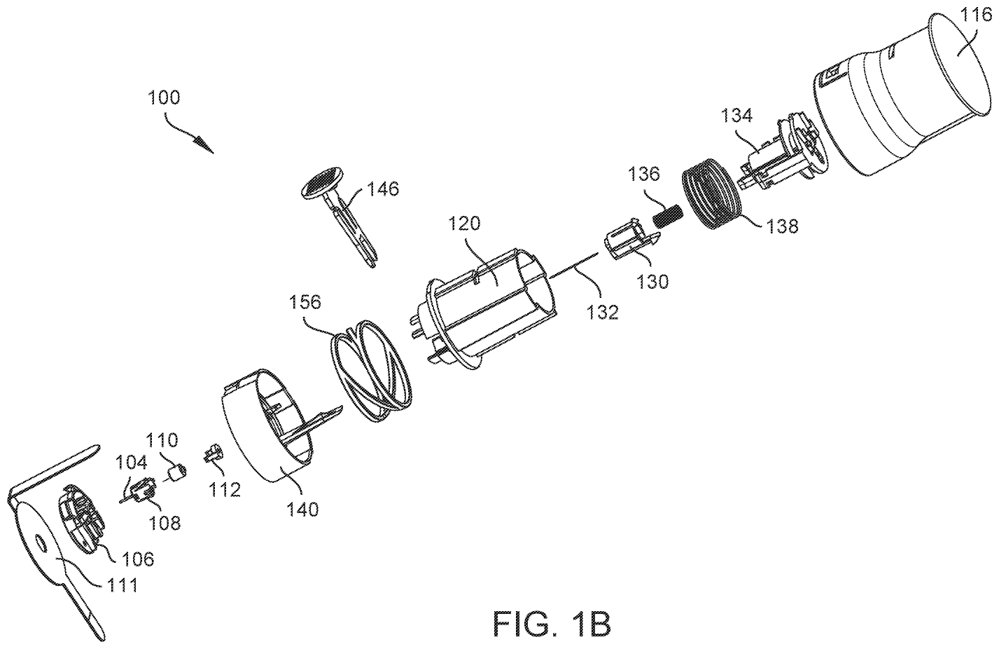

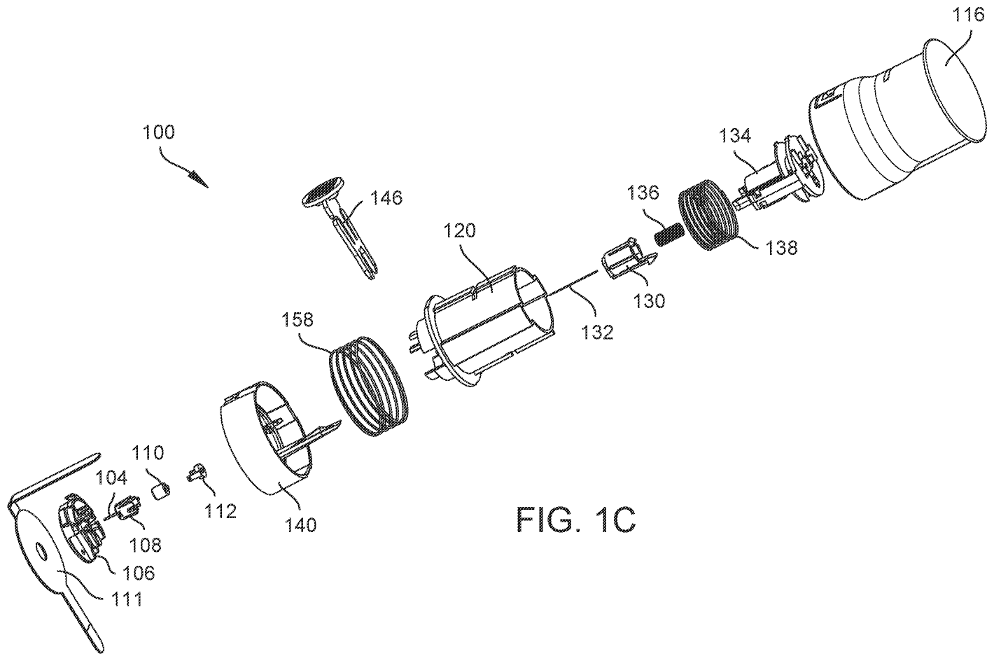

FIG. 1A depicts an exploded view of an embodiment of an example inserter assembly 100. Inserter assemblies such as inserter assembly 100 may be used to place an infusion set 102 onto an infusion site of a patient and introduce a cannula 104 of an infusion set 102 into the patient's body. In some embodiments, inserter assemblies 100 may be used to place other patient care assemblies onto the body of a patient. For example, certain inserter assemblies 100 may be operated to place physiological monitors into working relationship with a patient's body. In certain examples, an analyte sensor may be placed onto a patient with an inserter assembly 100. Infusion sets 102 may be used to supply a drug from an infusion pump to a particular location (e.g. subcutaneously) within a patient's body.

Drugs or other agents supplied may include drugs or agents which are generally supplied as a continuous or substantially continuous infusion though other drugs or agents may also be used. This may include small molecules, biologicals, recombinantly produced pharmaceuticals, and analogs thereof. Hormones such as insulin or glucagon may be administered through an infusion set 102. Other drugs such as peptides (e.g. amylin) may be provided. Drugs affecting the cardiovascular system may also be provided via an infusion set 102. As another example, vasodilators such as Treprostinil may be delivered to a patient with an infusion set 102. Chemotherapy drugs may additionally be used. Exemplary physiological monitors may include blood glucose monitors such as continuous glucose monitors. Any other type of body analyte monitor such as interstitial fluid analyte monitors may also be used.

In some embodiments, inserter assemblies 100 may place an infusion set 102 on a site as well as at least partially assemble the infusion set 102. For example, the infusion set 102 may be provided as a number of portions (e.g. separate components, subassemblies, or combinations thereof) within an inserter assembly 100. Actuation of the inserter assembly 100 may cause each portion of the infusion set 102 to be coupled together to complete the assembly of an infusion set 102. For example, assemblage of an infusion set 102 may occur as an initial stage of the actuation of the inserter assembly 100 or may occur as part of an insertion stage of inserter assembly 100 actuation which results in the cannula 104 being introduced into the patient.

As shown in the exploded view in FIG. 1A, the inserter assembly 100 contains components of an infusion set 102. The inserter assembly 100 may not be provided with an assembled infusion set 102 installed therein. The infusion set 102 may include a first portion and a second portion which are separate from one another, but coupled together during actuation of the inserter assembly 100 to form the infusion set 102. The first portion may include a base 106 which may be applied to the skin of a patient and may couple to a fluid pathway (e.g. via a terminal connector on the pathway) which is part of or extends from an infusion pump. Example infusion pumps may include any one or more disclosed in the various references incorporated by reference above, though in various embodiments, any infusion pump may be used. The base 106 may be provided with an adhesive (e.g. adhesive pad) which retains the infusion set 102 in place on the patient. The adhesive may be covered by an adhesive backing 111, liner, or film which is removed to expose the adhesive before use.

The second portion of the infusion set 102 may be a subassembly 114 of two or more components of the infusion set 102. The second portion may include a cannula 104, septum housing 108, septum 110, and septum retainer 112 for example. In some embodiments, though not all, one or more components of the second portion may be provided integrated to one another such that the components are manufactured as a single, monolithic part during, for example, a single molding operation. Any attachment, fastening, bonding, fitting together, or other assembly of these parts after manufacture may thus be avoided. The cannula 104 and the septum housing 108 are shown as such a single continuous unitary part in the example embodiment. This cannulated housing may be a molded part which is constructed of a single material such as, PTFE, Teflon, polypropylene, etc. for example. Certain components may also be joined to one another during manufacture. For example, the septum retainer 112 may be over molded onto the septum 110 or vice versa.

As shown in FIG. 1A, an insertion assembly 100 may include a number of additional components. For example, the insertion assembly 100 may include an exterior housing 116. The exterior housing 116 may enclose various components of the inserter assembly 100 and serve as the portion of the inserter assembly 100 which the user grips during operation. The exterior housing 116 in the example embodiment has a cross sectional shape which is round, though other embodiments may have different shapes such as any type of polygonal shape. In certain examples described elsewhere in the specification, a rectangular cross-sectional shape which easily fits within a pocket may be used. The cross sectional area in the example embodiment also varies with the bottom section (that most proximal the skin when in use) of the exterior housing 116 being wider than the top. The exterior housing 116 may include various ergonomic features which facilitate grasping of the inserter assembly 100 in which it is included. For example, texturing or a finger or thumb depression may be included on the outer surface of the exterior housing 116. Alternatively or additionally, a region of the external housing 116 may be thinner in width than the remaining portion of the external housing 116. This may make firm grasping of the inserter assembly 100 easier.

The exterior housing 116 may include a marking, tab, embossed section, recessed section, textured section, protuberance, color coding, appliqué, or other indicia which serves to indicate position and/or orientation of the infusion set 102 within the inserter assembly 100. For example, the exterior housing 116 in FIG. 1A includes a raised rib 118 on the outer surface of the exterior housing 116. The raised rib 118 in the example extends substantially parallel to a direction of elongation of the exterior housing 116, but may be disposed on any or partially on any exterior face(s) of the exterior housing 116 in alternative embodiments. The rib 118 is disposed to indicate the orientation of a portion of the infusion set 102 to which a fluid conduit from the infusion device may be connected. This may allow a user to position the inserter assembly 100 in a desired orientation so as to allow for a run of infusion tubing to be routed in a planned manner once the infusion set 102 is attached to the user.

An inserter assembly 100 may also include an interior housing 120. The interior housing 120 may be disposed inside of the external housing 116 when the inserter assembly 100 is assembled. Various interior housings 120 may have at least one segment which is asymmetrically designed. In the exemplary embodiment shown in FIG. 1A, the interior housing 120 includes a railed segment 122 which includes a number of rails 124. The rails 124 extend substantially parallel to one another and may be of at least two different widths. The interior face of the exterior housing 116 may include tracks or slots which cooperate with the rails 124. Due to the differing rail 124 widths, a keyed arrangement may be provided such that the interior housing 120 may only be nested within the exterior housing 116 in a prescribed orientation. The interaction of the rails 124 within the tracks may also inhibit rotation of the interior housing 120 and exterior housing 116 relative to the other. Though rails 124 are shown on the interior housing 120 in the example, the rails 124 may instead be present on the interior face of the exterior housing 116 in some embodiments. In such examples, the tracks may be located on the interior housing 120. Additionally, as shown, at least some of the rails 124 may also form channels or tracks in the interior face of the interior housing 120.

In other embodiments, a rail and track type arrangement may not be used. One of the interior housing 120 or exterior housing 116 may include at least one projection such as a tab which interfaces with a recess or guide in the other. This may similarly provide a keyed engagement and prevent relative rotation. In other embodiments, the cross sectional shape of the interior housing 120 and external housing 116 may only allow for the parts to be placed together in one orientation and may inhibit any relative rotation. For example, the cross section may be tear drop shaped or various asymmetric polygonal shapes.

The interior housing 120 may also include an infusion set base interfacing segment 126. This base interfacing segment 126 may include a number of projections 352 which may ensure that the base 106 may only be inserted into the inserter assembly 100 in a desired orientation. The projections 352 may also optionally aid in retention of the base 106 within the inserter assembly 100 and some friction between the projections 352 and surfaces of the base 106 may be present when the base 106 is installed in the inserter assembly 100. For example, the base 106 may be press fit into the projections 352. The tightness of the fit may be minimal so as to allow removal of the base 106 from the base interfacing segment 126 with little force. The projections 352 may also aid in maintaining the base 106 in a level orientation within the base interfacing segment 126.

An inserter assembly 100 may further include a sharp holder 130. The sharp holder 130 may retain an insertion sharp 132 thereon. The insertion sharp 132 may be glued or otherwise bonded into the sharp holder 130 so as to be fixedly located relative to the sharp holder 130. Any suitable type of sharp 132 may be used. For example, the sharp 132 may be a hollow or solid needle, stylet, or other pointed member which may be made of a metal material such as steel. A sharp retractor 134 and a number of springs 136, 138 may also be included in an inserter assembly 100. A retainer base 140 may serve to couple to a bottom portion of the inserter assembly 100 to hold the various components in place within the inserter assembly 100. In the example, the retainer base 140 includes retaining interfaces 142 which may snap into cantilevered retainer arms 144 included on the exterior housing 116. Other couplings are also possible such as a bayonet mount, interference fit, snap fit, adhesive, glue, threads, solvent bonding, welding, etc. When coupled together, the exterior housing 116 and retainer base 140 may form a casing of the inserter assembly 100.

As will be further described later in the specification, a latch arrangement may be included in the inserter assembly 100 and may hold the sharp holder 130 and sharp retractor 134 in place prior to and during portions of the inserter assembly 100 actuation. The latch arrangement may include a number of catches. When free to move, the springs 136, 138, may displace the sharp holder 130 and sharp retractor 134 as well as components retained thereon to complete the insertion of the cannula 104 into the patient and attach the infusion set 102 onto an infusion site. Retraction of the sharp 132 into the inserter assembly 100 may also occur as part of the actuation so as to displace the sharp 132 to a point where it is pulled out of the infusion set 102 and protected from contact with a user.

When unpacked by a user, an insertion assembly 100 may be provided with a lock member 146. The lock member 146 may be inserted through fenestrations 148, 150 in the exterior housing 116 and interior housing 120 respectively so as to span the width of at least a portion the interior housing 120. While present in the inserter assembly 100, the lock member 146 may prevent actuation of the inserter assembly 100. Example lock members 146 may mechanically prevent displacement of one or more component of the inserter assembly 100 which initiates the actuation action of the inserter assembly 100. In the example embodiment, the lock member 146 includes a flange 152 which may be grasped by a user during removal of the lock member 146.

As shown, a lock member 146 may include a number of raised sections 154 (e.g. ridges or bumps) thereon. These raised sections 154 may provide material which may help to bond to a portion of the adhesive backing 111 during a welding operation. As a result, the lock member 146 may be attached to the adhesive backing 111 such that a user would have a visual cue in the event that one of the lock member 146 or adhesive backing 111 is removed while the other is still in place. This may help to encourage removal of both components prior to an attempt to actuate the inserter assembly 100 making the device more intuitive.

Referring now also to FIG. 1B and FIG. 1C, in some examples, an inserter assembly 100 may include one or more additional springs 156, 158. As shown in FIG. 1C, the additional spring 158 may be a conventional metallic spring. Alternatively, and as depicted in FIG. 1B, a plastic spring 156 may be used. In certain embodiments, the plastic spring 156 may be injection molded, cut out of a tube of material (e.g. via laser cut), made via a material additive process, or any other suitable method. Such springs 156, 158 will be further described later in the specification.

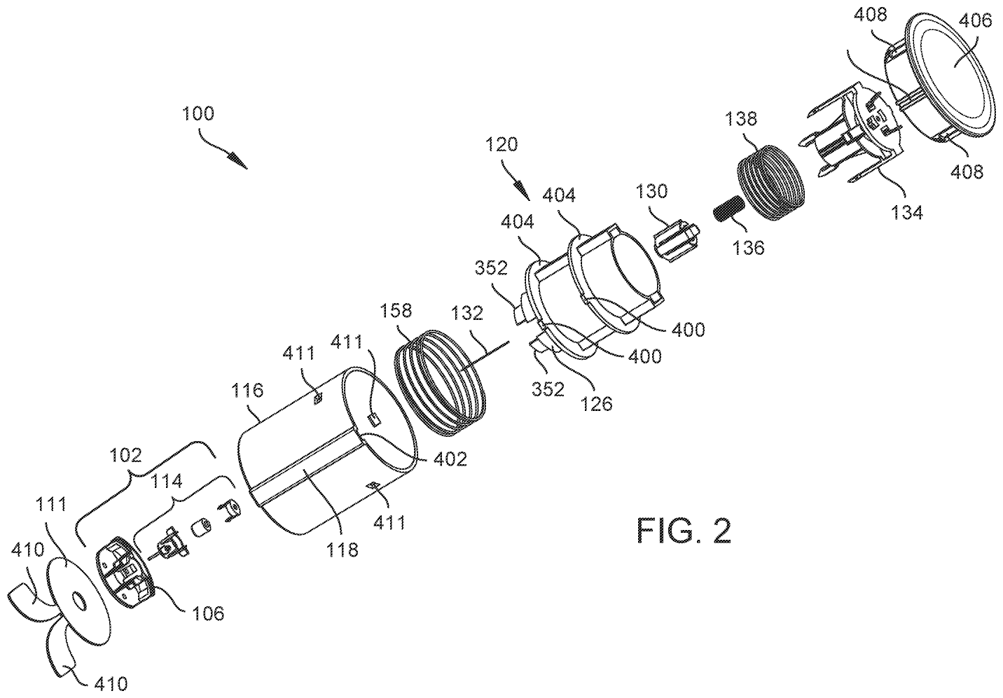

Referring now also to FIG. 2, another inserter assembly 100 is depicted. The insertion assembly 100 in FIG. 2 includes an exterior housing 116 which may enclose various components of the inserter assembly 100 and serve as the portion of the inserter assembly 100 which the user grips during operation. Though depicted as round, the exterior housing 116 may have other cross-sectional shapes or various ergonomic features as described above. The exterior housing 116 includes a position indicium in the form of a raised rib 118 extending off the outer surface of the exterior housing 116. The rib 118 may be disposed to indicate the orientation a portion of an infusion set 102 contained within the inserter assembly 100.

An interior housing 120 is also included in FIG. 2 and may be keyed so as to ensure it is assembled into the inserter assembly 100 in a prescribed orientation and prevent relative rotation. As in FIG. 2, the interior housing 120 may be made asymmetric by the inclusion of at least one projection 400 such as a tab which interfaces with a recess or guide 402 in the exterior housing 116. In the example, the guide 402 is provided by a channel formed by the raised rib 118 on the interior face of the exterior housing 116. The projections 400 on the inserter assembly 100 in FIG. 2 are included on spacing plates 404 which ensure that the interior housing 120 fits snuggly within the exterior housing 116. An infusion set base interfacing segment 126 is also included on the example interior housing shown in FIG. 2.

A sharp holder 130 which may be affixed to an insertion sharp 132 is shown in the example embodiment. Additionally, a sharp retractor 134 and a number of springs 136, 138 may also be included. A retainer cap 406 may serve to couple to a top portion of the inserter assembly 100 to hold the various components in place within the inserter assembly 100. In the example, the retainer cap 406 includes cantilevered retainer arms 408 which may snap into retaining interfaces 411 included on the exterior housing 116. Other couplings are also possible such as a bayonet mount, interference fit, snap fit, adhesive, glue, threads, solvent bonding, welding, etc. When coupled together, the exterior housing 116 and retainer cap 406 may form a casing of the inserter assembly 100.

As described in detail elsewhere herein, a latch arrangement may be included in the inserter assembly 100 may hold the sharp holder 130 and sharp retractor 134 in place prior to and during portions of the inserter assembly 100 actuation. The latch arrangement may include a number of catches. When free to move, the springs 136, 138, may displace the sharp holder 130 and sharp retractor 134 as well as components retained thereon to complete the insertion of the cannula 104 into the patient and attach the infusion set 102 onto an infusion site. Retraction of the sharp 132 into the inserter assembly 100 may also occur as part of the actuation.

In the example embodiment depicted in FIG. 2, the inserter assembly 100 does not include a lock member 146. In various embodiments, however, fenestrations similar to fenestrations 148, 150 in FIGS. 1A-1C in the exterior housing 116 and interior housing 120 may be included to accommodate a lock member 146. In these embodiments, the adhesive backing 111 could be bonded onto the lock member 146. In the example embodiment, however, the adhesive backing 111 includes two pull tabs 410 (though any suitable number may be included). These pull tabs 410 may be grasped by a user to facilitate removal of the adhesive backing 111. The additional spring 158 depicted in FIG. 1C is also included in FIG. 2.

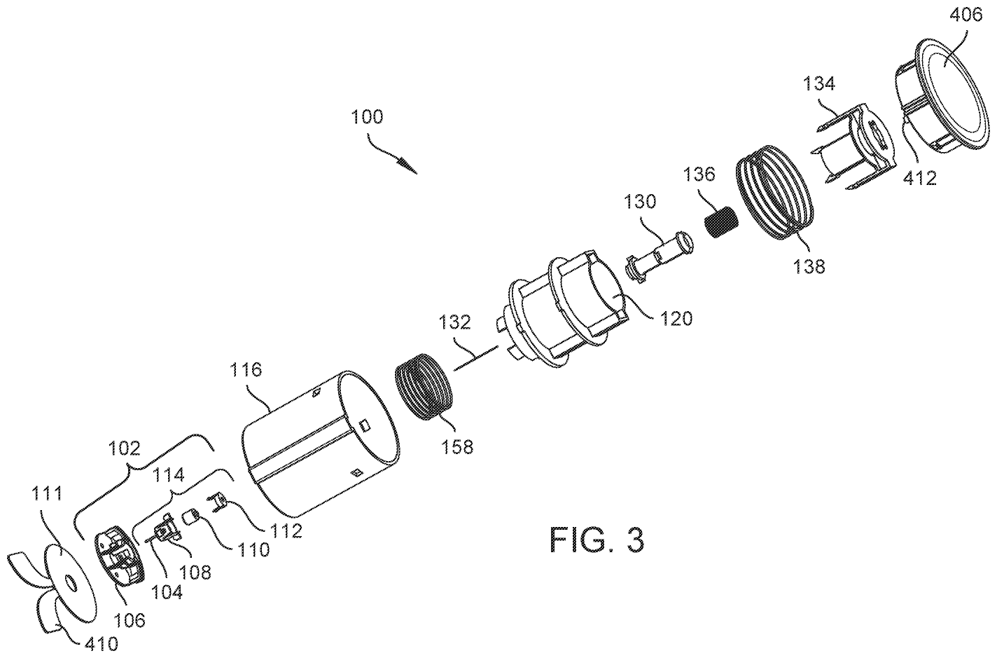

Referring now to FIG. 3, another example embodiment of an inserter assembly 100 is depicted. As shown, the inserter assembly 100 in FIG. 3 includes an exterior housing 116 and interior housing 120 similar to those shown in the example depicted in FIG. 2. The sharp holder 130 and sharp retractor 134 differ from those depicted in FIGS. 1A-2. A retainer cap 406 may serve to couple to a top portion of the inserter assembly 100 to hold the various components in place within the inserter assembly 100 and form a casing of the inserter assembly 100. The retainer cap 406 couples to the exterior housing 116 in a similar manner to the embodiment described in relation to FIG. 2, however, any other type of coupling may be used in alternative embodiments. The retainer cap 406 also includes a projection 412 which may fit within a portion of the sharp holder 130 when the inserter assembly 100 is fully assembled and ready for actuation.

As described in detail elsewhere herein, a latch arrangement may be included in the inserter assembly 100 and may hold the sharp holder 130 and sharp retractor 134 in place prior to and during portions of the inserter assembly 100 actuation. The latch arrangement may include a number of catches. When free to move, the springs 136, 138, may displace the sharp holder 130 and sharp retractor 134 as well as components retained thereon to complete the insertion of the cannula 104 into the patient and attach the infusion set 102 onto an infusion site. Retraction of the sharp 132 into the inserter assembly 100 may also occur as part of the actuation.

In the example embodiment depicted in FIG. 3, the inserter assembly 100 does not include a lock member 146. However, in various embodiments, fenestrations similar to fenestrations 148, 150 in FIGS. 1A-1C may be included in the exterior housing 116 and interior housing 120 to accommodate a lock member 146. In these various embodiments, the adhesive backing 111 may be bonded onto the lock member 146. In the example embodiment, however, the adhesive backing 111 includes two pull tabs 410. These pull tabs 410 may be grasped by a user to facilitate removal of the adhesive backing 111. The additional spring 158 depicted in FIG. 1C is also included in FIG. 3

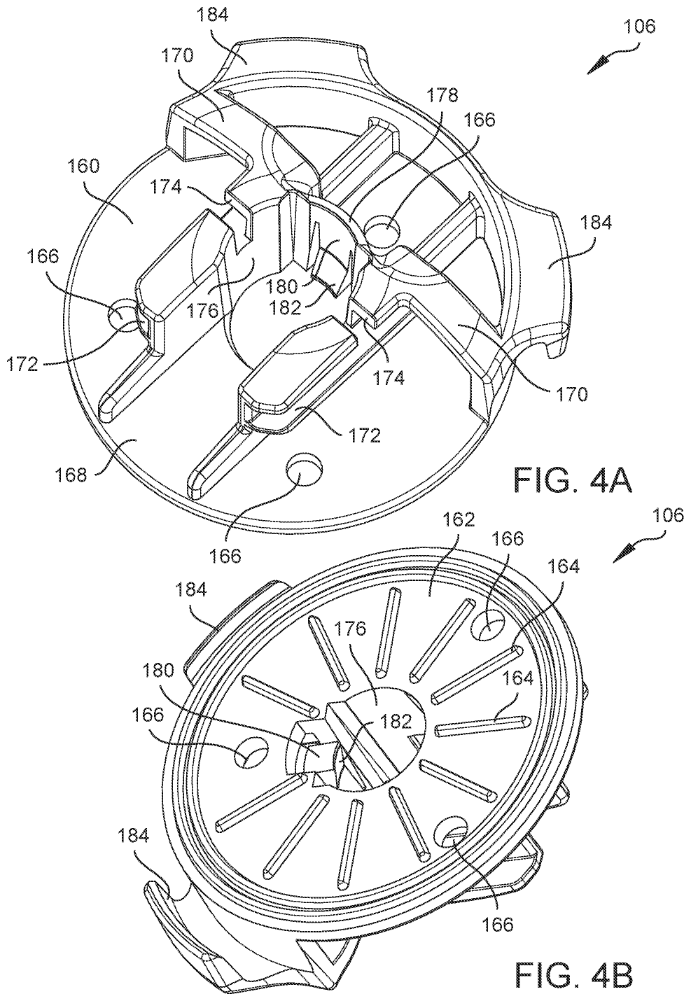

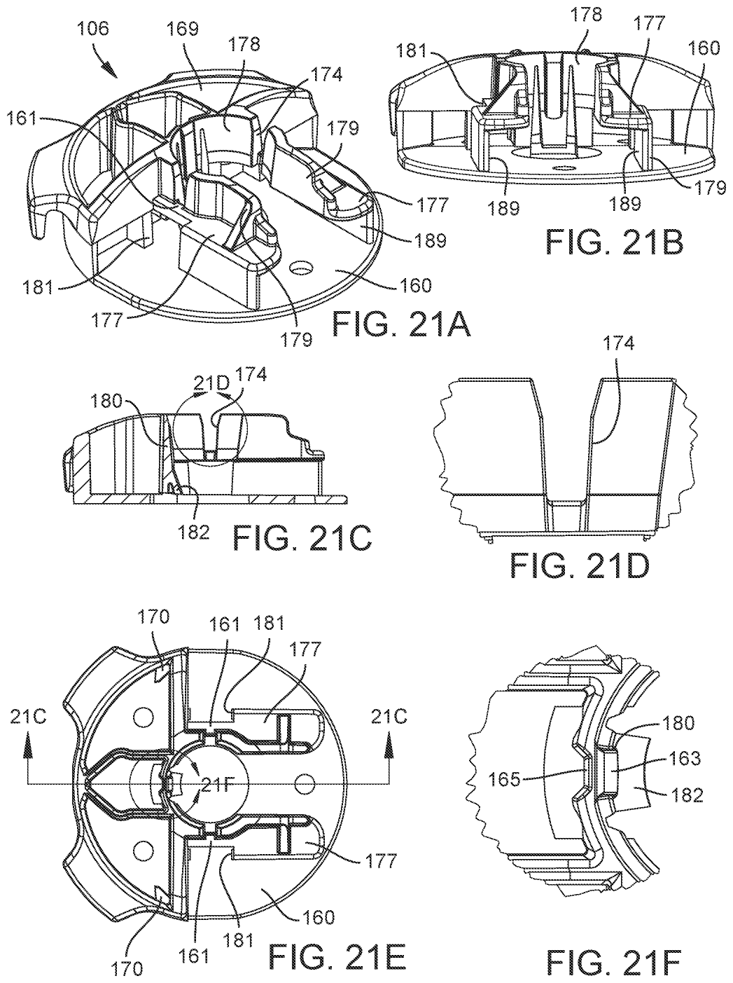

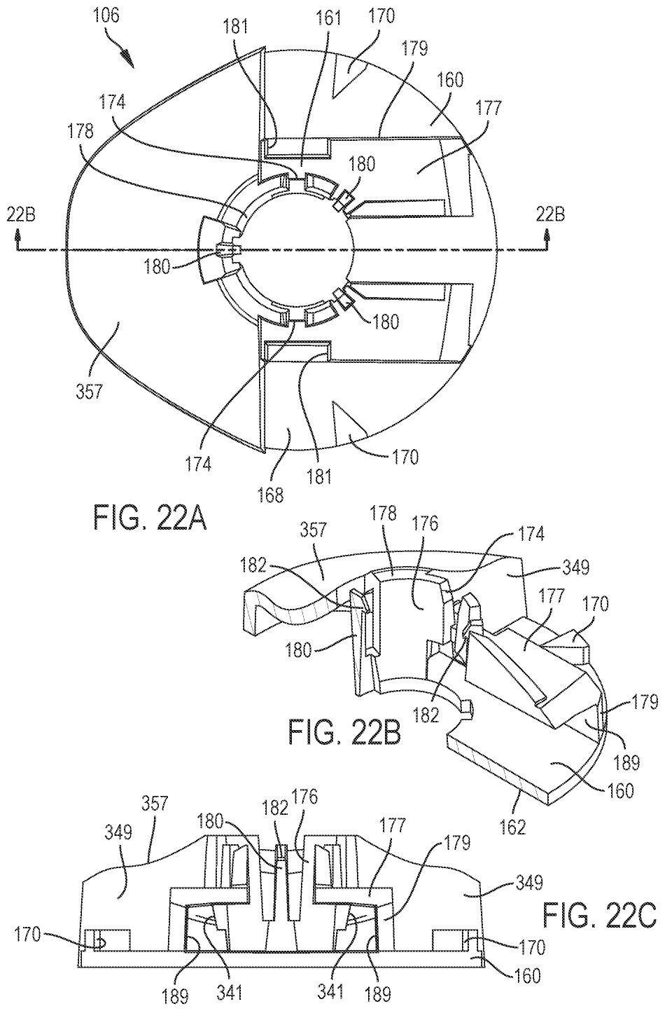

Referring now to FIG. 4A and FIG. 4B, top and bottom perspective views of an infusion set base 106 are respectively shown. As shown, the base 106 may have a platform portion 160. The platform portion 160 may be positioned against the skin of a patient while the infusion set 102 is in place on an infusion site. In the example embodiment, the platform portion 160 has a substantially round, and in this case generally circular, disc-like foot print. In other embodiments, the platform portion 160 may be obround (sec, e.g., FIGS. 21A-21F), egg shaped or ovate (see, e.g., FIGS. 22A-C). The platform portion 160 is also substantially flat. This, however, need not be the case in all embodiments.

In various embodiments, adhesive may be applied to the bottom face 162 of a platform portion 160. An adhesive backing 111 (see, e.g. FIG. 1A) may overlay the adhesive. In the example embodiment, the bottom face 162 of the platform portion 160 includes a number of raised segments 164. The exemplary raised segments 164 are depicted as radially arrayed ridges which are spaced at substantially regular angular intervals over a portion of the bottom face 162. Two concentric raised rings are also present around the periphery of the bottom face 162. In other embodiments, raised segments other than ridges may be included and/or the raised features may not be radially arrayed or regularly spaced. In some embodiments the raised segments 164 may be dots or round regions spaced around the bottom face 162. Any suitable pattern may be used in various embodiments. The raised segments 164 may allow for the adhesive to be bonded (e.g. ultrasonically or High frequency welded) onto the bottom face 162. For example, the adhesive may be included on a substrate which may be a plastic such as polypropylene or any other plastic which would be amenable to a welding operation. The raised segments 164 and substrate may melt together during the welding operation creating a bond which keeps the adhesive on the bottom face 162 of the infusion set base 106. In alternative embodiments, a bonding agent such as double-sided tape may couple the substrate to the bottom face 162 and the bottom face 162 may be devoid of raised segments 164.

In various embodiments, a platform portion 160 may include a number of pass throughs 166. Three circular pass throughs 166 are shown in FIGS. 4A-B though any number may be included in other examples and their shape may differ. Pass throughs 166 may be provided for case of manufacturing as they may allow for projections on a portion of an assembly line to interface therewith. This interface may aid in orienting or centering of the base 106 or any infusion set 102 subassemblies including the base 106 during manufacturing operations. Where the pass throughs 166 aid in orientation, it may be desirable that the pass throughs 166 be disposed in an asymmetric manner (though symmetric pass through 166 arrangements are also possible). In the example, the pass throughs 166 are disposed in a triangle type layout such that the base 106 would only mate into a set of corresponding projections in one orientation. Additionally, in some embodiments, any adhesive and adhesive liner 111 may include holes which align with the locations of pass throughs 166. Thus, even when the inserter assembly 100 is fully assembled and ready for triggering, the pass throughs 166 may be used to aid in the manufacturing process. The pass throughs 166 may also be engaged by a portion of any packaging that the inserter assembly 100 is provided in to help hold the inserter assembly 100 in place.

Though the pass throughs 166 may be helpful during manufacturing, the pass throughs 166 may also provide other benefits. For example, the pass throughs 166 may provide a window to view skin around the infusion site. As a result, a user may be able to assess the skin for signs of irritation or inflammation (e.g. rubor or redness). Additionally, the pass throughs 166 may provide a pathway through which ambient air may be in communication with space between any raised segments 164 on the bottom face 162 of the infusion set base 106. This may help to allow the area under an infusion set 102 to breathe while the infusion set 102 is adhered against the skin.

Extending from the periphery of the platform portion 160 may be a number of tubing retainers 184. The tubing retainers 184 may allow for infusion tubing 366 (see, e.g., FIG. 6) to be wrapped around a portion of the infusion set 102 and held in place. This may aid in inhibiting kinking of the tubing 366 and help a user to conveniently route infusion tubing 366 as needed. Tubing retainers 184 may be omitted in alternative embodiments of infusion set bases 106 described herein (sec, e.g., FIGS. 22A-C).

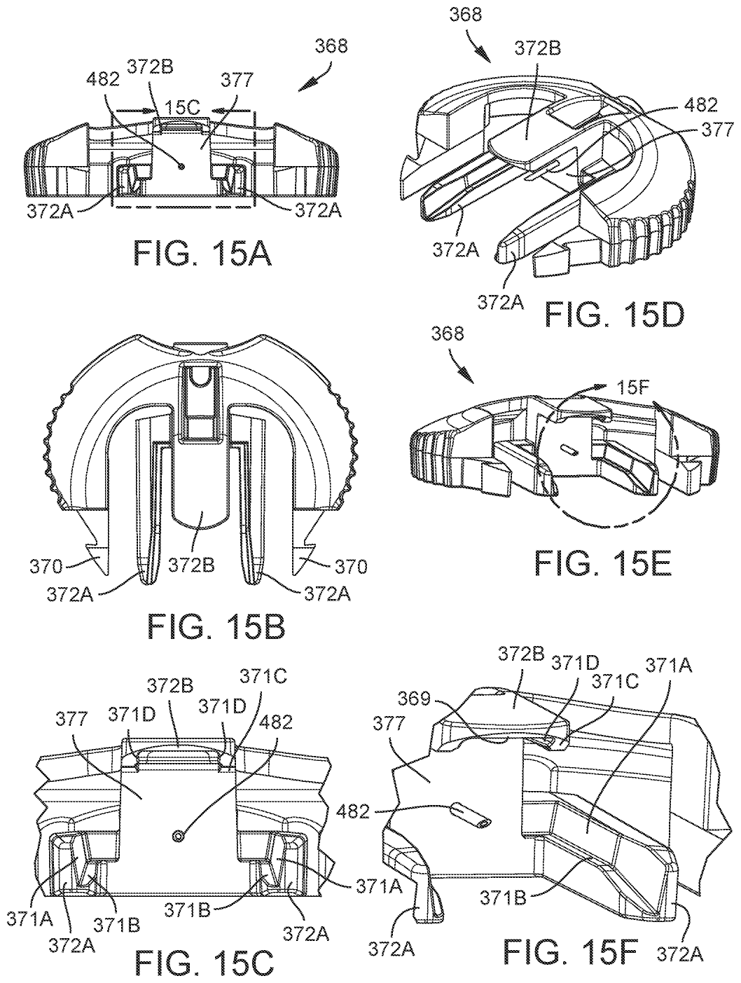

Referring now also to FIG. 6, the top face 168 of the platform portion 160 may include various receiving features extending therefrom which may mate or interface with a connector 368 included at a terminal end of infusion tubing 366 extending from an infusion pump. The infusion tubing 366 may be coupled to an infusion pump reservoir such as a syringe or an infusion pump outlet in various embodiments. Infusion tubing 366 may be coupled to an infusion pump via luer lock, or other mechanical coupling, adhesive, solvent boding, or in any other suitable fashion. The base 106 may include connector receivers 170 which cantilevered fingers 370 on the tubing set connector 368 may deflect around as the connector 368 is slid onto the base 106. Once past the connector receivers 170, the cantilevered fingers 370 may restore to their undeflected position and a bump or catch projection on the cantilevered fingers 370 may displace into latching engagement with a cooperating feature of the connector receivers 170. Sharp flanking projections 372 may also be present on the connector 368. These flanking projections 372 may extend substantially parallel to a sharp 482 (see, e.g., FIG. 15F) included on the connector 368 and may present an obstacle which helps block accidental contact between the sharp 482 and the user. A shielding wall 169 may be provided on the base 106 and may help to block fingers or objects from inadvertently dislodging the cantilevered fingers 370 out of engagement with the connector receivers 170. The shielding wall 169 may be continuous with the connector receivers 170.

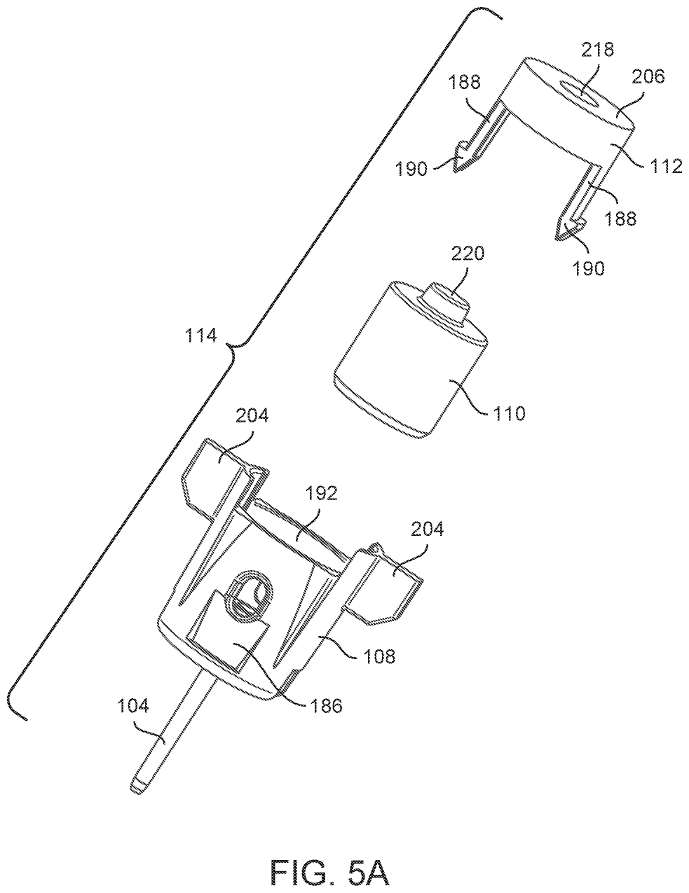

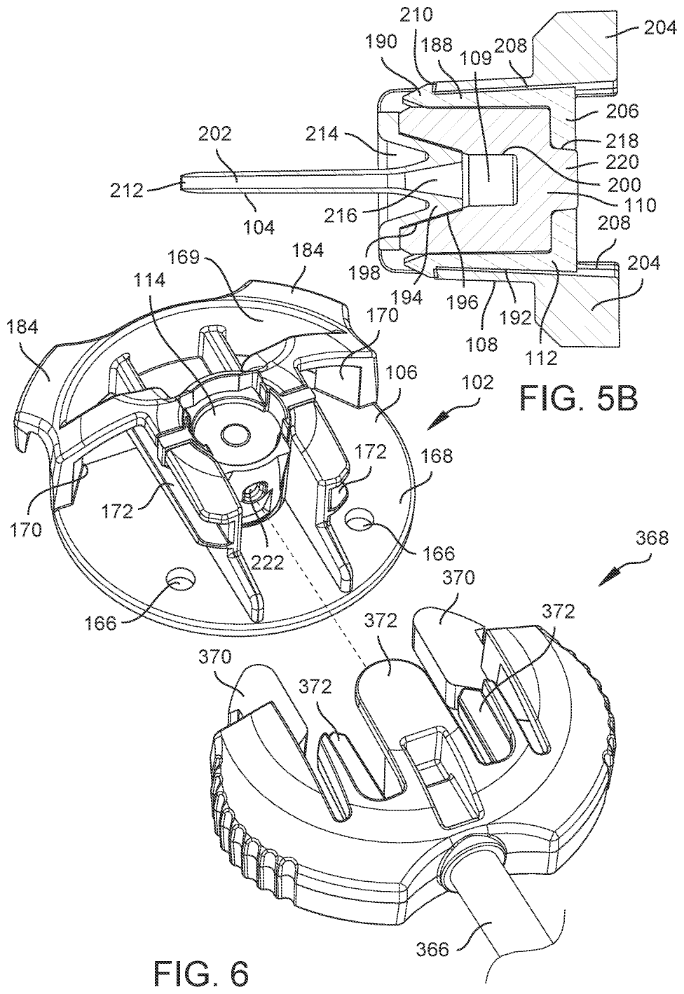

Guides 172 for each of the connector fingers 370 and the flanking projections 372 may also be included and in the example embodiment define a number of slots along which the connector fingers 370 and flanking projections 372 may be slid as the connector 368 is displaced into engagement with the infusion set 102. The guides 172 may make it easier for a user to couple the infusion set 102 and connector 368 together. Additionally, the guides 172 may help to ensure that a sharp 482 (see, e.g. FIG. 15F) on the connector 368 is introduced into the infusion set 102 along or close to a desired insertion axis. In the example, the guides 172 include a notch 174. Each notch 174 may be sized to accept a projection on a component of the inserter assembly 100. Notches 174 may also be sized to accept at least a portion of a projection included on a cannula subassembly 114 (see, e.g. FIG. 5A). The flanking projections 372 may be sized so as to extend from the connector 368 a distance shorter than the location of the notches 174 when the connector 368 is attached to the infusion set 102. As described in further detail later in the specification, the notches 174 may aid in facilitating release of the base 106 from the inserter assembly 100 during actuation.

In various embodiments, the base 106 may also include a receptacle 176 for mating with a cannula subassembly 114 (sec, e.g. FIG. 5A) of an infusion set 102. In the example, the receptacle 176 is generally centrally disposed on the base 106 and the receptacle 176 is flanked on each side by the guides 172. The receptacle 176 may be surrounded, at least partially, by a receptacle wall 178 which projects upwardly from the top face 168 of the platform portion 160. Thus the receptacle wall 178 and portions of the guides 172 including the notches 174 may define the receptacle 176. Part of the receptacle wall 178 may include a cantilevered section 180. The cantilevered section 180 may include a protuberance (e.g. barb or ramp) 182. In some embodiments, a portion of a receptacle wall 178 and/or guides 172 may include a tapered section (see, e.g. the embodiment depicted in FIGS. 10A-10B). The tapered section may be included at the portion of the receptacle wall 178 and/or guides 172 most distal to the platform 160. Such a tapered section may aid in guiding a cannula subassembly 114 into place as the infusion set 102 is assembled by funneling the cannula subassembly 114 into the receptacle 176. Additionally or alternatively, the notches 174 may be tapered (see, e.g., FIGS. 21A-21F) along at least a portion of their length. This may aid in guiding a cannula subassembly 114 into place as the infusion set 102 is assembled.

Referring now also to FIG. 5A, an example cannula subassembly 114 is depicted. As shown, the septum housing 108 may include a notch 186. The notch 186 may be recessed into an exterior face of the septum housing 108. During displacement of the cannula subassembly 114 into the receptacle 176 of the base 106, the cantilevered section 180 of the base 106 may deflect around the septum housing 108 until the protuberance 182 is free to spring into the notch 186. Once the cantilevered projection 180 has restored into its undeflected state and the protuberance 182 is disposed within the notch 186, the cannula subassembly 114 may be retained within the base 106. In the retained state, cars or nubs 204 of the septum housing 108 may at least partially reside within the notches 174 of the base 106 (shown in FIG. 6).

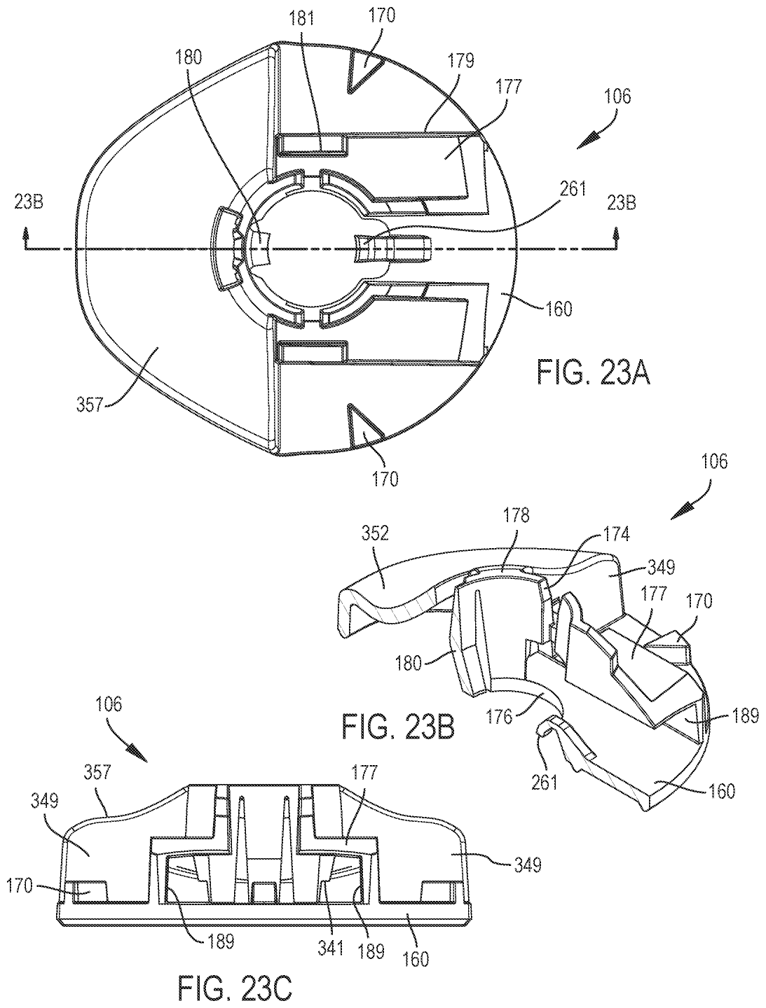

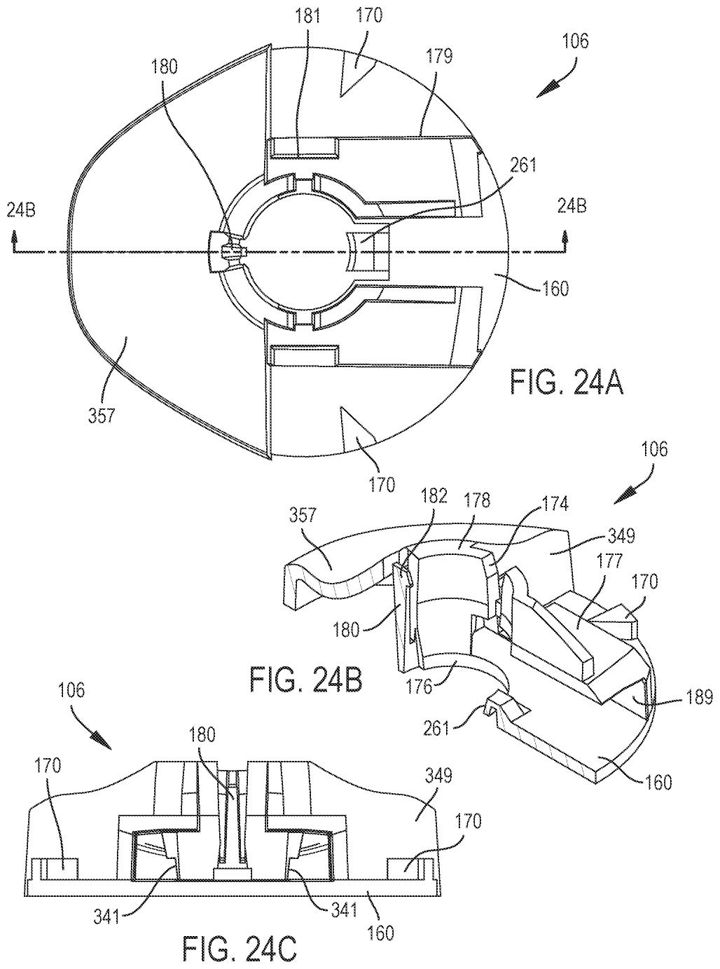

Referring now additionally to FIG. 5B, which depicts a cross sectional view of an assembled cannula subassembly 114, the cannula subassembly 114 may further include a septum 110 and a septum retainer 112 in certain embodiments. In alternative embodiments, and as described in relation to other examples herein, multiple septums 110 may be included in place of the single septum 110. The septum housing 108 may include a cup like receiving section or receptacle 192 into which the septum 110 may be introduced. The receiving section 192 may include a raised region 194 in a center thereof. In the example, the raised region 194 may have a conic frustum type shape though other shapes are also possible. Shapes such as those included on the septum interface regions 183 of cannulas 104 depicted in FIGS. 23A-24G could for example be used for alternative raised regions 194. The septum 110 may also have a cup like septum recess 196 included in the bottom face thereof. The septum recess 196 may include an enlarged or flared out section 198 (or section which otherwise corresponds to the shape of an example raised region 194) which is formed as a negative version of the raised region 194 in the receiving section 192 of the septum housing 108. Thus the enlarged section of the septum recess 196 may self-center the septum 110 as the septum 110 is introduced into the receiving section 192 and may be referred to herein as a centering wall of the septum recess 196. A second section 200 of the septum recess 196 most distal to the bottom face of the septum 110 may define, at least partially, a fluid introduction volume 109 into which a needle or sharp 482 (see, e.g., FIG. 15F) included on the tubing connector 368 may penetrate to deliver fluid into the infusion set 102. Fluid may pass from the fluid introduction volume 109 to the lumen 202 of the cannula 104 and into the patient. The second section 200 of the septum recess 196 may have any suitable cross section though in the example has a round, roughly circular cross section. The septum housing 108 may include a connector needle passage 222 in a sidewall thereof (as shown in FIG. 6) which may allow a needle or sharp 482 (sec, e.g., FIG. 15D) of the connector 368 access to the fluid introduction volume 109. In the example embodiment, the connector needle passage 222 is depicted as a fenestration extending though the septum housing 108. A connector needle passage 222 and notch 186 may (though need not be) be present on opposing sides of the septum housing 108 so as to allow the cannula subassembly 114 to be capable of being installed into the base 106 in two orientations. This may make assembly of the inserter assembly 100 simpler.

The receiving section 192 of the septum housing 108 may also receive a portion of a septum retainer 112. The septum retainer 112 may be constructed with a body 206 from which extends at least one cantilevered projection 188 including a terminal protuberance or latch member 190. In the example, the body 206 is substantially planar. Additionally, two cantilevered projections 188 which are disposed opposite one another and extend generally perpendicular to the body 206 are present. These cantilevered projections 188 may fit within guides 208 included on the interior surface of the receiving section 192 of the septum housing 108. The guides 208 shown are recessed into the interior surface of the receiving section 192 and are substantially in the same plane as the cars 204. The guides 208 are ramped such that distance between the two guides 208 decreases as distance from the cannula 104 decreases. This may deflect the cantilevered projections 188 of the septum retainer 112 toward the axis of extension of the cannula 104 as the septum retainer 112 is advanced into the receiving section 192. Once the septum retainer 112 has been advanced into the septum housing 108 a certain distance, the cantilevered projections 188 may spring outward such that the protuberance 190 on each cantilevered projection 188 enters into latching engagement with a catch 210 on the septum housing 108 as shown in FIG. 5B. In some embodiments, a portion of the cantilevered projections 188 may extend into or through an aperture included in the bottom of the septum housing 108 and enter into latching engagement with a catch adjacent to the aperture or formed by a wall of that aperture. With the septum retainer 112 latched into place within the cannula subassembly 114, the septum 110 may be placed into scaling relationship with the septum housing 108. Fluid contained in the fluid introduction volume 109 of the septum 110 may thus be provided a sealed fluid flow path to the outlet 212 of the cannula 104.

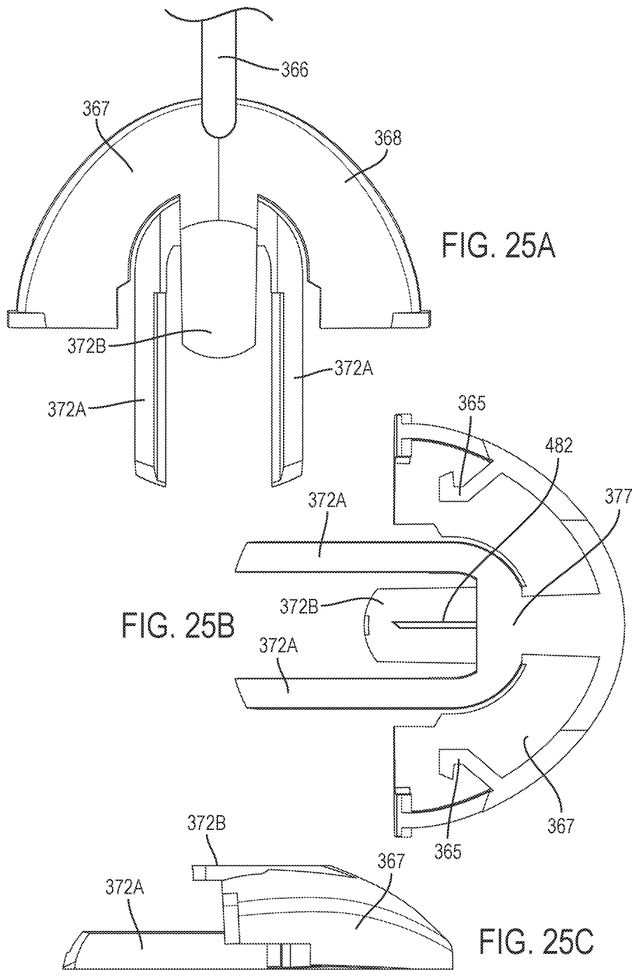

As shown, the septum retainer 112 includes a channel 218 which extends therethrough. The example channel 218 is disposed in substantially the center of the body 206. When the septum retainer 112 is locked in place within the cannula subassembly 114, a nub or projection 220 of the septum 110 may extend into the channel 218. Thus the channel 218 may provide an access pathway for an insertion sharp 132 of the inserter assembly 100 to extend though the infusion set 102 and out of the outlet 212 of the cannula 212. In the example shown, the nub 220 is in the shape of a conic frustum and the channel 218 is shaped in a cooperative manner to receive the nub 220. Other nub 220 shapes may be used in alternative embodiments such as any of those described herein (see, e.g., FIGS. 25A-25D).

The cannula 104 may include an insertion sharp guide section 216 which may aid in directing the insertion sharp 132 into the lumen 202 of the cannula 104. The insertion sharp guide section 216 may have a funnel like shape though other shapes are also possible. In the example embodiment, the insertion sharp guide section 216 includes relatively steep sides and is encompassed by a flat (or perhaps chamfered or rounded) peripheral edge which forms a wall of the fluid introduction volume within the infusion set 102. This peripheral edge may be the uppermost face of the raised section 192 in some embodiments. The insertion sharp guide section 216 may be continuous with the walls of the lumen 202 of the cannula 104 and may be wetted by any fluid delivered through the infusion set 102. The insertion sharp guide section 216 may also be continuous with the raised region 194 in the receiving section 192 of the septum housing 108.

Referring primarily to FIG. 5B, a recess 214 may be included in the septum housing 108 which surrounds, or at least partially surrounds, the cannula 104. This recess 214 may not be covered by adhesive when the adhesive is placed onto the infusion set base 106. The recess 214 may provide room for the cannula 104 to move relative to the base 106 if the infusion set 102 or a portion of the patient's body causes a force to be applied to the cannula 104. This may minimize shearing action on the cannula 104. Additionally, the recess 214 may serve as a volume into which an agent may be placed. For instance, an antiseptic, disinfectant, anti-inflammatory, anesthetic, or other topical agent such as an ointment may be contained in the recess 214. This agent may be medicinal, nutritional, or some other type of agent. Thus upon application of the infusion set 102, the agent may be introduced to the skin of the patient and may be held in contact with therewith. In some examples, the agent may be kept in contact with the recess 214. This may be desirable or beneficial for a number of reasons including that it may aid in preventing infection, inflammation, or pain and may provide a way to apply the agent in a convenient manner. In the example embodiment, the recess 214 is shaped in the form of a conic section (e.g. hyperbola or parabola) revolved around the center axis of the cannula 104.

In various embodiments, the cannula 104 may be tapered or non-tapered. In some embodiments, the cannula 104 may include one or more tapered section and one or more untapered or straight section. Any tapered sections may extend at an angle to the long axis of the cannula 104. In some embodiments, instead of being at some constant angle to this axis, a curvature may be present. The angle or the degree of curvature over a tapered section may also vary over the extent of a tapered section.

In some embodiments, a first portion of the cannula 104 proximal the outlet 212 is tapered. The taper present at this section results in a reduced wall thickness as proximity toward the outlet 212 of the cannula 104 increases. Additionally, a second portion of the cannula 104 adjacent the point on the septum housing 108 from which it extends is also tapered. The angle of the taper of the second portion may be substantially equal to the angle of the insertion sharp guide section 216 in certain embodiments. The taper at the second portion decreases the width of the cannula 104 at this section without substantially decreasing the thickness of the wall of the cannula 104 surrounding the lumen 202. A straight section may be disposed intermediate the first and second portions of the cannula 104 in the example embodiment. Alternatively, a slightly tapered section may be used as the intermediate segment. This section may be tapered to a lesser degree than the first and second portion of the cannula and may or may not be tapered in a manner which maintains a constant wall thickness along the length of the intermediate segment.

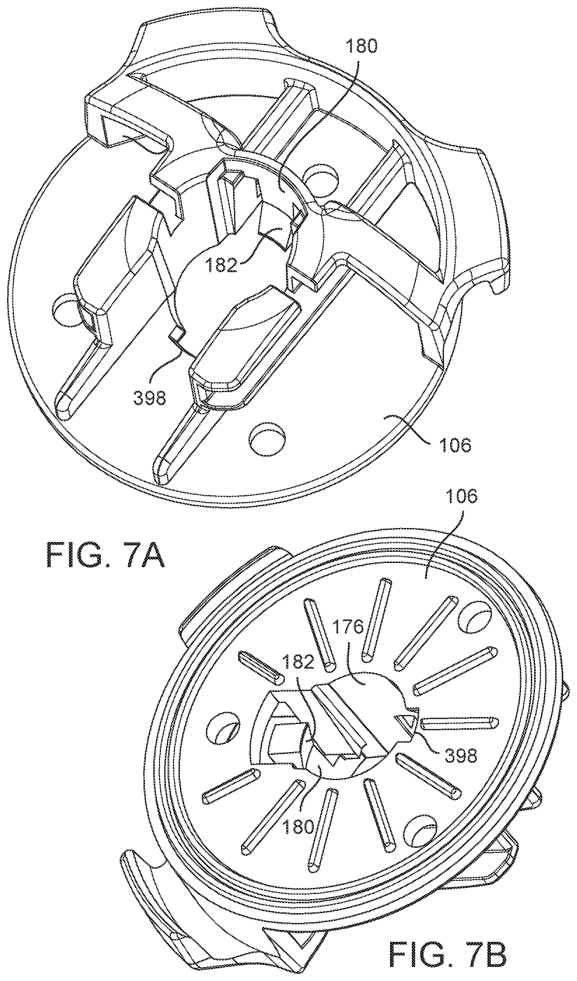

Referring now to FIGS. 7A-9 another example infusion set 102 is depicted. As shown, the cannula subassembly 114 of the example infusion set 102 is designed to allow production via straight pull molding. Features which may require molds with side actions or other special characteristics are not present on the example cannula subassembly 114. For example, in some embodiments, undercut features may not be present. This may allow the cannula subassembly 114 or components thereof to be constructed of a wider array of different materials.

As shown, the cannula subassembly 114 includes a septum 110 similar to that shown in FIGS. 5A-5B. In alternative embodiments, a plurality of septums 110 may be included in place of the single septum 110 shown. The septum housing 108 does not include notches 186. In place of the notches 186, the septum housing 108 includes salients 388 which extend outward from the outer surface of the septum housing 108. During joining of the cannula assembly 104 to the infusion set base 106, one of the salients 388 may deflect the cantilevered section 180 of the base 106 until the protuberance 182 is free to spring back into a position overhanging the salient 388. Once the cantilevered projection 180 has restored into its undeflected state and the protuberance 182 is over the salient 388, the cannula subassembly 114 may be retained within the base 106. In the retained state (sec, e.g. FIG. 9), cars or nubs 204 of the septum housing 108 may at least partially reside within the notches 174 of the base 106. The receptacle 176 portion of the infusion set base 106 is widened with respect to that shown in FIG. 4A-B so as to accommodate the greater footprint of the cannula subassembly 114. Additionally, the receptacle 176 includes a salient receiving region 398 opposite the cantilevered projection 180 so as to accept the salient 388 not engaged by the protuberance 182. This may allow the cannula subassembly 114 to be made symmetrically and allow for it to be installed into the infusion set base 106 in two orientations. This may aid in simplifying manufacturing and assembly. In other embodiments, a salient 388 may only be included on one side of the cannula subassembly 114 and the salient receiving region 398 of the base 106 may be omitted.

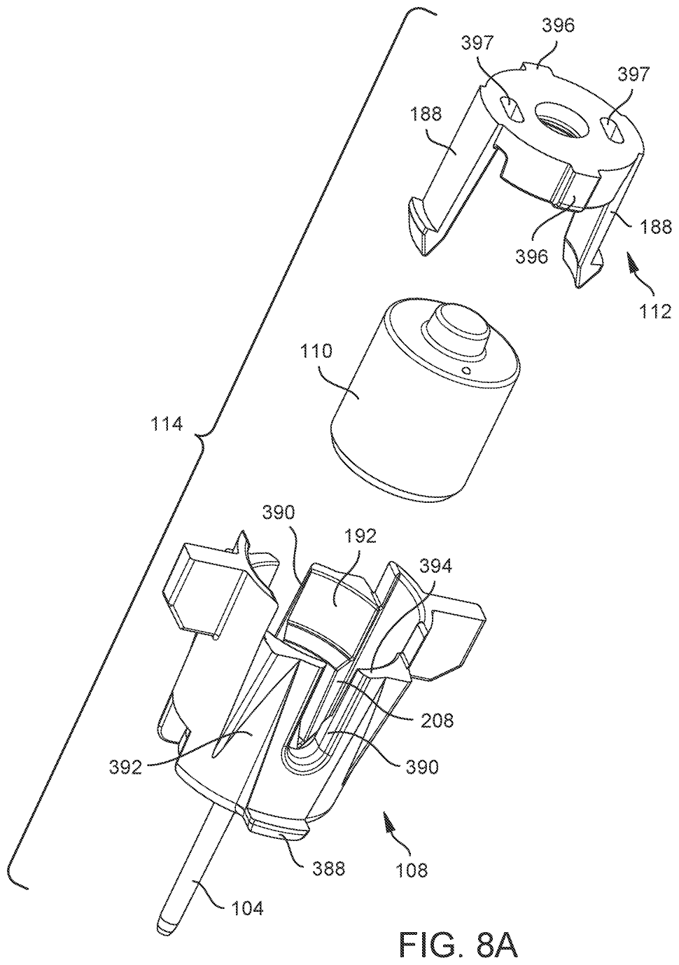

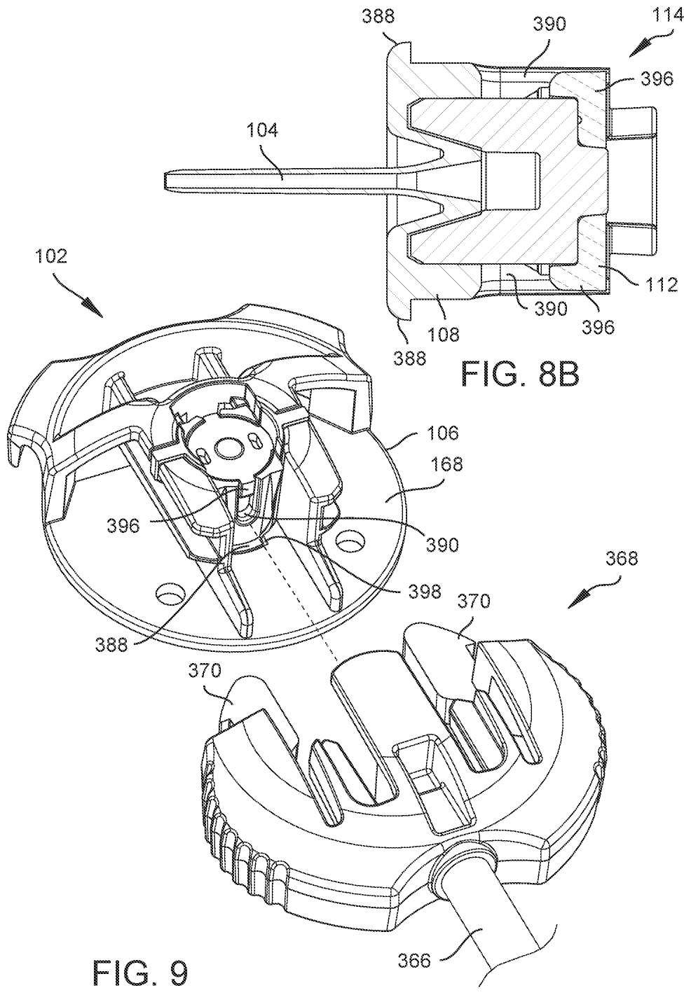

Additionally, in the exemplary embodiment, the septum housing 108 does not include fenestrations which form a connector needle passage 222 (see, e.g. FIG. 6) in a sidewall thereof. The septum housing 108 in FIGS. 8A-9 includes slots 390 in the side wall 392 of the septum housing 108 recessed into the top face 394 of the side wall 392. The septum retainer 112 may include projections 396 which align with the slots 390 when the cantilevered projections 188 are fitted within guides 208 included on the interior surface of the receiving section 192 of the septum housing 108. When the cannula subassembly 114 is coupled together (see, e.g. FIG. 8B), the projections 396 may occupy a portion of the slots 390. Thus an access hole for a sharp 482 (sec, e.g., FIG. 15D) or needle included on a tubing connector 368 may be provided in the cannula subassembly 114. The septum retainer 112 also includes a number of pass throughs 397. The pass throughs 397 may aid in manufacture and assembly similar to pass throughs 166 in the infusion set base 106.

In some embodiments, the slots 390 in the side wall 392 of the septum housing 108 may be used in place of the salient 388 as a retention arrangement which cooperates with a protuberance included as part of the base 106. The protuberance may catch against or engage with a wall of one of the slots 390 inhibiting removal of the cannula subassembly 114. In such embodiments, the salients 388 may not be included on the septum housing 108. This may help to further simplify production of the cannula subassembly 114.

Referring now to FIGS. 10A and 10B, another example base 106 is depicted. As in FIG. 6, the top face 168 of the platform portion 160 may include various receiving features extending therefrom which may mate or interface with a connector 368. The base 106 may include connector receivers 170 which may allow for coupling of the base 106 with cantilevered fingers 370 on the tubing set connector 368 as described above (see, e.g. description of FIG. 6). Guides 172 for each of the connector fingers 370 may also be included in the example embodiment. As shown, the exemplary guides 172 include a first section 171 and a second section 173. The first section 171 of each guide 172 may be disposed so as to have a portion which extends in a substantially parallel fashion to the plane of the platform 160 of the base 106. The second section 173 of each guide 172 may include a portion disposed at an angle with respect to the first section 171. In the example, each of the first and second portion 171, 173 include a ledge which projects from a main portion of that guide 172. The distance between the platform 160 and the proximal surface of the ledge of the second section 173 of the guide 172 may increase as the second section 173 extends distally from the ledge of the first portion 171 of the guide 172. This may allow the tubing set connector 368 to initially be introduced at a substantial angle to the plane of the platform 160 of the base 106. Further introduction may cause the connector fingers 370 to contact the platform 160 and be redirected to an appropriate displacement pathway for coupling of the tubing set connector 368 to the base 106. Thus, the guides 172 may aid in allowing a user to blindly insert the connector fingers 370 into the connector receivers 170 as coupling of a base 106 and tubing set connector 368 is performed. As shown, a second set of additional guide ledges 175 may be included on receptacle wall extensions 179. These additional guide ledges 175 may extend to the periphery of the base 106. In the example embodiments, the second set of guide ledges 175 extend substantially perpendicularly from the medial faces of the receptacle wall extensions 179 and toward the axis along which the sharp 482 (see, e.g., FIGS. 137-141) of the tubing set connector 368 is inserted into the cannula subassembly 114. A face of each of the second set of guide ledges 175 which is disposed near an end of each guide 172 most proximal to the periphery of the base 106 may include a ramped region.

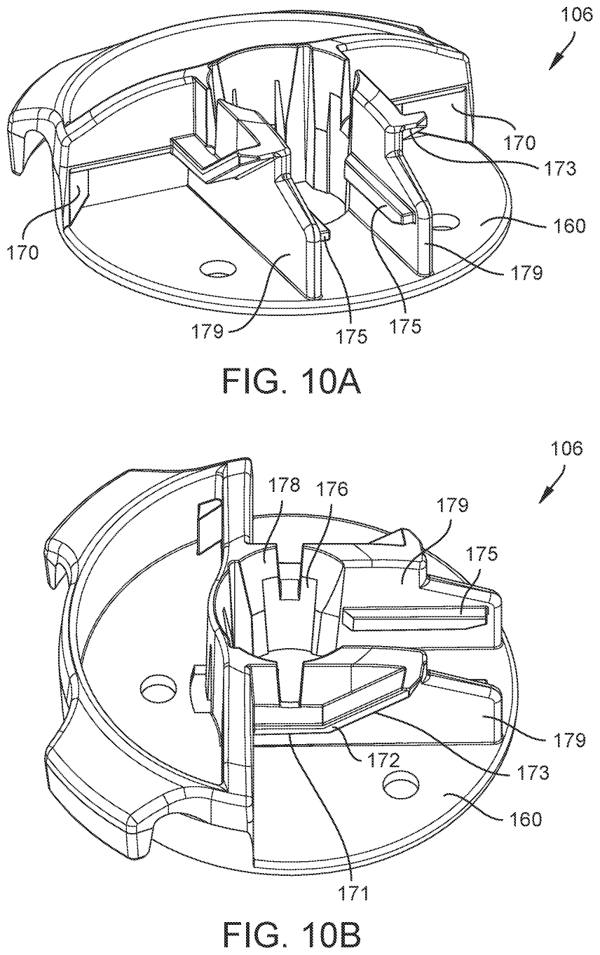

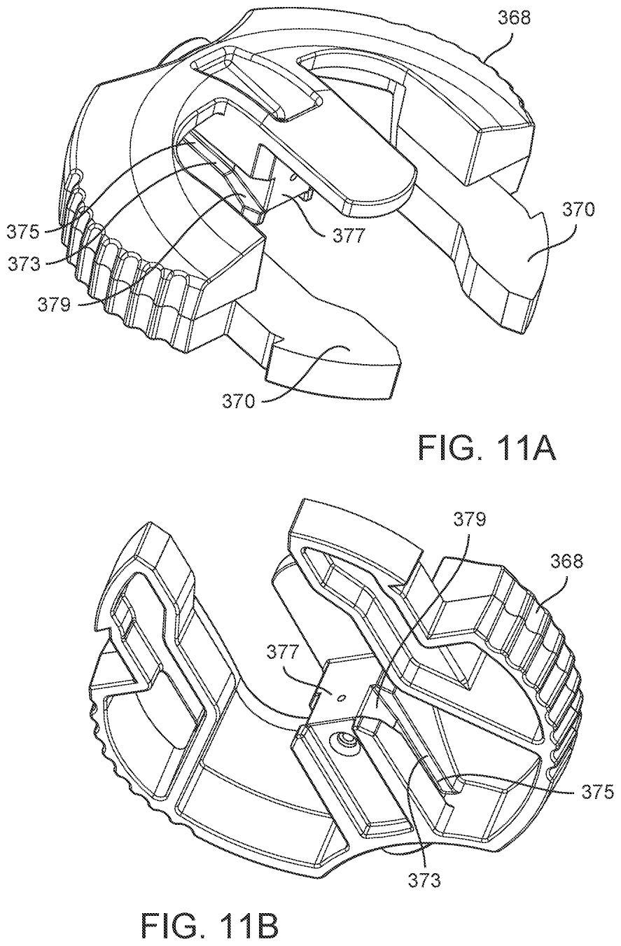

Referring now to also to FIGS. 11A-11B, the tubing set connector 368 may include a flow hub 377 to which tubing 366 and a sharp 482 (see, e.g., FIGS. 137-141) may be coupled. Fluid flowing through the connector 368 may pass through lumens 514 and 516 (sec, e.g., FIGS. 137-141) of the tubing 366 and sharp 482 respectively within the flow hub 377. As shown in FIGS. 11A-11B, the flow hub 377 may include alignment channels 375 recessed therein. As shown, the alignment channels 375 include a constant width segment 373 and a variable width segment 379. Upon coupling of a tubing set connector 368 and infusion set 102, the alignment channels 375 may interact with the second set of guide ledges 175 of the base 106. The ramped section of each of the second set of guide ledges 175 and the variable width section 379 of the alignment channels 375 may allow the tubing sharp connector 368 to initially be introduced at a substantial angle to the plane of the platform 160 of the base 106. Further introduction may cause the second set of guide ledges 175 to contact the wall of their respective alignment channels 375 and redirect the tubing set connector 368 toward an appropriate displacement pathway for coupling of the tubing set connector 368 to the base 106. The second set of guide ledges 175 in conjunction with the alignment channels 375 may thus aid in allowing a user to blindly couple the base 106 and tubing set connector 368.

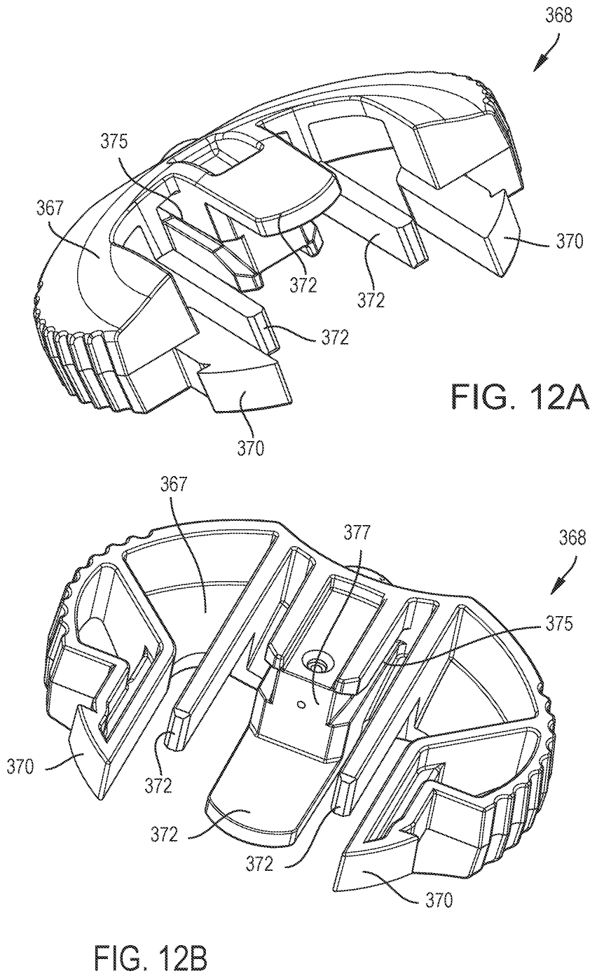

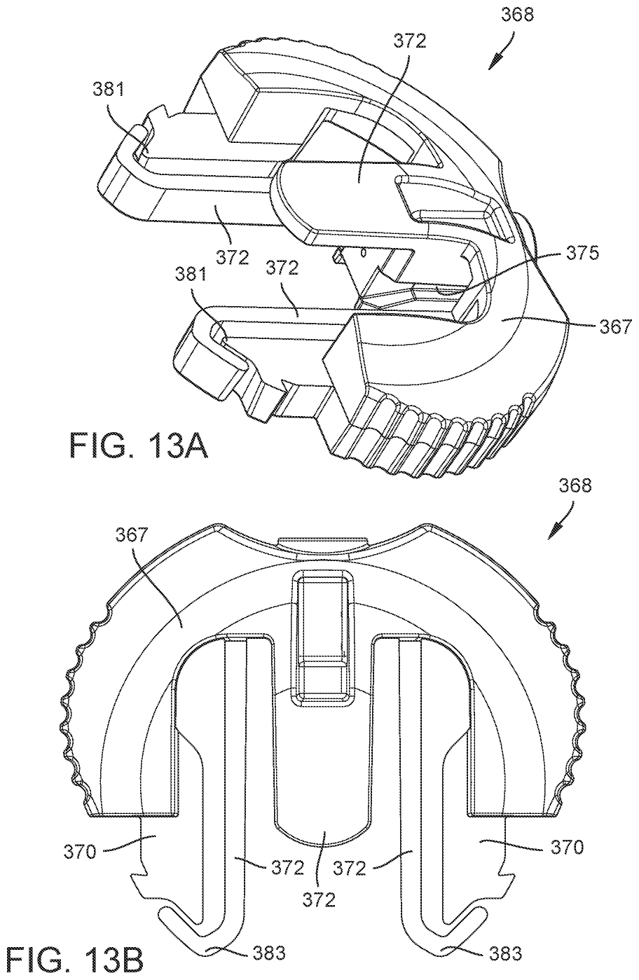

Referring now to FIGS. 12A-12B and FIGS. 13A-13B, in some embodiments, sharp flanking projections 372 may be included as part of the tubing set connector 368. As shown, the sharp flanking projections 372 may extend parallel to the axis of a sharp 482 (see, e.g., FIGS. 137-141) which may be included in the tubing set connector 368. The sharp flanking projections 372 may aid in preventing any user contact with the sharp 482 by providing an obstruction which inhibits a finger from reaching the sharp 482. The sharp flanking projections 372 may include a centrally disposed projection 372 which extends over the sharp 482 from a top face of the tubing set connector 368. The sharp flanking projections 372 may also include a set of projections which are in the same plane as the connector fingers 370 of the tubing set connector 368, but disposed more medially than the connector fingers 370. In the example embodiments, the connector fingers 370 and this set of projections 372 are in line with the bottom face of the tubing set connector 368. Each projection 372 of this set of projections 372 may be disposed so as to extend from a point on the body 367 of the tubing set connector 368 which is intermediate the flow hub 377 and the connector fingers 370. These projections 372 may be disposed so as to interact with guides 172 of a base 106 during coupling of the tubing set connector 368 to a corresponding base 106. In such embodiments, the connector fingers 370 may not interact with guides 172 on the base 106.

In some embodiments, and as shown in FIGS. 13A-13B, the pair of sharp flanking projections 372 which are in the plane of the connector fingers 370 may extend beyond the tips 381 of the connector fingers 370. These projections 372 may include a curved end region 383 at an end of the projections 372 opposite that attached to the body 367 of the tubing set connector 368. The curved end regions 383 may curve in a direction lateral to the axis of the sharp 482 (sec, e.g., FIGS. 137-141) of the tubing set connector 368. In the example, the curved end regions 383 may have a curvature which swings an are greater than 90°. In the example, the curved end regions 383 have a curvature such that the curved end regions 383 begin to extend back toward the body 367 of the tubing set connector 368. The radius of the curved end regions 383 may or may not be constant. Additionally, the curved end regions 383 may include one or more straight expanse. As shown best in FIG. 13B, the portion of the curved end region 383 leading to the terminus of the projection may be substantially straight. In the example, the curved end regions 383 curve around and in front of a portion of the respective connector fingers 370. The distance between the projections 372 may be slightly smaller than the spacing between the guides 172 of a corresponding base 106. The distance between the projections 372 at the curved end regions 383, however, may be slightly greater facilitating maneuvering of the projections 372 into their respective guides 172. Once the projections 372 are located within the guides 172, advancement of the tubing set connector 368 may cause the projections 372 to be resiliently splayed apart. Due to the resiliency of the projections 372, the projections 372 press against a surface of the guides 172 ensuring a tight and substantially wiggle free fit. This may help to further confine displacement of the tubing set connector 368 along a desired path as coupling of a tubing set connector 368 to a base 106 is completed.

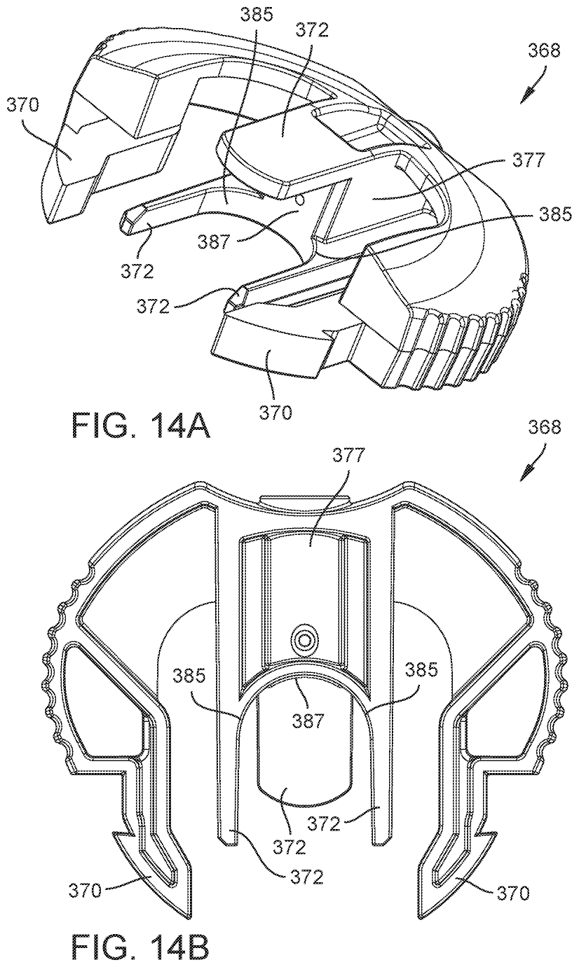

In certain examples, and referring now to FIGS. 14A-14B, a set of sharp flanking projections 372 in line with the connector fingers 370 may extend from the flow hub 377. This may decrease the gap between the sharp flanking projections 372 of the tubing set connector 368 and may help to further minimize the ability of the user to access space in the vicinity of the sharp 482 (see, e.g., FIGS. 137-141) with a finger. In the example embodiment depicted in FIGS. 14A-14B, a set of sharp flanking projections 372 which extend along and from opposing faces of the flow hub 377 lateral to the axis of the sharp 482 are included. These projections 372 may each include a support segment 385. These support segments 385 may be located in the region of the sharp flanking projections 372 which extends beyond the face 387 of the flow hub 377 from which the sharp 482 extends. As shown, the support segments 385 are disposed in the portion of this region most proximal to the flow hub 377. The support segments 385 in the example embodiment include an arched face which may lend additional robustness to the set of sharp flanking projections 372. The face 387 of the flow hub 377 from which the sharp 482 extends may also be arched which may further add robustness to the projections 372.

Referring now to FIGS. 15A-H, another exemplary tubing set connector 368 is depicted. As shown, a set of sharp flanking projections 372A which may be line with the connector fingers 370 may extend from the flow hub 377 similarly to FIGS. 14A-B. As in FIGS. 14A-B, this may aid in avoiding inadvertent contact with the sharp 482. A centrally disposed sharp flanking projection 372B which extends over the sharp 482 from a top face of the tubing set connector 368 may also be included.

The sharp flanking projections 372A, B may include at least one guide surface 371A-D. In some embodiments, each of the sharp flanking projections 372A, B may include at least one guide surface 371A-D. The sharp flanking projections 372A depicted in FIGS. 15A-H each include a lateral adjusting guide surface 371A and a height adjusting guide surface 371B. At least one of any lateral adjusting guide surface 371A and height adjusting guide surface 371B may include a sloped segment. As shown, the lateral guide surface 371A and height adjusting guide surface 371B may both include a first region proximal the flow hub 377 and a second region at the end of the sharp flanking projections 372A distal to the flow hub 377. The second regions may be sloped (ramped or curved) while the first regions may be substantially straight or sloped to a lesser degree.

The sharp flanking projection 372B may also include at least one height adjusting guide surface 371C and at least one lateral adjusting guide surface 371D. In the example, at least one of the height adjusting guide surface 371C and at least one lateral adjusting guide surface 371D may include a sloped section. In the example, the height adjusting guide surface 371C includes a first region proximal the flow hub 377 and a second region at the distal end of the sharp flanking projection 372B. The second region is sloped (ramped or curved) in the example embodiment. The sharp flanking projection 372B includes a centrally disposed trough 369 which runs along the underside (that closest to the sharp 482) of the sharp flanking projection 372B. The side walls of this trough 369 may form the two lateral guide surfaces 371D. A portion (e.g. that most distal the flow hub 377) of at least one of the lateral guide surfaces 371D may be sloped.

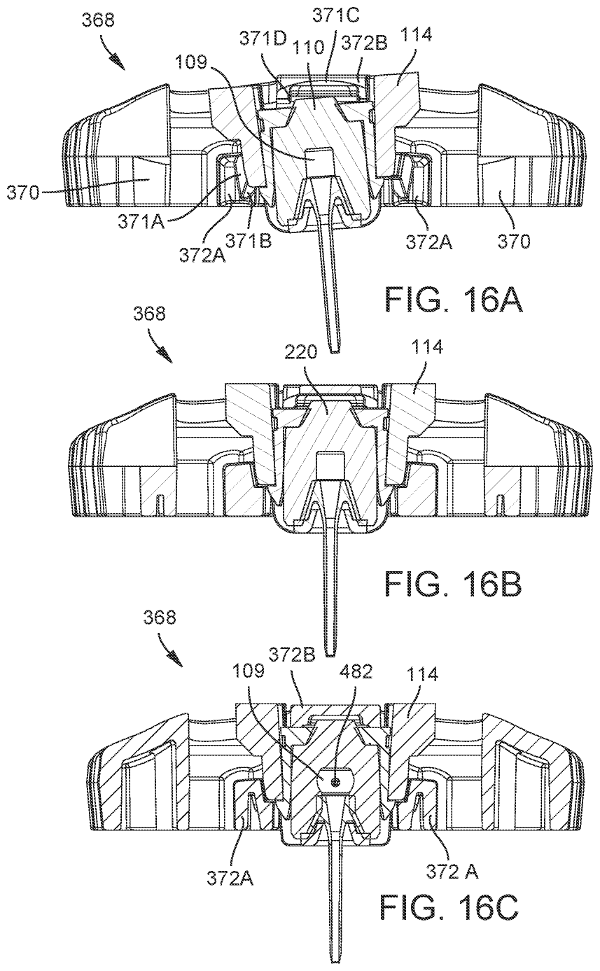

Referring now also to FIGS. 16A-16C, it may be desirable that portions of the infusion set base 106 and/or cannula subassembly 114 have relatively loose tolerance to facilitate case of manufacturing. Thus, in some embodiments, a cannula subassembly 114 may be slightly askew when retained in an infusion set base 106 (sec, e.g., FIG. 9) due to the loose tolerancing. Additionally, the cannula subassembly 114 may alter position slightly with repeated connection and disconnection of the tubing set connector 368 to the infusion set base 106.

FIGS. 16A-16C depict cross-sectional view through an exemplary cannula subassembly 114 which is held stationary as a tubing set connector 368 is progressively advanced closer to the cannula subassembly 114. As the tubing set connector 368 is docked to the infusion set base 106 (see, e.g., FIG. 9), the exemplary tubing set connector 368 shown in FIGS. 15A-15F may be displaced from an initial position (see, e.g., FIG. 16A) to an intermediary position just prior to the sharp 482 puncturing septum 110 material of the cannula subassembly 114 (sec, e.g. FIG. 16B). This range of displacement of the tubing set connector 368 may be referred to as a homing displacement range. As the tubing set connector 368 is displaced through the homing displacement range, any guide surfaces 371A-D of the tubing set connector 368 may contact various regions or surfaces of the cannula subassembly 114 in the event that the cannula subassembly 114 is askew. For illustrative purposes, a depiction of a cannula subassembly 114 in an exaggerated askew position is depicted in FIG. 16A. The infusion set base 106 to which the cannula subassembly 114 would be coupled is hidden for clarity.

As the tubing set connector 368 is further displaced though the homing displacement range, the guide surfaces 371A-D may push, direct, and/or adjust the cannula subassembly 114 into a home position. The sloped sections included on any of the guide surfaces 371A-D may facilitate this by helping to funnel the cannula subassembly 114 into or toward its home position. The cannula subassembly 114 is depicted adjusted into the home position in FIG. 16B (again infusion set base 106 hidden to show cannula subassembly 114 with greater clarity). The guide surfaces 371A-D may adjust the position of the cannula subassembly 114 along two axes which may be substantially perpendicular to one another and in a plane substantially perpendicular to the direction of elongation of the sharp 482 of the tubing set connector 368.

The tubing set connector 368 may then be displaced through a piercing displacement range in which the sharp 482 of the tubing set connector 368 pierces through the septum(s) 110 included in the cannula subassembly 114. At the end of the piercing displacement range and as shown in FIG. 16C, the tip of the sharp 482 may be disposed within the fluid introduction volume 109 of the septum 110 and the connector fingers 370 may enter into engagement with the connector receivers 170 on the infusion set base 106. During puncture of the septum(s) 110, the cannula subassembly 114 may be substantially inhibited from displacing by the guide surfaces 371A-D of the tubing set connector 368. The tubing set connector 368 may be considered to be in a fully docked position on the infusion set base 106 at the end of the piercing displacement range.

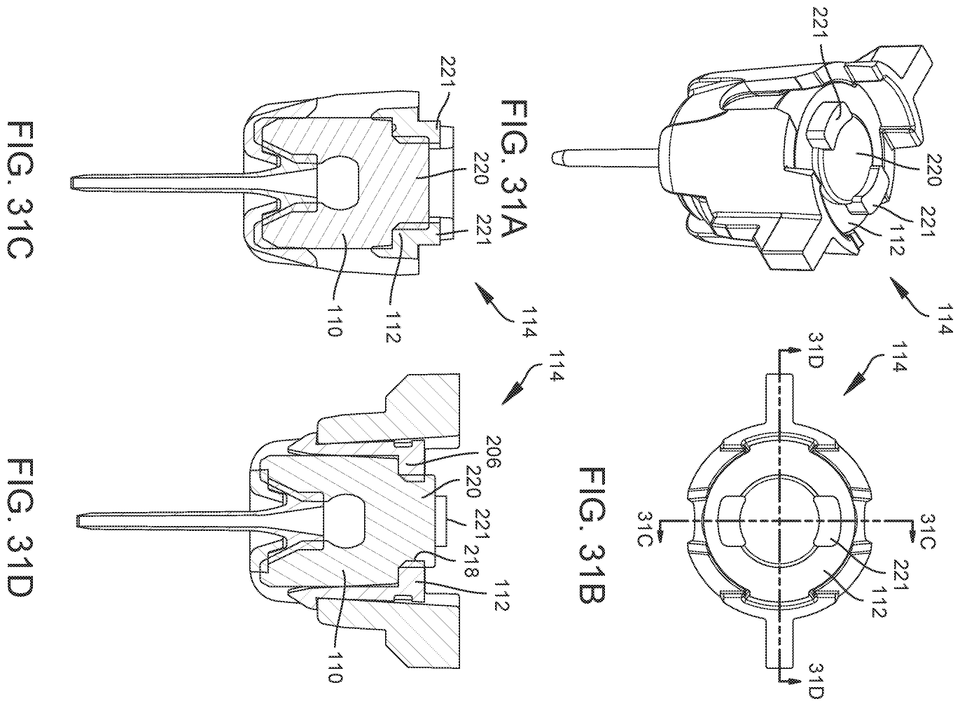

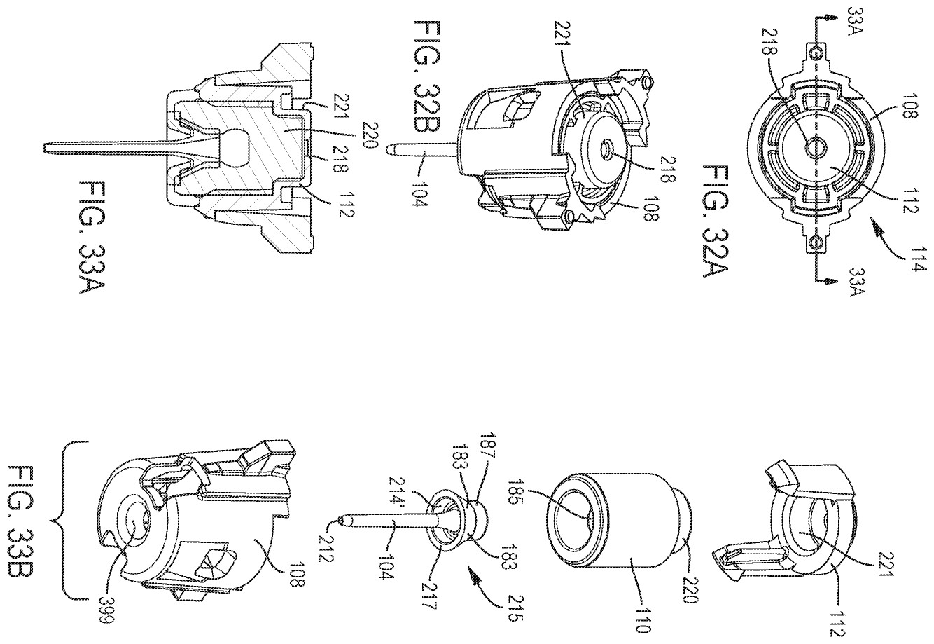

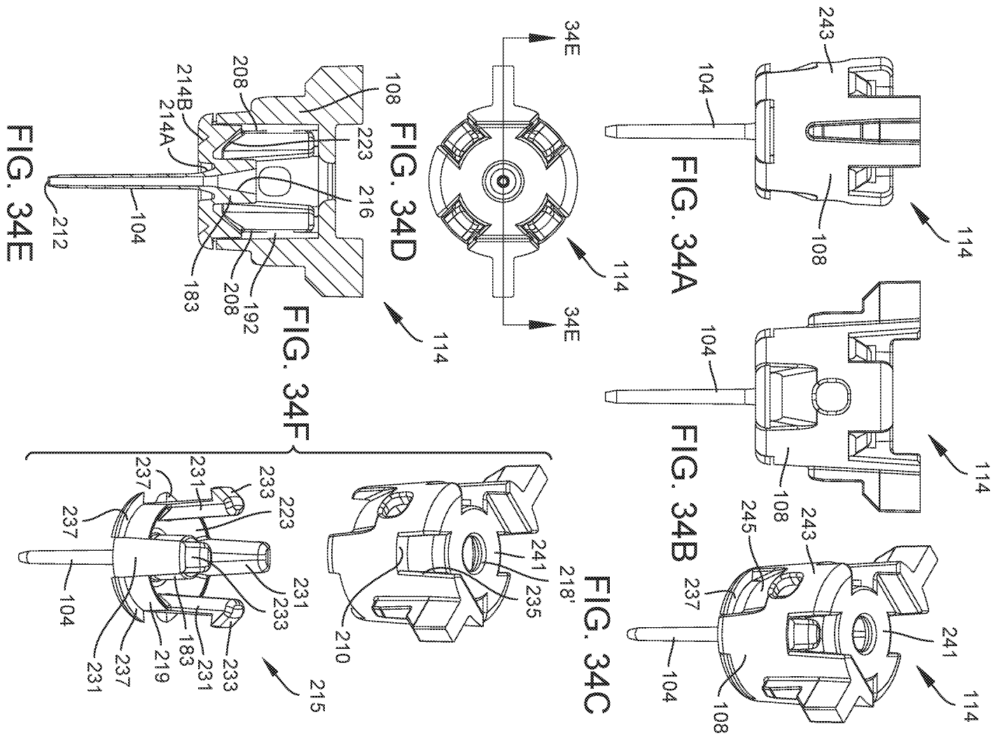

This two stage connection of the tubing set connector 368 to the infusion set base 106 may help to ensure that the cannula subassembly 114 septum(s) 110 are located in a consistent position prior to piercing of the septum(s) 110 by the sharp 482 of the tubing set connector 368. This may be true even in the event that loose tolerances are employed during manufacturing of certain components of the infusion set 102. The home position of the cannula subassembly 114 may place the septum(s) 110 in an appropriate position to allow the sharp 482 of the tubing set connector 368 to enter a fluid introduction volume 109 in the cannula subassembly 114. This may allow the fluid introduction volume 109 to be made smaller to minimize dead volume. Moreover, any connector needle passages 222 (see, e.g., FIG. 30B) or other openings in the septum housing 108 which allow the sharp 482 to pass through the septum housing 108 may be made smaller. Additionally, since the home position may be consistently achieved during each reconnection of the tubing set connector 368 to the infusion set base 106, the sharp 482 may puncture the septum(s) 110 in substantially the same location.

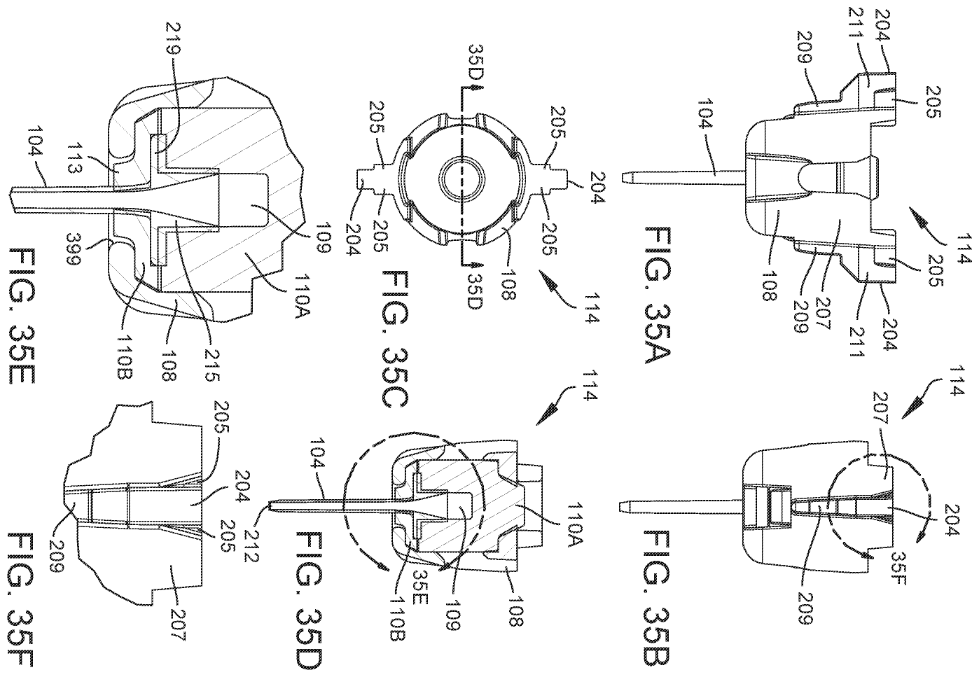

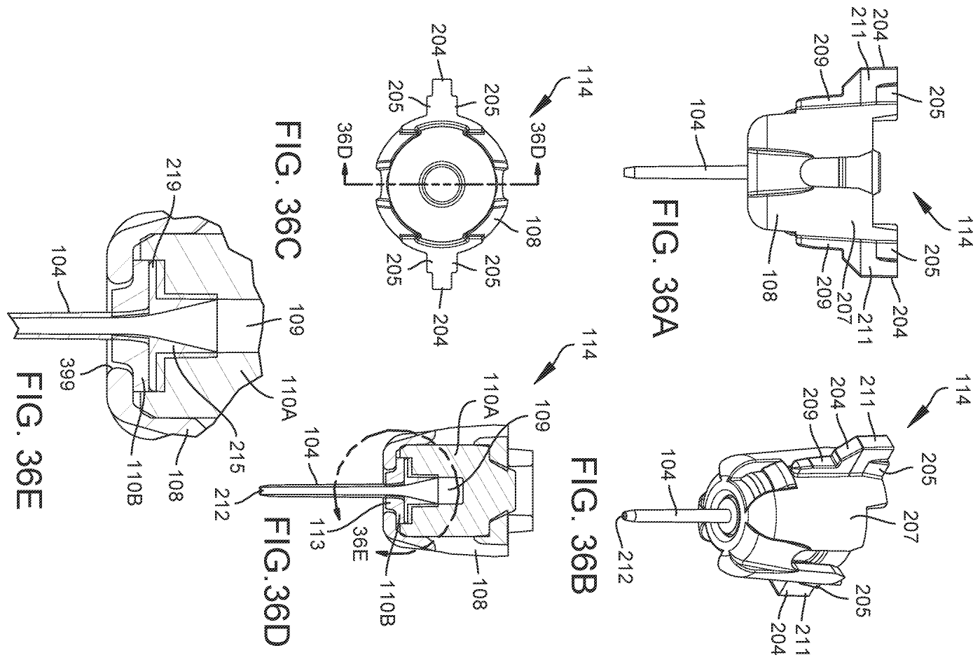

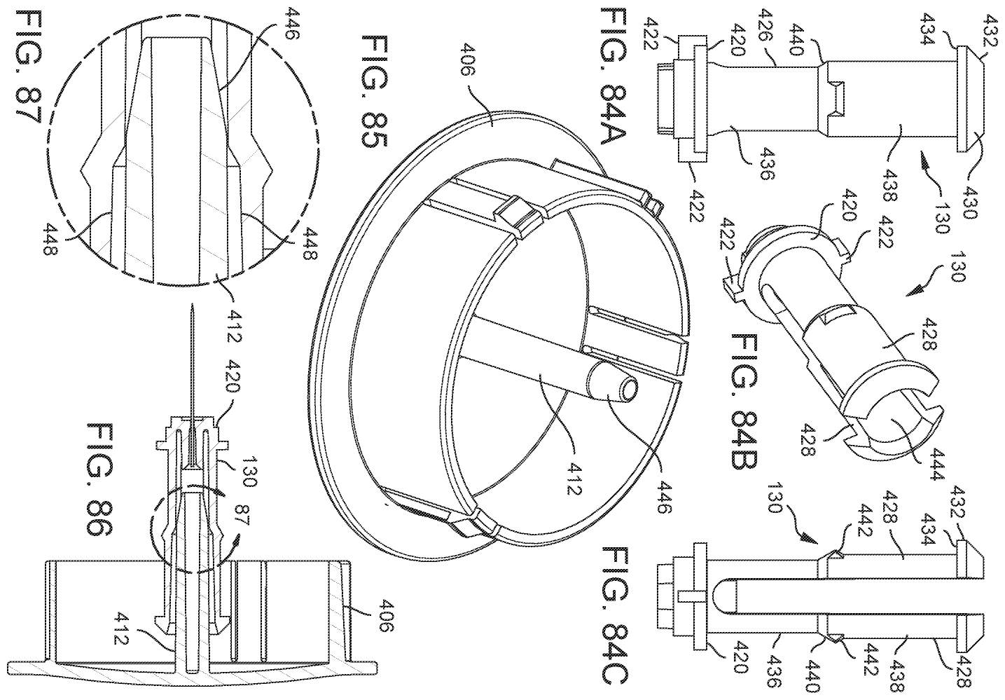

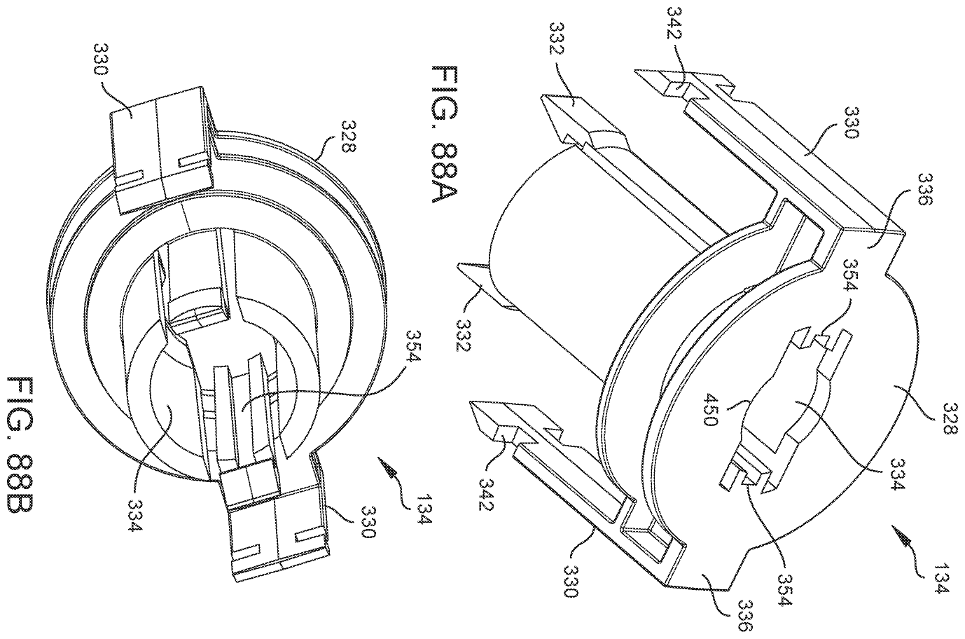

In certain examples, a mechanical interference may be present between at least one of the guide surfaces 371C-D on the sharp flanking projection 372B and the nub 220 of the septum 110. At least by the point that the tubing set connector 368 is displaced to the fully docked position, the mechanical interference may cause the nub 220 material to become compressed in a manner similar to the compression from projections 221 described in relation to FIGS. 31A-31D. Lateral adjusting guide surfaces 371D may, for example, cause the nub 220 material to compress toward the puncture pathway for the insertion sharp 132 (see, e.g., FIG. 74B). Height adjusting guide surfaces 371C may cause the nub 220 material to compress and seal against that guide surface 371C.

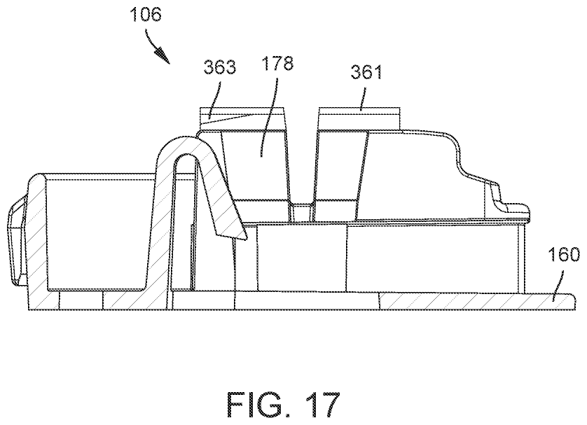

In some examples, and referring now primarily to FIG. 17, an infusion set base 106 may include a track 361 which may engage with and guide a sharp flanking projection 372 as a tubing set connector 368 (see, e.g., FIGS. 15A-15F) is docked to the infusion set base 106. The track 361 may include a deflector body 363 which may cause a sharp flanking projection 372 to resiliently deflect as a tubing set connector 368 and infusion set base 106 are coupled to one another. In the example shown in FIG. 17, the track 361 is arranged to interface with sharp flanking projection 372B (see, e.g., FIG. 15D) though tracks 361 for other sharp flanking projections may include deflector bodies 363 as well. The example track 361 extends from the portion of the receptacle wall 178 most distal to the platform portion 160 of the base 106. As shown, the deflector body 363 may be a ramp in certain examples. Upon contacting the deflector body 363, the sharp flanking projection 372B may be deflected and pressed against the nub 220 (sec, e.g., FIG. 5B) of the septum 110 of a cannula subassembly 114 installed in the infusion set base 106. Though the sharp flanking projection 372B may be deflected against the nub 220 in the example embodiment, sharp flanking projections 372A, B may be deflected against any exposed portion of a septum 110 of a cannula subassembly 114 in various embodiments.

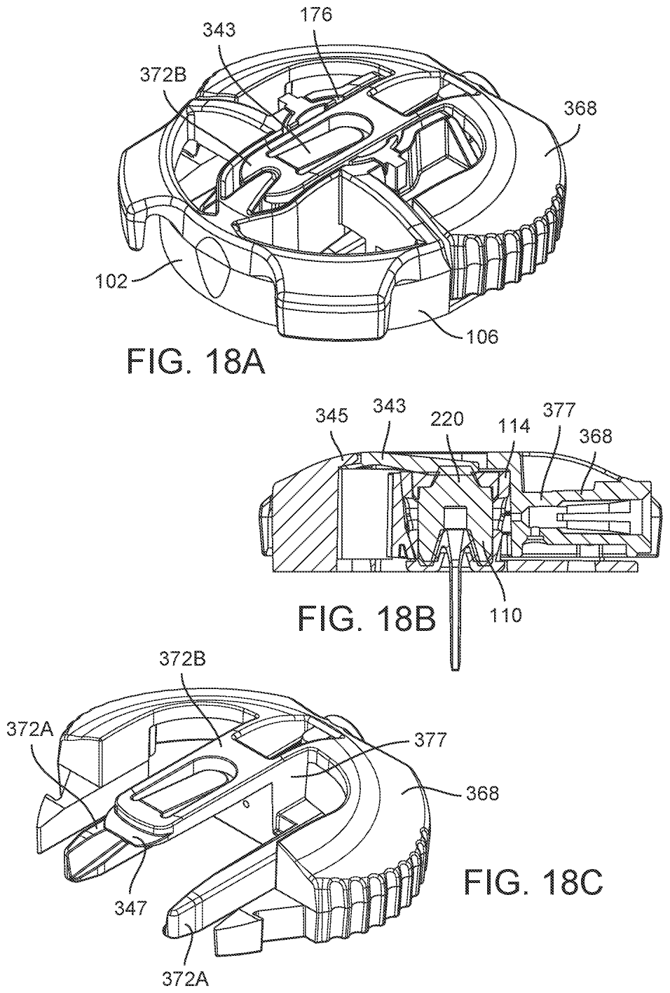

In some example embodiments, and referring now to FIGS. 18A-18C, a tubing set connector 368 may include a tine 343. The tine 343 may be included in a sharp flanking projection 372A, B of the tubing set connector 368. Such a tine 343 may be cantilevered with respect to the remainder of the sharp flanking projection 372A, B. The tine 343 may be constructed to have at least a sections which extends out of the plane of the remainder sharp flanking projection 372A, B. The tine 343 may be deflected toward the sharp flanking projection 372A, B, but may possess a degree of resiliently which urges the tine 343 to restore to its unstressed state when in a deflected state.

In the example embodiment, the tine 343 is included in sharp flanking projection 372B of the tubing set connector 368. The tine 343 is attached to the sharp flanking projection 372B in a region of the tine 343 which is most distal the flow hub 377 of the tubing set connector 368. The unsupported end of the tine 343 is included in a portion of the tine 343 which extends substantially parallel to the remainder of sharp flanking projection 372B. When the tubing set connector 368 is coupled to an infusion set 102, the tine 343 may be advanced over a cannula subassembly 114 coupled within a receptacle 176 of the infusion set base 106. The tine 343 may resiliently deflect as this occurs. When in the coupled state, tine 343 may be prevented from fully returning to the unstressed state by the cannula subassembly 114. Instead, the tine 343 may press against the nub 220 of the septum 110 of cannula subassembly 114.

In some embodiments, the end of the sharp flanking projection 372B most distal the flow hub 377 may include ramp 347. Such a ramp 347 may be included on a portion of the end (see, e.g., FIG. 18B) or may extend across the entirety of the end (sec, e.g., FIG. 18C). The infusion set base 106 may include a deflector 345 into which the ramp 347 may be advanced when the tubing set connector 368 is coupled to the infusion set 102. The deflector 345 may be a projection extending from a portion of the infusion set base 106 in some embodiments. Alternatively, the deflector 345 may be a slot in the infusion set base 106 into which the ramp 347 is displaced during coupling of a tubing set connector 368 to an infusion set assembly 102. As the ramp 347 is displaced into the slot 345, the sharp flanking projection 372B may be deflected toward the platform portion 160 of the infusion set base 106. This may, in turn, cause the tine 343 to be further pressed against the nub 220.

Though shown in relation to FIGS. 18A-18C, tines 343 and ramps 347 may be included on any tubing set connectors 368 described herein. Deflectors 345 may be included on any infusion set base 106 described herein.

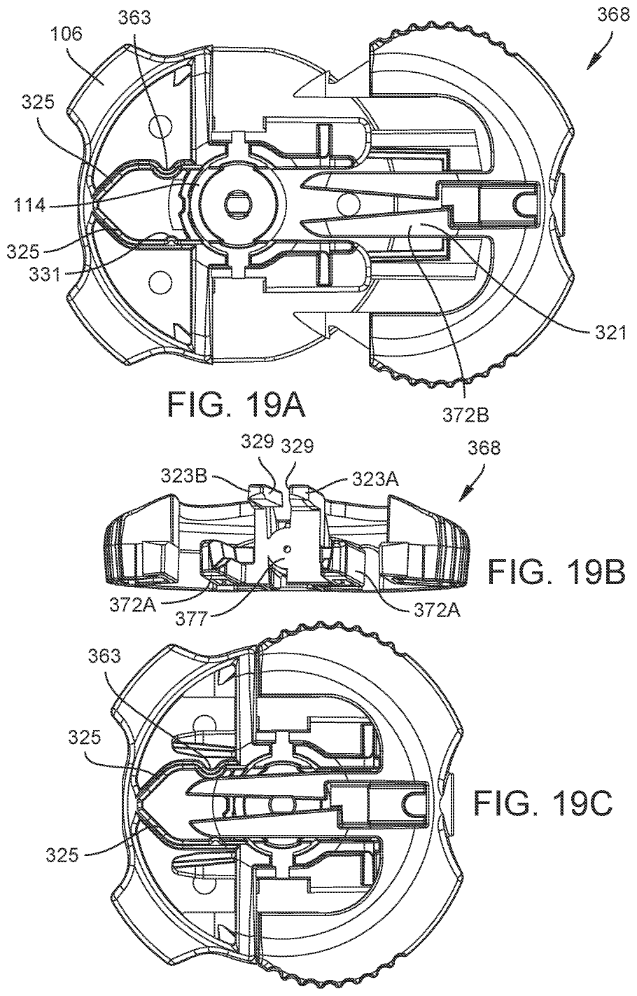

Referring now to FIGS. 19A-19C, in some embodiments a tubing set connector 368 may include a clamp 321. As a tubing set connector 368 including a clamp 321 is coupled into engagement with an infusion set assembly 102, the clamp 321 may act against an exposed portion of a septum 110 of a cannula subassembly 114 of the infusion set assembly 102. The clamp 321 may, for example, press into opposing sides of an exposed portion of a septum 110 (e.g. a nub 220) to compress the septum 110 material.

A clamp 321 may, in some examples, be defined in a sharp flanking projection 372 or may form a sharp flanking projection 372. As shown, sharp flanking projection 372B is formed as a clamp 321. The clamp 321 may include a first body 323A and a second body 323B which are spaced from one another by a gap. At least one of the first and second body 323A, B may be configured to displace toward the other of the first and second bodies 323A, B. Any suitable arrangement to facilitate such displacement may be used. In the exemplary embodiment and as best shown in FIG. 19B, the second body 323B is coupled to the flow hub 377 along a portion of an end of the second body 323B proximate the flow hub 377. This partial connection may allow the second body 323 to resiliently deflect or pivot toward the first body 323A. The first body 323A may be connected along the entirety of its second end to the flow hub 377. Thus, the example first body 323A may be resistant to deflection and may remain stationary.

The infusion set assembly 102 may include a deflector body 363. In the example embodiment, the infusion set base 106 includes a set of ribs 325. The deflector body 363 is defined as an outcrop or bump in one of the ribs 325. As the tubing set connector 368 is coupled into engagement with an infusion set assembly 102, a portion of the clamp 321 may be displaced into the deflector body 363. Further displacement of the tubing set connector 368 may cause deflector body 363 to deflect a part of the clamp 321. In the example embodiment, the second body 323B may be deflected toward the first body 323A as the second body 323B is driven into the deflector body 363. In turn, the clamp 321 may close about the nub 220 of the septum 110. Clamping surfaces 329 of the first and second bodies 323A, B may press against and compress the nub 220. To help inhibit turning or veering of the tubing set connector 368 as the clamp 321 contacts the deflector body 363, a stop member 331 may be included. The stop member 331 may present a mechanical interference to undesired movement of the tubing set connector 368 during coupling. In the example embodiment, the stop member 331 is a bulge on the rib 325 opposite the deflector body 363.

Referring to FIGS. 15A-19C, compression of the nub 220 of the septum 110 and ensuring a sharp 482 of a tubing set connector 368 punctures a septum 110 in substantially the same location as described above may be desirable for a variety of reasons. For example, these features may help extend a range over which a cannula subassembly 114 may resist leaks to pressures even further in excess of those expected during use. Compression of the nub 220 may allow for a wider variety of materials to be used for a septum 110 of a cannula assembly 114. Likewise, a greater range of material durometers may be used. Compression of the nub 220 via projections 221 (sec, e.g., FIGS. 31A-31D) or lowering potential compressive stress relaxation of the material forming the nub 220 (see discussion in relation to FIGS. 31A-31D) may also aid in accomplishing the above.

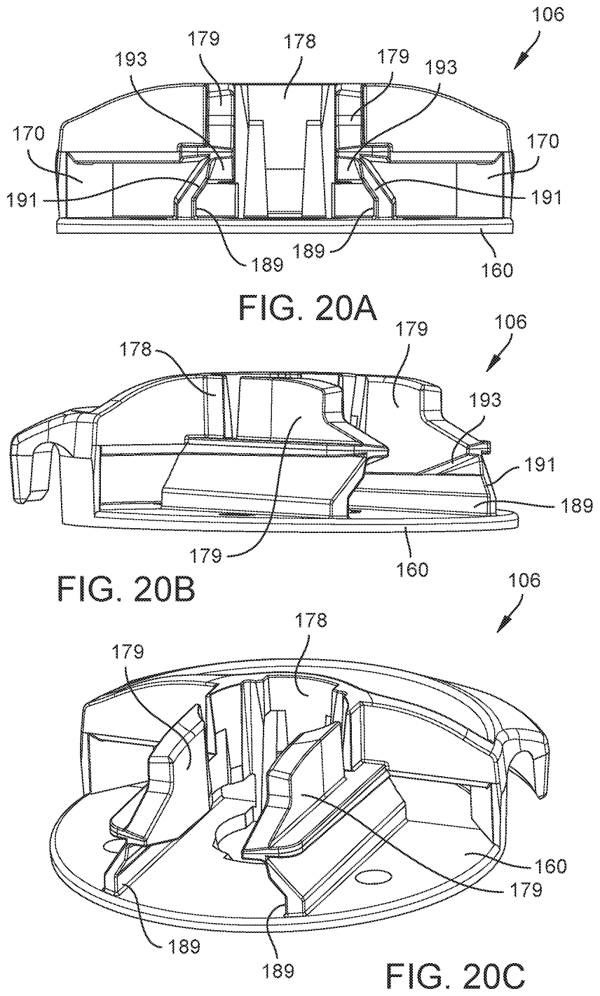

Referring now also to FIGS. 20A-20C, another example base 106 is depicted. The example base 106 may couple to a tubing set connector 368 such as that shown in FIGS. 14A and 14B. The base 106 may include connector receivers 170 which may allow for coupling of the base 106 with cantilevered fingers 370 on the tubing set connector 368 as described elsewhere herein. Though not shown in the example embodiment, guides 172 for each of the connector fingers 370 such as those shown in FIG. 6 and FIG. 10A may be included. Such guides 172 may also be included on any other infusion set base 106 shown or described herein. As shown, the base 106 may define receiver tracks 189 which may accept a set of sharp flanking projections 372 which are in the plane of the connector fingers 370. As shown, the receptacle wall extensions 179 and the portions of the receptacle wall 178 which extend parallel to one another may be separated from one another by a channel. The receiver tracks 189 are recessed into the receptacle wall extensions 179 and the portions of the receptacle wall 178 which extend parallel to one another adjacent the platform 160. Thus, the width of the channel between the receptacle wall extension 179 and portions of the receptacle wall 178 may be greatest adjacent the platform 160. The width of the channel may decrease as distance from the platform 160 increases. As shown, the receiver tracks 189 may each include a sloped wall 191. The sloped wall 191 may aid in locating of the sharp flanking projections 372 of a tubing set connector 368 in the receiver tracks 189. Additionally, the base 106 may include ramped sections 193 which are located at the open end of the receiver tracks 189. The ramped section 193 may, similarly to the second section 173 of the guides 172 in FIGS. 10A-10B, allow the tubing set connector 368 to initially be introduced at a substantial angle to the plane of the platform 160. Further introduction may cause the flanking projections 372 to be redirected into the receiver tracks 189. Thus, the sloped walls 191 and ramped section 193 may facilitate blind insertion of the tubing set connector 368 into coupled relationship with the base 106.

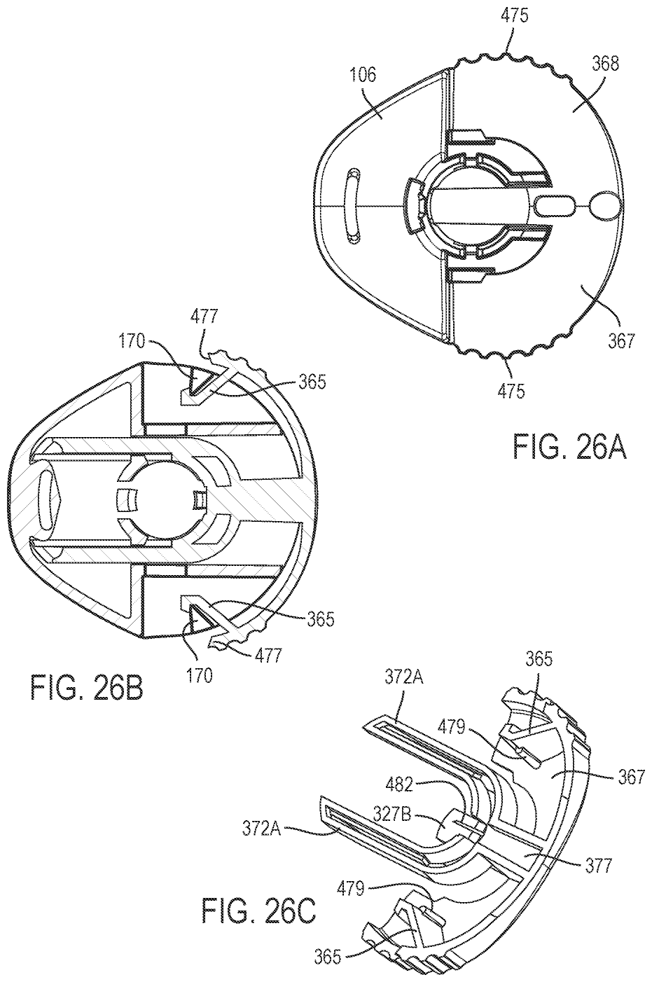

Referring now to FIGS. 21A-24C, additional example infusion set bases 106 are depicted. The bases 106 may include connector receivers 170 which may allow for coupling of the base 106 a tubing set connector 368. The example base 106 shown in FIGS. 21A-F may couple to a tubing set connector 368 such as that shown in FIGS. 15A-16C as described elsewhere herein. The example base 106 shown in FIGS. 22A-22C may couple to a tubing set connector 368 such as those shown in FIGS. 25A-26C.

As shown, the receptacle wall extensions 179 may be separated from one another by a channel. The receptacle wall extensions 179 and the portions of the receptacle wall 178 forming each side of the channel may also include a segment 177 which is disposed substantially parallel to the platform portion 160 of the infusion set base 106. Thus, the channel may include a wide section adjacent the platform portion 160 and a narrow portion distal to the platform portion 160. The wide section of the channel may define receiver tracks 189 which may accept a set of sharp flanking projections 372A. Though not shown in FIGS. 21A-21F and FIGS. 22A-22C, ramped sections 193 (see, e.g., FIGS. 20A-20C) may be included in some embodiments. The receiver tracks 189 may guide sharp flanking projections 372A as a tubing set connector 368 is coupled to a base 106. The channel may thus be referred to as a guide channel.

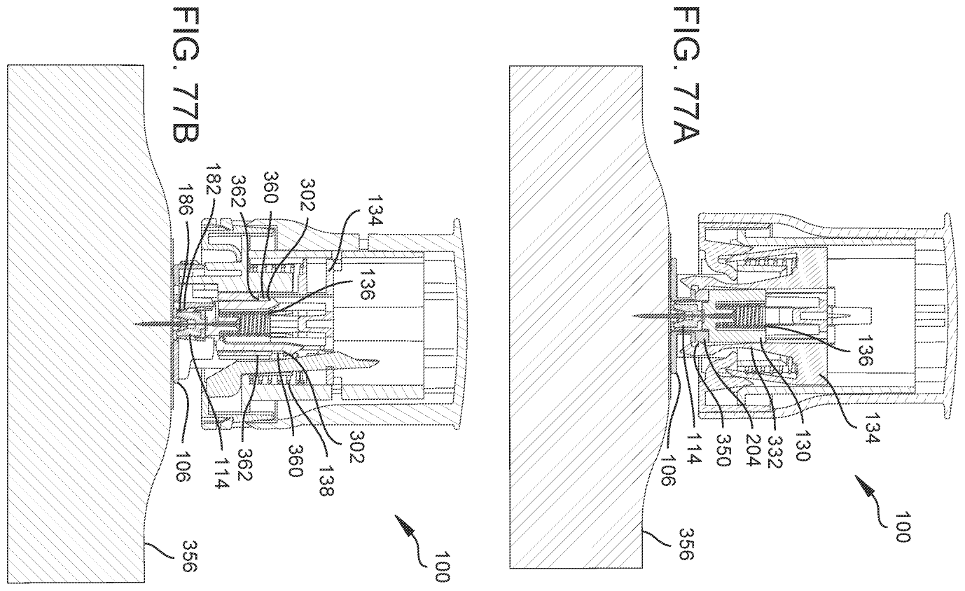

As shown, the receptacle walls 178 may include breaks 181 in the portion of the receptacle walls 178 defining the receiver tracks 189. The breaks 181 may be aligned with the notches 174 included in the receptacle walls 178. The breaks 181 may extend through a portion of the segments 177 of the receptacle wall 178 which is parallel to the platform portion 160. The more medial regions 161 of these segment 177 may be uninterrupted by the breaks 181. The breaks 181 may extend from the segments 177 to the platform portion 160. The breaks 181 may define receiving slots for a portion of a respective arm 332 (see, e.g., FIG. 59A) of part of an inserter assembly 100. These receiving slots may also be referred to as inserter interface slots. The medial regions 161 adjacent the breaks 181 may act as a catch for a ledge 348 defined on the arms 332 mentioned above (further described in relation to FIG. 59A). As best shown in FIG. 21D, the notches 174 in the receptacle wall 178 may be tapered along at least a portion of their length (e.g. portion most distal to the platform portion 160). Such tapered notches 174 may aid in guiding a cannula subassembly 114 into place as the infusion set 102 is assembled. In some embodiments, the entirety of each notch 174 may be tapered to varying degrees. For example, a portion of each notch 174 most distal the platform portion 160 may have a greater taper than a second portion of each notch 174 more proximal the platform portion 160. Such tapered notches 174 may be included on any of the infusion set bases 106 described or shown herein.

The example infusion set base 106 in FIGS. 21A-F 106 also includes a receptacle wall 178 including a cantilevered section 180 with a protuberance 182. The cantilevered section 180 may be constructed so as to resiliently deflect out of the way as a cannula assembly 114 is introduced, but resist deflection when a force exerted along the long axis of the cantilevered section 180 is applied. The cantilevered section 180 may include one or more ribs 163. The rib(s) 163 may project from the cantilevered section 180 into the receptacle 176 and may extend along the length of the cantilevered section 180. Though ribs 163 are shown, other projecting bodies may be included in alternative embodiments. One or more recess 165 may be disposed on a side of the cantilevered section 180 opposite that from which the protuberance 182 extends. A recess 165 may be disposed opposite each of the ribs 163 or otherwise thickened regions. As a cannula assembly 114 is introduced, the rib(s) 163 may help to locate the cannula subassembly 114 within the receptacle 176. The rib(s) 163 may also inhibit wiggling of the cannula subassembly 114 when the cannula subassembly 114 is within the receptacle 176 and coupled to the base 106.

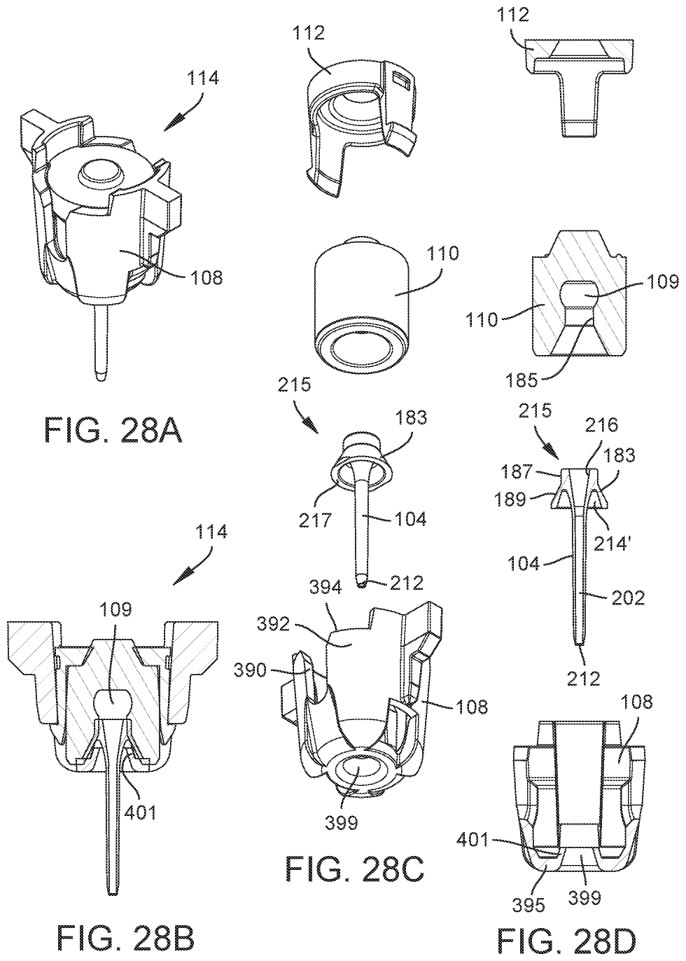

The example infusion set base 106 depicted in FIGS. 22A-22C includes a plurality of cantilevered sections 180 each including protuberances 182. The receptacle wall 178 includes a cantilevered section 180. This cantilevered section 180 projects from the receptacle wall 178 away from the platform portion 160 such that the unsupported end including the protuberance 182 is the portion of the cantilevered section 180 most distal to the platform portion 160. A pair of cantilevered sections 180 are also disposed opposite one another on the receptacle wall extensions 179 adjacent the receptacle wall 178. This pair of cantilevered sections 180 extend in a direction away from (e.g. substantially perpendicular to) the platform portion 160 such that the unsupported ends including the respective protuberances 182 are the portions of the cantilevered sections 180 most distal to the platform portion 160. As a cannula subassembly 114 is introduced, each of the cantilevered sections 180 may resiliently deflect out of the way to allow passage of the cannula subassembly 114 into the infusion set base 106. The ramped faces of the protuberances 182 may aid in facilitating this deflection. Once the cannula subassembly 114 has advanced beyond a threshold amount into the receptacle 176 of the infusion set base 106, the cannula subassembly 114 may be displaced clear of the protuberances 182. The cantilevered sections 180 may each restore to a less stressed or less deflected state and the protuberances 182 may each overhang a portion of the cannula subassembly 114 (e.g. the septum housing 108). Thus, a cannula subassembly 114 may be captured within the infusion set base 106. Though three cantilevered sections 180 are shown, differing numbers of cantilevered sections 180 may be included in alternative embodiments. The receptacle wall 178 may, for example, include a greater number of cantilevered sections 180. In alternative embodiments, at least some of the cantilevered sections 180 may project toward the platform portion 160 such that their unsupported, protuberance 182 bearing ends are the portions of the cantilevered sections 180 most proximal the platform portion 160 (sec, e.g., cantilevered section 180 of FIGS. 21A-F). The height of each cantilevered sections 180 is substantially the same in FIGS. 22A-22C. In alternative embodiments, the height of at least one cantilevered section 180 may differ. The heights may be selected such that the protuberances 182 overhang and capture the cannula subassembly 114 to be installed in the receptacle 176. Cannula subassemblies 114 for use with infusion set base 106 embodiments such as that shown in FIGS. 22A-22C may not include notches 186 (see, e.g., FIG. 5A) or salient 388 (sec, e.g., FIG. 8A). The top face 394 (see, e.g., FIG. 28C) of the side wall 392 of the septum housing 108 (see, e.g., FIG. 28C) may provide the engagement surface by which the protuberances 182 retain the cannula subassembly 114 in the base 106.

Referring now to FIGS. 23A-24C, two exemplary infusion set base embodiments are depicted. As shown, in certain examples, an additional retainer member 261 may be included and may project from the platform portion 160 of the infusion set base 106. The example embodiments depict the retainer members 261 on an infusion set base 106 similar to that depicted in FIGS. 22A-22C, however, such retainer members 261 may be included on any infusion set base 106 described herein (e.g. that shown in FIGS. 21A-F). The retainer members 261 are disposed adjacent the receptacle 176 and nearest the open end of the guide channel. In the example embodiments, the retainer members 261 are positioned opposite cantilevered sections 180. Such an example retainer members 261 may be hooked or hook like in shape. As a tubing set connector 368 is coupled to the exemplary infusion set bases 106, a sharp 482 (see, e.g., FIG. 15F) may travel along an approach path within the respective guide channels. The height of the retainer members 261 may be selected so as to not project into the approach path of the sharp 482, thus the retainer members 261 may not obstruct the sharp 482 during coupling of a tubing set connector 368. As shown in various embodiments described herein, a cannula subassembly 114 may include a set of notches 186 (see, e.g., FIG. 5A or FIG. 24E). The notches 186 may be disposed on opposing sections of the exterior side wall of a portion of the cannula subassembly 114.

With reference to FIGS. 23A-23C, the cantilevered section 180 of the receptacle wall 178 may snap into a first of the notches 186 retaining the cannula subassembly 114 within the receptacle 176 after the cannula subassembly 114 has been advanced into the receptacle 176 beyond a certain distance. The retainer member 261 may deflect as the cannula subassembly 114 is advanced into the receptacle 176. The retainer member 261 may restore to an undeflected (or at least less deflected) state and snap into the second of the notches 186 when the cannula subassembly 114 has been advanced into the receptacle 176 beyond a certain distance. In various embodiments, the cantilevered section 180 and retainer member 261 may snap into the notches 186 at substantially the same time. In the example embodiment shown in FIGS. 24A-24C, the protuberance 182 of the cantilevered member 180 may clip in place against the top face 394 of the septum housing 108 of the cannula subassembly 114. The retainer member 261 may help to limit jostling of the cannula subassembly 114 within the receptacle 176 and potentially allow for looser tolerancing. Additionally, the retainer member 261 may assist in centering the cannula subassembly 114 within the receptacle 176.

Referring now to FIGS. 25A-26C, example tubing set connectors 368 which may be used, for example, with an infusion set base 106 such as those shown in FIGS. 22A-24C are depicted. The example tubing set connectors 386 include sharp flanking projections 372A, B, but do not include connector fingers 370. The example tubing set connectors 368 include a set of connector latches 365. Though shown in relation to FIGS. 25A-26C, connector latches 365 may be included in place of connector fingers 370 of other embodiments of tubing set connectors 368 described herein. The example connector latches 365 may be disposed on an undersurface of the body 367. As the tubing set connectors 368 are coupled to an infusion set 102, the connector latches 365 may engage the connector receivers 170 on the infusion set base 106. The body 367 of the tubing set connector 368 may double as a shield which may help to block fingers or objects from inadvertently disengaging the connector latches 365 from the connector receivers 170. To facilitate this, and referring now also to FIGS. 22A-24C, the connector receivers 170 may be on opposing portions of the base 106 intermediate the closed and open ends of the guide channel and lateral to the receptacle wall extensions 179. The portions of the body 367 including the connector latches 365 may attach to the flow hub 377 so as to be deflectable relative to the flow hub 377 and may be referred to herein as deflectable body segments. Squeezing force toward the axis of the sharp 482 exerted on these segments of body 367 may displace the connector latches 365 out of engagement with the connector receivers 170. The tubing set connector 368 may then be separated from the infusion set 102 if desired. These segments of the body 367 may include uneven, scalloped, ruffled, etc. gripping regions 475 to assist in grasping of the tubing set connector 368. In some examples (best shown in FIG. 26C), the connector latches 365 may include stop bodies 479. The stop bodies 479 may limit deflection of the deflectable body segments as they may abut against the receptacle wall extensions 179 when deflected beyond a certain amount. Additionally, as best shown in FIG. 26B, in certain example tubing set connectors 368, the tubing set connector 368 may have an angled face 477 on each deflectable body segment. When the deflectable body segments are squeezed toward one another, the angled faces 479 may be displaced against the connector receivers 170. The angled faces 479 may cause the tubing set connector 368 to push away from the engaged state as the angled faces 479 are driven into the connector receivers 170 on the infusion set base 106. Thus, the angled faces 479 may facilitate decoupling of a tubing set connector 368 from the infusion set base 106.

Still referring to FIGS. 22A-26C, as the example tubing set connector 368 covers the couplings between the connector latches 365 and the connector receivers 170, a shielding wall 169 (see, e.g., FIG. 21A) is not included in the example infusion set base 106 of FIGS. 22A-24C. When coupled together, the example tubing set connector 368 and base 106 may form a low profile, contoured assembly which mitigates potential for snagging of, for example, clothing.

Still referring primarily to FIGS. 22A-24C, the portion of the example base 106 opposite the open end of the guide channel, may be formed by a contoured wall 357. The contoured wall 357 may extend from the top of the receptacle wall 178 to a portion of the periphery of the base 106 opposite the open end of the guide channel. The contoured wall 357 may trend toward dropping in the direction of the plane of the platform portion 160 as proximity to the periphery of the base 106 increases. The platform portion 160 may not continue under the contoured wall 357. Instead, a cavity may be present on the underside of the contoured wall 357 which may help facilitate molding. As shown in FIG. 22B for instance, a transition wall 349 may extend from the platform portion 160 to the edge of the contoured wall 357 most proximal the open end of the guide channel. The transition wall 349 may be disposed generally perpendicular to the axis of the guide channel in some embodiments.

Referring now also to FIGS. 25A-C, the transition wall 349 may include passages 341 which extend through the transition wall 349 and into the cavity on the underside of the contoured wall 357. When the tubing set connector 368 is coupled to the infusion set base 106, the sharp flanking projections 372A may extend through passages 341 in the transition wall 349. The unsupported ends of the sharp flanking projections 372A may project into the cavity. The contoured wall 357 of the infusion set base 106 may also be substantially even with the exterior surface of the body 367 of the tubing set connector 368. The deflectable body segments of the tubing set connector 368 body 367 may seat against the transition wall 349 of the infusions set base 106 such that no substantial gap is present between the transition wall 349 and the tubing set connector 368 when the base 106 and tubing set connector 368 are connected.

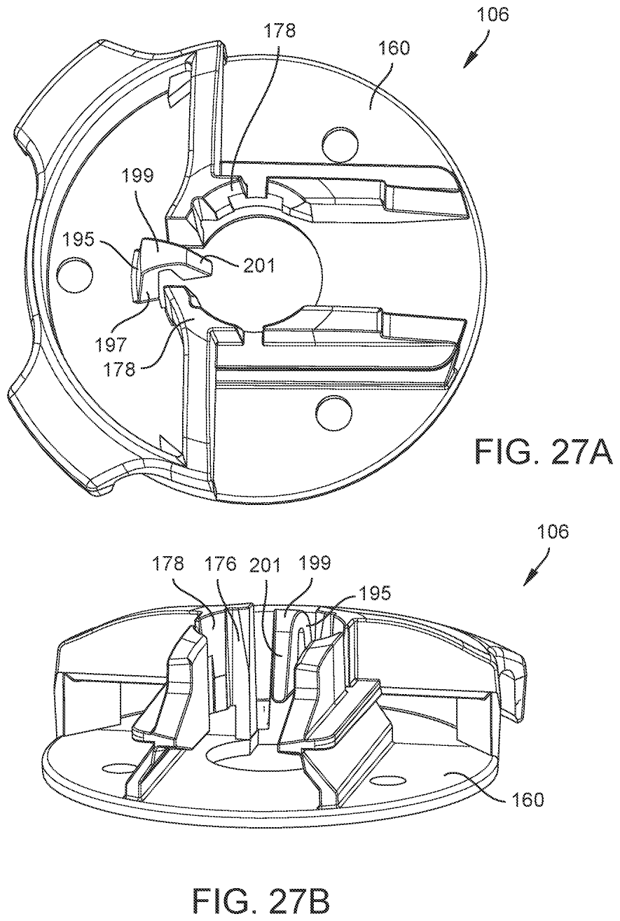

Referring now to FIG. 27A and FIG. 27B, another example infusion set base 106 is shown. In certain embodiments, a base 106 may not include a receptacle wall 178 which has a cantilevered projection 180 (sec, e.g., FIG. 4A) defined therein. As shown in FIG. 27A and FIG. 27B, in some examples, a base 106 may include a retainer member 195 which extends from the platform 160. The retainer member 195 may be freestanding and not supported by or continuous with the receptacle wall 178. In FIG. 27A and FIG. 27B, the retainer member 195 is depicted as a hooked body which extends from the platform and is located in an opening between the two sections of the receptacle wall 178. In other examples, a hooked body may not be used. Instead, a cantilevered member including a protuberance 182 such as a barb or ramp which engages a portion of a cannula subassembly 114 may be used. The hooked body may include a shank region 197 which is attached to and continuous with the platform 160. The shank region 197 may extend from the platform 160 to a bend region 199. The shank region 197 may be perpendicular to the platform 160 or may be angled so as to extend toward the receptacle 176. The hooked body may also include a catch segment 201 which extends from the bend region 199. The catch segment 201 may be disposed orthogonally to the shank region 197. In the example, the catch segment 201 extends at an acute angle with respect to the shank 197. For example, the catch segment 201 may be disposed at an angle of 35-40° with respect to the shank 197.

Upon introduction of a cannula subassembly 114 into the base 106, the septum housing 108 (see, e.g., FIG. 8A) may contact the catch segment 201 and resiliently deform the retainer member 195 such that the catch segment 201 is bent out of the way. After advancement of the cannula subassembly 114 into the receptacle 176 beyond a certain point, an engagement surface of the septum housing 108 may pass the end of the catch segment 201. At this point, the catch segment 201 may be free to spring back from its deflected position. The catch segment 201 may include a least a portion which overhangs the engagement surface of the septum housing 108 once sprung back to its unstressed state. Thus, the catch segment 201 may inhibit removal of the cannula subassembly 114 from the base 106. The engagement surface of the septum housing 108 may be any suitable engagement surface on any portion of the cannula subassembly 118. For example, the engagement surface may be a notch 186 (see, e.g. FIG. 5A) in the septum housing 108, a wall of a fenestration in the septum housing 108 (see, e.g. connector needle passage 222 of FIG. 6), a wall of a slot 390 included in the septum housing 108, or a salient 388 included as part of the septum housing 108.

Referring now to FIG. 28A-28D, various views of an example embodiment of a cannula subassembly 114 are depicted. The example cannula subassembly 114 includes a septum housing 108, cannula 104, septum 110, and a septum retainer 112. The septum housing 108 includes slots 390 in the side wall 392 of the septum housing 108 recessed into the top face 394 of the side wall 392. When installed into the cannula subassembly 114, the septum retainer 112 may block access to the sides of the septum 110 in a region proximal the top face 394 of the septum housing 108. A region of the septum 110 between the septum retainer 112 and the end of the slot 390 may be exposed to provide an access for a sharp 482 (see, e.g., FIG. 15D) included on a tubing connector 368. The terminal end of the slot 390 may provide an engagement surface for a cantilevered projection 180 (see, e.g., FIG. 4A) or retainer member 195 (sec, e.g., FIG. 27A) which may capture the cannula subassembly 114 within an infusion set base 106.

As shown, the cannula 104 and the septum housing 108 are depicted as separate discrete components in FIGS. 28A-28D. Though shown as discrete components, the cannula 104 and septum housing 108 may be integral to one another as discussed elsewhere herein. The example cannula 104 may include an enlarged end region 215. The enlarged end region 215 may include an insertion sharp guide section 216 which may be continuous with the walls of the lumen 202 of the cannula 104. The insertion sharp guide section 216 may aid directing an insertion sharp 132 (sec, e.g., FIG. 1A) into the lumen 202 of the cannula 104 during assembly. The enlarged end region 215 may also include a septum interface region 183 upon which a corresponding portion 185 of the septum 110 may be seated. The septum interface region 183 may surround the insertion sharp guide section 216. The septum interface region 183 may contact the corresponding portion 185 of the septum 110 and provide a sealing surface against which the septum 110 may be compressed to form a fluid tight seal when the cannula subassembly 114 is assembled.

The septum interface region 183 may have a number of different geometries depending on the embodiment. In the example, the septum interface region 183 may have a substantially straight walled portion 187 along a section most distal the outlet of the cannula 104. The straight walled portion 187 may extend substantially parallel to the long axis of the cannula 104. The straight walled portion 187 may transition to a conic frustum type shape section 189 which widens as the septum interface region 183 extends toward the cannula 104 outlet 212. In alternative embodiments, the entire septum interface region 183 may be straight walled (see, e.g., FIG. 30E) or may be in the shape of a conic frustum.

A recess 214′ may be included in a face of the enlarged portion 215 which surrounds, or at least partially surrounds, the cannula 104. In the example embodiment, the recess 214′ is approximately shaped in the form of a conic section (e.g. hyperbola or parabola) revolved around the center axis of the cannula 104. The septum housing 108 includes a passage 399 which extends through the bottom wall 395 of the septum housing 108. A cannula seat 401, which may be a raised wall or post surrounding the passage 399, may also be included in the septum housing 108. The recess 214′ in the enlarged portion 215 of the cannula 104 may accept at least a portion of the cannula seat 401 and may aid in locating and retaining the cannula 104 in place within the cannula subassembly 114. The bottom face 217 (that most proximal the outlet 212 of the cannula 104) of the enlarged portion 215 may rest against the bottom wall 395. When the cannula assembly 114 is assembled, the cannula 104 may extend through the passage 399 such that the outlet 212 is external to the septum housing 108. A volume may be present between the exterior of the cannula 104 and the side walls of the passage 399. This volume may provide room for the cannula 104 to displace relative to the base 106 if the infusion set 102 or a portion of the patient's body causes a force to be applied to the cannula 104. This may minimize shearing action on the cannula 104. Additionally, this volume may serve as a volume into which an agent may be placed. Any agent(s) discussed in relation to FIG. 5B may, for example, be used.

As shown, the septum 110 may include a fluid introduction volume 109. The example fluid introduction volume 109 includes at least one rounded sidewall. In the example, the fluid introduction volume 109 is in the shape of a spherical slice in which opposing sphere caps have been removed. In other embodiments, a generally globe shaped or otherwise rounded volume with no straight side walls may be used. Such a shape may help to ensure a relatively large fluid introduction volume 109 exists even under distortion of the volume which may occur when the septum 110 becomes compressed during assembly of the cannula subassembly 114. Additionally, such a shape may allow for a larger fluid introduction volume 109 without any change in the size of the enlarged portion 215 of the cannula 104. This may result in the fluid introduction volume 109 being a larger target for a sharp 482 of a tubing set connector 368 (sec, e.g., FIGS. 15A-16C). Consequently, it may be possible to construct components of the infusion set 102 or tubing set connector 368 with looser tolerancing thus simplifying manufacturing.

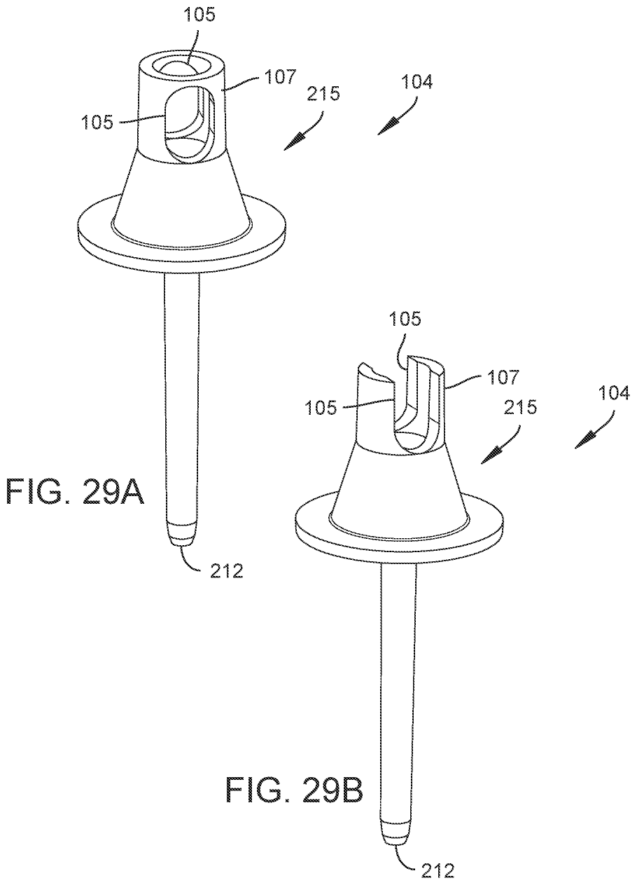

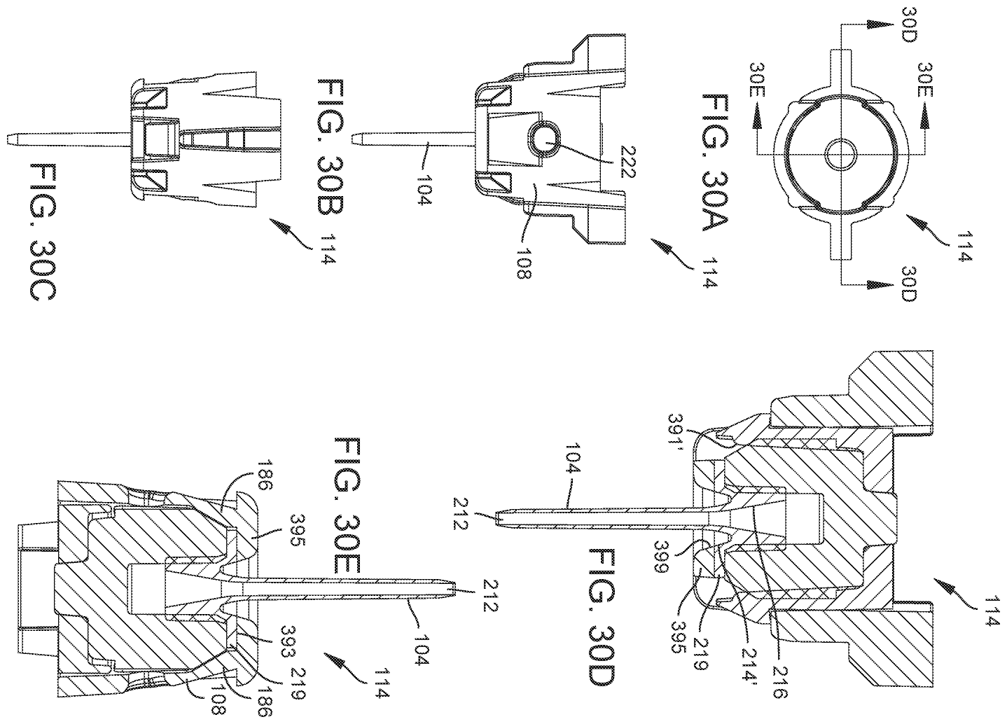

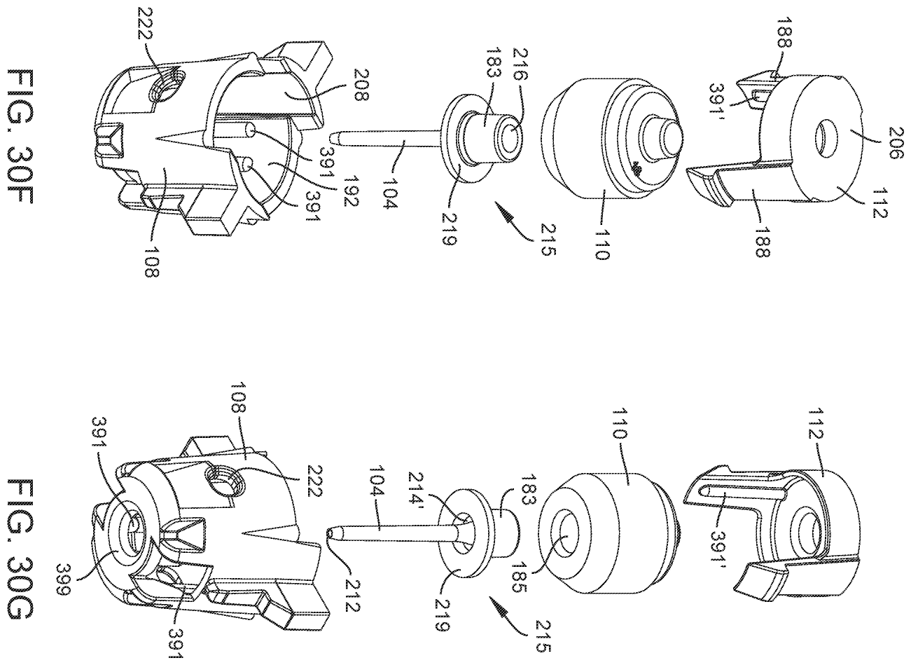

Referring now also to FIGS. 29A-29B, two example cannula embodiments are depicted. In some embodiments, there may be a fluid introduction volume reinforcing body 107 at an end of the enlarged region 215 most distal the outlet 212 of the cannula 104. The reinforcing body 107 may extend into the fluid introduction volume 109 and may abut walls sidewalls of the fluid introduction volume 109. The reinforcing body 107 may prevent distortion of the fluid introduction volume 109 when the septum 110 becomes compressed. The reinforcing body 107 may include passages 105 which may align with the slots 390 (see, e.g., FIG. 28C or connector needle passage (see, e.g., FIG. 6). This may allow for a sharp 482 (sec, e.g., FIG. 15C) on a tubing set connector 368 (see, e.g., FIG. 15C) a path through the reinforcing body 107 and into the fluid introduction volume 109 during connection of the tubing set connector 368 to an infusion set base 106. As shown, the passages 105 may be fenestrations (obround in the example, but could be any suitable shape) or slots. Such reinforcing bodies 107 may be included on any of the cannulas 104 described and/or shown herein.

Referring now to FIGS. 30A-30G, another exemplary cannula assembly 114 is depicted. As in FIGS. 28A-28D, the example cannula subassembly 114 includes a cannula 104 and septum housing 108 which are separate discrete components (though they could be integral with one another in alternative embodiments). The cannula 104 may have an enlarged region 215 which includes a septum interface region 183 that surrounds the insertion sharp guide 216 of the cannula 104. When assembled, a corresponding portion 185 of the septum 110 may be seated against the septum interface region 183. The enlarged region 215 may also include a flange 219 which may be disposed at an end of the enlarged region 215 most proximal the outlet 212 of the cannula 104. As best shown in FIG. 30E, the bottom wall 395 of the septum housing 108 may include a seat for the cannula 104. In the example, the seat is shown as a receptacle 393 into which the flange 219 may be seated. The receptacle 393 may aid in centering the cannula 104 within the septum housing 108.

A recess 214′ may be included in a face of the enlarged portion 215 which surrounds, or at least partially surrounds, the cannula 104. In the example embodiment, the recess 214′ is approximately shaped in the form of a conic section (e.g. hyperbola or parabola) revolved around the center axis of the cannula 104. The septum housing 108 includes a passage 399 which extends through the bottom wall 395 of the septum housing 108. The walls of the passage 399 may be angled or contoured to match and form a continuation of the recess 214′. This may create a volume which may be filled with agent (described elsewhere herein) and/or allow for some movement of the cannula 104 with respect to the infusion set base 106. The receptacle 393 (or other seat) may surround the passage 399.

The septum housing 108 may include a number of protuberances 391 (best shown in FIG. 30F) which extend from the wall of the receiving section 192 of the septum housing 108 toward the axis of elongation of the cannula 104. In the example embodiment, these protuberances 391 are in the form of substantially straight ridges or ribs. Each of the protuberances 391 may be the same length or at least one of the protuberances 391 may have a length which differs from at least one of the other protuberances 391. In certain embodiments, ends of the protuberances 391 most distal the cannula 104 outlet 212 may be ramped or curved (shown in FIG. 30F). Each of the connector needle passages 222 (an aperture in the example shown but potentially slots 390 in alternative examples where slots 390 are present in the septum housing 108) may be flanked by protuberances 391 which may run directly adjacent each side of the connector needle passages 222. The protuberances 391 may contact the septum 110 (or at least one of the septums 110 in embodiments with a plurality of septums 110) when the septum 110 is installed in the septum housing 108. Spaces between the protuberances 391 may provide a volume into which material of the septum 110 may be squeezed when the cannula subassembly 114 is assembled and the septum 110 is under compression. The septum retainer 112 also includes a set of protuberances 391′ included on the cantilevered projections 188 that extend from the body 206 of the septum retainer 112. When the cannula subassembly 114 is fully assembled, the protuberances 391, 391′ may contact the septum 110 and provide additional compression of the septum 110 to further increase robustness of the fluid tight seals formed by the septum 110. Such protuberances 391, 391′ may also allow for less stringent tolerancing to be used in order to help simplify manufacturing.

Referring now to FIGS. 31A-31D, another example cannula subassembly 114 is depicted. The example cannula subassembly 114 includes a septum retainer 112 which includes a channel 218 substantially centrally disposed in the body 206 of the septum retainer 112. A nub 220 of the septum 110 may project into and perhaps partially through the channel 218. The nub 220 of the septum 110 may have a diameter which is slightly larger than the diameter of the channel 218. Thus, the nub 220 may be compressed by the channel 218. Additionally, the channel 218 may have a diameter which extends across a large portion or a majority of the diameter of the body 206 of the septum retainer 112. This may spread compression exerted by the channel 218 walls over a relatively large amount of septum 110 material. As a result, compressive stress relaxation of the septum 110 material may be decreased over the shelf life of an inserter assembly 100.

The insertion sharp 132 of an inserter assembly 100 may extend through the nub 220 (sec, e.g. FIG. 74B) before the inserter assembly 100 is used. The septum 110 may be completely self-sealing upon removal of the insertion sharp 132 and leak-proof thereafter. In certain examples, the average width or diameter of the nub 220 may be selected such that the diameter of the insertion sharp 132 is less than 10% the average width of the nub 220. This may further aid in reducing stress relaxation of the septum 110 material during storage while an inserter assembly 110 is awaiting use.

As shown, the channel 218 may include sidewalls which are primarily straight and extend parallel to the axis of elongation of the cannula 104. Thus, the compression exerted by the sidewalls of the channel 218 against the nub 220 may be directed primarily normal to the axis of elongation of the cannula 104. This may maximize compression exerted by the channel 218 toward the puncture path of the insertion sharp 132. Moreover, by decreasing compressive stress relaxation of the septum 110, a greater degree of resiliency may be maintained in the septum 110 material. Thus, the channel 218 of FIGS. 31A-31D may help may resist leaks at pressures even further in excess of those expected during use. Additional potential benefits are described above in relation to FIGS. 15A-16C and FIG. 17.

Example septum retainers 112 may also include one or more projection 221 raised from the exposed surface of the septum retainer 112 which may at least partially surround the channel 218. In some embodiments, the entirety of the channel 218 may be surrounded. In the example embodiment, the projections 221 are included as part of a crenellated wall surrounding the channel 218. In the example, only two merlons and associated crenels are shown. The merlons may be disposed in opposition to one another about the channel 218. In other embodiments, any suitable number of crenellations may be included and the crenellations may or may not be spaced at regular angular intervals. In the example, the at least one projection 221 extends substantially perpendicular to the main body 206 of the septum retainer 112. In other embodiments the at least one projection 221 may extend toward the axis of the channel 218 or have a portion which extends toward the axis of the channel 218.

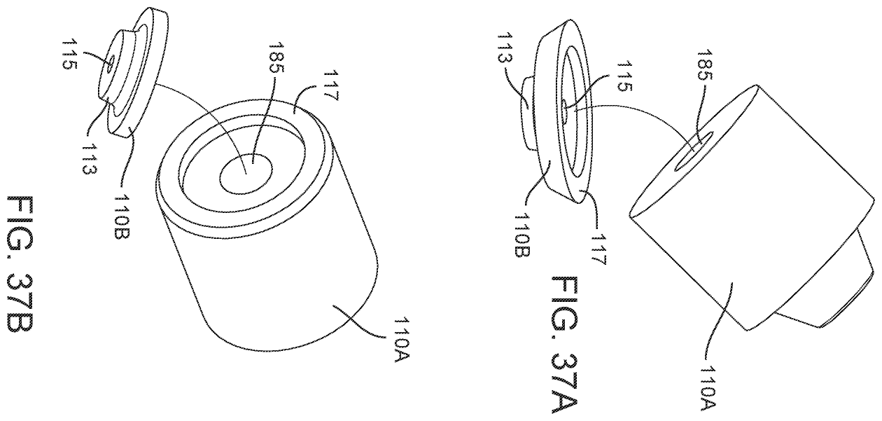

Referring now also to FIGS. 32A-33B, in some examples, the channel 218 may be entirely surrounded by a projection 221 having at least a portion angled toward the axis of the channel 218. Thus, the projection 221 may block all but a small portion of the nub 220. The projection 221 in FIGS. 32A-33B forms a cap or cover over the nub 220 and may be referred to herein as a cap or cover portion of the septum retainer 112. The portion of the projection 221 most distal to the outlet 212 of the cannula 104 is substantially planar and oriented perpendicularly to the axis of the channel 218. In certain examples, the wall of projection 221 adjacent the nub 220 may include one or more protuberance similar to protuberances 391 described in relation to FIG. 30F.

As the nub 220 of the septum 110 is compressed during assembly of a cannula subassembly 114, the material may have a tendency to “mushroom” out as it projects through the channel 218. The projections 221 may aid in preventing this “mushrooming” and force septum 110 material toward the puncture pathway for the insertion sharp 132 (see, e.g., FIG. 74B). This may help the cannula subassembly 114 resist leaks at pressures even further in excess of those expected during use. Additional potential benefits are described above in relation to FIGS. 15A-16C and FIG. 17.

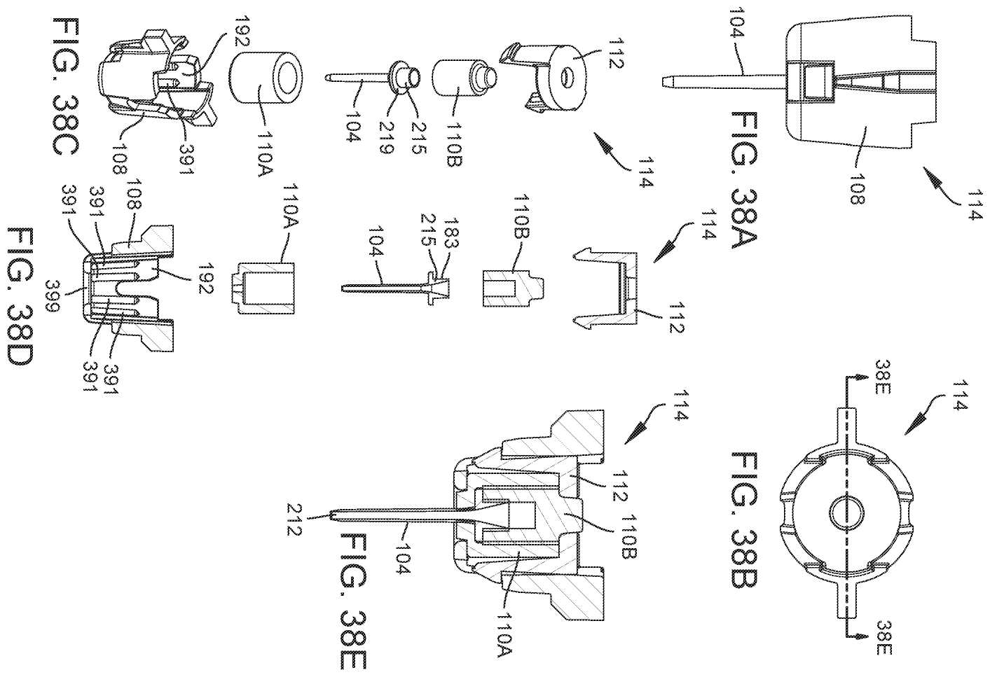

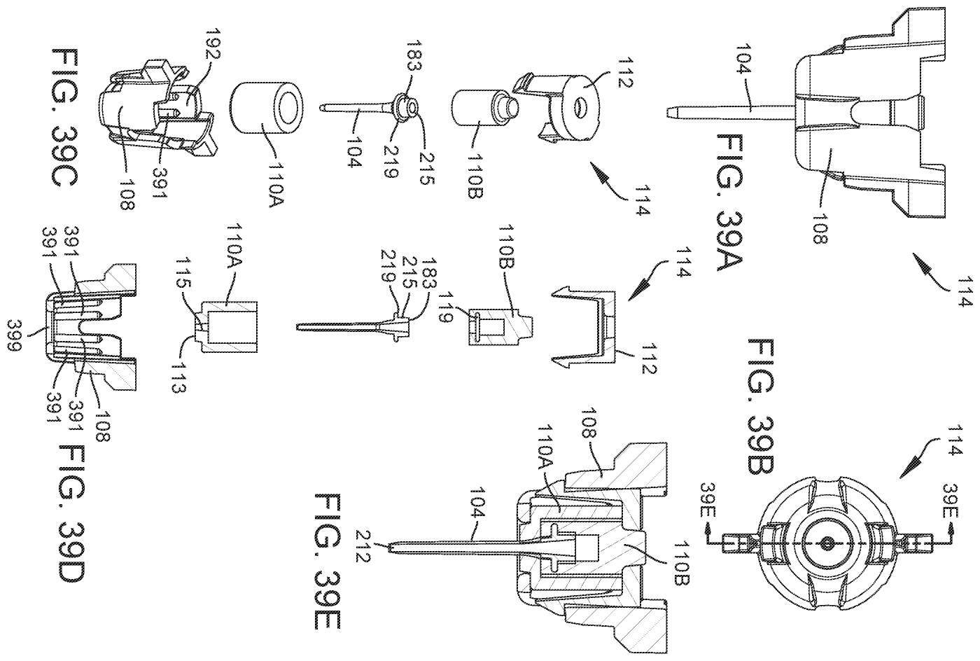

Referring now to FIGS. 34A-34F, another example cannula subassembly 114 is depicted. As shown, the cannula subassembly 114 includes a cannula 104 and a septum housing 108. No septum retainer 112 (see e.g. FIG. 31A) is included in the embodiment depicted in FIGS. 34A-34F. This may lower part count and may aid in simplifying manufacture of a cannula assembly 114.

The example cannula 104 includes an enlarged region 215 which includes a septum interface region 183 surrounding the insertion sharp guide 216 of the cannula 104. When assembled, a corresponding portion 185 of the septum 110 (no septum shown in FIGS. 34A-34F, see e.g., FIG. 31D) may be seated against the septum interface region 183. The enlarged region 215 may also include an outwardly extending flange 219. A surface of the flange 219 most distal the cannula outlet 212 may include a depression 223 which may receive an end of a septum 110. The depression 223 may also include contoured surfaces which may aid in centering or locating the septum 110 in the cannula subassembly 114. The opposing surface of the flange 219 may include a set of recesses 214A, B. Recess 214A may provide a space for the portion of the cannula 104 extending from this surface to move relative to an infusion set base 106 minimizing shearing action on the cannula 104. Agent may be provided in either recess 214A, B as described elsewhere herein.

At the periphery of the flange 219 a number of cantilevered arms 231 may be included. A terminal protuberance or latch member 233 may be included at the unsupported end of each of the cantilevered arms 231. Thus, the cantilevered arms 231 may be referred to as latch arms. During assembly, the latch member 233 of each cantilevered arm 231 may slide within a respective guide 208 for the arm 231 included on the interior surface of the receiving section 192 of the septum housing 108. The guides 208 shown are recessed into the interior surface of the receiving section 192 of the septum housing 108. The cantilevered arms 231 may be resiliently deflected toward the axis of elongation of the cannula 104 when the latch members 233 are within the guides 208. As the septum housing 108 and cannula 104 are displaced toward one another beyond a certain distance, the latch members 233 may exit the guides 208 and pass through apertures 235 included in the septum housing 108. This may allow the cantilevered arms 231 to resiliently restore and spring outward such that the latch member 233 on each cantilevered arm 231 enters into latching engagement with a catch surface 210 defined by each of the apertures 235. In the example embodiment, the cannula 104 includes a set of four cantilevered arms 231. The cantilevered arms 231 are also spaced from one another at substantially regular angular intervals though need not be in all example embodiments. In other embodiments, a greater or lesser number of cantilevered arms 231 may be included. The apertures 235 in the example are notches which are recessed into a first end wall 241 of the septum housing 108 and partially into the sidewall 243 of the septum housing 108. The first end wall 241 may close one end of the septum housing 108 and the flange 219 of the cannula 104 may close the second end of the septum housing 108 when the cannula 104 is coupled to the septum housing 108. The septum 110 (not shown) may be captured between the flange 219 and the first end wall 241 within the septum housing 108 when the cannula subassembly 114 is assembled. The first end wall 241 may include a channel 218′ which extend therethrough and accepts a nub 220 of a septum 110 (sec, e.g., FIG. 31D).

As shown, the flange 219 may also include a number of ledge members 237 which may extend outwardly from the periphery of the flange 219. When the cannula subassembly 114 is assembled, the ledge members 237 may provide a retention interface which may, for example, engage a protuberance 182 on a cantilevered projection 180 (see, e.g., FIG. 4A) of an infusion set base 106. Thus, the ledge members 237 may aid in coupling of a cannula subassembly 114 into an infusion set base 106. The septum housing 108 may include a recess 245 adjacent at least one of the ledge members 237.

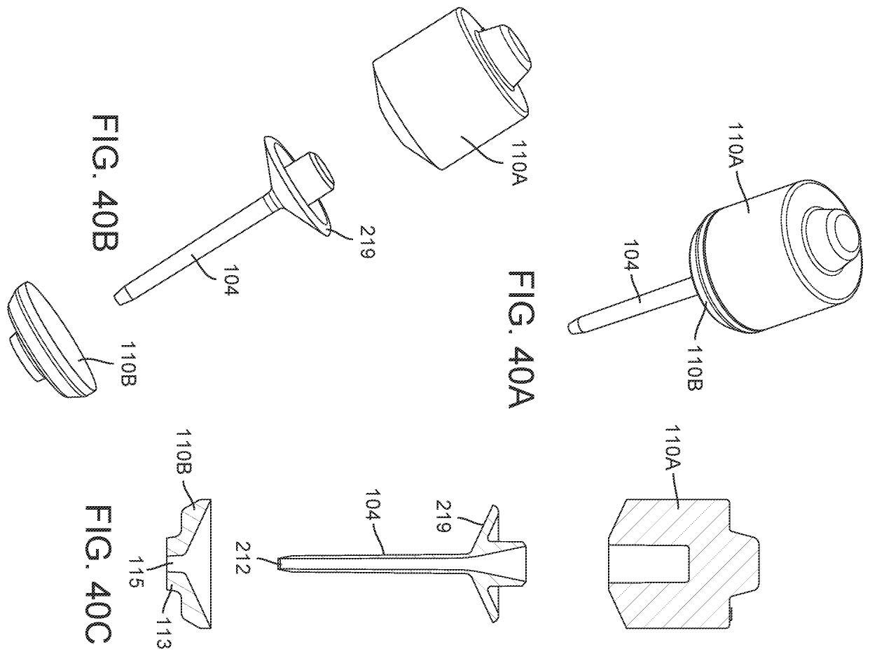

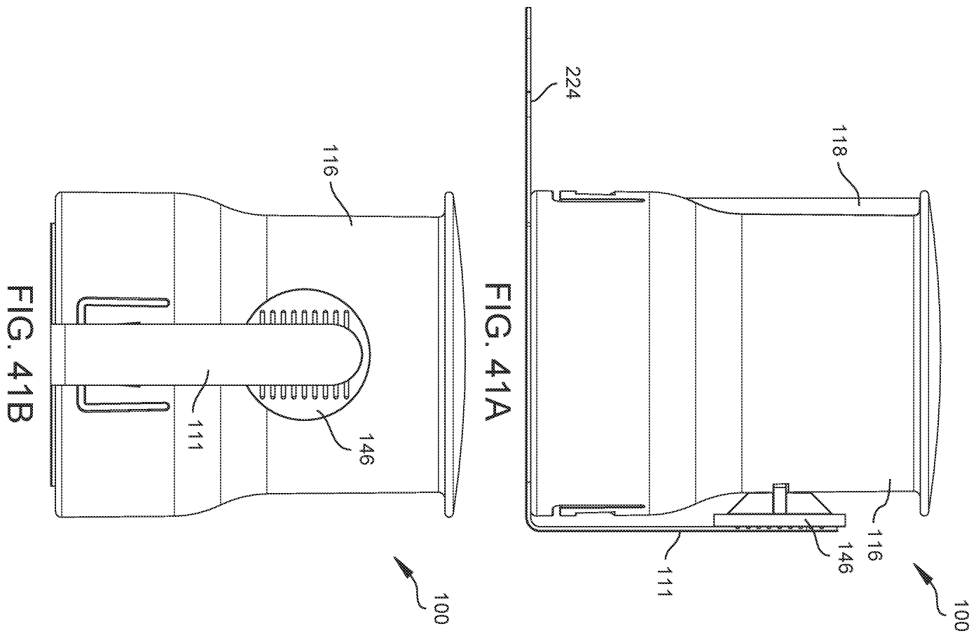

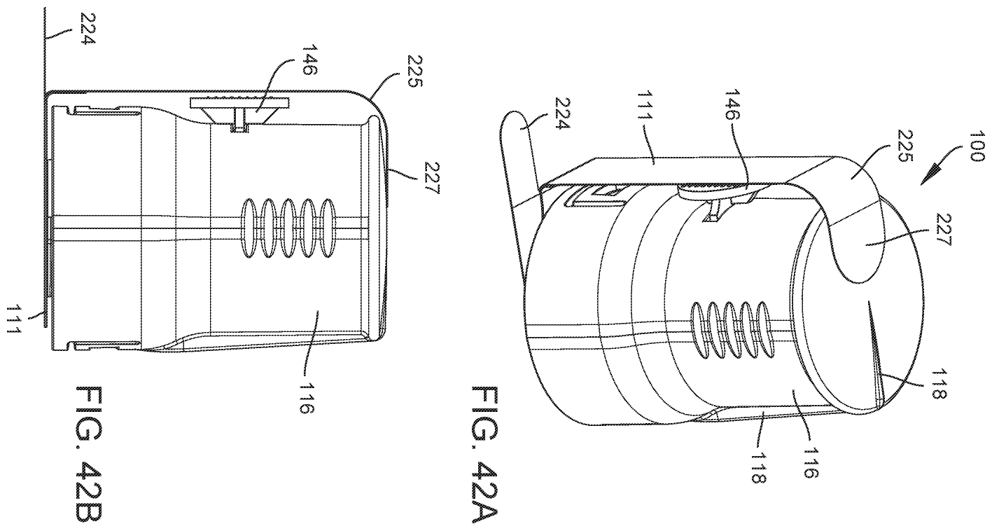

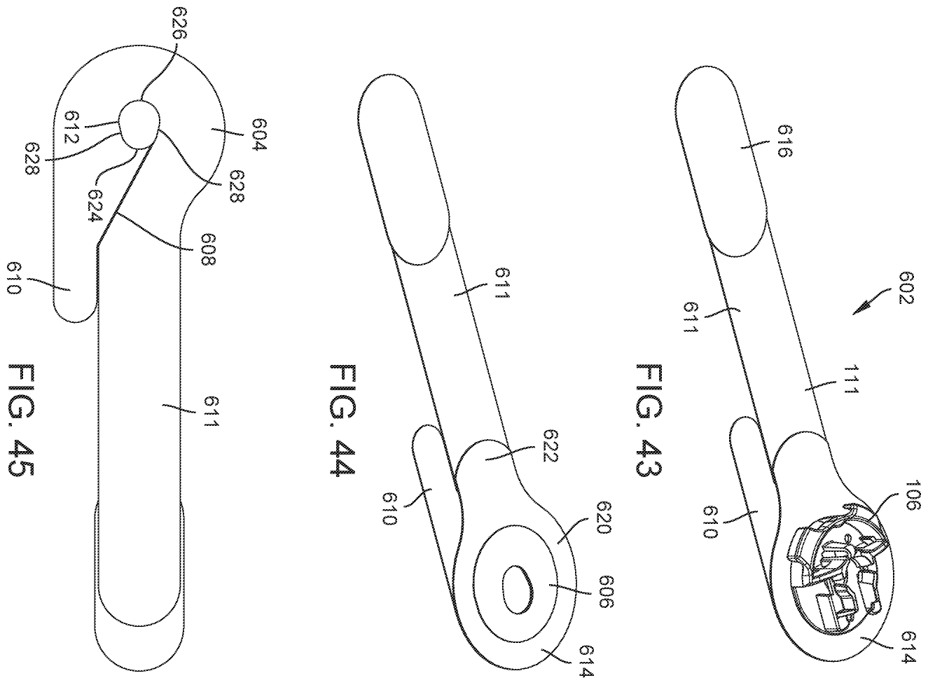

Referring now to FIGS. 35A-35F and FIGS. 36A-36E, additional example cannula subassembly 114 embodiments are depicted. The cars 204 extending from the main portion of the example septum housing 108 may include wedge bodies 205. Such wedge bodies 205 may be included on the cars 204 of any other cannula subassemblies 114 shown and described herein. The wedge bodies 205 may be disposed on the portion of each of the cars 204 which is most distal to the outlet 212 of the cannula 104 and may be disposed in opposition to one another on opposing faces of the cars 204. The thickness of the wedge bodies 205 may decrease as distance to the outlet 212 of the cannula 104 decreases. The wedge bodies 205 may extend from the sidewall 207 of the septum housing 108.

Additionally, the cars 204 may include an expanse 209 which extends from the main portion 211 of the cars 204 toward the outlet 212 of the cannula 104. Such an expanse 209 may be included on other cannula subassemblies 114 shown and described herein. The expanse 209 may be tapered such that width of the expanse 209 decreases with increasing proximity to the outlet 212 of the cannula 104. As the cannula subassembly 114 is displaced into an infusion set base 106, the taper on the expanse 209 may aid in directing the cannula subassembly 114 into the notches 174 (sec, e.g., FIG. 4A) of the infusion set base 106. In certain examples, an end of the expanse most distal the main portion 211 of the cars 204 may thinned so as to be potentially sacrificial. In the event that the expanse 209 is too long (e.g. due to tolerancing), this sacrificial portion of the expanse 209 may deform as it is displaced into an interfering structure of a base 106. Thus, the sacrificial portion of the expanse 209 may allow a cannula subassembly 114 to couple to the base 106 without issue even if relatively loose tolerances are used. The wedge bodies 205 may assist in ensuring that the cannula subassembly 114 is snuggly retained within the notches 174 (sec, e.g., FIG. 4A) during assembly and may aid in centering the cannula subassembly 114 within the receptacle 176 (see, e.g., FIG. 4A). The wedge bodies 205 may allow the notches 174 and/or septum housing 108 to be made with less stringent tolerancing while minimizing potential for the cannula subassembly 114 to have room to wiggle when retained within the receptacle 176 (sec, e.g., FIG. 4A). The wedge bodies 205 may also aid in guiding of the cannula subassembly 114 into place during assembly into a base 106.

Still referring to FIGS. 35A-36E and additionally to FIGS. 37A-37B, in certain embodiments where the cannula 104 and septum housing 108 are discrete components, a plurality of septums 110A, 110B may be included. A multiplicity of septums 110 may be included in other embodiments as well. The exemplary septums 110A, B may each be made of the same or a different material and/or durometer material. The cannula 104 may include an enlarged region 215 and a flange 219 as described, for example, in relation to FIGS. 30A-30G. The flange 219 may be captured in fluid tight manner between each of the septums 110A, 110B. In the example embodiment, a first of the septums 110A may include a fluid introduction volume 109 and may include a corresponding portion 185 which seats against the septum interface region 183 of the cannula 104. A second of the septums 110B may be disposed in opposition to the first septum 110A. The second septum 110B may include a protruding section 113 which extends into a passage 399 of the septum housing 108. An orifice 115 may be included in a central region of the protruding section 113. The cannula 104 may extend through the orifice 115 (best shown in FIGS. 37A-37B) to the exterior of the cannula subassembly 114. The orifice 115 may also seal against the surface of the cannula 104. As the septum 110B material may be elastomeric, the septum 110B may allow for the cannula 104 to move in relation to relative to an infusion set base 106 minimizing shearing action on the cannula 104.

Referring primarily to FIGS. 37A-37B, views of the septums 110A, B from FIGS. 35A-35F and FIGS. 36A-36E are respectively shown. At least one of the septums 110A, B may include a peripheral rim 117 on a side of the septum 110A, B closest to the opposing septum 110A, B. The peripheral rim 117 may form a fluid tight seal and extend adjacent the edge of the flange 219 of the enlarged portion 215 of the cannula 104 when the cannula subassembly 114 is assembled. The peripheral rim 117 may also surround a receptacle region of the septum 110A, B in which it is included. During assembly, the receptacle region may accept the flange 219 of the cannula 104.

Referring now to FIGS. 38A-38E and FIGS. 39A-39E, further exemplary cannula subassembly 114 embodiments are depicted. As shown, the cannula subassemblies 114 include a plurality of septums 110A, B. In the example embodiment, the first septum 110A is cup-like. The second septum 110B is also cup like in shape, but inverted with respect to the first septum 110A when the septums 110A, B are installed into the receptacle 192 of the septum housing 108. As may be true of any other cannula assembly 114 shown and described herein, the receptacle 192 of the septum housing 108 may include protuberances 391 (further described above in relation to FIGS. 30A-30G)

In the examples shown in FIGS. 38A-39E, the second septum 110B is sized so as to nest within the cup or cavity formed by the first septum 110A. The interior walls of the cup of the first septum 110A may establish a fluid seal against the exterior sidewall of the second septum 110B. The second septum 110B may seal against the septum interface portion 183 of the enlarged region 215 of the cannula 104. The flange 219 of the cannula 104 may be captured in fluid tight scaling relationship between the two septums 110A, B (see FIGS. 38A-38E). Alternatively, the second septum 110B may include a recess 119 defined on the interior wall of the cup portion of that septum 110B (sec FIGS. 39A-39E). The flange 219 of the cannula 104 may seat within this recess 119 when the cannula subassembly 114 is assembled and the recess 119 may establish a fluid tight seal against the flange 219. The first septum 110A may include a protruding section 113 which projects into a passage 399 defined in the septum housing 108. The cannula 104 may extend through an orifice 115 in the protruding section 113 and out of the septum housing 108. The elastomeric material of the septum 110A may allow for displacement of the cannula 104 relative to the infusion set base 106 and minimize any potential shearing on the cannula 104 during use.

Referring now to FIGS. 40A-40C, in some embodiments, a flange 219 may extend from a cannula 104 in a non-radial manner and may not be substantially planar. The flange 219 may extend from the cannula 104 such that distance from the outlet 212 of the cannula 104 increases in a linear (or not linear in alternative examples) relationship as the flange 219 widens. Thus, the flange 219 may be in the shape of a frustum of a hollow cone. In other embodiments, the relationship between the distance from outlet 212 to the width of the flange 219 need not be linear and at least a portion of the flange 219 may have a curved contour when viewed in cross-section. The first and second septums 110A, B may include cooperatively angled or contoured surfaces which mate against opposing sides of the flange 219 to form a fluid tight seal. The second septum 110B may include a protruding portion 113 with an orifice 115 therein through which the cannula 104 may extend. The flange 219 may establish a ball and socket type relationship with the septums 110A, B allowing for greater motion of the cannula 104 relative to the rest of an infusion set 102 while maintaining a robust seal. Such a ball and socket type arrangement between the cannula 104 and septums 110A, B may be used in any embodiment where a cannula 104 is not monolithically formed together with the septum housing 108.

A variety of embodiments of infusion set bases 106, tubing set connectors 368, and cannula subassemblies 114 are described herein. Though certain features of these components may be described in the context of a particular example embodiment, the scope of the disclosure is not limited thereto. It is contemplated that features of any infusion set base 106, tubing set connector 368, or cannula subassembly 114 may be incorporated into other infusion set base 106, tubing set connector 368, or cannula subassembly 114 embodiments without departing from the scope of this disclosure. Additionally, any of the tubing set connectors 368 described herein may also be constructed as cap bodies which do not include sharps 482 (see, e.g., FIG. 15D), attached infusion tubing 366 (see, e.g., FIG. 6), and/or any flow passages, tubing receptacles 512 (see, e.g., FIG. 143), adhesive introduction apertures 520 (sec, e.g., FIG. 143), etc. Such cap bodies may be placed on an infusion set assembly 102 when the infusion set assembly 102 is not connected to a tubing set connector 368 (e.g. when an infusion pump is disconnected during bathing).

Referring now to FIG. 41A and FIG. 41B, two side views of an inserter assembly 100 are depicted. The inserter assembly 100 in FIG. 41B has been rotated clockwise 90° from its orientation in FIG. 41A. The example inserter assembly 100 is an assembled version of the exploded view depicted in FIG. 1A. The two views may be representative of the appearance of the inserter assembly 100 before use and after the inserter assembly 100 has been removed from its shipping/storage packaging. As shown, the exterior housing 116 of the inserter assembly 100 is visible and the lock member 146 is in place. Additionally, the adhesive backing 111 is present and covers an adhesive layer present on the bottom face 162 of the infusion set base 106. The adhesive backing 111 may include a gripping region 224 which may be in the form of a flange, tab, or other projection of backing material which extends beyond the footprint of the inserter assembly 100. This gripping region 224 may be grasped by a user and used to peel the adhesive backing 111 off of the infusion set 106. Once the backing 111 has been removed, the inserter assembly 100 may be placed against the desired infusion site using the raised rib 118 to aid in alignment. The lock member 146 may then be pulled out of the inserter assembly 100 to allow for the inserter assembly 100 to be actuated. As the example adhesive backing 111 is attached to the lock member 146, extraction of the lock member 146 may complete disassociation of the adhesive backing 111 from the inserter assembly 100. In the event that the user is unsatisfied with the location the inserter assembly 100 has been placed, the user may leave the lock member 146 in the inserter assembly 100 and pull the inserter assembly 100 off of the body. The presence of the lock member 146 may prevent actuation as this occurs and may allow the user to choose another infusion site.

Referring now also to FIG. 42A and FIG. 42B, two additional views of an inserter assembly 100 are depicted. The example adhesive backing 111 includes a strip 225 which extends from the portion of the adhesive backing 111 covering the adhesive layer present on the bottom face 162 of the infusion set base 106 (see, e.g., FIG. 43-45). The strip 225 may, in some embodiments, extend substantially parallel to and adjacent a gripping region 224 of the adhesive backing 111. In the example shown in FIG. 42A and FIG. 42B, the adhesive backing 111 is coupled to the exterior housing 116 of the inserter assembly 100 instead of the lock member 146. The strip 225 may have a length which is greater than the height of the inserter assembly 100. Thus, a terminal end region 227 of the strip 225 may be coupled to a top surface of the exterior housing 116. The terminal end region 227 may, for example, be attached via heat stake, weld (e.g. sonic weld), or adhesive (e.g. double sided tape or other sticking agent). The terminal end region 227 may be coupled to a spot which does not obstruct view of any raised alignment ribs 118 present on the top surface of the exterior housing 116. The strip 225 may be attached to other portions (e.g. side surface of exterior housing 116 and/or lock member 146) of the inserter assembly 100 as well in certain embodiments. In other embodiments, the length of the strip 225 may differ and the terminal end region 227 may be coupled to another portion of the inserter assembly 100 (e.g. a side surface of the exterior housing 116 or the lock member 146).

Referring now to FIGS. 43-45, an exemplary adhering assembly 602 is depicted. The example adhering assembly 602 shown in FIGS. 43-45 may be installed on an inserter assembly 100 in the manner depicted in relation to FIGS. 42A-42B. The exemplary adhering assembly 602 is depicted with an infusion set base 106 in FIG. 43. The infusion set base 106 is not depicted in FIGS. 44-45. In some examples, the infusion set base 106 may be installed into an inserter assembly 100 and the adhering assembly 602 may then be coupled to the base 106.

As shown, the adhering assembly 602 may include a liner 111 or backing. The liner 111 may cover and protect an adhesive patch 614 affixed to the infusion set base 106. The liner 111 may be constructed of any suitable material such as a polymer or waxed paper and may be removed from the infusion set base 106 prior to use of an inserter assembly 100 in which the base 106 is included. Optionally, adhering assemblies 602 may include an adhesive body 616 separate from the adhesive patch 614 on another region of the liner 111. This adhesive body 616 may be used to couple the liner 111 to a portion of an inserter assembly 100 such as an exterior housing 116 of an inserter assembly 100 or a flange 152 of a lock member 146.

As shown, the liner 111 may have substantially flat and elongate shape (e.g. die cut, laser cut, etc. from a larger sheet of material) though may be flexible and bent or folded so as to be routed as shown in FIGS. 42A-42B. One end of the liner 111 may include an enlarged region 604. The enlarged region 604 may be an adhesive patch covering section of the liner 111 which may cover adhesive of an adhesive patch 614 attached to the infusion set base 106. The enlarged region 604 and adhesive patch 614 may have an at least partially round footprint which may be aesthetically preferable. The liner 111 may also include an appendage or strip portion 611 which may extend from the enlarged region 604 to the opposing end of the liner 111. The strip portion 611 may, though need not, be substantially straight and may, though need not, have a generally uniform width. The end opposite the enlarged region 604 may be rounded. The strip portion 611 may have a width which is smaller than the enlarged region 604. The strip portion 611 may have a length which is greater than a height of an inserter assembly 100.

The liner 111 may also include a tab or flap 610. The flap 610 may be connected at a first end to the enlarged region 604. The flap 610 may extend to a second end opposite the first in a direction substantially parallel to the strip portion 611. The flap 610 may extend directly adjacent the section of the strip portion 611 most proximal the round region 604. This may give the liner 111 a compact look and help save material during manufacture.

Example adhesive patches 614 may be constructed of a substrate which bears a skin compatible adhesive on one side thereof. An adhesive patch 614 may include a main region 620 and a protruding region 622. The main region 620 may be flat and round and may have a foot print greater than that of the infusion set base 106 to which it is attached. The protruding region 622 may extend from the periphery of the main region 620 and partially along the strip portion 611 of the liner 111. A segment of the strip portion 611 closest to the enlarged region 604 may cover adhesive on the protruding region 622. When the infusion set base 106 is attached to an infusion site, the protruding region 622 may serve as a removal tab which may facilitate peeling of the adhesive patch 614 and infusion set base 106 off of the infusion site.

The adhesive patch 614 may be attached to a bottom face 162 (sec, e.g., FIG. 4B) of the infusion set base 106 in any suitable manner. In certain examples and as best shown in FIG. 44, a double sided tape 606 or other adhesive may be used to couple the adhesive patch 614 to the base 106. In alternative embodiments, the adhesive patch 614 may include a substrate which may be heat staked or welded (e.g. sonically, RF) to the bottom face 162 of the base 106. The adhesive patch 614, liner 111 and any double sided tape 606 may include an aperture extending therethrough.

Referring primarily to FIG. 45, the liner 111 may include a partition which divides a portion of the liner 111. In the example embodiment, a slit 608 which extends through the liner 111 is included though perforations and/or scoring may alternatively be used in place of the slit 608. In the example embodiment, a single slit 608 is used and extends across a portion of the enlarged region 604. The slit 608 may be oriented to encourage the liner 111 to peel off the adhesive patch 614 in a single piece and limit the potential for regions of high stress to form in the liner 111 as the liner 111 is removed. The shape of the aperture 612 of the liner 111 may also be selected to encourage this.

The slit 608 orientation and aperture 612 shape may facilitate simple, quick, and unitary removal of the liner 111 from the adhesive patch 614. This may be so even if a user aggressively removes the liner 111 or only roughly/loosely follows a prescribed removal motion when peeling the liner 111 from the adhesive patch 614. Additionally, the slit 608 orientation and aperture 612 shape may facilitate use of a wider range of materials for the liner 111.

As shown, the slit 608 may extend from a point between the first end of the flap 610 and the strip portion 611 of the liner 111. The slit 608 may extend to the aperture 612 in the liner 111. The slit 608 may be angled so as to be tangent to the edge wall of the aperture 612. The slit 608 may extend in a straight line. The slit 608 may be angled such that the slit 608 is at a tangent to a far side of the aperture 612 with respect to the flap 610. The aperture 612 may be constructed so as to have a minimum number of corners or regions where the sidewall of the aperture 612 otherwise redirects from extending in one direction to another differing direction. As shown, the sidewall of the aperture 612 includes a first rounded span 624 and a second opposing rounded span 626. These spans 624, 626 may be the only spans of the aperture 612 wall where the wall redirects. The first rounded span 624 may have a tighter curvature (e.g. smaller radius) than the second rounded span 626. The aperture 612 may also include two substantially straight sidewall spans 628 which connect the first and second rounded spans 624, 626. Transitions between each of these spans 624, 626, 628 is gentle. The first rounded span 624 may be more proximal the flap 610 than the second rounded span 626. The slit 608 may end at a point of tangency to the first rounded span 624. In some embodiments, the slit 608 may be collinear with one of the straight sidewall spans 628 of the aperture 612.

As the liner 111 is unfurled from the adhesive patch 614, the example liner 111 may redirect around a portion of the aperture 612. In the example embodiment, the liner 111 may make a turn around the second rounded span 626 of the aperture 612 as the liner 111 is peeled from the adhesive patch 614. As the second rounded span 626 has a gentler curvature (e.g. wider radius), the turn made by the liner 111 may be relatively gentle. This may mitigate opportunity for the liner 111 to develop points of high stress during removal. Additionally, the shortest distance between a point on the second rounded span 626 and the periphery of the liner 111 may be shorter than the shortest distance between the peripheral edge of the liner 111 and any other portion of the aperture 612. As a result, a relatively small surface area of the liner 111 may be attached to the adhesive patch 614 in the region where the liner 111 makes a turn about the aperture 612 during removal of the liner 111. Due to the lower surface area, the liner 111 may be least firmly or relatively weakly adhered to the adhesive patch 614 in this region. This may further aid in limiting stress on the liner 111 material as the liner 111 is peeled off the adhesive patch 614 around the second rounded span 626.

As the liner 111 is unfurled around the aperture 612, the orientation of the slit 608 may help ensure that the only portions of the liner 111 adhered to the adhesive patch 614 are disposed ahead of the area where the liner 111 is disassociating with the adhesive patch 614. Thus, the direction of pulling by a user to remove the liner 111 would not need to differ significantly from the direction of the lagging portion of the liner 111. This may prevent the generation of points of high stress on the liner 111 material where regions lagging behind the area where the liner 111 is disassociating from the adhesive patch 614 are still attached to the adhesive patch 614.

As a user pulls on the flap 610 to peel the liner 111 from the adhesive patch 614, the tangent orientation of the slit 608 with respect to the far side of the aperture 612 may be preferred as it may aid in allowing the liner 111 to be removed easily and unitarily. The orientation of the slit 608 may encourage a user to naturally peel the liner 111 along the direction of extension of the slit 608. A straight pull of the liner 111 along this direction may substantially inhibit the formation of stress points at the edge of the liner 111 along the second rounded span 626. A situation where the liner 111 is being pulled by a user in a direction significantly different than the direction of adjacent downstream portions of the liner 111 may be avoided as the liner 111 is disassociated from the adhesive patch 614 around the second rounded span 626. Thus, the region of the liner 111 which redirects around the second rounded span 626 may be more easily peeled off the adhesive patch 614.

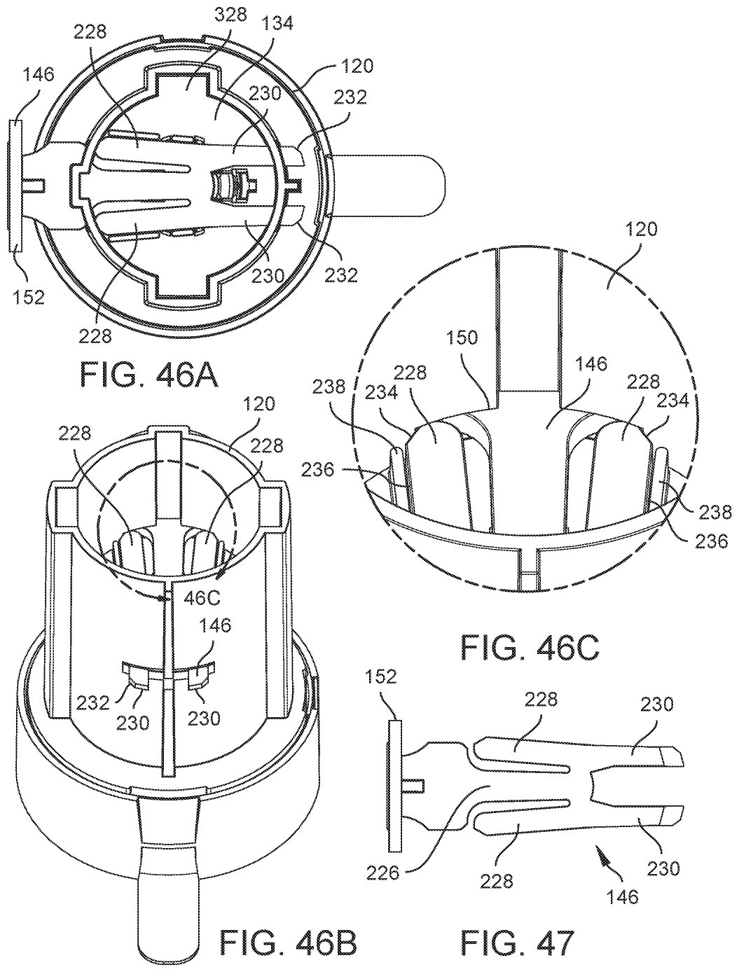

Referring now also to FIGS. 46A-47, various views of the inserter assembly 100 with the exterior housing 116 removed and a view of the lock member 146 in isolation (FIG. 47) are depicted. As shown, when in place within the inserter assembly 100, the lock member 146 may act as a safety. The inserter assembly 100 may be designed such that it cannot fire until the lock member 146 has been removed. The lock member 146 may extend across the entire width of the interior housing 120 and be above other components of the inserter assembly 100. The lock member 146 may be directly adjacent to at least one of the other components so as to prevent its movement.

The lock member 146 may include a flange 152 which may be grasped to aid in extraction. The lock member 146 may also include a stem portion 226 or appendage which projects away from the flange 152. The stem portion 226 may support a number of arms 228, 230. In the example embodiment, the arms 228, 230 are arranged in an “H” like pattern and the stem portion 226 is connected to the arms 228, 230 via the cross piece of the “H”. Arms 230 may reside in the fenestration 150 of the interior housing 120 most distal to the flange 152. Arms 230 may also include a chamfer feature 232 which may aid in guiding the lock member 146 during its installation into the inserter assembly 100.

As shown, arms 228 may be cantilevered so as to be able to deflect inward toward the stem portion 226. The distance between the outer edges of the arms 228 may be greater than the width of the fenestration 150 through which they pass when the lock member 146 is installed into the inserter assembly 100. During installation, the arms 228 may deflect toward the stem portion 226 to allow the arms 228 to pass through the fenestration 150. Once through the fenestration 150, the arms 228 may spring back outward to their unstressed state. Thus, as best shown in FIG. 46C, the width between the outer edges of the arms 228 may be wider than the width of the fenestration 150 when the lock member 146 is in place. This may ensure that some force may be required to remove the lock member 146 from the inserter assembly 100 and may inhibit the lock member 146 from being inadvertently dislodged. As shown, the arms 228 may include a chamfer region 234. The chamfer region 234 may abut a wall of the fenestration 150 of the interior housing 120 most proximal to the flange 152. The chamfer region 234 may aid in deflection of the arms 228 toward the stem portion 226 during extraction of the lock member 146 from the inserter assembly 100. Though an angled chamfer is shown, a rounded or curved region may be included in alternative embodiments.

In various examples, at least one component of the inserter assembly 100 may include at least one lock member constraining member such as raised bumpers 238. In the example embodiment, the bumpers 238 are included on the needle retractor 134 and extend from a top plate 328 thereof. The raised bumpers 238 may flank or be positioned aside or adjacent at least a portion of the lock member 146. The bumpers 238 may thus prevent any wobbling or pivoting of the lock member 146 within the inserter assembly 100. The bumpers 238 may also aid in redirecting the lock member 146 during installation if the lock member 146 is introduced into the inserter assembly 100 crookedly. A bumper 238 may also be provided to limit the depth which the lock member 146 may be pressed into the inserter assembly 100.

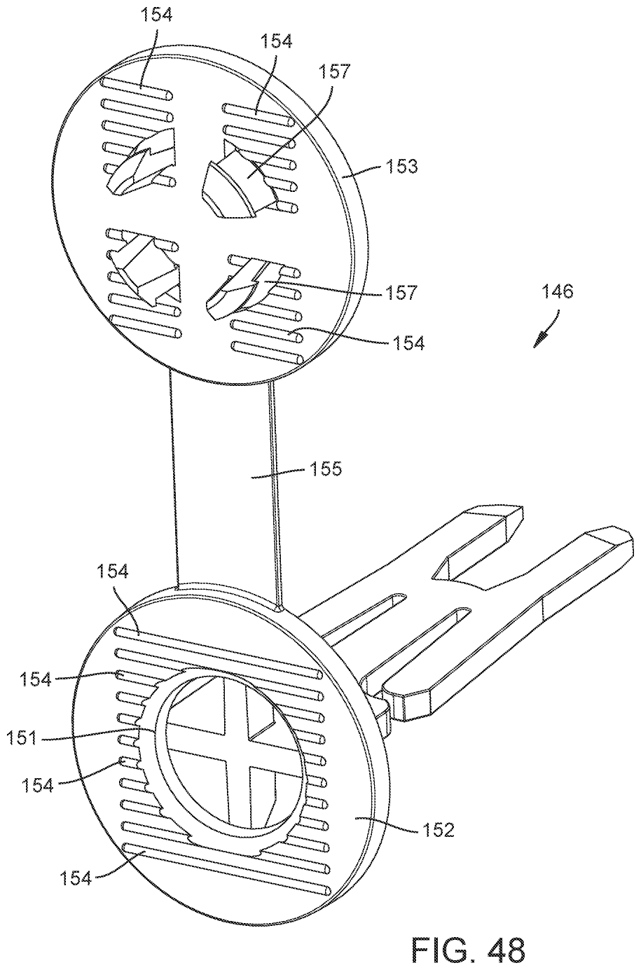

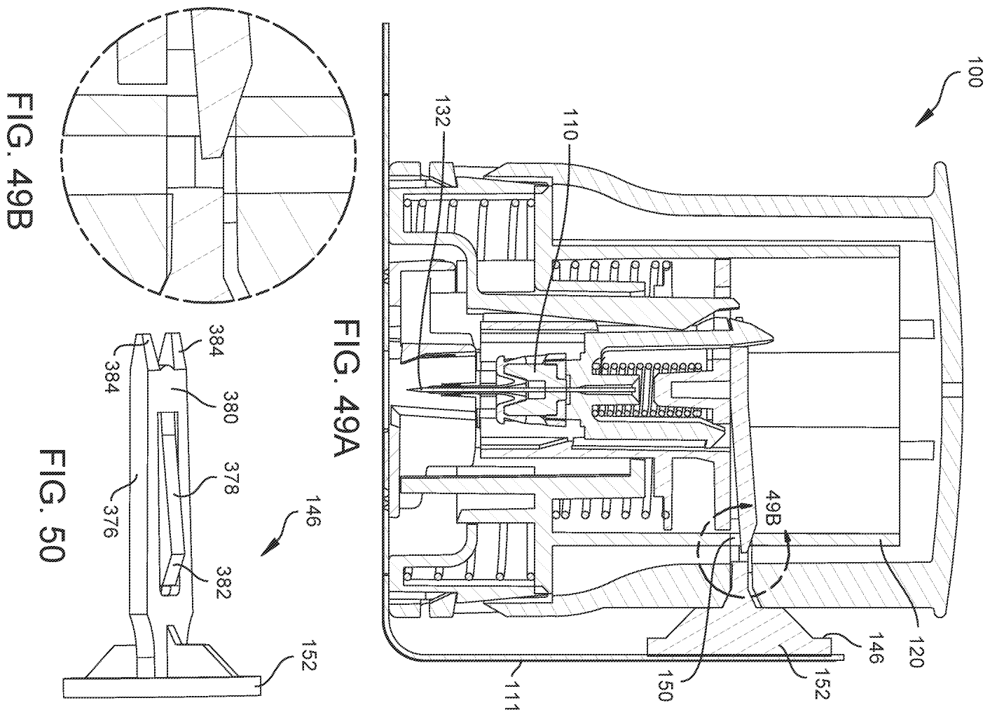

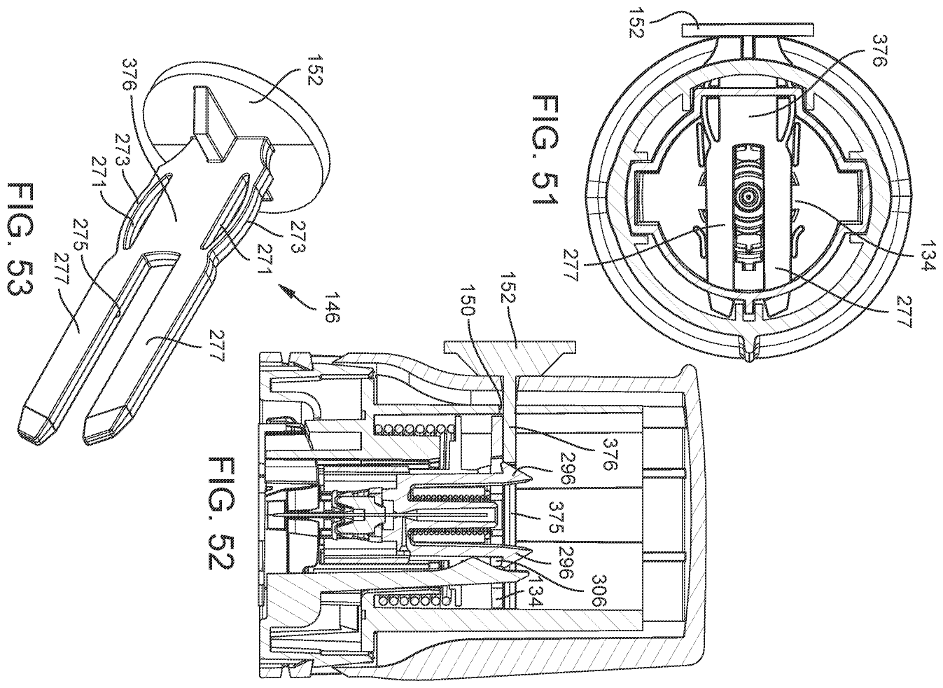

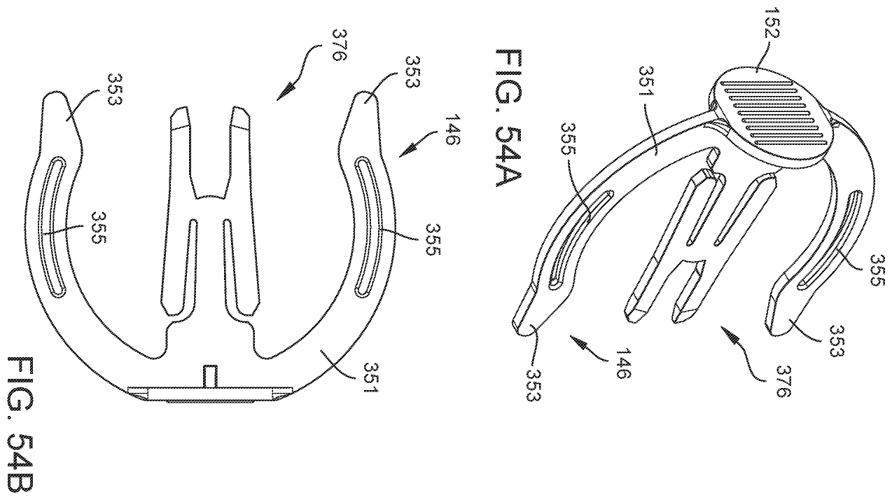

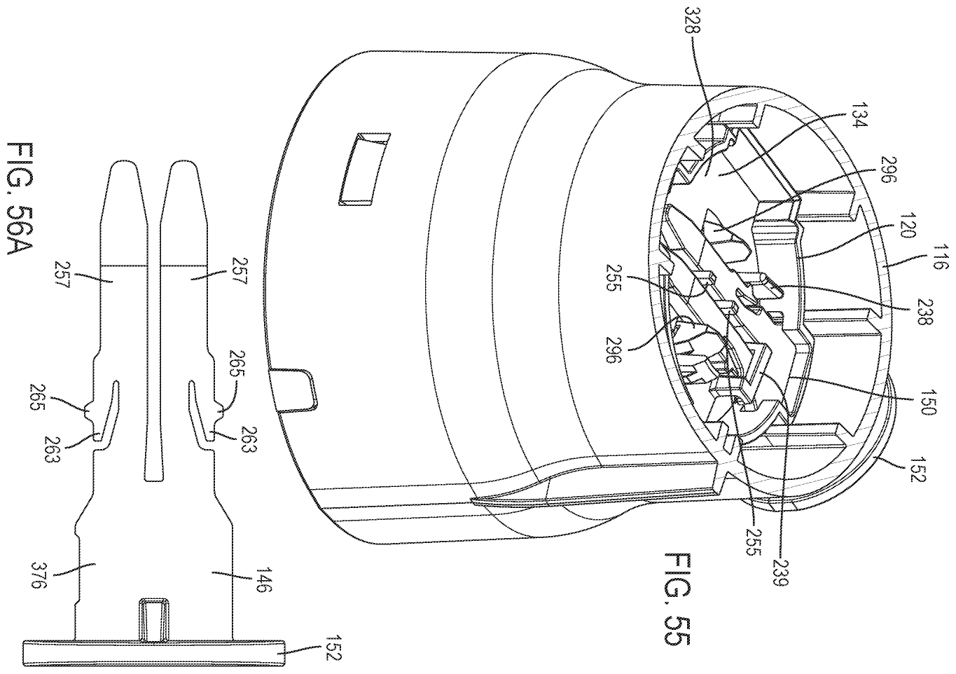

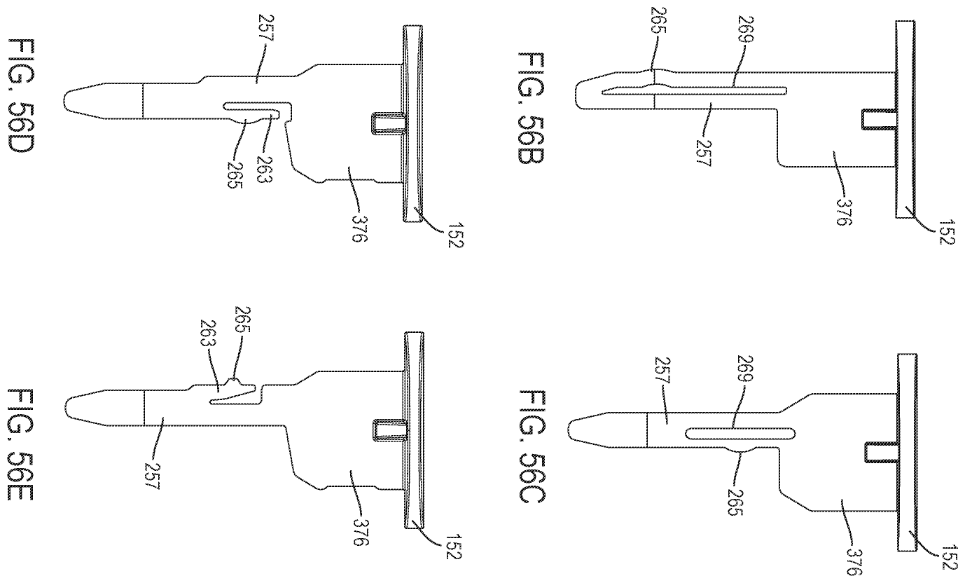

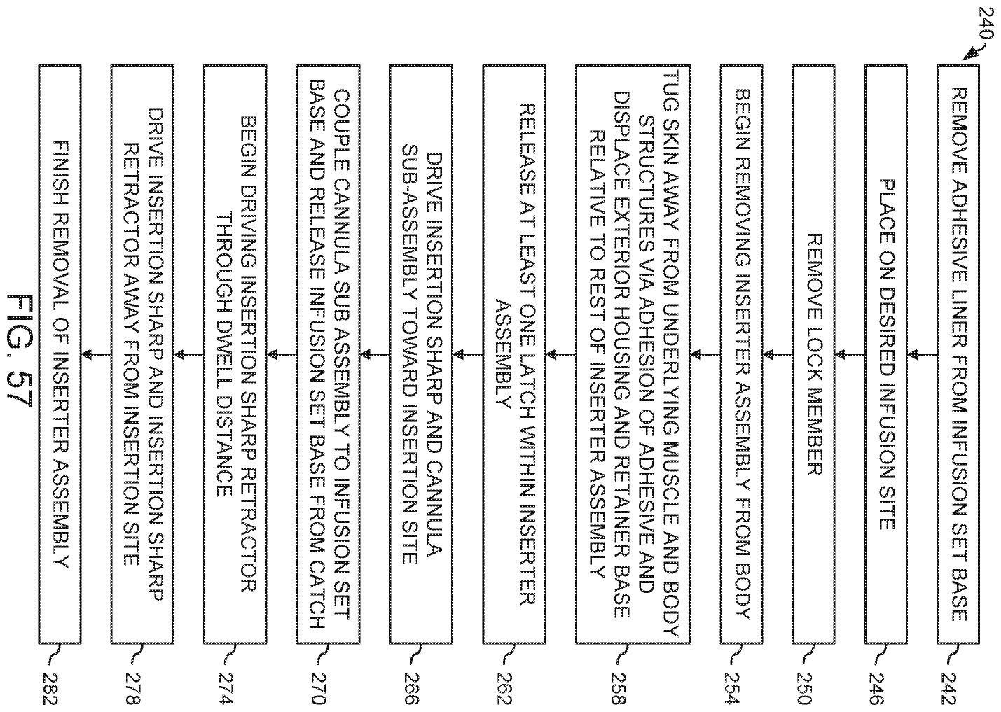

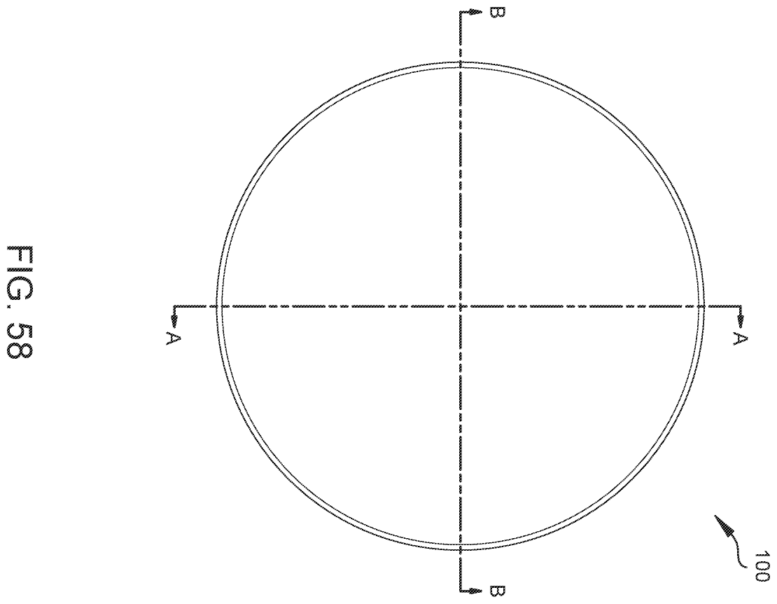

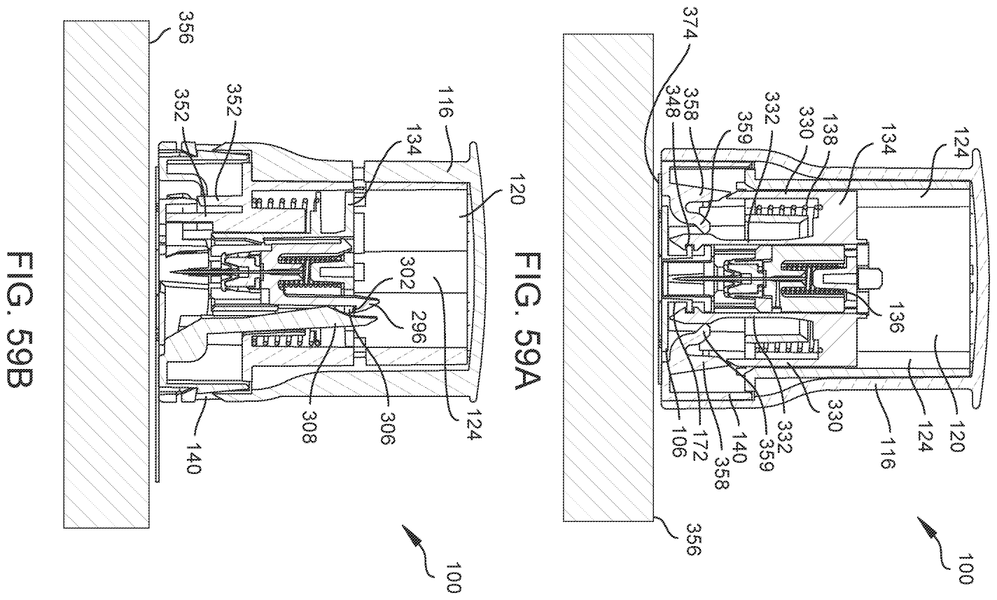

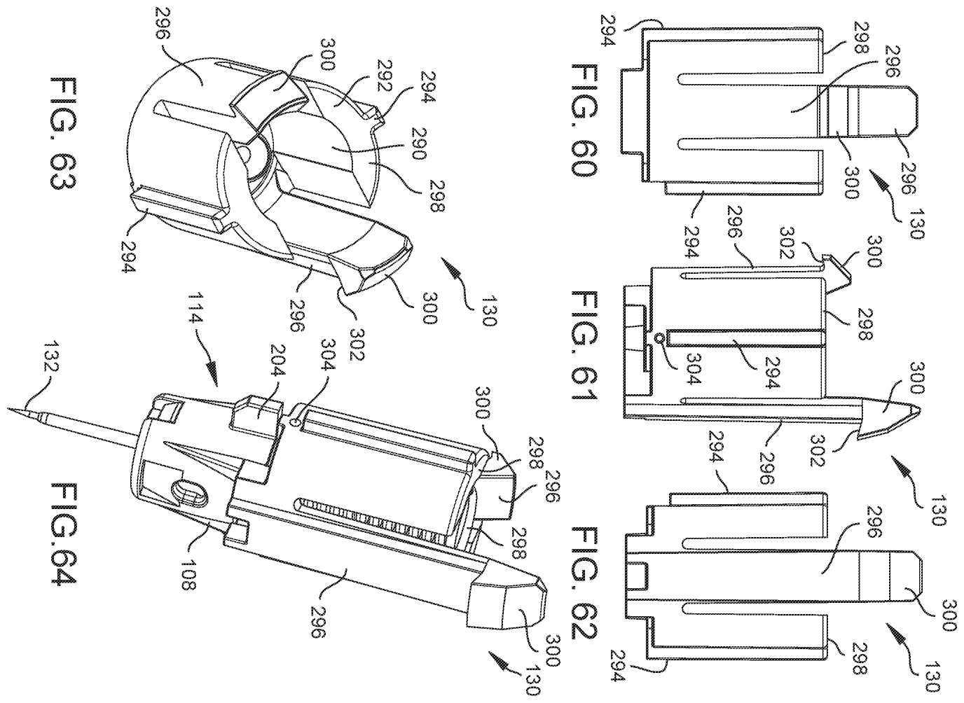

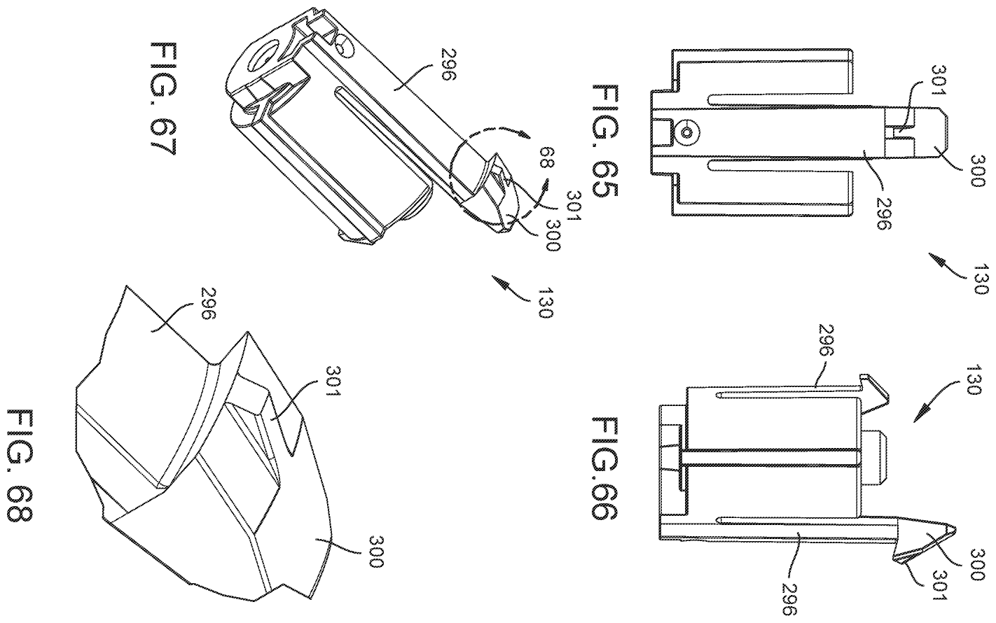

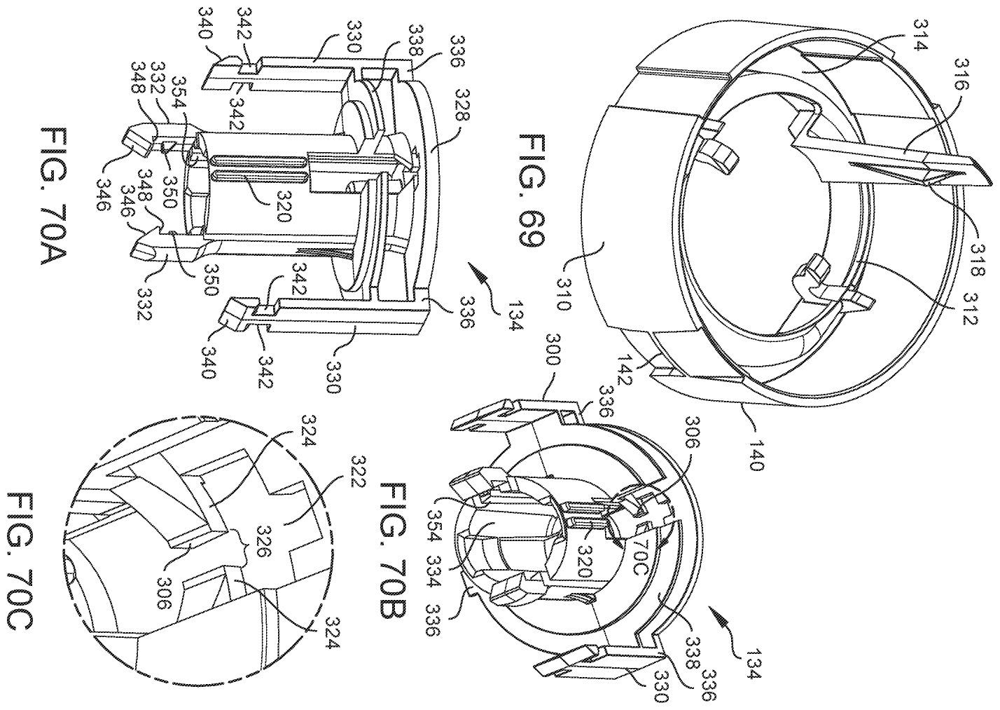

Referring now to FIG. 48, an alternative embodiment of the lock member 146 shown in FIGS. 46A-47 is depicted. As shown, the flange 152 of the lock member 146 may include an aperture 151 which may extend through the flange 152. The aperture 151 may be centrally disposed within the flange 152. A cap body 153 may be coupled to the lock member 146 by a strand or strip 155 of flexible material. In the example embodiment, a strip 155 of material extending from the periphery of the flange 152 to the periphery of the cap body 153 is used. The flexible strand or strip 155 may allow the cap body 153 to be displaced relative to the flange 152 while being leashed to the flange 152. In other embodiments, the cap body 153 may be a separate component. The cap body 153 includes a number of latch fingers 157 which project from the cap body 153. The latch fingers 157 may be arranged to deflect as they are introduced into the aperture 151 via a first side of the flange 152. After being displaced into the aperture 151 beyond a certain distance, the latch fingers 157 may resiliently restore and catch against the opposing face of the flange 152. In certain examples, and adhesive liner 111 (see, e.g., FIGS. 41A-B) may be captured between the cap body 153 and the flange 152 when the cap body 153 is engaged with the flange 152. Thus, no tape, adhesive, heat staking, RF welding, ultrasonic welding, etc. may need to be used to attach the liner 111 to the flange 152. In some embodiments, the liner 111 may include passages therein through which the latch fingers 157 may project. In other embodiments, the passages may not be included and the liner 111 may be crushed into the aperture 151 when the cap body 153 is coupled to the flange 152. Additionally, cap body 153 and flange 152 may include raised sections 154. The raised sections 154 may be spaced so as to interdigitate with one another when the cap body 153 is coupled to the flange 152. This may aid in firmly retaining a liner 111 between the cap body 153 and flange 153. Though sets of straight ridges are shown for the raised sections 154, other embodiments may include round raised sections (e.g. concentric circles) which may similarly capture the liner 111 therebetween when the cap body 153 and flange 152 are coupled. Cap bodies 153 and optionally the strand or strip 155 may be included for any other lock member shown or described herein. Any lock member 146 embodiments described herein may be modified to include a leashed or separate cap body 153.

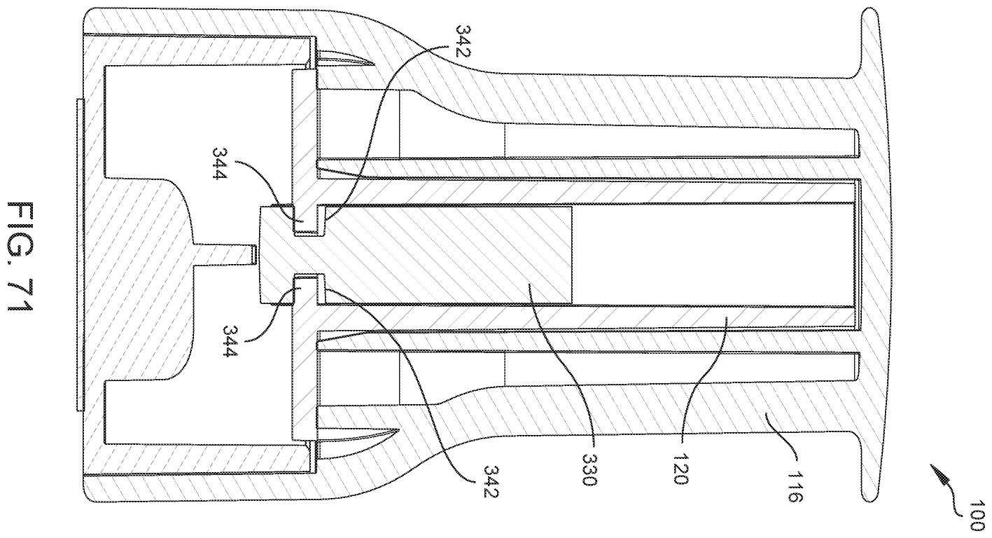

Referring now also to FIGS. 49A-50, various views of an inserter assembly 100 with an alternative lock member 146 and a view of the alternative lock member 146 in isolation (FIG. 50) are depicted. As above, when in place within the inserter assembly 100, the lock member 146 may act as a safety, preventing firing until the lock member 146 has been removed. The example lock member 146 extends across a portion, but not the entirety of the width of the interior housing 120 and is located above other components of the inserter assembly 100 to prevent movement. As shown best in FIG. 49A, the example lock member 146 is also directly adjacent an arm 296 of the sharp holder 130 and presents a mechanical interference to displacement of the arm 296 in the general direction of the flange 152. The interior housing 120 may only include one fenestration 150 instead of a set of opposing fenestrations 150.

The lock member 146 may include a flange 152 which may be grasped to aid in extraction. The lock member 146 may also include an appendage 376 which projects away from the flange 152. A tine 378 may be included within the appendage 376. The tine 378 is cantilevered to the appendage 376 at a portion of the tine 378 most distal to the flange. The tine 378 is also constructed to as to naturally project above a face 380 of the appendage 376 but be flexible when force is applied to the unsupported end of the tine 378. As shown, the unsupported end of the tine 378 includes a ramped region 382. The end of the appendage 376 most distal to the flange 152 may also include a chamfer feature 384 which may aid in guiding the lock member 146 during its installation into the inserter assembly 100. As the lock member 146 is installed into the inserter assembly 100, the top wall of the fenestration 150 may deflect the tine 378 toward the surface of the appendage 376 such that the tine 378 may pass through the fenestration 150. After completing introduction of the lock member 146, the tine 378 may spring back toward its initial unstressed state. Thus, as best shown in FIG. 49B, a portion of the tine 378 may be disposed above the top wall of the fenestration 150 when the lock member 146 is in place. This may ensure that some force may be required to remove the lock member 146 from the inserter assembly 100 and may inhibit the lock member 146 from being inadvertently dislodged. As shown, the ramped section 382 may abut the top wall of the fenestration 150. The ramped section 382 may aid in deflection of the tine 378 toward the surface 380 of the appendage 376 during extraction of the lock member 146 from the inserter assembly 100. Though an angled section 382 is shown, a rounded or curved region may be included in alternative embodiments. Lock member constraining features similar to the raised bumpers 238 shown in FIGS. 46A-47 may be included as well.

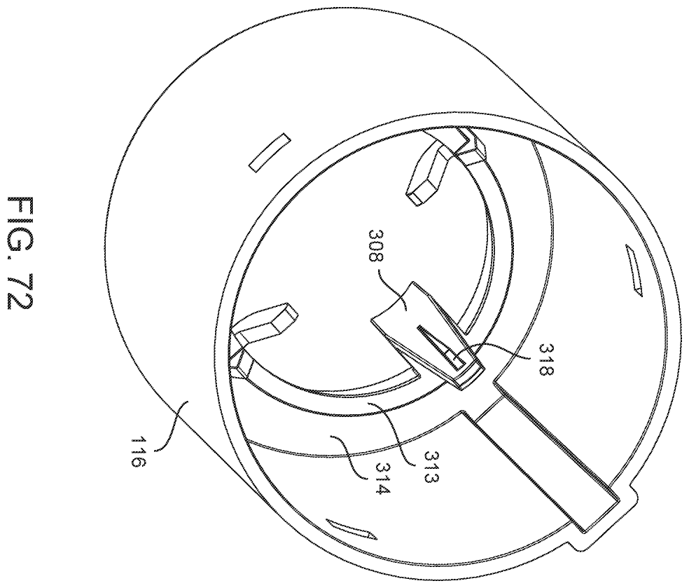

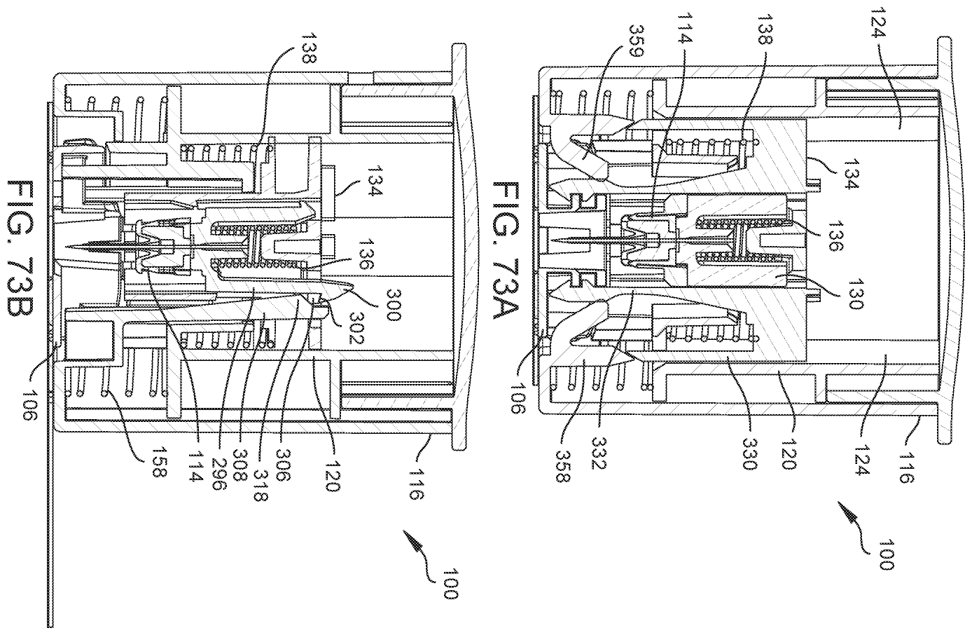

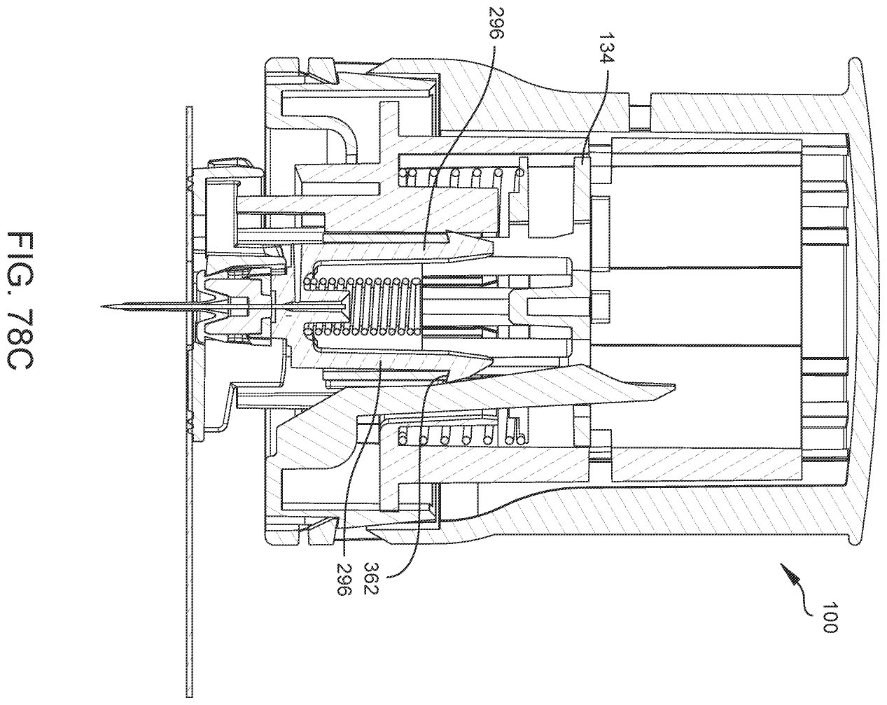

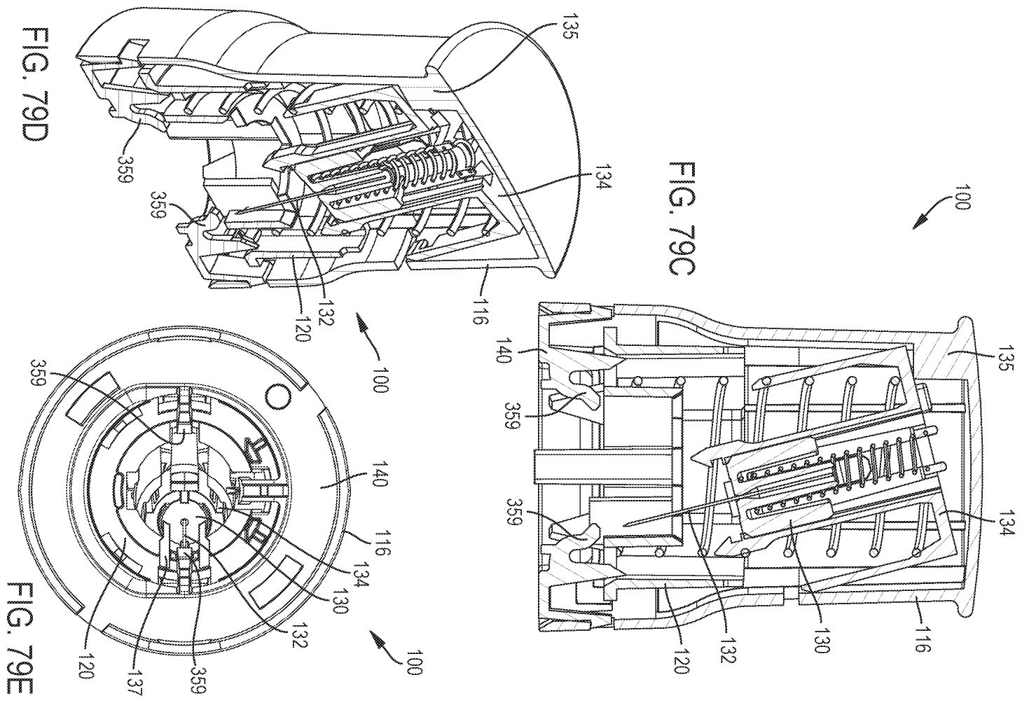

Referring now also to FIGS. 51-53, various views of an inserter assembly 100 with another alternative lock member 146 and a view of the alternative lock member 146 in isolation (FIG. 53) are depicted. As above, when in place within the inserter assembly 100, the lock member 146 may act as a safety, preventing firing until the lock member 146 has been removed. The example lock member 146 may include a flange 152 which may be grasped to aid in extraction. The lock member 146 may also include an appendage 376 which projects away from the flange 152. The appendage 376 may include a set of prongs 277 spaced apart by a slot 275 at a region of the appendage 376 most distal to the flange 152. As best shown in FIG. 52, the sharp holder 130 (further described in relation to FIGS. 60-68) may be symmetric and include two cantilevered arms 296 of substantially equal height. The slot 275 may accommodate these arms 296.

The portion of the appendage 376 most proximal to the flange 152 may include a set of arcuate members 273. The arcuate members 273 may be disposed on opposing sides of the appendage 376. An opening 271 between each arcuate member 273 and a main body 279 of the appendage 376 may be present. The distance between the outer sides of the arcuate members 273 may be greater than the width of the fenestration 150 through which they pass when the lock member 146 is installed into the inserter assembly 100. During installation, openings 271 may allow the arcuate members 273 may resiliently deflect toward the main body 279 of the appendage 279 such that the lock member 146 may fit through the fenestration 150. The arcuate members 273 may restore to their unstressed state once through the fenestration 150. This may ensure that some force may be required to deflect the arcuate members 273 to remove the lock member 146 from the inserter assembly 100 and may inhibit the lock member 146 from being inadvertently dislodged. Alternatively, when fully installed, the arcuate members 273 may not be advanced fully through the fenestration 150 and the arcuate members 273 may be in a deflected state pressing against the side wall of the fenestration 150. This may again help to ensure some force may be required to remove the lock member 146 and aid in preventing inadvertent dislodgement. When installed in the inserter assembly 100 the edge of the slot 275 most proximal the flange 152 may be directly adjacent an arm 296 of the sharp holder 130. This may help ensure that a cantilevered arm 296 of the sharp holder 130 is firmly held in place on a catch 306 of the sharp retractor 134 while the lock member 146 is in place.

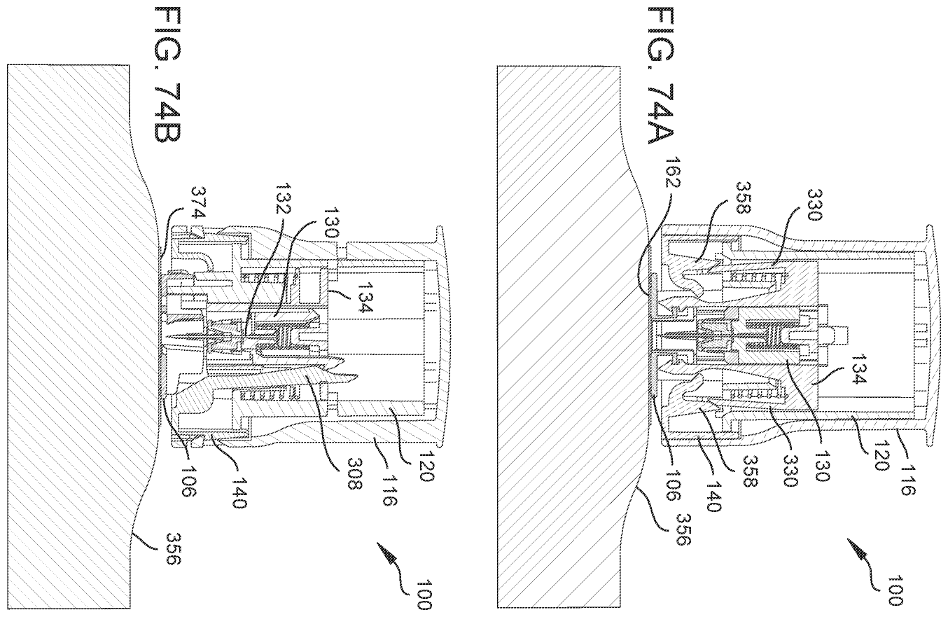

Referring now to FIGS. 54A-54B, yet another example lock member 146 is depicted. The exemplary lock member 146 includes an arcuate body 351. An appendage 376 may extend from the arcuate body 351. As shown, the appendage 376 extends from a midpoint of the arcuate body 351. In the example embodiment, the appendage 376 is that shown in FIGS. 46A-47 though any appendage 376 shown in embodiments herein may be used. When the lock member 146 is in place on an inserter assembly 100, the appendage 376 may extend into the inserter assembly 100 and prevent triggering of the inserter assembly 100. The arcuate body 351 may at least partially wrap around the exterior housing 116 of the inserter assembly 100. The end regions 353 of the crescent shaped body 351 may be ramped so as to aid in installation of the lock member 146 into the inserter assembly 100. As the lock member 146 is advanced toward an inserter assembly, the ramped end regions 353 may aid in centering the inserter assembly 100 with respect to the appendage 376. The arcuate body 351 may deflect such that the end regions 353 are spread apart from on another during a portion of the installation of the lock member 146 into the inserter assembly 100. The lock member 146 includes a flange 152 which may be grasped to aid in extraction.

The arcuate body 351 may include one or more slots 355. In the example embodiment two slots are included. The slots 355 may be disposed adjacent the end regions 353 of the arcuate body 351. The slots 355 may be sized such that a portion of a liner 111 may be fed through one of the slots 355. The liner 111 may then be coupled to a portion of the inserter assembly 100 (see, e.g., FIG. 42A). In the event that a user attempts to remove the lock member 146 prior to removal of the liner 111, the portion of the liner 111 passing through the slot 355 may inhibit extraction of the lock member 146. This may present a cue to a user which encourages a user to remove the liner 111 from an inserter assembly 100 prior to removal of the lock member 146.

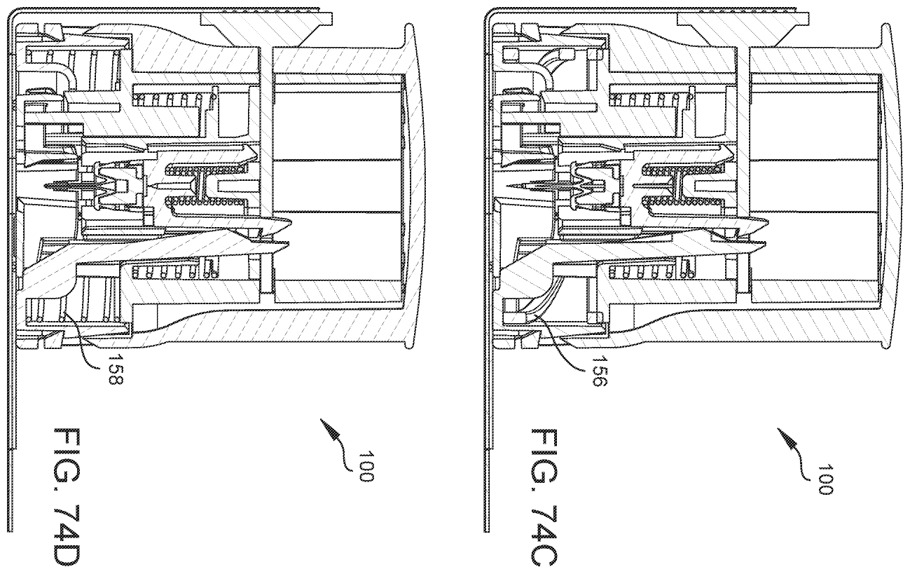

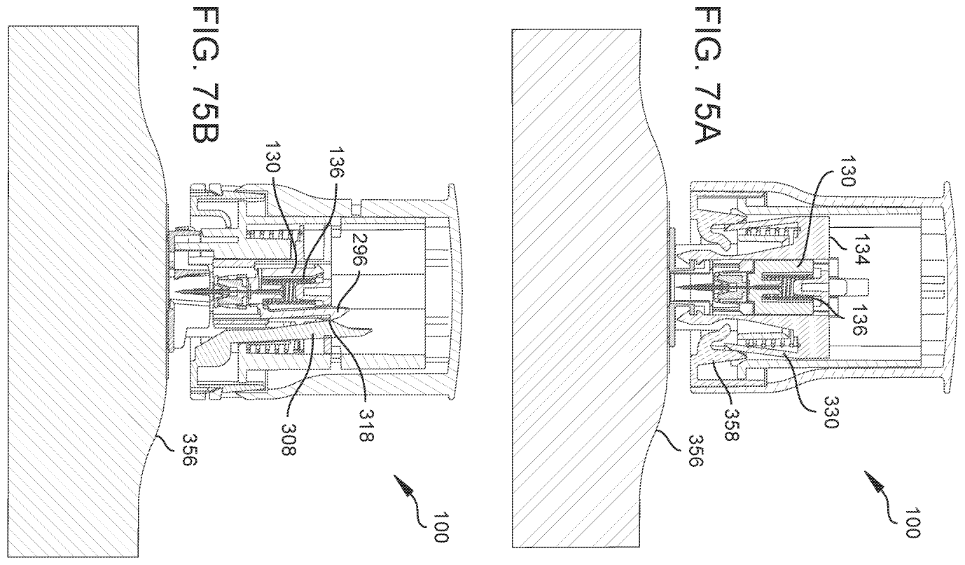

Referring now to FIGS. 55-56A, another example inserter assembly 100 and lock member 146 are depicted. As shown, the fenestrations 148, 150 in the exterior housing 116 and interior housing 120 respectively may be disposed to provide an introduction path for the lock member 146 which extends between arms 296 of the sharp holder 130. The lock member 146 may include a flange 152 from which an appendage 376 projects. At a portion of the appendage 376 opposite the flange at least one spreader projection 257 may be included (see, e.g., FIGS. 56B-56E for example embodiments with a single spreader projection 257). In the example embodiment, two spreader projections 257 are included and extend generally parallel to one another such that the exemplary lock member 146 is substantially symmetrical. The symmetrical nature of the lock member 146 may allow the lock member 146 to be installed in multiple orientations facilitating assembly. When installed in example inserter assemblies 100, the lateral faces of the spreader projections 257 may block deflection of the arms 296 of the sharp holder 130 toward a midplane of the inserter assembly 100. Thus, the example lock member 146 may keep the arms 296 spread apart from one another. In some embodiments, the spreader projections 257 may contact and press the arms 296 outward from the midplane when the lock member 146 installed in the inserter assembly 100. This may help to ensure that the arm 296 of the sharp holder 130 is retained in place on a catch 306 of the sharp retractor 134 while the lock member 146 is in place.

In the example embodiment, the sharp retractor 134 includes at least one interference body 255. In the example embodiment a set of interference bodies 255 is included. The interference bodies 255 may reside in a gap between the spreader projections 257 when the lock member 146 is installed. The interference bodies 255 may block displacement of the spreader projections 257 toward one another. In some embodiments, the interference bodies 255 may cause the spreader projections 257 to splay apart. The interference bodies 255 may aid in robustly retaining the arm 296 in engagement with the catch 306 (see, e.g., FIG. 52).

In the example embodiment, the top plate 328 of the sharp retractor 134 includes a bumper 238 (further described in relation to FIG. 46C) and passage through which a portion of the lock member 146 extends. In the example, the passage is defined by a bridge 239. At least one of the spreader projections 257 may extend under the bridge 239 when installed in the inserter assembly 100. This may inhibit movement of the sharp retractor 134 along the axis of the inserter assembly 100 when the lock member 146 is installed and may assist in preventing relative displacement of components within the inserter assembly 100. A portion of the bridge 239 may be received between the spreader projections 257 and double as an interference body 255 in some examples.

In various examples, the spreader projections 257 may each include a deflectable region. In the example embodiment, the spreader projections 257 each include a deflectable arm 263. As the lock member 146 is installed into the inserter assembly 100, one of the deflectable arms 263 may contact a surface of the bridge 239. The deflectable arms 263 may include nubs 265 such that further advancement of the lock member 146 toward the installed position may cause the deflectable arm 263 to bend toward the spreader projection 257 from which it extends. Upon the lock member 146 reaching the fully installed position, the nub 265 may be displaced clear of the bridge 239 and the deflectable arm 263 may restored to an undeflected state. Audible feedback (e.g. a click or slapping sound) may be generated as the deflectable arm 263 restores to the undeflected state. Tactile feedback may also be perceptible by the use as the arm 263 restores to the non-deflected state. The nub 265 may also inhibit inadvertent removal of the lock member 146.

Referring now also to FIGS. 56B-56E, a number of additional lock members 146 are depicted. Each of the lock members 146 includes a flange 152 from which an appendage 376 extends. A single spreader projection 257 which projects from a portion of the appendage 276 opposite the flange 152 is included in each example lock member 146. Each example lock member 146 could alternatively be symmetrical with two substantially parallel spreader projections 257. The spreader projections 257 of the lock members 146 each include deflectable regions. The examples shown in FIGS. 56D-56E include deflectable arms 263 with nubs 265 as described above in relation to FIGS. 55-56A. The spreader projections 257 in the example embodiments depicted in FIGS. 56B-56C include slots 269. The slots 269 may allow for regions of the spreader projections 257 adjacent the slots 269 to be deflected in the direction of the slot 269. With respect to FIG. 56C, as the lock member 146 is installed into an inserter assembly 100, a nub 265 on the spreader projection 257 may contact a surface of the bridge 239. Further advancement toward the installed position may cause a portion of the spreader projection 257 adjacent the slot 269 to bow or deflect inward. The nub 265 may be clear of the bridge 239 when the lock member 146 is in the fully installed position and the deflected portion of the spreader projection 257 may be free to restore to an undeflected state. With respect to FIG. 56B, the nub 265 may abut an arm 296 of the sharp holder 130 in engagement with the catch 306 (see, e.g., FIG. 52). When in the fully installed position, a portion of the spreader projection 257 adjacent the slot 269 to bow or deflect inward and the nub may press against the arm 296 holding the arm 296 in engagement with the catch 306.

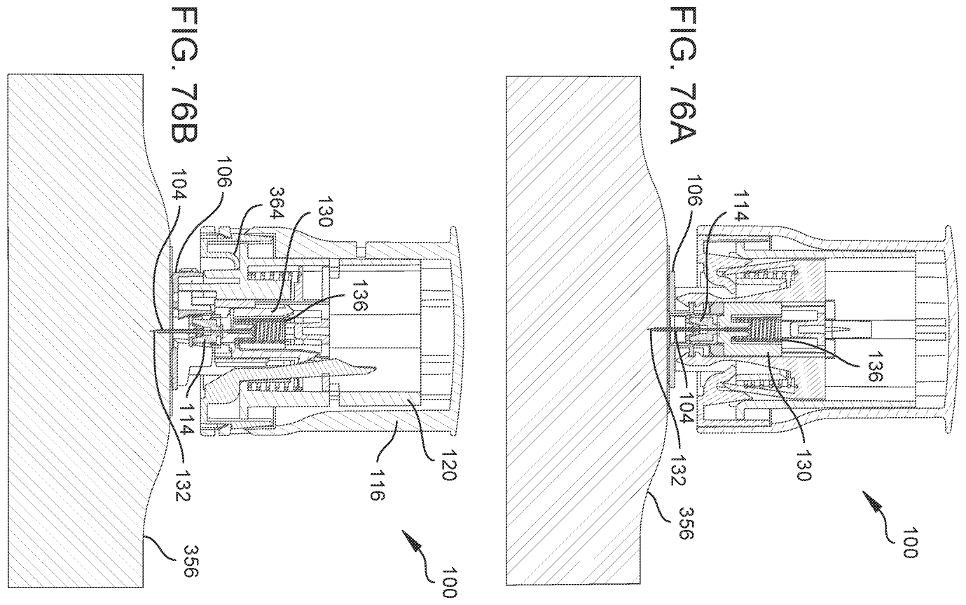

Referring now to FIG. 57, a flowchart 240 depicting a number of example actions which may be executed to place an infusion set 102 on a patient with an inserter assembly 100 is shown. Certain inserter assemblies 100, such as that shown in FIG. 1A, may be placed on the skin and be designed to prevent actuation until the skin has been displaced from its normal, resting position on the body. To this end, an inserter assembly 100 may include an actuation restricting arrangement (described elsewhere herein). Actuation of an inserter assembly 100 may be precluded until some degree of displacement of the skin has occurred. Actuation of an inserter assembly 100 may be prohibited until a certain amount of relative displacement between components of an inserter assembly 100 has occurred. This relative displacement may be effected as the skin is lifted and the inserter assembly 100 is withdrawn away from the body. The adhesion of the base 106 of the infusion set 102 to the skin may cause certain components (e.g. at least one component coupled to the base 106) to be restricted in their displacement as the user withdraws the inserter assembly 100. As the inserter assembly 100 is withdrawn, the elasticity of the skin may exert a force on the base 106 (and any coupled component) pulling it toward or holding it closer to the body. At least one other component of the inserter assembly 100 may be free to displace or have greater freedom to displace as the inserter assembly 100 is removed. Thus the inserter assembly 100 may be broken into a first unit and a second unit which is more constrained in its ability to displace as the inserter assembly 100 is pulled away from the body than the first unit. Relative movement may, in certain examples, be inhibited until a certain force is exerted against the base 106 by the skin. The user may need to pull the inserter assembly 100 away from the body with enough force to stretch the skin to a degree that relative movement of the different portions of the inserter assembly 100 is initiated. With the user pulling the inserter assembly 100 away from the infusion site and the elasticity of the skin pulling the second unit toward the infusion site in an opposing direction, a threshold force compelling separation of the first and second unit may be supplied. Once the threshold force is supplied, relative motion may begin.

A trigger for the inserter assembly 100 may be kept from actuation until the skin has been tugged away from the rest of the body a distance sufficient to generate the force required to begin relative movement. Triggering may not be possible until a requisite amount of relative displacement has occurred. Once triggered, an inserter actuation assembly (exemplary embodiments described below) of the inserter assembly 100 may be freed to complete placement of an infusion set 102 at the desired infusion site. The actuation assembly may include a trigger arrangement which, once released, causes an insertion sharp 132 to be driven into an infusion site. The actuation assembly may also retract the insertion sharp 132 back into the inserter assembly 100. The actuation assembly may couple a cannula subassembly 114 to an infusion set base 106. The actuation assembly may also uncouple the infusion set 102 from the inserter assembly 100 once the infusion set 102 has been installed at a desired infusion site.

In some embodiments, inserter assemblies 100 may be placed on the skin and trigger actuation as the inserter assembly 100 is lifted up so as to be removed. No other depression, twisting, squeezing, etc. of a trigger, button, housing sleeve or other portion of an inserter assembly 100 by a user may be needed to provoke the actuation, however, the actuation may still be under the control of the user. The relative movement of the free component(s) of the inserter assembly 100 with respect to the restricted component(s) may trigger actuation, by, for example, displacing or dislodging a latch and freeing one or more bias members to begin driving actuation. Thus, a trigger internal to the inserter assembly 100 may be actuated as a result of the removal action of the inserter assembly 100 from the body. The trigger may be automatically actuated, for example, when skin at the infusion site has been lifted at least a certain distance from underlying anatomy. From the perspective of a user, such an inserter assembly 100 may simply be placed on the skin and then withdrawn to execute placement of the infusion set 102.

In alternative embodiments, a discrete manual triggering action may be employed to trigger actuation of an inserter assembly 100. Any arrangement which would be apparent to one skilled in the art may be used to facilitate manual triggering. An inserter assembly 100 may include, for example, one or more button which when displaced may trigger actuation by dislodging a latch within the inserter assembly 100. Alternatively, a portion of the inserter assembly 100 may be deformable and squeezing the inserter assembly 100 may press a projection which displaces with the deformable section into a latch to dislodge the latch. The button or deformable section may, for instance, be included on exterior housing 116 in certain examples. A twisting action may be employed to trigger actuation of an inserter assembly 100. Such a twisting action of one portion of the inserter assembly 100 (e.g. the casing) relative to another (e.g. the remainder the inserter assembly 100) may sweep a projection of the inserter assembly 100 into a latch to dislodge the latch. In other embodiments, a pin or similar member may be pulled out of the inserter assembly 100 after the inserter assembly has been pulled away from the skin to trigger actuation. In embodiments including a lock member 146 (sec, e.g., FIG. 47), the lock member 146 may be kept in place until the user has begun withdrawing the inserter assembly 100 from the skin. Actuation may be triggered when removal of the lock member 146 allows components of the inserter assembly 100 to displace relative to one another such that, for example, a latch may be released. Various combinations of manual triggering arrangements may also be used. A button press or squeeze followed by a twist or vice versa may trigger actuation for instance. The manual triggering action may not be possible until after the skin has been displaced from a resting position and/or until a certain degree of relative movement between the free and restricted components of the inserter assembly 100 has occurred. Any arrangement which would be apparent to one skilled in the art may be used to facilitate such a lockout. An interlock, for example, may prevent button displacement, twisting, squeezing, etc. until the skin has been displaced or until relative movement beyond a threshold magnitude has occurred. Alternatively, an interlock may block access to a latch within the inserter assembly 100 preventing it from being dislodged until the skin has been displaced or until relative movement beyond a threshold magnitude has occurred. In some embodiments, button displacement, twisting, squeezing, etc. may be possible but rendered impotent by the interlock until the skin has been displaced or until relative movement beyond a threshold magnitude has occurred. As one skilled in the art would appreciate, the inserter assembly 100 embodiments described herein could be otherwise modified to allow for various types of additional manual actuation schemes.

While such designs may make triggering actuation simple, intuitive, and more foolproof, other advantages may also be realized. For example, as the inserter assembly 100 is lifted, the inserter assembly 100 may be designed so as to tug the skin to which the base 106 of the infusion set 102 is attached away from the underlying muscle and other body structures or anatomy. Thus, when inserted, the cannula 104 of the infusion set 102 may be more reliably placed within a subcutaneous layer of adipose tissue. This may reduce pain upon insertion, help minimize bruising, increase the potential body area over which infusion sites may be chosen, and may lead to more predictable absorption of agents such as insulin. The skin may also be pulled taut facilitating easy penetration of the insertion sharp 132 through the skin. As the skin is passively lifted along with the inserter assembly 100, no pneumatic vacuum is required to be generated. This may allow an inserter assembly 100 to be less complicated and made with fewer parts. Additionally, pneumatic seals either against the skin or within the inserter assembly 100 may be omitted. Lifting of the skin may be more reliably accomplished as the contour of the body at the infusion site (which could present a sealing challenge) may be largely irrelevant. Furthermore, no pinching of the skin may be needed to pull the skin away from the underlying structures. This may help to make the insertion more comfortable, may limit bruising, and may more reliably pull the skin away from underlying structures. The inserter assembly 100 may also ensure that insertion of the cannula 104 into the skin occurs at a prescribed orientation. The skin may be held in place so as to be parallel or perpendicular to a reference plane or axis (e.g. parallel to the bottom face 162 of the base 106 of the infusion set 102 or perpendicular to the axis of the insertion sharp 132 or insertion sharp displacement path) which moves with the inserter assembly 100. Thus, the angle of the inserter assembly 100 or path along which the inserter assembly 100 is pulled away with respect to the body may not alter insertion angle. Example embodiments shown herein depict an insertion angle which is substantially perpendicular to the skin, however, insertion at any angle (just over 0° to 90°, e.g. 30°, 45°, 60° etc.) may be similarly ensured by fixing the skin relative to a reference plane or axis which moves with the inserter assembly 100. Another potential benefit is that there may be less psychological concern associated with the triggering of the actuation. As depression, twisting, squeezing, etc. of some actuator by the user may not be necessary, there may be less anxiety built up in anticipation of triggering the actuation. The exact moment of actuation as the inserter assembly 100 is withdrawn may not be known to the user. This may help to limit psychological concerns and may lower perceived pain.

As shown in FIG. 57, in block 242, an adhesive liner or backing 111 may be removed from an infusion set base 102 retained within the inserter assembly 100. The inserter assembly 100 may then be placed on a desired infusion site in block 246. This may cause the adhesive on the infusion set base 102 to stick to the skin of the patient. It may be desirable to press the inserter assembly 100 against the skin to ensure a robust attachment of the adhesive to the skin. A lock member 146 may be removed from the inserter assembly 100 in block 250. The inserter assembly 100 may begin to be removed from the body in block 254. In block 258, the skin may be tugged away from underlying muscle and body structures via the adhesion of the adhesive. The exterior housing 116 and retainer base 140 may also be displaced relative to the rest of the inserter assembly 100 in block 258. As mentioned above, relative displacement may not occur until the skin has been lifted from underlying anatomy and the elasticity of the skin exerts enough force to overcome built in resistance to the relative displacement. In block 262, at least one latch within the inserter assembly 100 may be released. As discussed above, this may occur automatically, or as a result of some manual triggering action. An insertion sharp 132 and cannula subassembly 114 may be driven towards the insertion site in block 266. The cannula subassembly 114 may couple into the infusion set base 106 in block 270. Additionally, a catch may be released from the infusion set base 106 in block 270. The act of coupling the cannula subassembly 114 into the set base 106 may cause release of the catch from the infusion set base 106. This may free the infusion set base 106 from the rest of the inserter assembly 100. The catch may hold an insertion sharp retractor 134 in place when the catch is engaged with the infusion set base 106. With the catch released, the insertion sharp retractor 134 may be driven through a dwell distance in block 274 (described in further detail later in the specification). The insertion sharp retractor 134 and insertion sharp 132 may be driven away from the infusion site in block 278. The removal of the inserter assembly 100 may be completed in block 282. With the removal of the inserter assembly 100 completed, the infusion set 102 may be fully assembled and in place on the infusion site. Additionally, the cannula 104 may be in place in the subcutaneous layer of skin.

Referring now to FIG. 58, a top down view of an inserter assembly 100 with a first and second cut plane superimposed thereon is depicted. The first cut plane is labeled A-A. The second cut plane is labeled B-B. This figure is provided for reference purposes in relation to a number of the forthcoming figures. Several of these figures are cross-sectional views of the inserter assembly 100 taken at the location of one or the other of these planes and depict the inserter assembly 100 in various stages of operation. Unless described otherwise, where one of the following inserter assembly 100 cross-sections is given with a figure numeral followed with the letter A, the figure is depicting a cross-section of the inserter assembly 100 at the location of plane A-A. Where one of the following inserter assembly 100 cross-sections is given with a figure numeral followed with the letter B, the figure is depicting a cross-section of the inserter assembly 100 at the location of plane B-B.

Referring now to FIGS. 59A-59B, two cross-sectional views of an inserter assembly 100 are depicted. The inserter assembly 100 is depicted just as it is about to be applied to the skin 356. The lock member 146 (see, e.g., FIG. 50) and adhesive backing 111 (sec, e.g., FIG. 1A) have been removed in FIGS. 59A-59B. The adhesive 374 is depicted on the bottom face 162 of the infusion set base 106. As shown, both springs 136, 138 may be in an energy storing state, which in this particular embodiment is a compressed state. In the example, spring 136 serves as an insertion driving bias member while spring 138 serves as an insertion sharp retraction driving bias member. Spring 136 is held in compression between the insertion sharp holder 130 and the insertion sharp retractor 134 and when released drives the sharp holder 130 and components carried there on from a raised state to a forward state. Spring 138 is held in compression between the interior housing 120 and the sharp retractor 134. Upon release, spring 138 drives the sharp retractor 134 and sharp holder 130 from a post insertion state to a retracted state.

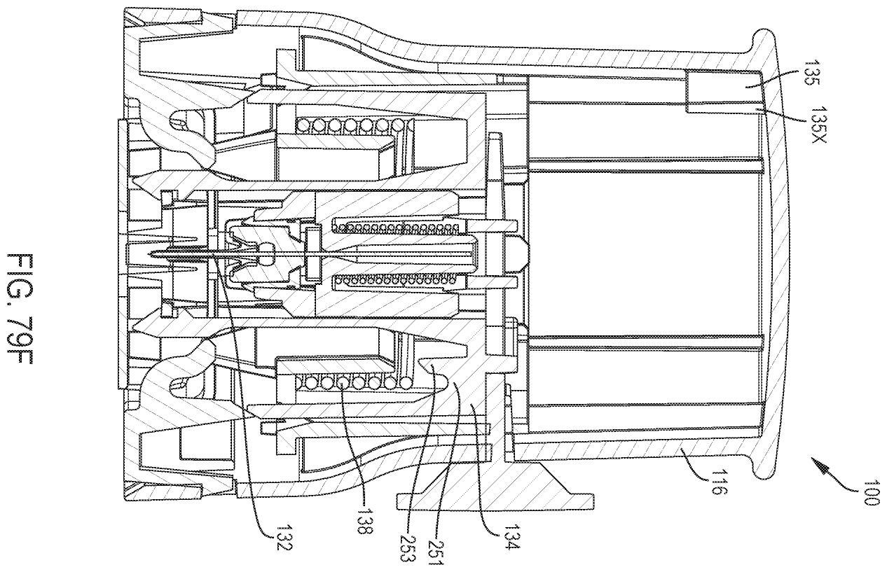

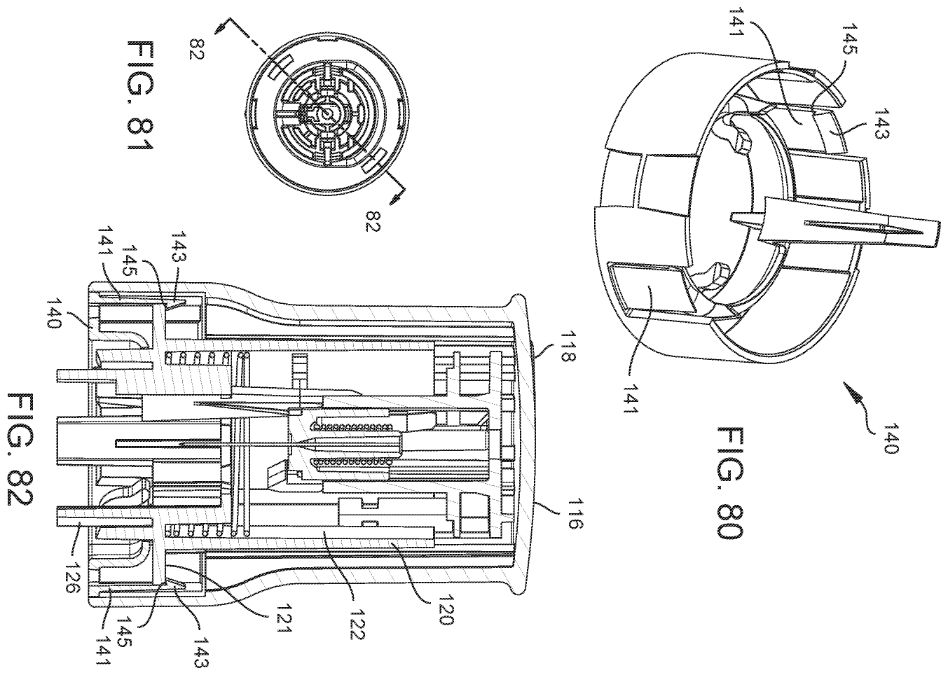

Referring now also to FIGS. 60-64, a number of views of a sharp holder 130 are depicted. The sharp holder 130 may include a bias member receiving bay 290 in which the spring 136 may be disposed. The sharp holder 130 may also include a wall 292 which surrounds the bias member receiving bay 290. The wall 292 may include two projections 294 on an exterior face thereof. The two projections 294 in the example embodiment are rails which are disposed opposite one another on the sharp holder 130. These rails may ride along guides 354 (sec, e.g., FIG. 70A-70B) on a portion of the sharp retractor 134. The projections 294 may extend from the sharp holder 130 so as to match the width of the cannula subassembly 114 at a plane of the cannula subassembly 114 including the cars 204 of the septum housing 108.

In various embodiments, the wall 292 may also include interrupted regions which create one or more cantilevered arms 296. In the example embodiment, the cantilevered arms 296 are disposed opposite one another on the sharp holder 130. One of the cantilevered arms 296 may have a greater length than the other of the cantilevered arms 296. In other embodiments, both cantilevered arms 296 may be identical mirror images (see, e.g., FIG. 78C) to allow for easier assembly of the inserter assembly 100. Both of the cantilevered arms 296 may extend above a top face 298 of the remainder of the wall 292. Each of the cantilevered arms 296 may include a protuberance 300 disposed at an unsupported or terminal end thereof. A ledge section 302 may be defined by a portion of each of the protuberances 300. At least one of the ledges 302 may extend substantially perpendicular to the cantilevered arms 296. At least one of the ledges 302 may be angled with respect to the cantilevered arm 296 on which it is included such that the undercut region has a triangular cross section. In the example embodiment, the ledge 302 on the longer of the two cantilevered arms 296 is so undercut. A sharp holder 130 may also include a port 304. The port 304 may be used to supply glue or adhesive into the sharp holder 130 to fixedly retain the insertion sharp 132 into the sharp holder 130.

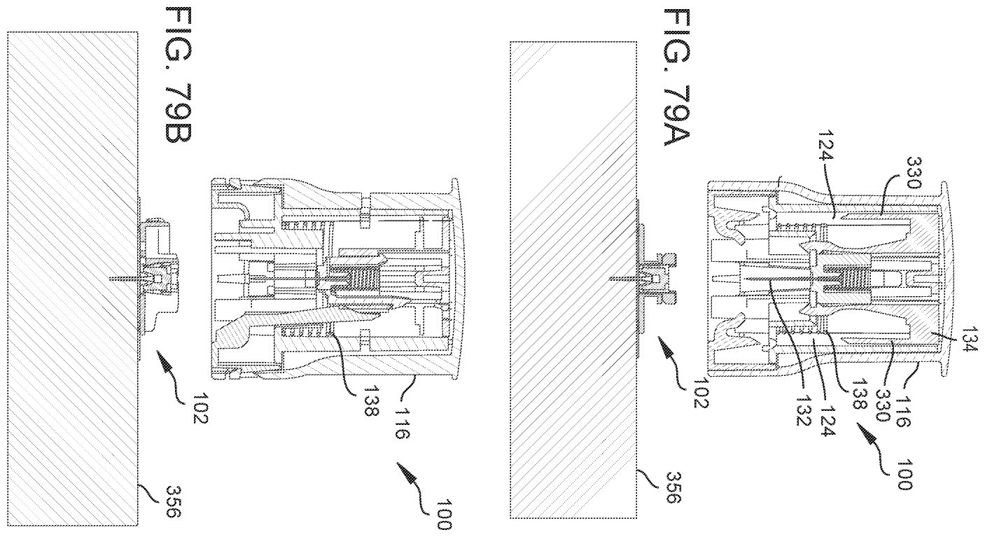

An alternate embodiment of a sharp holder 130 is shown in FIGS. 65-68. As shown, the longer of the two cantilevered arms 296 of the sharp holder 130 includes a protuberance 300 with a ridge 301. The ridge 301 may be medially located on the protuberance 300 though may be located in other positions in alternative embodiments. As shown, the protuberance 300 may be double beveled. The portion of the protuberance 300 adjacent the ledge section 302 may have a steeper bevel than the portion of the protuberance 300 distal to the ledge section 302. The ridge 301 may be formed on the portion of the protuberance adjacent the ledge section 302. In the example, the ridge 301 is formed as an extension of the bevel angle from the portion of the protuberance 300 distal to the ledge section 302 which extends to the portion of the protuberance 300 adjacent the ledge section 302. Such a ridge 301 may be included on any of the sharp holder 130 embodiments depicted herein.

As best shown in FIG. 59B, the ledge 302 on the longer of the cantilevered arms 296 may rest on a catch 306. The catch 306 in the example embodiment is included on the sharp retractor 134. This may inhibit the release of energy stored in spring 136 and hold the spring 136 under compression in the example embodiment. The catch 306 may have an angle which cooperates with any angle of the ledge 302 to firmly retain the ledge 302 on the catch 306. A finger 308 extending from the base retainer 140 of the insertion assembly 100 may extend through a void 322 (sec, e.g., FIG. 70C) adjacent the catch 306. Displacement of the finger 308 relative to the catch 306 may cause disengagement of the ledge 302 with the catch 306. This may trigger release of the sharp holder 130 and freeing of the spring 136 from its energy storing state. Though depicted as an upstanding finger 308, other types of actuation projections may be used. Additionally, in some embodiments, the finger 308 or other actuation projection may be included on another part of the inserter assembly 100. For example, a finger 308 may project from the interior surface of the top face of the exterior housing 116. The engagement of the catch 306 and ledge 302 is an example of a trigger (sec, e.g., description of FIG. 57) which may be operated to begin actuation of an inserter assembly 100.

Referring now also to FIGS. 69-70C, a number of views of a retainer base 140 and sharp retractor 134 are depicted. As shown, the finger 308 of the retainer base 140 (FIG. 69) is formed as a continuous part of the retainer base 140. The example retainer base 140 includes a first ring portion 310 and a second ring portion 312. The first ring portion 310 and second ring portion 312 are connected by an intermediary region 314 and may be concentric with one another. The intermediary region 314 may be generally flat and may serve as a skin contacting portion of the inserter assembly 100 which is substantially level with the bottom face 162 of the infusion set base 106. The finger 308 extends from the second ring portion 312. As shown, the finger 308 includes an upstanding segment 316 and a fin 318.

In various embodiments, the sharp retractor 134 may include a guide 320 along which the fin 318 may slide during assembly. The guide 320 may also, in certain embodiments, ride along the fin 318 during at least a portion of the retraction of the sharp retractor 134. The example guide 320 is formed as two raised parallel ribs. The catch 306 includes two supports 324 which the ledge 302 of the cantilevered arm 296 may engage with. The supports 324 may include a gap 326 therebetween. The gap 326 may have a width equal to or wider than the width of the fin 318. The fin 318 may be advanced through the gap 326 during lifting of the inserter assembly 100 from the skin 356. This may cause the fin 318 to come into abutment with the protuberance 300 on the cantilevered arm 296 and force the cantilevered arm 296 to bend inward. Once the fin 318 has progressed a certain distance, the cantilevered arm 296 may be deflected to the point that the ledge 302 no longer engages the catch 306 and the spring 136 may be released and actuation may be triggered. The location of the fin 318 on the finger 308 and/or the height of the finger 308 may be adjusted to alter the displacement distance at which release of the ledge 302 from the catch 306 occurs. In embodiments where the protuberance 300 includes a ridge 301 (see, e.g., FIGS. 65-68), the ridge 301 may be sized so as to fit through the gap 326. Thus, the ridge 301 may increase the total amount that the cantilevered arm 296 may be deflected by the fin 318 and provide extra assurance that ledge 302 is fully displaced off of the catch 306. As a result, the range of acceptable tolerances on various features related to the catch 306 and sharp holder 130 may be greater.

Still referring to FIGS. 59A-59B in conjunction with FIGS. 69-70B, spring 138 may be held in an energy storing state by at least one latching engagement as well. As shown, the sharp retractor 134 may include a first set of arms 330 and a second set of arms 332. The first set of arms 330 may extend from a top plate 328 of the sharp retractor 134. These arms 330 may extend substantially parallel to one another and may be disposed in opposing fashion on the sharp retractor 134. Additionally, the first set of arms 330 may extend laterally to a central cavity 334 of the sharp retractor 134. In some embodiments, the first set of arms 330 may be attached to the top plate 328 of the sharp retractor 134 at outcropped regions 336 of the top plate 328. The arms 330 may thus fit within channels formed by rails 124 of the interior housing 120 to prevent rotation and guide any displacement of the sharp retractor 134 during actuation. Additionally, outcropped regions 336 may be included in an asymmetric fashion on the sharp retractor 134 to ensure that the sharp retractor 134 is assembled into the inserter assembly 100 in a prescribed orientation. The first set of arms 330 may be cantilevered and attached to the sharp retractor 134 at the top plate and a second plate 338 which may extend parallel to the top plate 328 and below the top plate 328. Thus, the arms 330 may form resilient projections or members which may deflect if sufficient force is exerted against them. The unsupported end of the arms 330 may include a curved or ramped section 340. This section 340 may abut against a complimentarily contoured face of deflector members 358 to facilitate and or direct such deflection. A notch or pair of notches 342 may also be present on each of the first set of arms 330. In the example, each arm 330 includes a pair of notches 342 located near the unsupported ends of the arms 330.

FIG. 71 depicts a cross-section of an inserter assembly 100 taken through a plane in one of the arms 330 which extends along the length of the arm 330. As shown, each of the one or more notches 342 may engage with a cooperating projection 344. The cooperating projection 344 may be included on the interior housing 120. Thus, the interaction of the notch(es) 342 and cooperating projection(s) 344 may maintain the spring 138 under compression and serve as a retraction prevention latch. This may be particularly helpful during assembly as the infusion set base 106 may not be in place and the spring 138 may otherwise be free to relax. Additionally, this engagement may ensure that the top plate 328 of the sharp retractor 134 is disposed slightly below the fenestrations 148, 150 to allow for introduction of a lock member 146. Though notches 342 are shown, the arms 330 and interior housing 120 may engage in other ways. For example, the arms 330 or interior housing 120 may include a projection which forms a ledge. The ledge may engage with a catching recess in the other of the arms 330 or interior housing 120.

Again referring now primarily to FIGS. 59A-59B in conjunction with FIGS. 69-70B, in some embodiments additional latches may be included in an inserter assembly 100 which aid in maintaining one of the springs 136, 138 in an energy storing state. In the example embodiment, spring 138 is held in an energy storing state by an additional latch arrangement. As shown, the second set of arms 332 of the sharp retractor 134 extend from a bottom portion of the wall defining the central cavity 334. The second set of arms 332 may be disposed in opposing relationship to one another and may be cantilevered. Each of the second set of arms 332 may include a protuberance 346 disposed at an unsupported end thereof. Each protuberance 346 may form a ledge 348 on the arm 332 on which it is included. Additionally, the arms 332 may include a nub 350 or raised ramp which increases in thickness as distance from the cavity 334 increases. The nub 350 may be disposed intermediate the unsupported end of the arm 332 and its attachment point to the remainder of the sharp retractor 134.

As best shown in FIG. 59A the ledge 348 may capture a portion of the infusion set base 106. Specifically, the ledges 348 may catch on an outcropped portion of the infusion set base 106. The base 106 may include rails, step features, nubs or any other suitable protrusions to provide a complimentary catch surface for the ledges 348. In certain embodiments, the ledges 348 may catch on guides 172 (see, e.g., FIG. 4A) of the infusion set base 106. Thus the infusion set base 106 may be retained within the inserter assembly 100. In alternative embodiments, such as those in which an infusion set base 106 of the type depicted in FIGS. 21A-21F is used, a portion of each arm 332 may extend at least partially into a respective receiving slot defined in the base 106. The ledges 348 may engage a catch present at an end of the respective receiving slots so as to retain the base 106 within the inserter assembly 100.

Ledges 348 may be angled with respect to the cantilevered arm 332 on which it is included such that the undercut has a triangular cross section. The portion of, for example, the guides 172 (or any other catch feature) on which each ledge 348 catches may be angled in a cooperating manner to help ensure a robust engagement. The retainer base 140 (or the exterior housing 116 in embodiments like that shown in FIG. 73A-73B) may include retaining projections 359 which may abut or nearly abut arms 332 and hold the arms 332 from displacement out of engagement with the infusion set base 106 prior to inserter assembly 100 actuation. This may help prevent any accidental firing of the inserter assembly 100.

In certain embodiments, only one arm 332 may be included. In some embodiments, the arms 332 on the sharp retractor 134 may not engage the infusion set base 106. The arms 332 may engage a portion of the interior housing 120 so as to prevent premature retraction of the sharp retractor 134. The interior housing 120 may also include latch which may interface with the guide 172 or another cooperative portion of the infusion set base 106 to retain the infusion set base 106 in place.

A number of standoffs or alignment projections 352 may be included in the inserter assembly 100 to aid in ensuring that the infusion set base 106 is assembled into the inserter assembly 100 in a desired orientation. The standoffs 352 may be disposed on an interior housing 120 of the inserter assembly 100 as shown. While retained by the arms 332 of the sharp retractor 134, the infusion set base 106 may be held such that surfaces of the infusion set base 106 are adjacent to the standoffs 352. This may prevent infusion set base 106 and the sharp retractor 134 on which it is retained from displacing into the inserter assembly 100 due to the presentation of a mechanical interference by the standoffs 352. As a consequence, the catching of the ledges 348 of the arms 332 on the infusion set base 106 may also aid in holding the spring 138 in an energy storing state. The standoffs 352 may also ensure that the infusion set base 106 is positioned within the inserter assembly 100 such that the adhesive 374 may be pressed against the skin 356. In the example, the standoffs 352 ensure that the infusion set base 106 is substantially even with the skin contact face on the retaining base 140.

With the infusion set base 106 so positioned, the infusion set base 106 may also act as a protective barrier. As the cannula subassembly 114 and insertion sharp 132 may be internal to the inserter assembly 100, when the infusion set base 106 in the initial retained position, the user may be protected from accidental contact with the insertion sharp 132. This may additionally help to keep the cannula 104 or insertion sharp 132 from coming into contact with contaminants. Though a void for receipt of the cannula subassembly 114 may extend through the entirety of the infusion set base 106, the void may be sized to prevent finger ingress (e.g. have a cross-section smaller than that of a finger). Thus the infusion set base 106 may present an obstacle which blocks unintentional access to the insertion sharp 132 and cannula 104. Additionally, as the cannula subassembly 114 is internal to the inserter assembly 100, any adhesive backing 111 provided on the infusion set base 106 need not include an interruption to allow for passage of the cannula 104 therethrough. Thus, the void in the infusion set base 106 for the cannula subassembly 114 may be blocked by the adhesive backing 111 until just prior to use. This may further prevent finger ingress and may mitigate potential for detritus to enter the inserter assembly 100. In some examples, the adhesive 374 may also extend over this opening and may be punctured through during insertion of the cannula 104 into the skin 356.

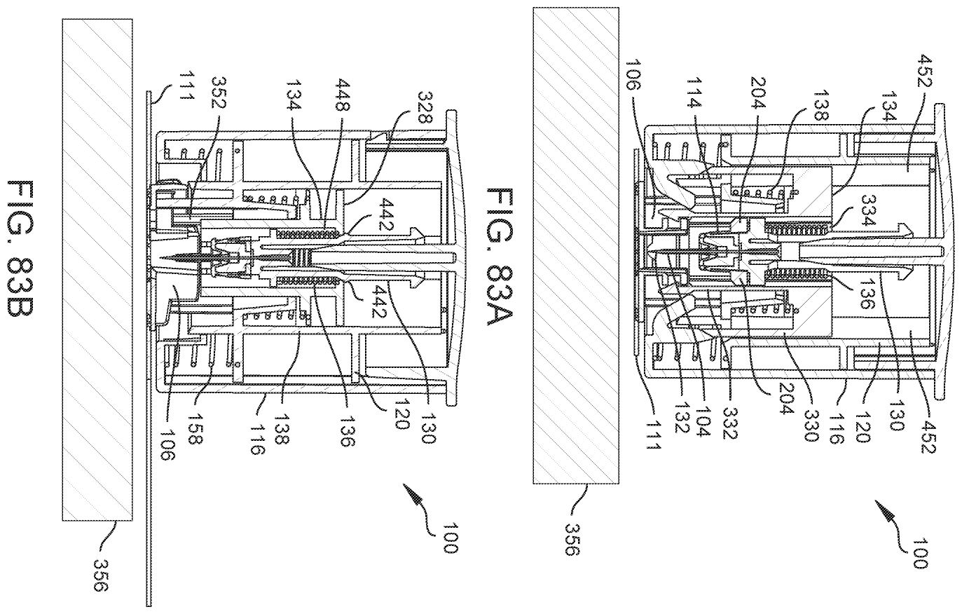

Referring now to FIG. 72, an isolated view of the alternative exterior housing 116 depicted in the inserter assembly 100 embodiment of FIG. 2 is shown. As shown, instead of a retainer base 140, various features of the retainer base 140 may be included in the exterior housing 116. These features may all be formed unitarily and integral to one another as a single monolithic component during a molding operation for example. As shown, the finger 308 and fin 318 are included as part of the exterior housing 116. The finger 308 may extend from a central ring portion 313 connected by an intermediary region 314 to the rest of the exterior housing 116. The deflector members 358 are similarly included as part of the exterior housing 116.

Cross sectional views of the example embodiment of the inserter assembly 100 in FIG. 2 are shown in FIGS. 73A-73B. This inserter assembly 100 includes the exterior housing 116 depicted in FIG. 72. The views in FIGS. 73A-73B show this alternate embodiment of the inserter assembly 100 in the same state as the inserter assembly 100 depicted in FIGS. 59A-B and FIG. 71. The inserter assembly 100 shown in FIGS. 73A-73B may operate similarly to the inserter assembly 100 depicted in FIGS. 59A-B and FIG. 71. For sake of brevity, the following describes various operational states of the inserter assembly 100 shown in FIGS. 59A-B and FIG. 71.

Referring now to FIGS. 74A-74B, two cross sectional views of an inserter assembly 100 are shown. In FIGS. 74A-74B, the example inserter assembly 100 has been placed against the skin 356 at a desired infusion site and is beginning to be withdrawn by a user. As shown, the adhesive 374 on the bottom face 162 of the infusion set base 106 may adhere to the skin 356 resulting in the skin 356 being tugged upward with the inserter assembly 100 as the inserter assembly 100 is pulled away from the body by the user. The adhesive 374 may be selected to be aggressive enough to maintain adhesion to the skin 356 while the skin 356 is lifted from the underlying anatomy. The exterior housing 116 and retainer base 140 which may form a casing for the inserter assembly 100 may displace together with the hand of the user as the user removes the inserter assembly 100 from their body. The other components of the inserter assembly 100 may not be constrained to displace as a unit with the exterior housing 116 and retainer base 140 (at least during a portion of the inserter assembly 100 removal action). As the infusion set base 106 is in latching engagement with the sharp retractor 134 and the sharp retractor 134 is in latching engagement with the interior housing 120 and sharp holder 130, these components may be held behind. Thus, these components may form a first unit of the inserter assembly 100 which is more constrained in its ability to displace than a second casing unit of the inserter assembly 100. As mentioned elsewhere herein, these components may move in tandem with the casing for at least an initial part of the inserter assembly 100 removal motion. The first and second unit may be releasably engaged with one another for this portion of the removal motion. For example, an interference or interfering relationship may be present between the first and second units. This interference may prevent relative motion of the first and second units until more than a threshold force is applied. When more than the threshold force is applied, the interference may be overcome and the first and second unit may begin to displace relative to one another.

Still referring to FIGS. 74A-74B, during removal, the exterior housing 116 and retainer base 140 may displace away from the skin 356 relative to the components of the second unit substantially along the axis of the insertion sharp 132. This may cause deflector members 358 included on the retainer base 140 to bend resilient members or arms 330 inward. It may also cause the finger 308 on the retainer base 140 to advance into the void 322 (see, e.g. FIG. 70C). Thus, the finger 308 may eventually displace a distance sufficient to trigger actuation of the inserter assembly 100. The resiliency of the arms 330 and the interference presented by the deflector members 358 may cause the entire inserter assembly 100 to move as a unit for at least a portion of the inserter assembly 100 withdrawal motion from the body. The deflector members 358 may bend or deflect the arms 330 out of the interfering relationship with the deflector members 358. Portions of the inserter assembly 100 may displace relative to one another once the force exerted by the elasticity of the skin exceeds a force threshold. Thus, the interference between the first and second unit preventing relative displacement of the first and second unit may be overcome by deflecting at least a part of one of the units out of an interfering relationship with the other of the units. This may occur after a threshold hold force is applied to pull the first and second unit apart. Separating force may be generated by a user displacing the casing away from the body and the elasticity of the tugged or lifted skin 356 pulling the first unit in the opposite direction.

In various embodiments, the resiliency of the resilient members or arms 330 may control, at least in part, a distance which a given user's skin 356 is tugged away from the body before actuation of the inserter assembly 100 occurs. As the skin 356 is tugged away from the body, the elasticity of the skin 356 may exert a pulling force which presses the arms 330 against the deflector members 358. The type, amount, and/or arrangement of adhesive 374 on the infusion set base 106 may be selected so as to withstand this force while maintaining adherence to the skin 356 and compatibility with the skin 356. Once this force overcomes the resiliency of the arms 330 and the components of the inserter assembly 100 described above begin to move relative to one another, the finger 308 may begin to advance into the void 322. Prior to this, triggering of actuation may be prohibited. Thus, the releasable engagement between the first and second units and the trigger may form an actuation restricting arrangement which may ensure that the skin is lifted prior to the inserter assembly being triggered. The resiliency may be chosen such that an inserter assembly 100 may be used on a wide range of individuals having different skin 356 properties (e.g. elasticity) while still ensuring the skin 356 is tugged at least some minimum distance before actuation is triggered. In some embodiments, inserter assemblies 100 may be produced with differing arm 330 resiliencies which may be suitable for different user groups. For example, arms 330 with less resiliency (e.g. an elderly resiliency) may be available for use for older user groups whose skin 356 has a tendency to be less elastic.

The steepness of the ramped section 340 on each arm 330 may be modified to alter the amount of force applied before relative movement occurs. Shallower angles on the ramped section 340 may be employed where more force before relative movement occurs is desired. Sharper angles on the ramped section 340 may be used where a lower force may be desirable. There may be a high (e.g. juvenile), medium (e.g. adult), and low (e.g. elderly) skin elasticity ramp angle in certain implementations. Additionally, the thickness of the arms 330 may be altered to change their resiliency. Thinner arms may be used where less force is desired and thicker arms may be used where more force is desired. Various supporting features such as buttresses may also be included and may support the arms 330 against deflection at a point near the supported end of the arms 330. The location of the second plate 338 may also be modified in certain examples to alter the length of the deflectable portion of the arms 330 making them more or less resilient. The material used to form the arms may also be selected based on its resiliency properties.

The length of the finger 308 and location of the fin 318 on the finger 308 may play a role in controlling, at least in part, the distance a user's skin 356 is tugged away from the body. These parameters may be modified to alter this distance.

Referring now to FIGS. 74C-D two additional cross-sections of an example inserter assembly 100 are shown. Each of the example inserter assemblies 100 includes an additional spring 156, 158. These springs 156, 158, are described above in relation to FIGS. 1A-1C. An additional spring 156, 158 may be used to adjust the amount of force build up before the fin 318 triggers the beginning of inserter assembly 100 actuation (e.g. via disengagement of a catch 306 and ledge 302, see, e.g., FIG. 59B). Springs 156, 158 may also help to remove any mechanical slop which may be present due to tolerancing of various components of the inserter assembly 100. Varying the characteristics described in the above paragraphs may allow one to empirically determine appropriate designs for various patient populations (e.g. juvenile, adult, elderly or high skin elasticity, medium skin elasticity, low skin elasticity). A skin turgor test, elastomer, or other testing may be used to match an appropriate inserter assembly 100 type to a particular patient.

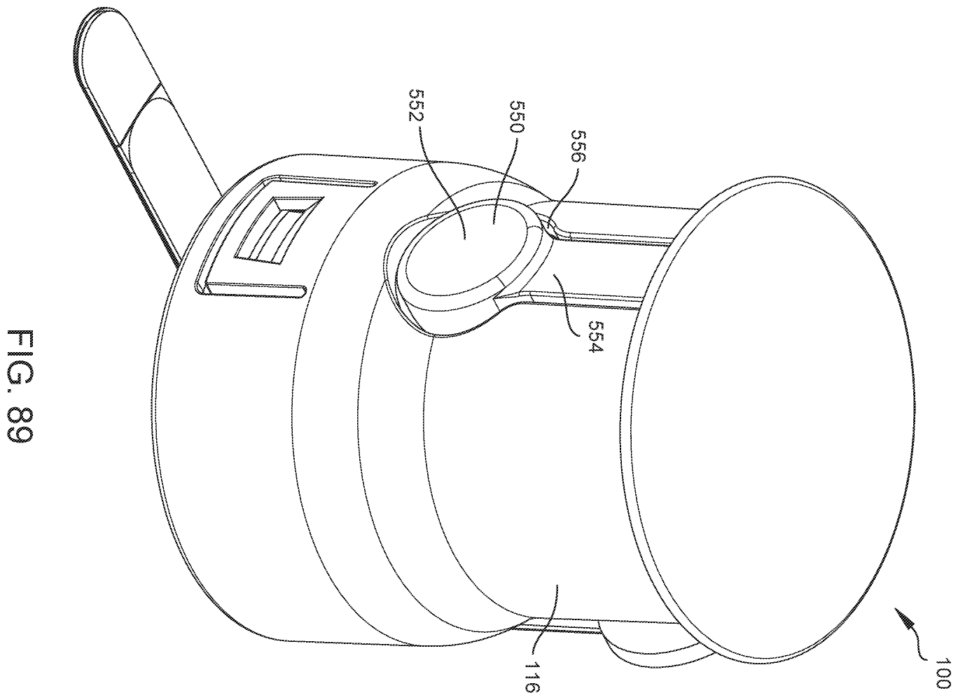

Referring now to FIGS. 75A-75B two cross sectional views of an inserter assembly 100 are shown. In FIGS. 75A-75B, the example inserter assembly 100 has been further withdrawn from the skin 356. The exterior housing 116 and retainer base 140 have continued to displace away from the skin 356 relative to the rest of the inserter assembly 100. The deflector members 358 included on the retainer base 140 have continued to flex or deflect the arms 330 inward. The finger 308 on the retainer base 140 has advanced into the void 322 (sec, e.g. FIG. 70C) such that the fin 318 of the finger 308 has dislodged the cantilevered arm 296 of the sharp holder 130 from the catch 306 (sec, e.g., FIG. 70C). Thus the movement of the exterior housing 116 and retainer base 140 has advanced the inserter assembly 100 to an insertion release point in FIG. 75A-75B. With disengagement of the cantilevered arm 296 from the catch 306, the spring 136 is free to release its stored energy and begin driving actuation of the inserter assembly 100.

Prior to the magnitude of relative displacement between the portions of the inserter assembly 100 increasing to this insertion release point threshold, the inserter assembly 100 may be precluded from triggering actuation. This may ensure that the skin is lifted some distance before actuation of the trigger arrangement (e.g. disengagement of a catch 306 and ledge 302, see, e.g., FIG. 59B) for the inserter assembly 100 can occur.

Referring now to FIGS. 76A-76B two cross sectional views of an inserter assembly 100 are shown. In FIGS. 76A-76B, the example inserter assembly 100 has been further withdrawn from the skin 356. As shown, the restoring action of the spring 136 may drive the sharp holder 130, insertion sharp 132, and cannula subassembly 114 toward the skin 356 along an insertion path. In the example embodiment, the sharp holder 130 at least partially extends out of the cavity 334 of the sharp retractor 130. As shown, the insertion sharp 132 and cannula 104 have just punctured through the skin 356. During puncture, the skin 356 may still be in a state in which it is tugged up away from muscle and other underlying body structures. The cannula subassembly 114 has also begun to be advanced toward the infusion set base 106. It should be noted that the spring 136 is still depicted in a compressed state in the forthcoming figures. This is for ease of illustration. As would be understood by one of skill in the art, the example spring 136 would expand over the course of the insertion movement. Also as shown, the retainer base 140 of the inserter assembly 100 may include a stop 364 which prevents relative movement beyond a certain point between the interior housing 120 and the casing formed by the exterior housing 116 and retainer base 140 (which may be connected to one another). This stop 364 may be included as a portion of the second ring 312 of the retainer base 140.

Referring now to FIGS. 77A-77B, two cross sectional views of an inserter assembly 100 are shown. As shown, the spring 136 has restored to a relaxed state and completed the insertion movement of the sharp holder 130, insertion sharp 132, and cannula subassembly 114 toward the skin 356. The relaxed state may be a completely relaxed state or a comparatively relaxed state where the spring 136 is still exerting some pressure against the sharp holder 130 to prevent it from jostling about. The notch 186 of the cannula subassembly 114 is shown in engagement with the protuberance 182 of the infusion set base 106 locking the cannula subassembly 114 in place and completing assembly of the infusion set 102. As shown, when the cannula subassembly 114 latches into the base 106, the cars 204 on the cannula subassembly 114 may press against the nubs 350 included on the arms 332. This may cause the arms 332 to be splayed apart resulting in disengagement of the arms 332 from the infusion set base 106. In turn, this may free spring 138 to begin releasing stored energy. Thus, FIGS. 77A-77B depict the insertion assembly 100 at a retraction release state and the arms 332 may serve as a retraction prevention latch.

In certain embodiments, retraction may not be automatic and/or may not be spring biased. For example, the insertion sharp 132 may remain in the advanced position and the removal action of the user may manually pull the insertion sharp 132 out of the cannula 104. In such embodiments, spring 138 may be omitted. In some embodiments, disengagement of the arms 332 from the infusion set base 106 may not be automatic. Any arrangement which would be apparent to one skilled in the art may be used to facilitate manual decoupling of the arms 332 from the infusion set base 106. A twisting action may be employed to free the arms 332 from the infusion set base 106 allowing the insertion sharp 132 to then automatically retract or be manually pulled out. One or more button may be included in alternative embodiments. Displacement of the one or more button may uncouple the arms 332 from the infusion set base 106 allowing the insertion sharp 132 to then be manually or automatically retracted out of the cannula 104. Squeezing of a deformable portion of the inserter assembly 100 may similarly cause uncoupling of the arms 332 from the infusion set base 106. As would be appreciated by one skilled in the art, the inserter assembly 100 embodiments described herein could be otherwise modified to allow for various types of other manual arm 332 release schemes.

In alternative embodiments where the infusion set base 106 is retained by a portion of the interior housing 120 and the arms 332 engage a portion of the interior housing 120, displacement of the sharp holder 130 may similarly cause release of the infusion set 102 and trigger retraction. For example, the cars 204 may collide with and cause displacement of catch features (e.g. spread them away from the infusion set 102) on the interior housing 120 resulting in them decoupling from the infusion set 102. Additional cars 204 or other projections on the cannula subassembly 114 may be included to cause disengagement of the arms 332 (e.g. via spreading of the arms 332) from the interior housing 120 to permit retraction.

In certain embodiments, there may be a dwell period during inserter assembly 100 actuation where retraction of the insertion sharp 132 has been triggered and the sharp retractor 134 is displacing, however, the insertion sharp 132 remains substantially static. During this dwell period, spring 136 may continue to exert pressure on the cannula subassembly 114 through the sharp holder 130. This may block any possible tendency of the cannula subassembly 114 to bounce or rebound as it is propelled into the infusion set base 106 and ensure it is firmly retained in the base 106. As shown, once the insertion movement is complete, a dwell gap 360 may be present between a stop 362 on the sharp retractor 134 and the ledge 302 on each cantilevered arm 296 of the sharp holder 130. Spring 136 may still have energy stored therein and continue to press against the sharp holder 130. The dwell gap 360 on each side may be equal in size.

Referring now additionally to FIGS. 78A-78B, which depict two cross sectional views of an inserter assembly 100, the dwell gap 360 may decrease until the stops 362 on the sharp retractor 134 contact the ledges 302 of the cantilevered arms 296 on the sharp holder 130. Once this occurs, the restoring action of spring 138 may begin to displace the sharp holder 130 and insertion sharp 132 affixed thereon along with the sharp retractor 134. This displacement may retract the insertion sharp 132 out of the cannula 104 and the infusion set 102. An alternative embodiment of an inserter assembly 100 at this stage of actuation is shown in FIG. 78C. In FIG. 78C the cantilevered arms 296 are equal length mirror images of one another. The stops 362 on the sharp retractor 134 may be at even height with one another in such embodiments.

As shown in FIGS. 79A-79B, once retraction has completed the sharp retractor 134 may be pressed against the exterior housing 116 by spring 138. For ease of illustration the spring 138 is still depicted in the compressed state, but would decompress to a relaxed state over the progression of the retraction as would be understood by one skilled in the art. As with spring 136, the relaxed state may be a completely relaxed state or a comparatively relaxed state where the spring 138 is still exerting some pressure against the sharp retractor 134 to prevent it from jostling about. After retraction, the insertion sharp 132 may be housed within the inserter assembly 100 to aid in protecting against unintentional finger sticks or the like. In the example embodiment, the insertion sharp 132 is housed further within the inserter assembly 100 after retraction when compared to its starting position. In other embodiments, the retracted location of the insertion sharp 132 may differ but may be at least housed as deep within the inserter assembly 100 as its initial starting location. The infusion set 102 may be held in place on the skin 356 with the cannula 104 indwelling in the patient.

Referring now also to FIGS. 79C-E, an alternative embodiment of an inserter assembly 100 is depicted. As shown in FIGS. 79C-D, the interior housing 120 of the inserter assembly 100 is shortened compared to, for example, that shown in FIG. 79B. In the exemplary embodiment shown in FIGS. 79A-B, the arms 330 of the sharp retractor 134 may ride within the rails 124 defined in the interior housing 120 as the sharp retractor 134 is retracted. Thus, the interior housing 120 may act as a guide body and the sharp retractor 134 may be guided to the retracted state by the interior housing 120. In the example shown in FIGS. 79C-D, the height of the interior housing 120 is such that the sharp retractor 134 is guided for a first portion of the retraction stroke and unguided for the remainder of the stroke. The at least partially unguided retraction displacement of the sharp retractor 134 may allow for some movement of the sharp retractor 134 within the exterior housing 116 as the retraction stroke transpires.

In certain embodiments, the exterior housing 116 may include at least one tipping projection 135 which obstructs retraction of a portion of the sharp retractor 134. This portion of the sharp retractor 134 may collide with the tipping projection 135 during the unguided portion of the retraction stroke. The tipping projection(s) 135 may have an end surface which is spaced a distance from an interior surface of the closed end of the exterior housing 116. An example tipping projection 135 may extend from the interior surface of the closed end of the exterior housing 116. The tipping projection(s) 135 may be disposed in an off-center position and, as shown, may be connected to both the interior surface of the closed end and side wall of the exterior housing 116. In some examples, the tipping projection(s) 135 may have a cross-section in the shape of the Latin character “T”. Example tipping projections 135 may include at least one nub body extending from the interior surface of the closed end of the exterior housing 116 to the open end of the exterior housing 116. In other embodiments, at least one ramped body may extend at an angle from the interior surface of the closed end of the exterior housing 116 to a point on the side wall of the interior of the exterior housing 116. In still other embodiments, the tipping projection(s) 135 may include at least one projection extending from a portion of interior side wall of the exterior housing 116 (e.g. near the closed end) into the interior volume of the exterior housing 116. Though such a tipping projection 135 may not directly connect with or contact the interior surface of the closed end of the exterior housing 116 such a tipping projection 135 may still be referred to as being disposed at the closed end of the exterior housing 116.

Thus, the sharp retractor 134 may be free to move all the way into contact with the interior surface of the exterior housing 116 on one side, but stopped shy on the other by the tipping projection 135. As the sharp retractor 134 displaces toward the closed end of the exterior housing 116, a portion of the sharp retractor 134 may contact the tipping projection 135. The tipping projection 135 may inhibit further displacement of that portion of the sharp retractor 134. Since the sharp retractor 134 may be unguided in a second stage of the retraction stroke, as the retraction displacement continues (e.g. as the retraction spring 138 continues to relax), the sharp retractor 134 may begin to tilt at an angle to the axis of the rest of the inserter assembly 100. As a result, the insertion sharp 132 on the sharp holder 130 would be tilted out of alignment with the axis of the inserter assembly 100. In alternative examples, the surface of the sharp retractor 134 most proximal the interior surface of the closed end of the interior housing 116 may be uneven so as to cause the sharp retractor 134 to tilt as described above. For example, tipping projection(s) 135 (e.g. ramped protrusions, nubs, “T” shaped projections, etc.) may be included on the sharp retractor 134 to engender tilting as the retraction stroke progresses.

When the retraction stroke completes, the insertion sharp 132 may be in a tilted state. This could further assist in stowing the insertion sharp 132 within the inserter assembly 100 after retraction. At the conclusion of the retraction stroke, the retraction spring 138 may not be fully relaxed. Thus, the retraction spring 138 may press the sharp retractor 130 against the closed end of the exterior housing 116 holding the insertion sharp 132 in the tilted state. This may also help prevent wiggling of the sharp retractor 134 when in the retracted state such that substantially no rattling occurs when the inserter assembly 100 is handled after use.

The location of the tipping projection(s) 135 may be selected to adjust the direction which the sharp retraction 134 tilts. In some examples including a tipping projection 135, the tipping projection 135 may be disposed such that the insertion sharp 132 is directed into a guard defined in another component of the inserter assembly 100. As shown best in FIG. 79D and FIG. 79E, the example tipping projection 135 is disposed such that tilting of the sharp retractor 134 displaces the insertion sharp 132 into a guide channel 137 of the interior housing 120 when the sharp retractor 134 is in the fully retracted state. Multiple tipping projections 135 arranged in a line or at least one tipping projection 135 with an elongate segment (e.g. cross-piece 135X of a “T” shaped tipping projection 135 shown in FIG. 79F) may assist in encouraging tilting of the insertion sharp 132 along a desired plane (e.g. perpendicular to the elongate segment or line of tipping projections 135). The guide channel 137 may assist in guiding an arm 332 of the sharp retractor 134 during a portion of the retraction stroke of the sharp retractor 134. The guide channel 137 may then serve as a guard into which the tip of the insertion sharp 132 is displaced as the sharp retractor 134 is tilted. In some embodiments, a retaining projection 359 of the retainer base 140 may further obstruct access to the tip of the insertion sharp 132 when the sharp retractor 134 is in the fully retracted state.

In still other embodiments, and referring now also to FIG. 79F, the sharp retractor 134 may include at least one standoff 251. When an inserter assembly 100 with such a standoff 251 is assembled, bias member 138 may be held within the inserter assembly 100 under greater deformation on the portion of the bias member 138 which abuts the standoff 251. The standoff 251 may also include an arm or hook body 253 which may help to retain the bias member 138 in contact with the standoff(s) 251. As the retraction stroke of the sharp retractor 134 occurs, the uneven compression of the bias member 138 (a compression spring in certain examples) afforded by the standoff(s) 251 may assist in tilting the sharp retractor 134 and insertion sharp 132 as further described above. The position of the standoff(s) 251 may further be selected to encourage tilting in a specific direction (e.g. into a guard such as guide channel 137). In the example embodiment shown in FIG. 79F, the standoff(s) 251 may be positioned substantially opposite any tipping projection(s) 135 on the exterior housing 116.

Referring now to FIGS. 80-82, in certain embodiments, after actuation certain components of the inserter assembly 100 may be locked in place. Alternatively, certain components may be prevented from displacement in particular directions or prevented from displacement beyond a predefined distance in particular directions. This may inhibit reuse of the inserter assembly 100 and may aid in protecting a user from unintended finger sticks. Some embodiments of retainer bases 140 may include one or more lock members 141. As shown, two lock members 141 are included though other embodiments, may include a greater or lesser number (e.g. three or four). The lock members 141 in the example embodiment are disposed opposite one another on the retainer base 140.

The exemplary lock members 141 in the example embodiment are depicted as cantilevered lock tabs. Each cantilevered tab includes a protuberance 143 which is disposed on a portion of that lock member 141 most distal to the skin contacting intermediary region 314 (sec, e.g., FIG. 69) of the retainer base 140. The protuberance 143 be ramped and may define a ledge region 145. As the exterior housing 116 and the retainer base 140 displace relative to the interior housing 120 during withdrawal of the inserter assembly 100 from the skin 356, a portion of the interior housing 120 may contact ramped portion of the protuberance 143. As shown, the interior housing 120 includes a radial flange 121 which separates the infusion set base interfacing segment 126 of the interior housing 120 from the railed segment 122 (see FIG. 1A) of the interior housing 120. This radial flange 121 may be the portion of the interior housing 120 which contacts the protuberances 143 of the lock members 141. Further relative displacement may cause deflection of the lock members 141 to allow for passage of radial flange 121 of the interior housing 120 passed the protuberance 143. As shown, in FIG. 82, once the radial flange 121 has displaced beyond the protuberances 143, the lock members 141 may resiliently restore back to their unstressed state. In the event that a user presses against the interior housing 120 after actuation, the radial flange 121 may abut against the ledges 145 of the lock members 141 may be unable to further displace into the exterior housing 116. Thus, the interior housing 120 may be constrained after actuation such a portion of the interior housing 120 is held a predefined distance from the tip of the insertion sharp 132. This portion may present an obstruction which may aid in blocking inadvertent contact of the insertion sharp 132 with a user after actuation.

Referring now to FIG. 83A-83B, two cross-sectional views of the example inserter assembly 100 depicted in FIG. 3 are shown. The inserter assembly 100 is depicted just as it is about to be applied to the skin 356. The adhesive backing 111 may be removed to expose the adhesive on the infusion set base 106. As shown, both springs 136, 138 may be in an energy storing state, which in this particular embodiment is a compressed state. In the example, spring 136 serves as an insertion driving bias member while spring 138 serves as an insertion sharp 132 retraction driving bias member. Spring 136 is held in compression between the insertion sharp holder 130 and the insertion sharp retractor 134 and, when released, drives the sharp holder 130 and components carried there on from a raised state to a forward state. Spring 138 is held in compression between the interior housing 120 and the sharp retractor 134. Upon release, spring 138 drives the sharp retractor 134 and sharp holder 130 from a post insertion state to a retracted state. An additional spring 158 is included in the example embodiment, though is optional. The additional spring 158 may alter the amount of force build up before actuation of the inserter assembly 100 is triggered and may help to remove any mechanical slop which may be present due to tolerancing of various components of the inserter assembly 100.

Referring now also to FIGS. 84A-87, a number of views of a sharp holder 130 and a retainer cap 406 are depicted. The sharp holder 130 may include a bias member receiving shelf 420 against which the spring 136 is held. The shelf 420 may include two projections 422 on a side thereof. The two projections 422 in the example embodiment are rails which are disposed opposite one another on the sharp holder 130. These rails may ride along guides 354 (sec, e.g., FIGS. 88A-88B) on a portion of the sharp retractor 134. The projections 422 may extend from the sharp holder 130 so as to match the width of the cannula subassembly 114 at a plane of the cannula subassembly 114 including the cars 204 of the septum housing 108.

A wall 426 may extend upward from the shelf 420. The exemplary wall 426 shown in FIGS. 84A-C includes interrupted regions which create one or more wall sections 428. The wall sections 428 may be crescent shaped as shown and are separated by an interrupt region formed by a U-shape recess which extends from the top face 430 of the sharp holder 130 nearly to the shelf 420. The recess may extend at least 90-95% or more of a distance extending from the top face 430 to the shelf 420. The recesses may allow deflection of the wall sections 428. The wall sections 428 may be disposed opposite one another on the sharp holder 130 and may be of equal length. In other embodiments, both wall sections 428 may not be identical mirror images. Each of the wall sections 428 may include a protuberance 432 at the end of the wall section 428 opposite the shelf 420. The protuberance 432 has a pileus type shape which generally widens as distance from the shelf 420 decreases. During assembly, this may facilitate installation of the sharp holder 130 into the insertion assembly 100 through the side of the sharp retractor 134 from which the arms 330, 332 (see, e.g. FIG. 83A) extend. A ledge section 434 may be defined by a portion of each of the protuberances 432. At least one of the ledges 434 may extend substantially perpendicular to the wall sections 428. At least one of the ledges 434 may be angled with respect to the wall section 428 on which it is included such that the undercut region has a triangular cross section.

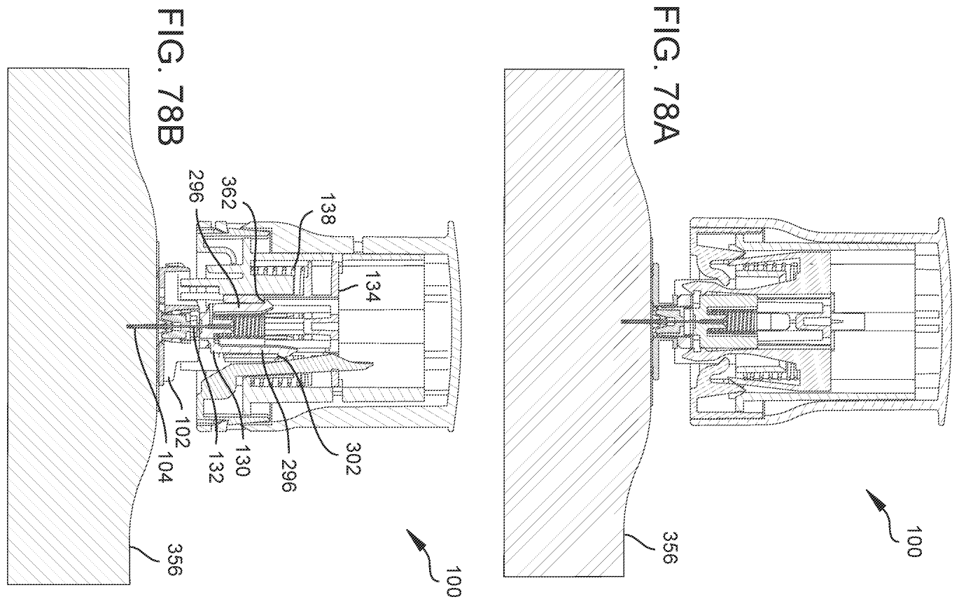

Additionally, each wall section 428 may include a first section 436 and a second section 438. The first section 436 may have a smaller width than the second section 438 and may be the more proximal of the regions to the shelf 420. The first section 436 and second section 438 may be connected by an intermediary region 440. The intermediary region 440 may be angled or curved so as to transition between the differing widths of the first section 436 and second section 438. A nub 442 may be included projecting from the intermediary section 440 or a portion one of the first section 436 and second section 438 adjacent to the intermediary section 440. Though not shown a glue or adhesive supply port similar to port 304 of FIG. 61 may be included.

Referring now primarily to FIGS. 85-87, a projection 412, which may be referred to herein as a spreader pin 412, extending from the cap retainer 406 of the insertion assembly 100 may project into a receiving void 444 between the wall sections 428. The spreader pin 412 may include a tapered region 446 on an end thereof and may or may not be hollow. The tapered region 446 may abut into the interior faces 448 of the wall sections 428 as the spreader pin 412 is advanced into the sharp holder 130 beyond a certain distance and may help guide the spreader pin 412 as it is displaced into the sharp holder 130. Further displacement of the spreader pin 412 into the sharp holder 130 may cause the wall sections 428 to be resiliently deflected apart or spread apart from one another thus widening the sharp holder 130 in order to accommodate the spreader pin 412 therein. This may also cause the distance between outward facing surfaces of the nubs 442 to increase when the spreader pin 412 is present.

Referring now primarily to FIG. 83B and FIGS. 88A-B, the nubs 442 may interact with another component of the inserter assembly 100 and prevent the force exerted by spring 136 against the shelf 420 from displacing the sharp holder 130 to a forward position. In the example shown, the nubs 442 are sized such that they may not fit into a cavity 334 included in the sharp retractor 134 when the spreader pin 412 is in place within the sharp holder 130. As spring 136 is captured between the shelf 420 of the sharp holder 130 and an interior face of the top plate 328 of the sharp retractor 134, the nubs 442 may inhibit release of energy stored in spring 136.

Still referring to FIGS. 83A-83B in conjunction with FIGS. 88A-88B, spring 138 may be held in an energy storing state by at least one latching engagement as well. As shown, the sharp retractor 134 may include a first set of arms 330 and a second set of arms 332. These arms 330, 332 may be substantially as described above in relation to FIGS. 70A-B. The sharp retractor 134 in this embodiment is depicted as having a symmetric design so as to allow the sharp retractor 134 to be assembled into an inserter assembly 100 in multiple orientations. This may help to simplify assembly. The sharp retractor 134 of FIGS. 70A-70B may also be constructed symmetrically by including voids 322 (see, e.g., FIG. 70C) on opposing sides of the cavity 334.

Each of the one or more notches 342 or the arms 330 may engage with a cooperating projection 344 (see, e.g. FIG. 71) on the interior housing 120. Thus, the interaction of the notch(es) 342 and cooperating projection(s) 344 may maintain the spring 138 under compression and serve as a retraction prevention latch. This may be particularly helpful during assembly. Though notches 342 are shown, the arms 330 and interior housing 120 may engage in other ways as described elsewhere herein.

Again referring now primarily to FIGS. 83A-83B in conjunction with FIGS. 88A-88B, in some embodiments additional latches may be included in an inserter assembly 100 which aid in maintaining one or more of the springs 136, 138 in an energy storing state. In the example embodiment, spring 138 is held in an energy storing state by an additional latch engagement provided by features of the arms 332 interacting with features of the infusion set base 106. This latch arrangement may be similar to as described above in relation to FIGS. 59A-59B. While retained by the arms 332 of the sharp retractor 134, the infusion set base 106 may be held such that surfaces of the infusion set base 106 are adjacent to the standoffs 352. This may prevent the infusion set base 106 and the needle retractor 134 on which it is retained from displacing into the inserter assembly 100 due to the presentation of a mechanical interference by the standoffs 352. As a consequence, the engagement of the arms 332 with the infusion set base 106 may also aid in holding the spring 138 in an energy storing state. The standoffs 352 may also ensure that the infusion set base 106 is positioned within the inserter assembly 100 such that the adhesive 374 may be pressed against the skin 356. In the example, the standoffs 352 ensure that the infusion set base 106 is substantially even with the skin contact face on the retaining base 140. As described elsewhere herein, with the infusion set base 106 so positioned, the infusion set base 106 and/or adhesive liner 111 may also act as a protective barrier.

Still referring primarily to FIGS. 83A-83B, as the retainer pin 412 is withdrawn from the sharp holder 130, the wall sections 428 may restore back to a non-deflected state. This may decrease the distance between the nubs 442 so as to allow passage of the nubs 442 into the cavity 334 in the sharp retractor 134. In turn, this may allow the spring 136 to begin releasing energy and displace the sharp holder 130 toward a forward position. As shown, in FIGS. 83A-83B, the example inserter assembly 100 is configured such that the entire inserter assembly 100 may move as a unit for at least a portion of the inserter assembly 100 withdrawal motion from the body. This may cause the skin 356 to be lifted a certain distance before the retainer pin 412 begins to be withdrawn and actuation is triggered. The amount the skin 356 is lifted or the force applied before portions of the inserter assembly 100 displace relative to one another may be modified as described in relation to the discussion of FIGS. 74A-74B.

In various embodiments, the ledge section 434 of the sharp holder 130 may prevent displacement of the sharp holder 130 beyond a certain amount. Alternatively, the ledge section 434 may be disposed on the sharp holder 130 such that a dwell gap 360 (sec, e.g., FIG. 77B) is present after the sharp holder 130 is propelled to the forward position. As described elsewhere herein, a dwell gap 360 may help to ensure the cannula subassembly 114 is firmly retained in the base 106.

In various embodiments, as the sharp holder 130 displaces, the cannula subassembly 114 may be coupled into the infusion set 106 as described elsewhere herein. Additionally, cars 204 of the cannula subassembly 114 may cause the arms 332 to spread apart as the sharp holder 130 is displaced leading to release of the infusion set base 106 from the arms 332. This may release spring 138 and begin retraction of the insertion sharp 132. During retraction, the dwell gap 360 may decrease (if present) until top plate 328 of the sharp retractor 134 contacts the ledges 434 on the sharp holder 130. Once this occurs, the restoring action of spring 138 may begin to displace the sharp holder 130 and insertion sharp 132 affixed thereon along with the sharp retractor 134. This displacement may retract the insertion sharp 132 out of the cannula 104 and the infusion set 102. Once retraction has completed the sharp retractor 134 may be pressed against the exterior housing 116 by spring 138 and the insertion sharp 132 may be housed within the inserter assembly 100 to aid in protecting against unintentional finger sticks or the like. The infusion set 102 may be held in place on the skin 356 with the cannula 104 indwelling in the patient.

Referring now to FIG. 89, another embodiment of an inserter assembly 100 including a manual trigger is depicted. The example inserter assembly 100 includes a button 550 which when displaced may trigger actuation. The button 550 may be included as a separate part which is assembled into the inserter assembly 100. Alternatively, and as shown, the button 550 may be formed monolithically with another portion of the inserter assembly 100. In the example embodiment, the button 550 is formed as a continuous part of the exterior housing 116. In the example embodiment, the button 550 includes an enlarged region that may include a depression 552 which may be substantially centrally located therein. The depression 552 may facilitate interaction with a user's finger. The button 550 may also include a cantilevered beam portion 554 which connects the button 550 to the rest of the exterior housing 116. In the example embodiment, the enlarged portion of the button 550 is disposed at a terminal unsupported end of the cantilevered beam portion 554, though may be located anywhere along the length of the cantilevered beam portion 554 in other embodiments. The cantilevered beam 554 extends in a direction substantially parallel to the longitudinal axis of the inserter assembly 100 and toward the top of the inserter assembly 100 in the example embodiment. In other embodiments, the cantilevered beam section 554 may extend in any direction. The cantilevered beam portion 554 may include a curvature which matches that of the exterior housing 116. A gap 556 surrounding the button 550 may be included to provide clearance for displacement or deflection of the button 550 during use. During actuation, the button 550 may be displaced at least partially into the inserter assembly 100 to trigger the insertion.

In some embodiments, the button 550 may not include a cantilevered beam section 554 and may instead be formed as a strip of material defined by cutouts flanking both sides of the strip. The enlarged section may be placed at or near the center of the strip. The strip may be deflected inward toward the inside of the inserter assembly 100 with application of pressure to trigger the insertion.

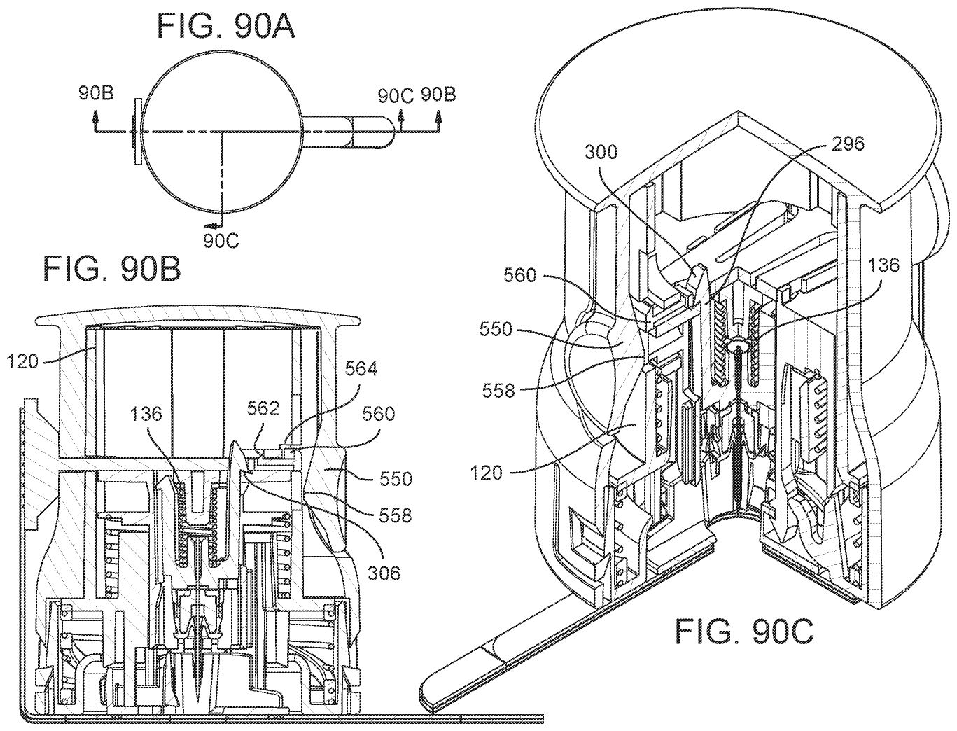

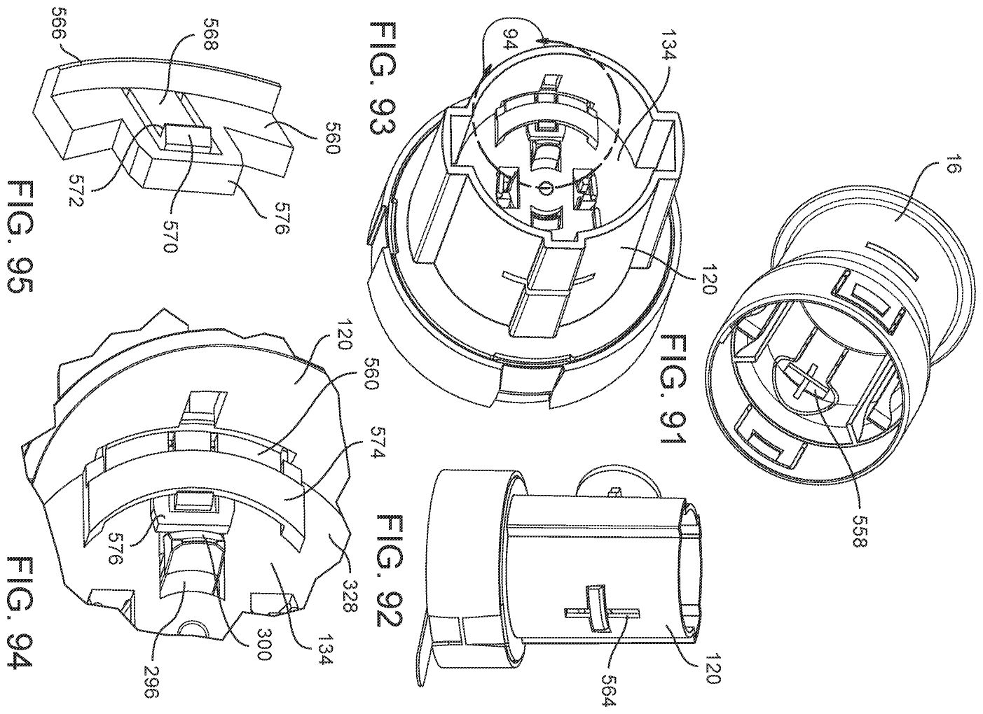

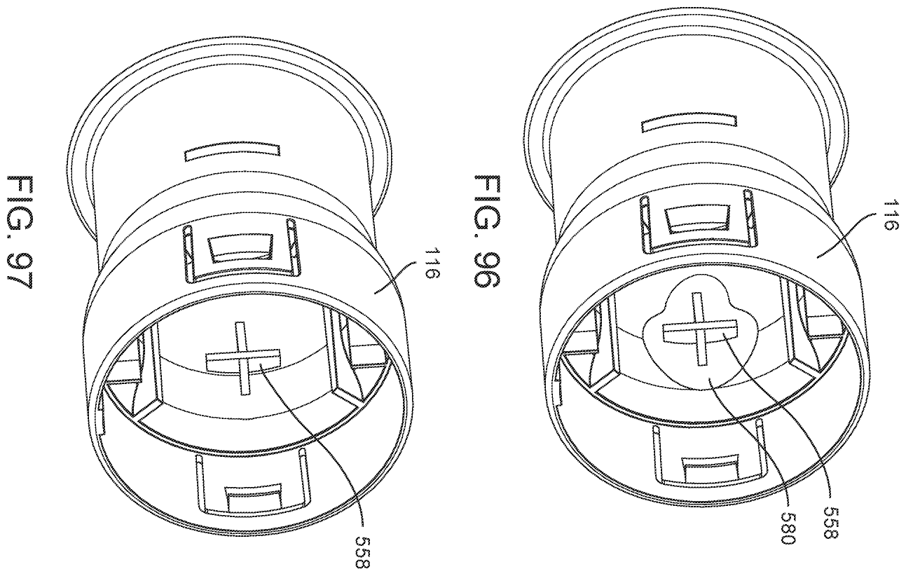

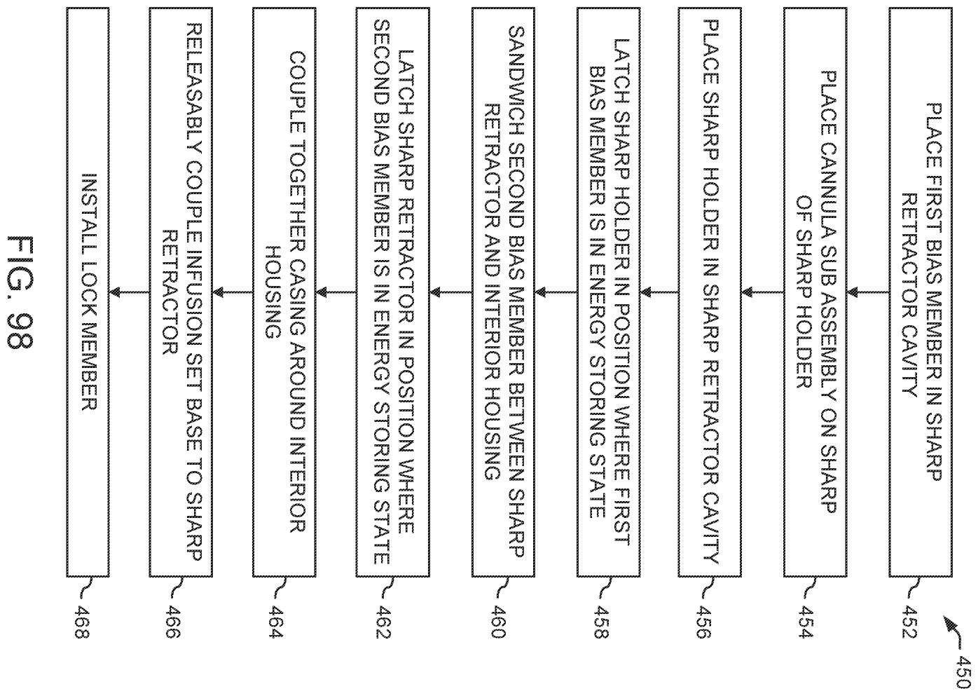

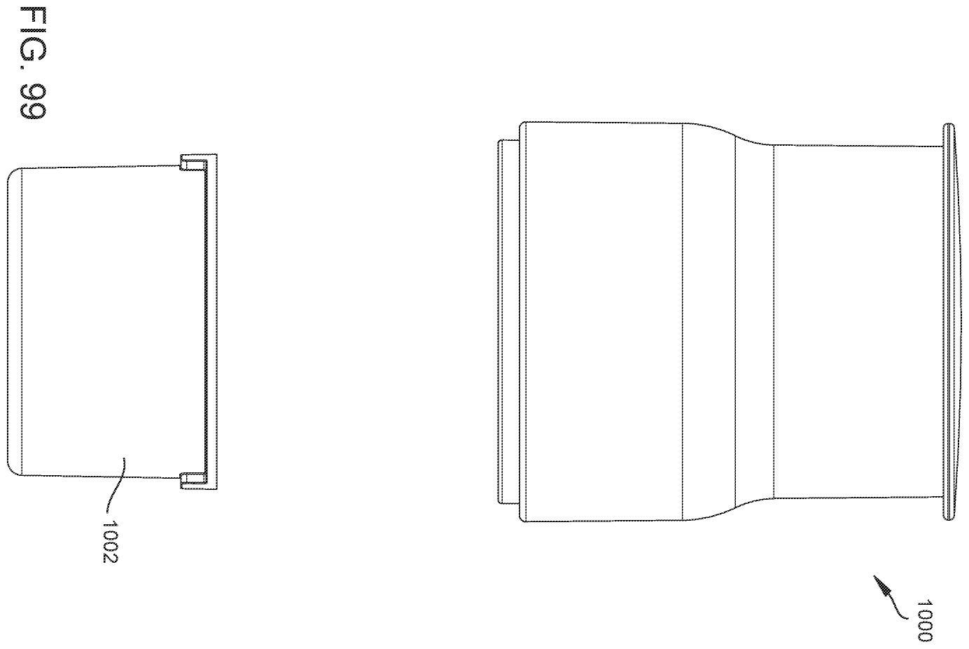

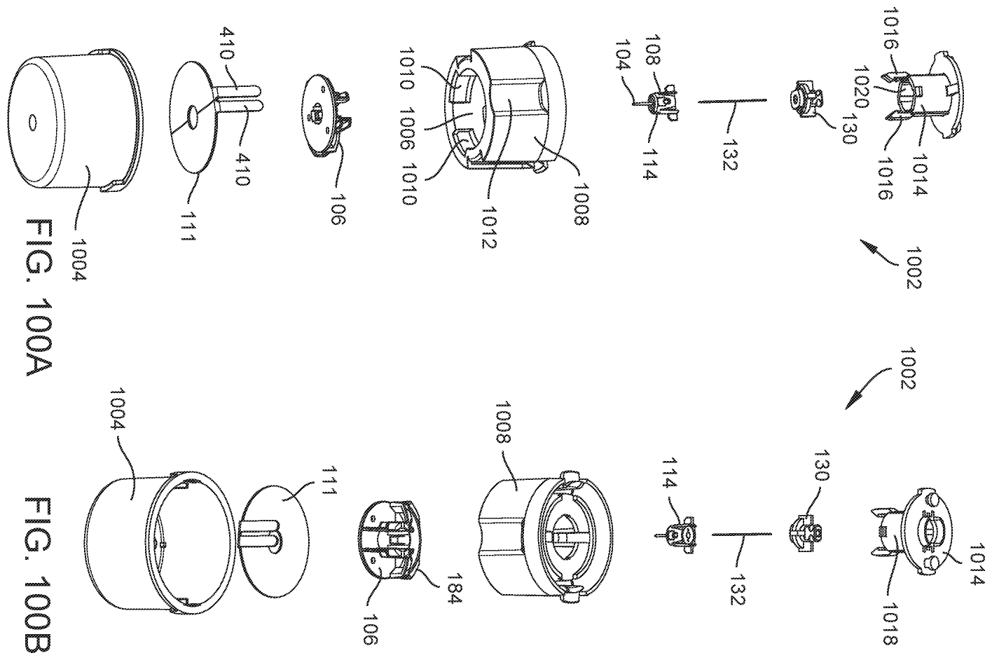

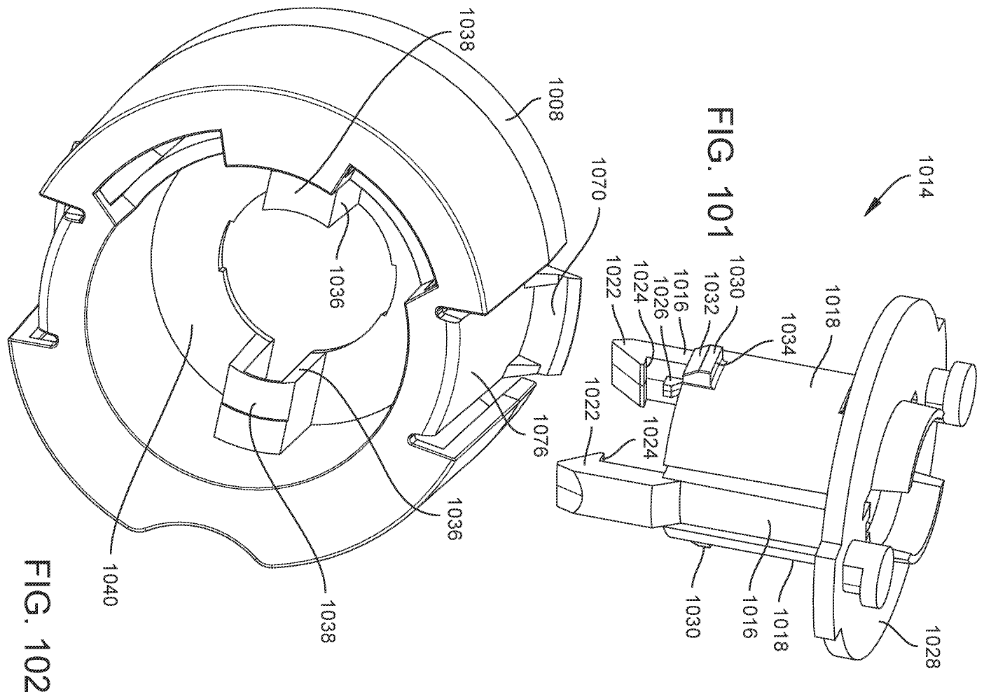

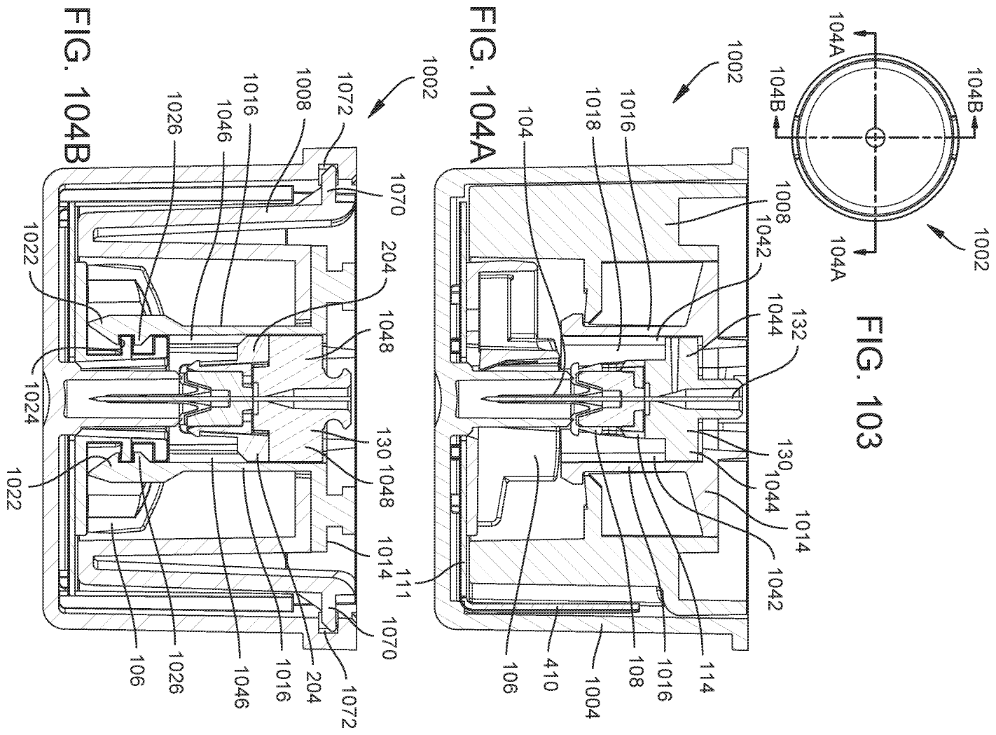

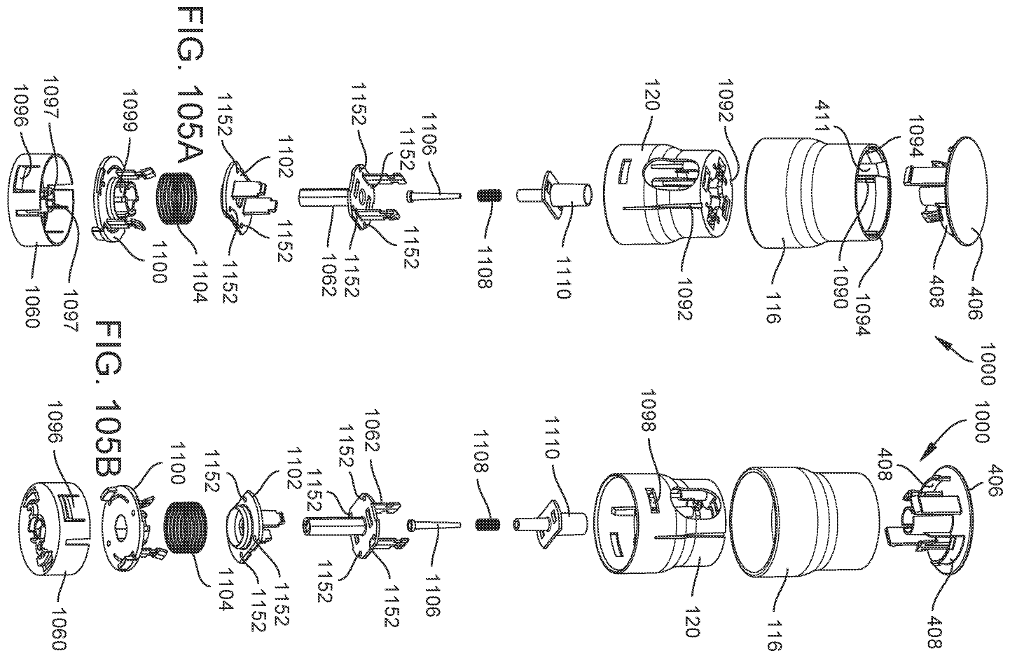

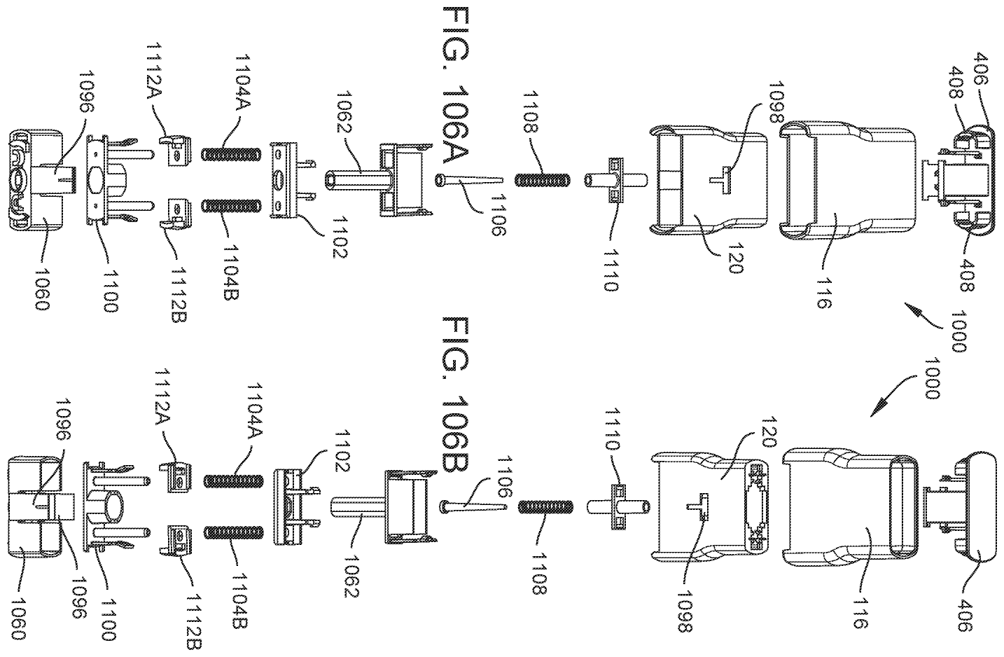

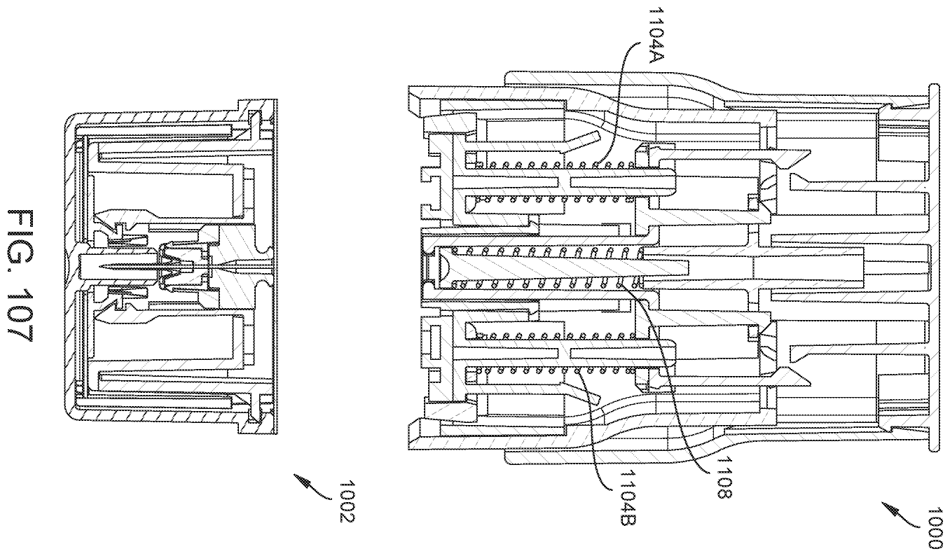

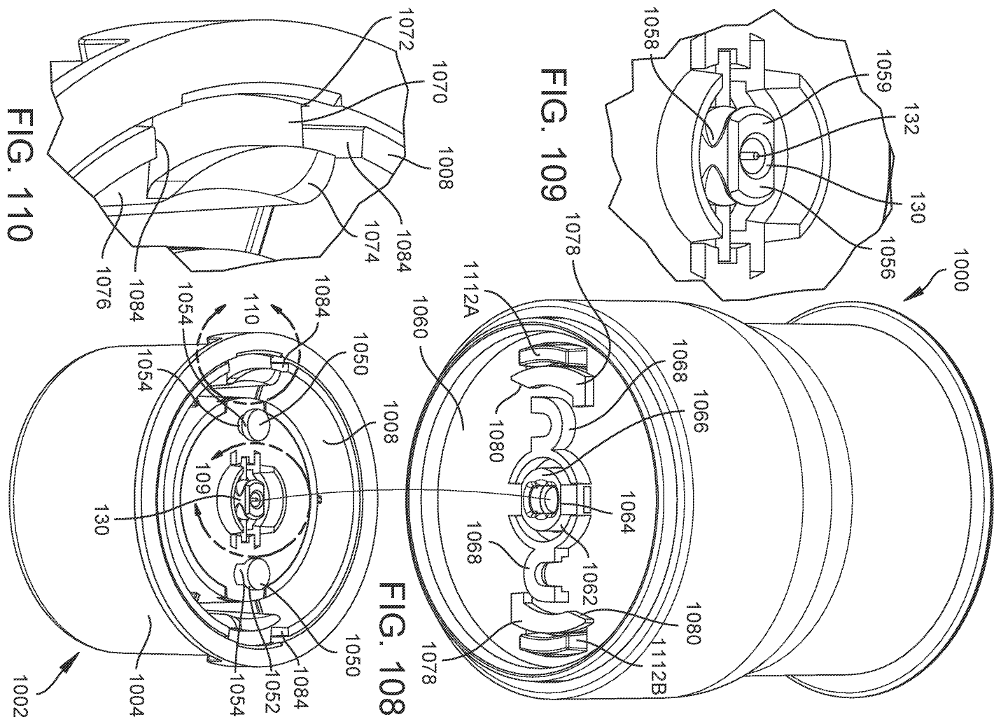

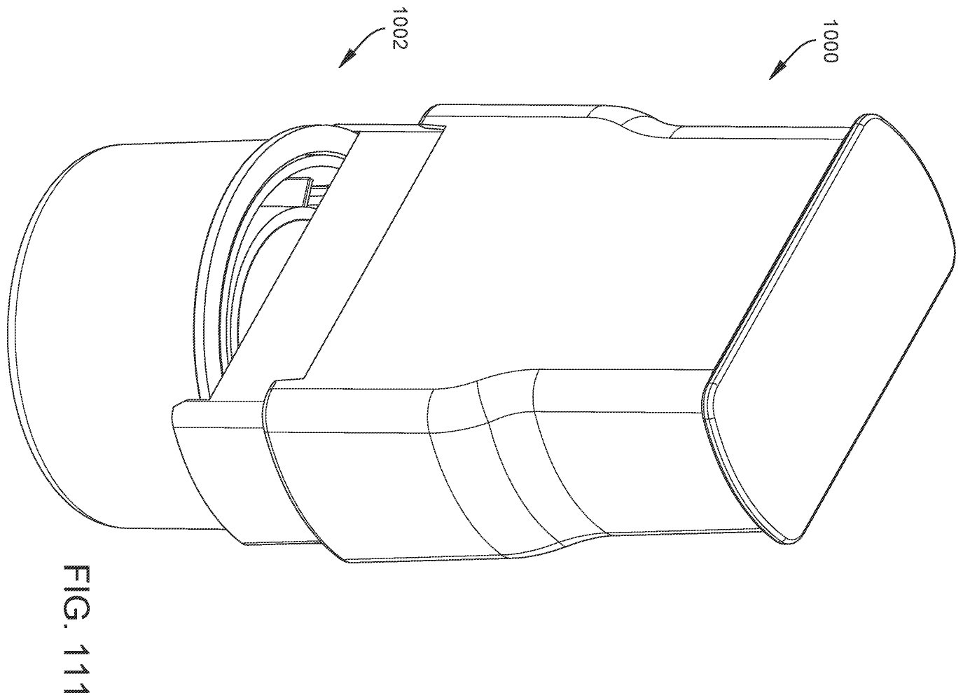

Referring now also to FIGS. 90A-90C and FIG. 91, an interlock may be included which prevents the button 550 from actuating the inserter assembly 100 until the skin has been displaced or until relative movement beyond a threshold magnitude has occurred. As shown, the button 550 may include a protuberance 558 (best shown in FIG. 91) on an inward face thereof.

The protuberance 558 may be disposed opposite the depression 552 of the enlarged region of the button 550 as shown. In an initial state, the protuberance 558 may be out of alignment with a sled member 560 as shown in FIG. 90B for example. As the inserter assembly 100 is withdrawn from the body, the skin is lifted, and relative displacement of portions of the inserter assembly 100 occurs, the protuberance 558 may be brought into alignment with the sled 560. At this point, portions of the inserter assembly 100 may be disposed similarly to as shown in FIG. 75B. Once in the aligned state, the button 550 may be displaced so as to come into contact with the sled 560. Button 550 displacement may then drive the sled 560 into the protuberance 300 disposed at the terminal end of a cantilevered arm 296 on the sharp holder 130. As the sled 560 continues to advance, the protuberance 300 may be displaced off the catch 306 freeing bias member 136.

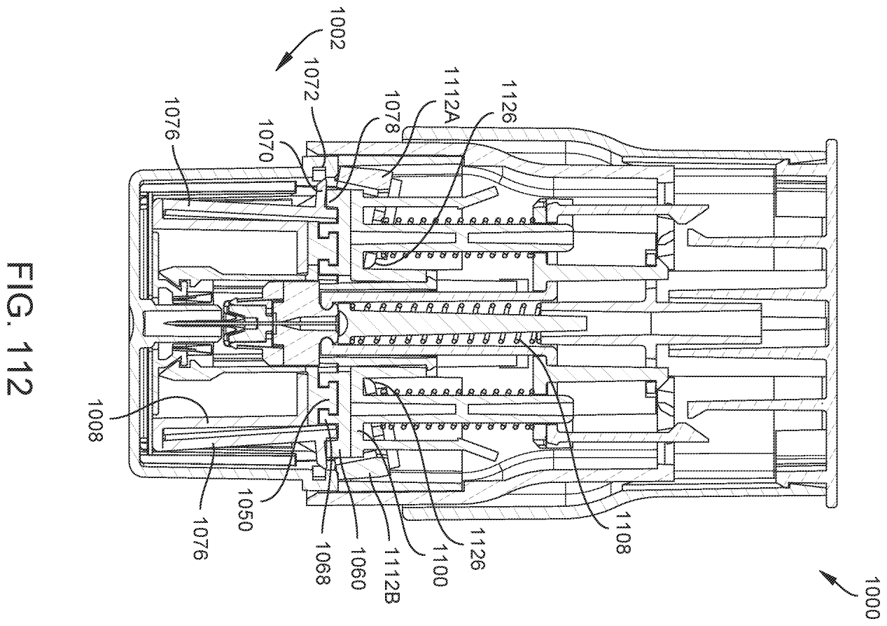

Referring primarily to FIGS. 91-94, in the initial state, the sled 560 may be may be disposed within a receiving void 564 included in the interior housing 120. The sled 560 may have a curved wall 566 which is substantially flush with the exterior face of the interior housing 120 in the initial state. As shown, the receiving void 564 is shaped so as to receive the protuberance 558 of the button 550. In the example embodiment, the protuberance 558 is cruciform in shape and the receiving void 564 mimics this shape. Until the inserter assembly 100 is in the aligned state, the wall of the interior housing 120 presents a mechanical interference which prevents the button 550 from being depressed. Once in the aligned state, the protuberance 558 may pass into the receiving void 564 and into the sled 560. Though a cruciform shape is used, any other type of shape may be used. For example, a star or asterisk type shape may be used in alternative embodiments. As shown best in FIG. 91, the shape chosen may include a ramped portion or portions which extend substantially parallel to the longitudinal axis of the inserter assembly 100. This may allow for increased tolerancing on the sled 560 and protuberance 558 and aid in ensuring smooth operation as the inserter assembly 100 is withdrawn from the body.

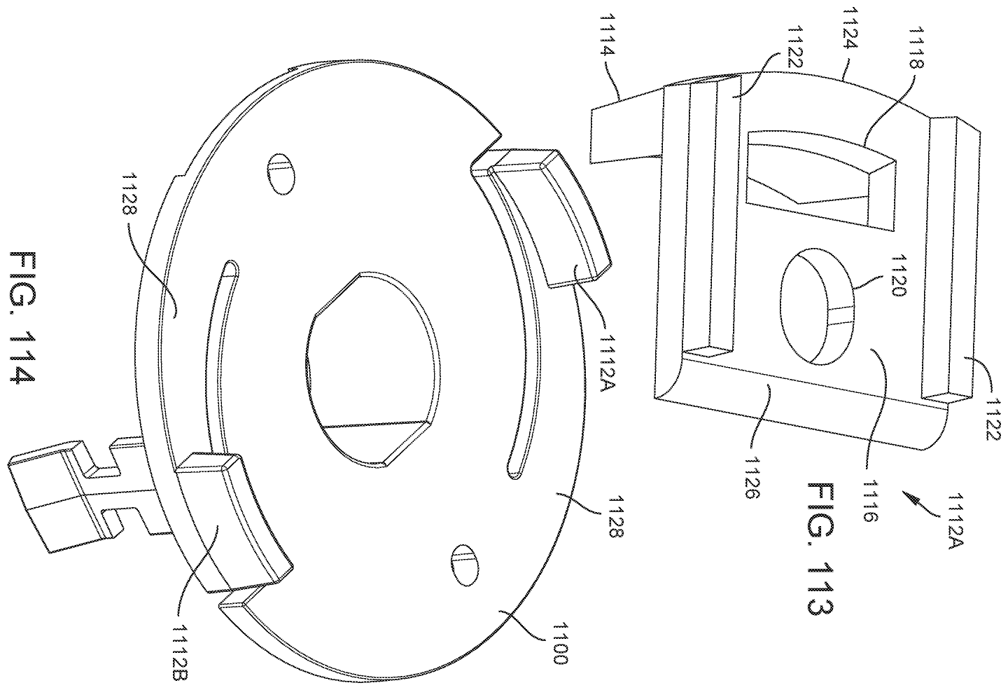

Referring primarily to FIGS. 94-95, the sled 560 may include a cantilevered member 568 including a sled protuberance 570 at a terminal unsupported end thereof. The cantilevered member 568 may be included within an actuation projection 576 of the sled 560. The sled protuberance 570 may form a ledge 572 which may catch on a component of the inserter assembly 100 to retain the sled 560 within the inserter assembly 100. As shown, the top plate 328 of the sharp retractor 134 includes a bridge 574. The bridge 574 forms an underpass which is in line with the receiving void 564 of the interior housing 120 when the inserter assembly 100 is assembled. During assembly, the sled 560 may be passed through the receiving void 564 and an actuating projection 576 of may travel through the underpass formed by the bridge 574. The ledge 572 of the cantilevered member 568 may catch on a surface of the bridge 574 retaining the sled 560 within the inserter assembly 100. The sled protuberance 570 on the cantilevered member 568 may be ramped so as to facilitate deflection of the cantilevered member 568 around the bridge 574 during installation of the sled 560. The bridge 574 may also act as a guide providing a displacement channel for the sled 560 as the sled 560 is driven into the protuberance 300 of the cantilevered arm 296.

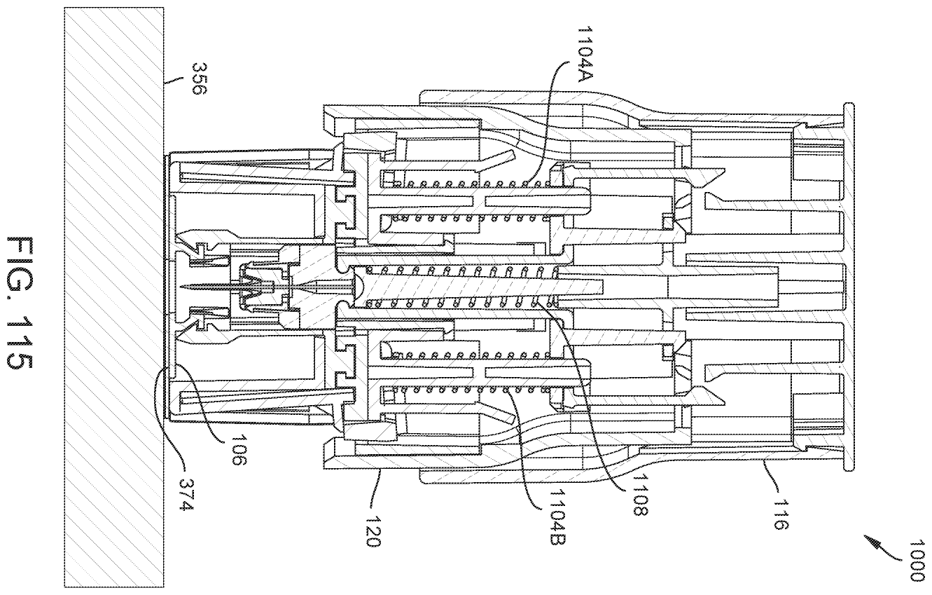

Referring now also to FIG. 96, in some embodiments, a button 550 may not be included, however, a section of the exterior housing 116 may be deformable. In the example shown in FIG. 96, the exterior housing 116 includes a deformable region 580 including a protuberance 558 thereon. The deformable region 580 may be coupled to (e.g. over molded onto or otherwise adhered) the exterior housing 116 in certain examples. In embodiments including a deformable region, once the inserter assembly 100 is in an aligned state, a user may squeeze the exterior housing 116 at the deformable region 580 to drive the protuberance 558 into the sled 560 (see, e.g., FIG. 94) to trigger actuation.

In some embodiments, and referring now to FIG. 97, the interior face of the exterior housing 116 may include a protuberance 558. No button 550 or deformable region 580 may be included. During the withdrawal action of the inserter assembly 100 from the body, relative displacement of components of the inserter assembly 100 may automatically trigger the inserter assembly 100. The relative displacement may bring the protuberance 558 into contact with the sled 560 (sec, e.g., FIG. 94) and drive the sled into 560 the protuberance 300 (see, e.g. FIG. 90B) on the cantilevered arm 296 (sec, e.g. FIG. 90B) which may then be displaced off the catch 306 (sec, e.g. FIG. 90B) freeing bias member 136 (sec, e.g. FIG. 90B). The interior housing 120 may include an appropriately sized channel which accommodates the protuberance 558.

Referring now to FIG. 98, a flowchart 450 depicting a number of exemplary actions which may be executed to assemble an inserter assembly 100 such as that depicted in FIG. 59A-59B is shown. In block 452, a first bias member such as spring 136 may be placed into a cavity 334 in a sharp retractor 134. A cannula subassembly 114 may be placed on a sharp 132 attached to a sharp holder 130 in block 454. The sharp holder 130 may be placed into a sharp retractor 134 cavity 334 in block 456. In block 458, the sharp holder 130 may be latched into a position where the first bias member is in an energy storing state. This may be a compressed state in various examples. A second bias member such as spring 138 may be sandwiched between the sharp retractor 134 and an interior housing 120 in block 460. The sharp retractor 134 may be latched in a position in which the second bias member is in an energy storing state in block 462. This may be a compressed state in various embodiments. In block 464, a casing (which may be formed of an exterior housing 116 and retainer base 140) may be coupled together around the interior housing 120. An infusion set base 106 may be releasably coupled to the sharp retractor 134 in block 466. A lock member 146 may be installed into the inserter assembly 100 in block 468. As mentioned elsewhere herein the lock member 146 may be welded to the adhesive liner 111 coving adhesive on the infusion set base 106 in certain embodiments. During assembly of the components, any rails, guides, keyed features, fingers etc. included on a component may be aligned with cooperating features of the components in which they are being placed. Latching together of the various components of the inserter assembly 100 may simplify assembly of the inserter assembly 100 as the components may hold themselves in the proper orientation or position against any spring bias. Thus an assembler (human or robotic) may simply bring components into latching engagement with one another and be able to release the components thereafter. This may allow partially assembled portions of inserter assemblies 100 to be moved about a production facility or shipped to other facilities and may simplify any fixturing used during assembly.

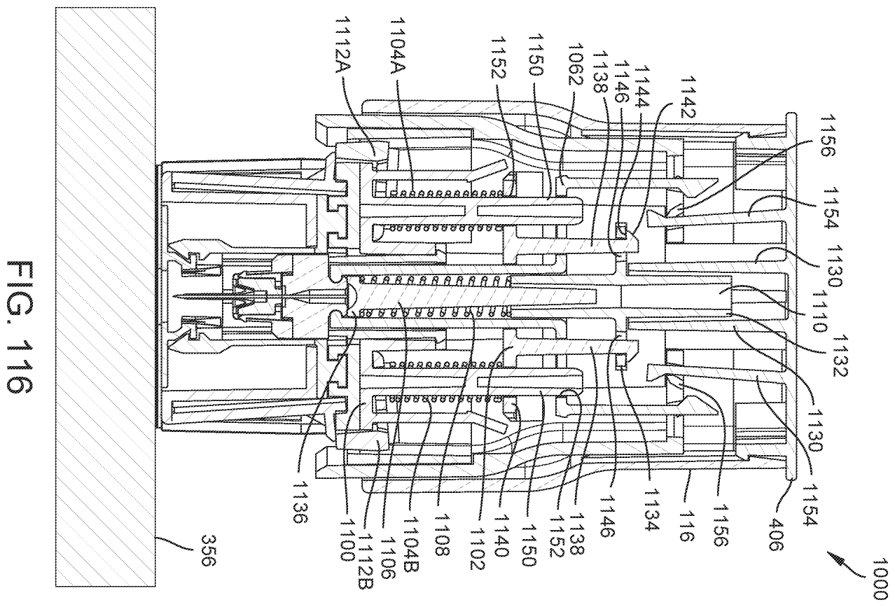

Referring now to FIG. 99, in some examples, an inserter assembly 1000 may be reusable. After actuation, certain inserter assembly 1000 embodiments, may be reset and used to install a next infusion set 102 (or analyte sensor in certain examples). Thus, the same inserter assembly 1000 may be used to install a plurality of different infusion sets 102 (or other patient care assemblies). For example, a container of infusion sets 102 may include a single inserter assembly 1000 (or a number of inserter assemblies 1000 which is less than the number of infusion sets 102). The single inserter assembly 1000 may be intended to be used to install each of the infusion sets 102 in the container onto the patient. Thus, the waste and cost associated with site changes may be reduced. In embodiments where the inserter assembly 1000 is a multi-use device, infusion sets 102 may be provided in separate set cartridges 1002. Similarly, where the inserter assembly 100 is configured to apply a sensor to the patient, sensors may be provided in sensor cartridges which are separate from the inserter assembly 1000. As in other embodiments described herein, the infusion set 102 may be provided partially assembled within the set cartridges 1002. A set cartridge 1002 is depicted separate from the inserter assembly 1000 in FIG. 99. The set cartridge 1002 may be coupled to the inserter assembly 1000. Once coupled, the inserter assembly 1000 may be actuated to install the infusion set 102 at a desired infusion site. After actuation, the spent cartridge 1002 may be separated from the inserter assembly 1000 and disposed of. The inserter assembly 1000 may be reset when another set cartridge 1002 is coupled thereto and used to install another infusion set 102.

Depending on the embodiment, actuation of the inserter assembly 1000 may also cause assembly of the infusion set 102 to be completed. The infusion set 102 or other patient care assembly may be provided as a number of portions (e.g. separate components, subassemblies, or combinations thereof) within a set cartridge 1002. Actuation of the inserter assembly 1000 may cause each portion of the infusion set 102 to be coupled together to complete the assembly of an infusion set 102. For example, assemblage of an infusion set 102 may occur as an initial stage of the actuation of the inserter assembly 1000 or may occur as part of an insertion stage of inserter assembly 1000 actuation which results in the cannula 104 being introduced into the patient.

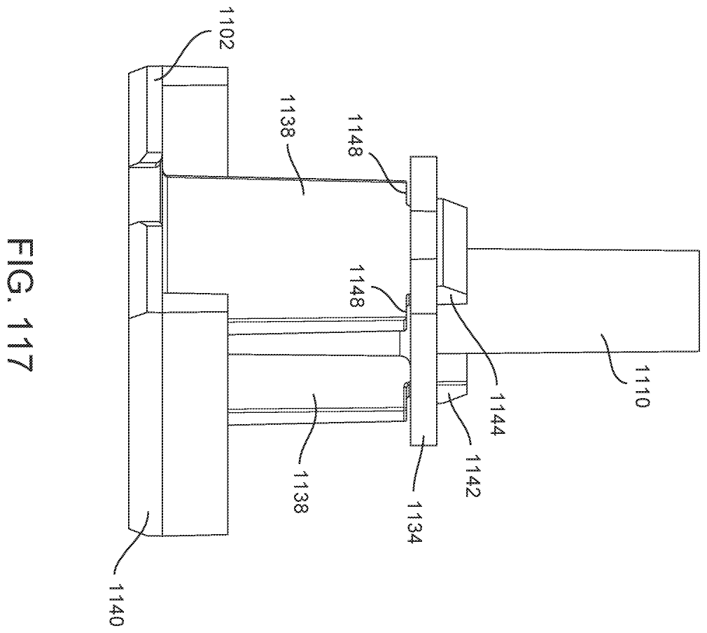

Referring now to FIGS. 100A-100B, exploded views of an exemplary set cartridge 1002 are depicted. As shown, the set cartridge 1002 may include an exterior housing 1004. When the set cartridge 1002 is assembled, the exterior housing 1004 may contain the other components of the set cartridge 1002. The exterior housing 1004 may thus also be referred to herein as a container. In the example, the exterior housing 1004 is shown as a cup and includes an open top. Depending on the embodiment, when fully assembled, the open top of the exterior housing 1004 may be covered by a barrier member so as to completely enclose the components of the set cartridge 1002. This barrier member may be permeable to a sterilizing agent to allow for sterilization of the set cartridge 1002 after assembly. A barrier member may also help minimize contact of components in the exterior housing 1004 with the surrounding environment or a user.

As shown, an infusion set 102 (or other patient care assembly) may be contained within the set cartridge 1002 as a first portion and a second portion which are separate from one another, but coupled together during actuation of the inserter assembly 1000 to form the infusion set 102. The first portion may include a base 106 which may be applied to the skin of a patient and may couple to a fluid pathway (e.g. via a terminal connector on the pathway such as connector 368 shown in FIGS. 15A-F) which is part of or extends from an infusion pump. An adhesive backing, film, or liner 111 may be included and may be applied over adhesive 374 included on the infusion set base 106. The infusion set base 106 may seat within a receiving bay 1006 of an interior housing 1008 of the set cartridge 1002. The receiving bay 1006 may include notches 1010 which may accept tube retainers 184 included on the infusion set base 106. In the example embodiment, the notches 1010 may also serve to ensure that the base 106 may only be installed within the receiving bay 1006 in a desired orientation. As shown in FIGS. 100A-B, the interior housing 1008 may include an indention 1012. The indention 1012 may be sized so as to accept pull tabs 410 included on the adhesive liner 111. As the indention 1012 may be visible to the user, the indention 1012 may be used as an orientation indicator. This may help the user install the infusion set 102 in a manner which accommodates their planned routing pathway for infusion tubing which is to be coupled to the infusion set 102. Indicators which show the orientation of the infusion set 102 may be included on portions of the inserter assembly 1000 as well. This may be particularly true in embodiments where the set cartridge 1002 may only be coupled to the inserter assembly 1000 in a single orientation.

The second portion of the infusion set 102 may be a subassembly 114 of two or more components of the infusion set 102. The second portion may include a cannula 104, septum housing 108, septum 110, and septum retainer 112 for example (an exemplary cannula subassembly 114 is shown exploded apart in FIG. 1A). Any cannula subassembly 114 described herein may be used. The cannula 104 and the septum housing 108 are shown a single continuous unitary part in the example embodiment. This cannulated housing may be a molded part which is constructed of a single material such as, PTFE, Teflon, polypropylene, etc. for example. When the set cartridge 1002 is assembled, the insertion sharp 132 may extend through the cannula subassembly 114 and the cannula subassembly 114 may be disposed against the sharp holder 130. In sensor cartridge embodiments, the cannula subassembly 114 and infusion set base 106 may be replaced by a sensor assembly.

A set cartridge 1002 may further include a sharp holder 130. The sharp holder 130 may retain an insertion sharp 132 thereon. The insertion sharp 132 may be glued or otherwise bonded into the sharp holder 130 so as to be fixedly located relative to the sharp holder 130. The insertion sharp 132 may alternatively be press fit into the sharp holder 130 or the sharp holder 130 and insertion sharp 132 may be joined in an overmolding process. Any suitable type of sharp 132 may be used. For example, the sharp 132 may be a hollow or solid needle, stylet, or other pointed member which may be made of a metal material such as steel.

Referring now primarily to FIG. 101, an infusion base retainer 1014 may also be included in a set cartridge 1002. The infusion base retainer 1014 may include a set of retainer arms 1016. The retainer arms 1016 may extend from an end plate 1028 of the infusion base retainer 1014. The arms 1016 may be separated from a wall 1018 defining the central cavity 1020 of the infusion base retainer 1014 by interrupt regions included in the wall 1018. The wall 1018 may also include a set of protuberances 1030. The protuberances 1030 may be disposed opposite one another on the wall 1018 and may be roughly spaced 90° from the arms 1016. The protuberances 1030 may include a ramped face 1032 on a portion of each protuberance 1032 most distal to the end plate 1028. The protuberances 1030 may also define a catch face 1034 on a portion of the protuberances 1030 most proximal to the end plate 1028.

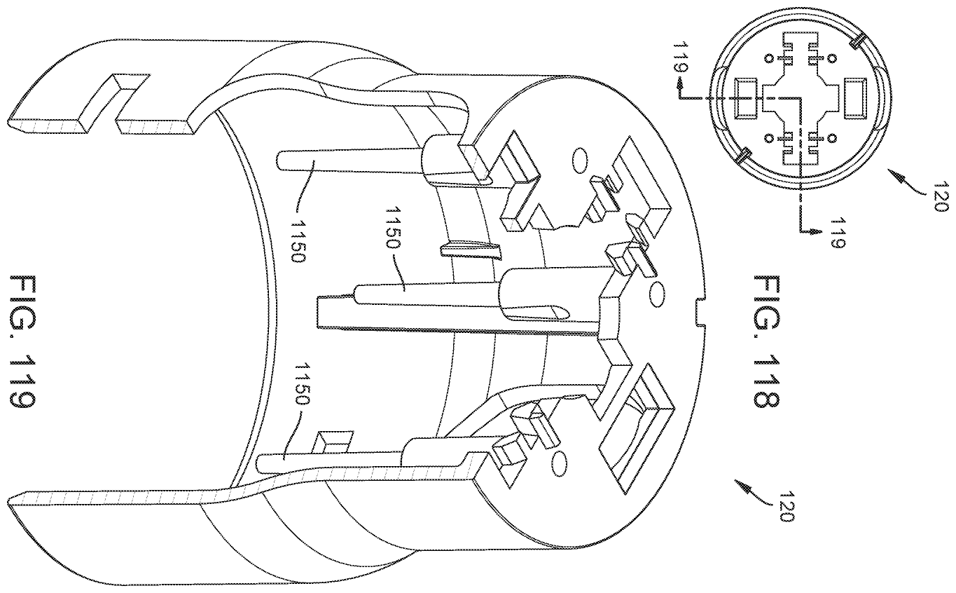

Referring now also to FIG. 102, during assembly of the infusion base retainer 1014 into the interior housing 1008, the protuberances 1030 may travel along respective channels 1036 defined in the interior housing 1008. As shown, a terminus of the channels 1036 may be closed by a stop wall 1038. As the protuberances 1030 are driven into the stop wall 1038 the portions of the wall 1018 on which the protuberances 1030 are included may resiliently deflect inward so as to allow passage of the protuberances 1030 beyond the stop wall 1038. The ramped face 1032 may facilitate this deflection. Once the protuberances 1030 have passed the stop wall 1038, the portions of the wall 1018 may restore back to an unstressed state. When the portions of the wall 1018 are in the unstressed state, the catch faces 1034 of the protuberances 1030 may be latched against the stop walls 1038 of the channels 1036 preventing the infusion base retainer 1014 from being pushed out of the interior housing 1008. The end plate 1028 of the infusion base retainer 1014 may abut a rim 1040 which prevents further displacement of the infusion base retainer 1014 into the interior housing 1008. Thus the infusion base retainer 1014 may be fixed in place within the interior housing 1008.

Referring again primarily to FIG. 101, the set of arms 1016 of the infusion base retainer 1014 may be disposed in opposing relationship to one another and may be cantilevered from the end plate 1028. Each of the arms 1016 may include a protuberance 1022 disposed at an unsupported end thereof. Each protuberance 1022 may form a ledge 1024 on the arm 1016 on which it is included. Additionally, the arms 1016 may include a nub 1026 or raised ramp which increases in thickness as distance from the cavity 1020 (see, e.g., FIG. 100A) increases. The nub 1026 may be disposed intermediate the unsupported end of the arm 1016 and its attachment point to the remainder of the infusion base retainer 1014.

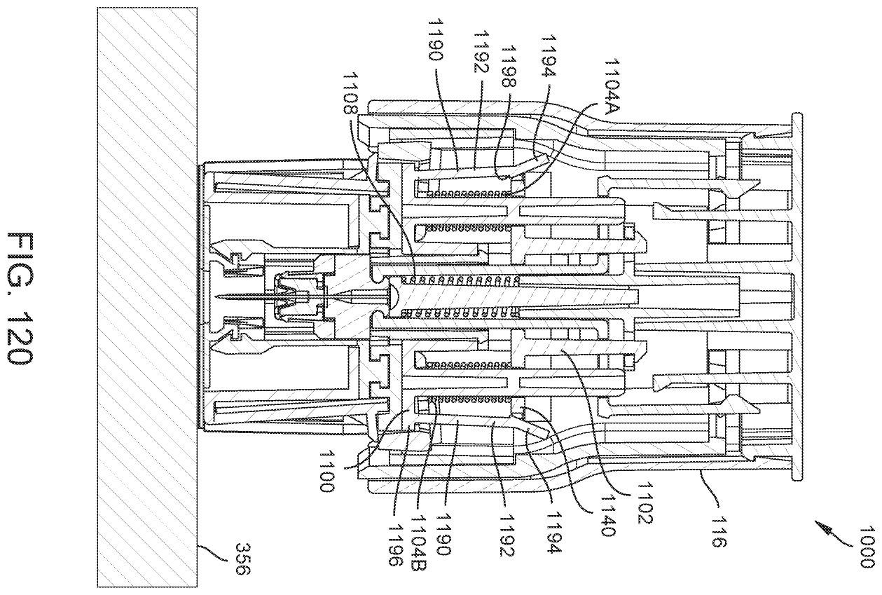

Referring now to FIGS. 103-104B and as best shown in FIG. 104B, each ledge 1024 may capture a portion of the infusion set base 106. Specifically, the ledges 1024 may catch on an outcropped portion of the infusion set base 106. The base 106 may include rails, step features, nubs or any other suitable protrusions to provide a complimentary catch surface for the ledges 1024. In certain embodiments, the ledges 1024 may catch on guides 172 of the infusion set base 106. In other examples, walls of cutouts (see, e.g. breaks 181 of the example infusion set base 106 shown in FIGS. 21A-F) in an infusion set base 106 may provide a catch with the ledges 1024 may engage. Thus the infusion set base 106 may be retained within the set cartridge 1002. Ledges 1024 may be angled with respect to the cantilevered arm 1016 on which they are included such that the undercut has a triangular cross section. The portion of, for example, the guides 172 (or any other catch feature) on which each ledge 1024 catches may be angled in a cooperating manner to help ensure a robust engagement.

In certain embodiments, only one arm 1016 may be included. In some embodiments, the interior housing 1008 may also or instead include latch which may interface with the guide 172 or another cooperative portion of the infusion set base 106 to retain the infusion set base 106 in place.

With the infusion set base 106 retained by the arms 1016, the infusion set base 106 may also act as a protective barrier. As the cannula subassembly 114 and insertion sharp 132 may be internal to the set cartridge 1002, when the infusion set base 106 is in the initial retained position, the user may be protected from accidental contact with the insertion sharp 132. This may additionally help to keep the cannula 104 or insertion sharp 132 from coming into contact with contaminants. Though a void for receipt of the cannula subassembly 114 may extend through the entirety of the infusion set base 106, the void may be sized to prevent finger ingress (e.g. have a cross-section smaller than that of a finger). Thus the infusion set base 106 may present an obstacle which blocks unintentional access to the insertion sharp 132 and cannula 104. Additionally, as the cannula subassembly 114 is internal to the set cartridge 1002, any adhesive backing 111 provided on the infusion set base 106 need not include an interruption to allow for passage of the cannula 104 therethrough. Thus, the void in the infusion set base 106 for the cannula subassembly 114 may be blocked by the adhesive backing 111 until just prior to use. This may further prevent finger ingress and may mitigate potential for detritus to enter the set cartridge 1002. The exterior housing 1004 may also present a barrier which inhibits a user from interacting with the insertion sharp 132 and/or cannula 104.

As shown in FIGS. 104A-104B, the infusion base retainer 1014 may include at least one guide in various embodiments. In the example embodiments, the infusion base retainer 1014 includes a set of sharp holder guides 1042 (see, e.g., FIG. 104A). The sharp holder guides 1042 may be recessed into a cavity facing side of the wall 1018 of the infusion base retainer 1014. The sharp holder 130 may include fins 1044 which may ride within the sharp holder guides 1042 during displacement of the sharp holder 130 within the set cartridge 1002. In alternative embodiments, the sharp holder 130 may include recesses and the wall may include rails projecting from the cavity facing side of the wall 1018. The rails may be received within the recesses of the sharp holder 130 and guide displacement of the sharp holder 130 as it displaces within the set cartridge 1002.

The infusion base retainer 1014 may also include a set of septum housing guides 1046. The septum housing guides 1046 (see, FIG. 104B) may be recessed into the cavity facing sides of the arms 1016. Ears 204 of the septum housing 108 may ride within the septum housing guides 1046 during displacement of the cannula subassembly 114 within the set cartridge 1002. As shown, the sharp holder 130 may also include fins 1048 which are in line with the cars 204 and which may also ride within the septum housing guides 1046. When the cannula subassembly 114 latches into the base 106, the cars 204 on the cannula subassembly 114 may press against the nubs 1026 included on the arms 1016. This may cause the arms 1016 to be splayed apart resulting in disengagement of the arms 1016 from the infusion set base 106. In turn, this may free the now assembled infusion set 102 from the set cartridge 1002.

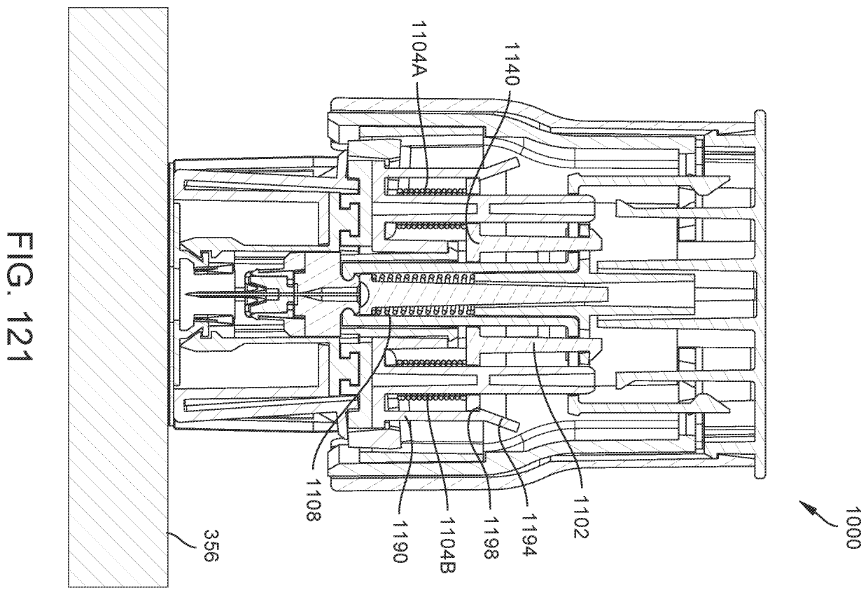

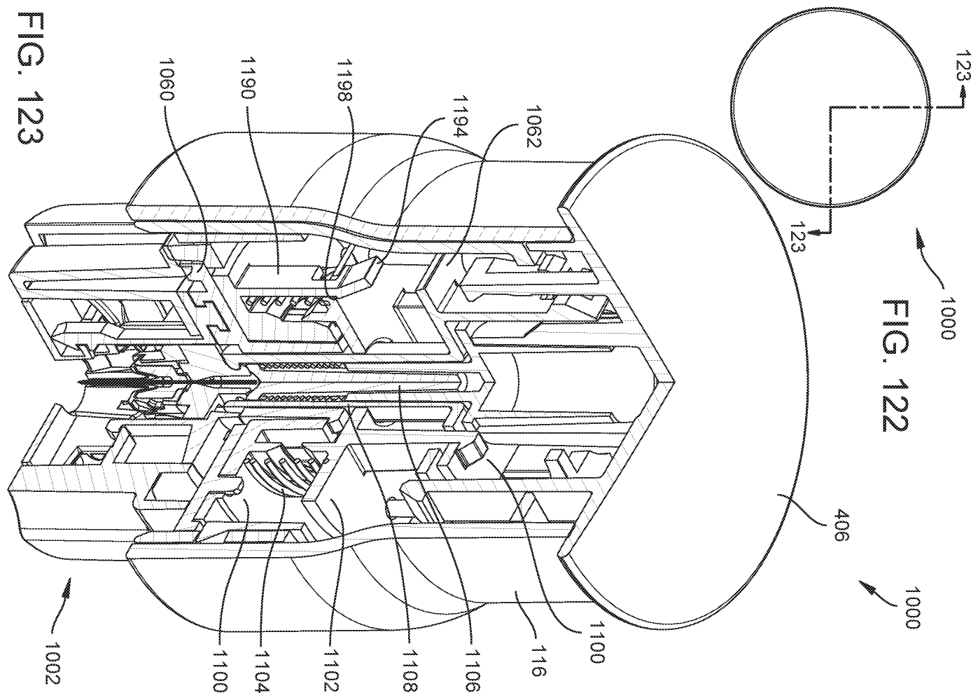

Referring now to FIGS. 105A-106B, exploded views of two example embodiments of inserter assemblies 1000 are depicted. Inserter assemblies such as those shown in FIGS. 105A-106B may be coupled to set cartridges 1002 and subsequently used to place an infusion set 102 onto an infusion site of a patient and introduce a cannula 104 of an infusion set 102 into the patient's body. As mentioned elsewhere, some inserter assemblies 1000 may be used to place other patient care assemblies onto the body of a patient. For example, certain inserter assemblies 1000 may be operated to place physiological monitors or analyte sensors into working relationship with a patient's body. Blood sugar monitors such as continuous glucose sensors may be placed using an inserter assembly 1000. In certain embodiments, the same inserter assembly 1000 may be mated with a set cartridge 1002 or sensor cartridge depending on the type of device a patient intends to place on their body. In some embodiments, inserter assemblies 1000 may also be coupled to lancet cartridges (e.g. a set cartridge 1002 without a cannula subassembly and infusion set base 106) to create a skin puncture for collection of a body sample with an analyte testing strip.

As shown in the exploded views in FIGS. 105A-106B, an insertion assembly 1000 may include an exterior housing 116. The exterior housing 116 may enclose various components of the inserter assembly 1000 and serve as the portion of the inserter assembly 1000 which the user grips during operation. The exterior housing 116 in the example embodiment of FIGS. 105A-105B has a cross sectional shape which is round, though other embodiments may have different shapes such as any type of polygonal shape. In certain examples, a rectangular or obround cross-sectional shape such as that shown in FIGS. 106A-106B may be used. Such a shape may more easily fit within a pocket or may more easily fit into smaller pockets which may, for example, be more typical of women's clothing. The cross sectional area in the example embodiments also vary with the bottom section (that most proximal the skin when in use) of the exterior housing 116 being wider or having a greater cross-sectional area than the top. An exterior housing 116 may include various ergonomic features which facilitate grasping of the inserter assembly 1000 in which it is included. For example, texturing or a finger or thumb depression may be included on the outer surface of the exterior housing 116. Alternatively or additionally, a region of the external housing 116 may be thinner in width than the remaining portion of the external housing 116. This may make firm grasping of the inserter assembly 1000 easier.

Example exterior housings 116 may include a marking, tab, embossed section, recess section, textured section, protuberance, color coding, appliqué, or other indicia which serves to indicate position and/or orientation of the infusion set 102 within the insertion assembly 1000. A raised rib 118 such as that shown in FIG. 1A for example, may be included. This may allow a user to position the inserter assembly 1000 in a desired orientation so as to allow for a run of infusion tubing 366 (see, e.g. FIG. 6) to be routed in a planned manner once the infusion set 102 is attached to the user.

A retainer cap 406 may serve to couple to a top portion of the inserter assembly 1000 to hold the various components in place within the inserter assembly 1000. In the examples shown in FIGS. 105A-106B, the retainer caps 406 include cantilevered retainer bodies 408 which may snap into retaining interfaces 411 (sec, e.g. FIG. 105A) included on the exterior housing 116. Other couplings are also possible such as a bayonet mount, interference fit, snap fit, adhesive, glue, threads, solvent bonding, welding, etc. When coupled together, the exterior housing 116 and retainer cap 406 may form a casing of the inserter assembly 1000. The exterior housing 116 and the retainer cap 406 together may form a first unit of the inserter assembly 1000. The remaining components of the inserter assembly 1000 may be referred to as a second unit of the inserter assembly 1000.

Still referring to FIGS. 105A-106B, an inserter assembly 1000 may also include an interior housing 120. The interior housing 120 may be disposed inside of the external housing 116 when the inserter assembly 1000 is assembled. The first unit of the inserter assembly 1000 may be displaceable relative to the interior housing 120 and other components of the second unit of the inserter assembly 1000 contained therein. Various exterior housings 116 may have at least one keying feature which constrains the interior housing 120 such that it may only be installed in a limited number of orientations within the exterior housing 116. The cross-sectional shape may be chosen so as to dictate such a constraint (sec, e.g., FIGS. 106A-106B). Alternatively, the exterior housing 116 may include at least one rail 1090 (see, e.g., FIGS. 105A-105B). In the exemplary embodiment shown in FIGS. 105A-105B, two rails 1090 are included directly opposite one another (only one is visible) on the interior facing surfaces of the exterior housing 116. The rails 1090 extend substantially parallel to one another. If it is desired to limit the interior housing 116 to a single installation orientation, the rails 1090 may be of different widths. The exterior face of the interior housing 120 may include tracks 1092 which cooperate with the rails 1090. The interior housing 120 may be inhibited from displacing into the exterior housing 116 until the rails 1090 are aligned with the tracks 1092. The interaction of the rails 1090 within the tracks 1092 may also inhibit rotation of the interior housing 120 and exterior housing 116 relative to the other. Though rails 1090 are shown on the exterior housing 116 in the example, the rails 1090 may instead be present on the exterior face of the interior housing 120 in some embodiments. In such examples, the tracks 1092 may be located on the exterior housing 116.

As shown in FIGS. 105A-105B, the exterior housing 116 may also include stop protrusions 1094 on the interior face of the exterior housing 116. The stop protrusions 1094 may be nubs, pins, rails or any suitable feature. The stop protrusions 1094 may limit the travel of the interior housing 120 along the axis of the exterior housing 116. In the example embodiment, the stop protrusions 1094 extend from an end of the exterior housing 116 in a direction parallel to the rails 1090. In certain examples, the retainer arms 408 of the retainer cap 406 may also act as stops which limit travel of the interior housing 120 relative to the exterior housing 116.

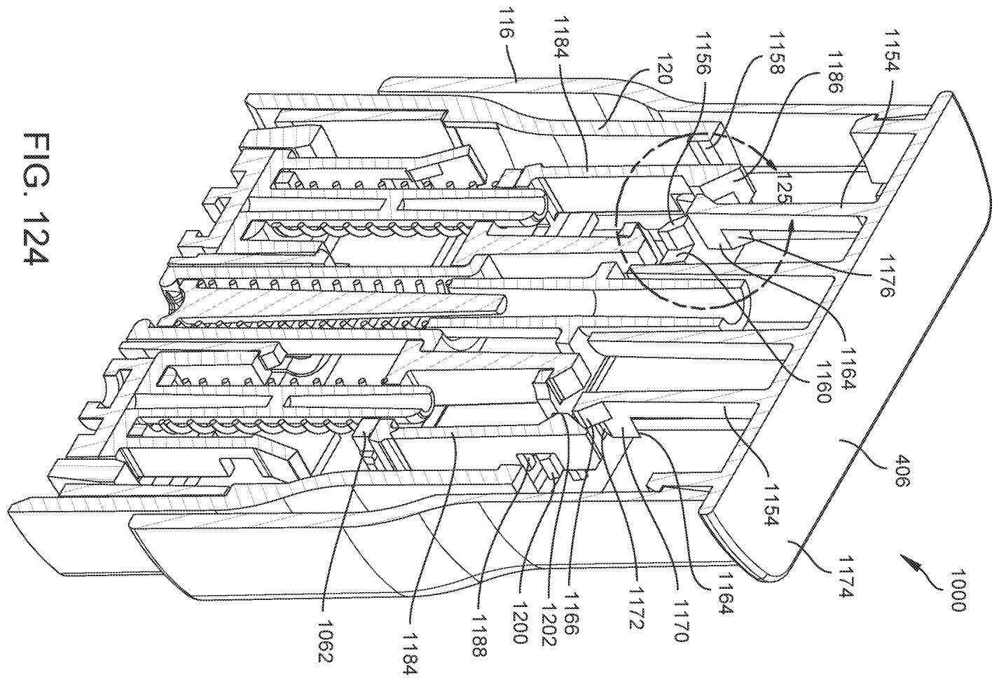

A receptacle body 1060 (described in greater detail in relation to FIGS. 108-110, and FIG. 112) may serve to couple to a bottom portion of the inserter assembly 1000 to hold the various components in place within the inserter assembly 1000. In the examples shown in FIGS. 105A-106B, the receptacle bodies 1060 include cantilevered retention arms 1096 which may snap into retainer interfaces 1098 included on the interior housing 120. Other couplings are also possible such as a bayonet mount, interference fit, snap fit, adhesive, glue, threads, solvent bonding, welding, etc. Lock members 1112A, B which may be part of another component (e.g. the retraction latch body 1100 in FIGS. 105A-105B) or as individual separate components (see, e.g., FIGS. 106A-106B) may be included and may project through the receptacle body 1060.

A retraction latch body 1100 and a retracting spring retainer 1102 may also be included. As will be further described later in the specification, the retraction latch body 1100 and retraction spring retainer 1102 may engage with one another to hold a bias member such as spring 1104 (or springs 1104A, B of FIGS. 106A-106B) and spring 1108 in an energy storing state during portions of the inserter assembly 1000 actuation. The retraction latch body 1100 may be coupled in place on the receptacle body 1060 via interaction of latch fingers 1097 of the receptacle body 1060 with catch bodies 1099 on the retraction latch body 1100.

An insertion driver 1062 may also be included in an inserter assembly 1000. As will be further described later in the specification, the insertion driver 1062 may have a plunger 1106 and a spring 1108 housed in a portion of the insertion driver 1062. An assembly resetting body 1110 may be included. As further described later in the specification, the resetting body 1110 may act on various components of the inserter assembly 1000 to place the components in a ready state in preparation for an actuation. When freed to transition from an energy storing state to a relaxed state, the spring 1108, may displace the insertion driver 1062 to cause insertion of a cannula 104 from an attached set cartridge 1002 and complete assembly of the infusion set 102 of the set cartridge 1002. Retraction of the sharp 132 into the set cartridge 1002 may also occur as spring 1104 (or springs 1104A, B of FIGS. 106A-106B) is freed to transition from an energy storing state to a relaxed state. During this transition, the spring 1104 may in some embodiments, drive the retraction spring retainer 1102 towards a retracted state within the inserter assembly 1000. This may cause the insertion driver 1062 (which may be attached to the sharp holder 130, see e.g. discussion of FIG. 108) to be driven to a retracted state.

Referring FIG. 107, a cross-sectional view of the inserter assembly 1000 of FIGS. 106A-106B is depicted. The inserter assembly 1000 is depicted in a relaxed or storage state. As shown, no set cartridge 1002 is attached to the inserter assembly 1000. In some embodiments, an end cover may be included for storage and may be placed over the end of the inserter assembly 1000 to which a cartridge 1002 may be coupled. Additionally, in FIG. 107, all of the bias members 1108, 1104A, B are shown in a relaxed or unstressed state. The inserter assembly 1000 may be in the relaxed state between usages of the inserter assembly 1000. As the inserter assembly 1000 may typically only be used during site changes (e.g. every three days), the inserter assembly 1000 may be in the relaxed state for the vast majority of its usage life. Thus, the bias members 1108, 1104A, B may only be required to be in an energy storing state or stressed state (a compressed state in the example embodiment) for short periods of time. Bias members 1108 and 1104 of the inserter assembly 1000 of FIGS. 105A-105B may similarly be in a substantially unstressed state when the inserter assembly 1000 is in its storage state.

A storage state in which bias members 1108, 1104A, B are in an unstressed state may facilitate use of a wide variety different of bias members 1108, 1104A, B or bias member 1108, 1104A, B materials. For example, spring relaxation and/or creep may be less of a concern allowing materials such as various polymers to more easily be utilized in construction of the bias members 1108, 1104A, B. Additionally, other components of an inserter assembly 1000 may not be subjected to sustained stress exerted by bias members of the inserter assembly 1000 being in a stressed state when the inserter assembly 1000 is being stored (e.g. during shipping or when sitting in stock). Consequentially, any creep engendered by this sustained stress may be removed. This in turn may allow for greater design flexibility in other components of the inserter assembly 1000. For example, a greater variety of materials may be used or certain components may be made smaller. It should be noted that in certain examples, the set cartridge 1002 (or sensor cartridge or lancet cartridge) may not include any bias members. Instead, all bias members may be included in the inserter assembly 1000. As a result, the components of the set cartridge 1002 may also be stored in a state where they are not subjected to sustained stress. This may similarly assist in providing greater design flexibility for components of cartridges 1002.

Referring now to FIGS. 108, a view of an example cartridge 1002 exploded away from an exemplary inserter assembly 1000 is depicted. As shown, the end plate 1028 of infusion base retainer 1014 (see, e.g., FIG. 101) may include at least one mating pin 1050. In the example embodiment two mating pins 1050 are included. The mating pins 1050 may be disposed opposite one another on the top plate 1028 and in the example are shown 180° apart from one another. The mating pins 1050 may include an enlarged head portion 1052 which is connected to the end plate 1028 via a pin body 1054. A portion of the sharp holder 130 is also visible in FIG. 108. As best shown in the detailed view in FIG. 109, the sharp holder 130 may include a mating section 1056. The mating section 1056 may include a thinned region 1058 which is adjacent a terminal flange 1059 that forms an end of the sharp holder 130. The terminal flange 1059 may have a length dimension which is longer than its width dimension. In the example embodiment, the top flange 1059 is obround in shape.

To couple the cartridge 1002 to the inserter assembly 1000, the cartridge 1002 may be placed against a receptacle body 1060 included within the inserter assembly 1000. A sharp driver 1062 including a port 1064 for the mating section 1056 of the sharp holder 130 may be accessible through the receptacle body 1060. In an initial coupling state, the mating section 1056 may be oriented in an aligned position with the port 1064 such that the mating section 1056 may pass into the port 1064. The mating pins 1050 may act as standoffs which limit the amount that the mating section 1056 may be displaced into the port 1064. In the example embodiment, the mating pins 1050 may limit displacement of the mating section 1056 into the port 1064 such that the thinned section 1058 is in line with a rim 1066 surrounding the port 1064. The cartridge 1002 may then be rotated from the initial coupling state to a fully coupled state. As the thinned section 1058 is in line with the rim 1066, the mating section 1056 may be free to rotate. During coupling, the terminal flange 1059 may be swept over an interior face of the rim 1066 into an orientation in which it may no longer pass through the port 1064. The top flange 1059 may be biased against the interior face of the rim 1066 by at least one bias member 1108 (see, e.g. FIG. 112). This may aid in inhibiting additional rotation of the terminal flange 1059. The mating pins 1050 may also be displaced into a mating interface such as retention shoes 1068 included on the receptacle body 1060. The retention shoes 1068 may be formed as “U” shaped bodies which are raised proud of the receptacle body 1060. The “U” shaped bodies may vary in width in a stepwise fashion as distance from the receptacle body 1060 increases. Adjacent the receptacle body 1060, the width may be sized to accept the heads 1052 of the mating pins 1050. The portion of the shoes 1068 most distal to the receptacle body 1060 may have a width sized to accept the pin body 1054 of the mating pins 1050. Thus, when the cartridge 1002 is rotated to the fully coupled state, the heads 1052 of the mating pins 1050 may be overhung by a portion of the shoes 1068 such that the mating pins 1050 may not be translationally displaced out of the shoes 1068 in a direction parallel to the longitudinal axis of the inserter assembly 1000. In alternative embodiments, the mating pins 1050 (or another mating projection) may be included on the receptacle body 1060. Retention shoes 1068 (or another mating interface) may be included as part of the cartridge 1002.

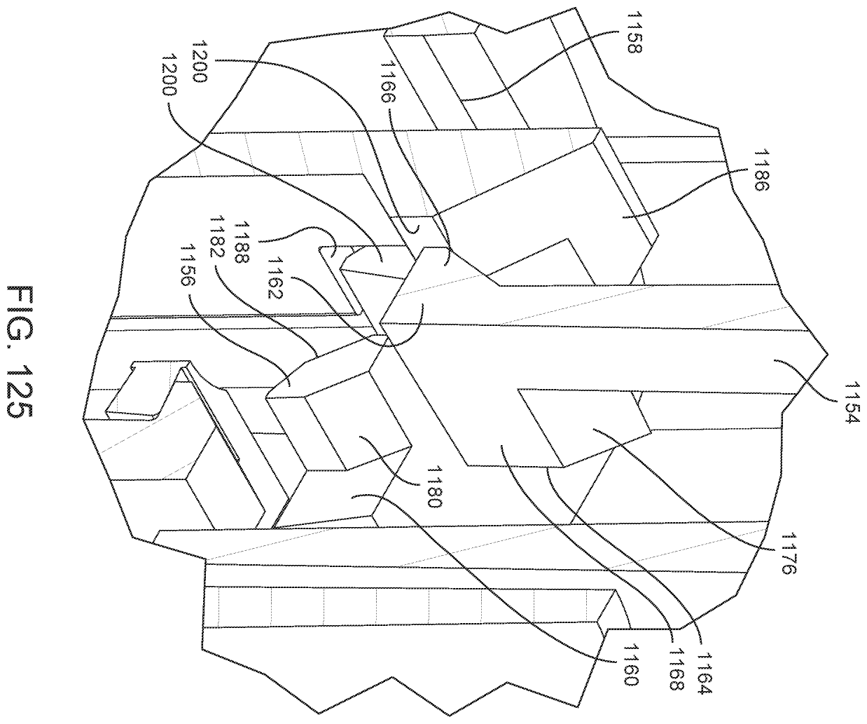

Referring now also to FIG. 110, in some embodiments, rotation of the cartridge 1002 to the fully coupled state may actuate housing tabs 1070 of the interior housing 1008 out of engagement with receiving slots 1072 (see also, e.g., FIG. 104B) of the exterior housing 1004. In the example embodiment, the housing tabs 1070 each include a ramped projection 1074 which may be disposed on the medial portions of the housing tabs 1070. As best shown in FIG. 102, each housing tab 1070 may be disposed on a cantilevered arm 1076. During coupling of the cartridge 1002 to the inserter assembly 1000, a portion of the receptacle body 1060 may interact with the housing tab 1070 and deflect the cantilevered arm 1076 toward the center of the cartridge 1002. This deflection may cause the housing tab 1070 to be displaced out of the receiving slot 1072 (best shown in FIG. 104B) of the exterior housing 1004. In the example embodiment, the receptacle body 1060 includes a set of deflector members 1078 which are raised from the receptacle body 1060. The deflector members 1078 may include a ramped portion 1080. As the cartridge 1002 is rotated to the fully coupled state, the ramped portions 1080 of the deflector members 1078 may be displaced into abutment with the ramped projections 1074 of the housing tabs 1070. As further rotation occurs the deflector members 1078 may push the ramped projections 1074 out of the displacement path of the deflector members 1078 by bending the cantilevered arms 1076. In turn, this may actuate the housing tabs 1070 out of the receiving slots 1072. The exterior housing 1004 may then be separated from the set cartridge 1002.

Still referring primarily to FIG. 108, the inserter assembly 1000 may include one or more lock members 1112A, B. In the example embodiment, two lock members 1112A, B which are diametrically opposed or spaced 180° from one another are included on the inserter assembly 1000. Other embodiments may include a greater or lesser number of lock members 1112A, B. When a cartridge 1002 is coupled to the inserter assembly 1000 and the exterior housing 1004 is removed, the lock members 1112A, B may be in a state in which they projection from the receptacle body 1060. When in the projecting state, the lock members 1112A, B may be positioned between a portion of the edge walls 1084 on either side of the cantilevered arms 1076. This may inhibit rotation of the interior housing 1008 of the cartridge 1002 relative to the inserter assembly 1000 as the edge walls 1084 may act as stop surfaces which present a mechanical interference blocking displacement of the lock members 1112A, B. Thus, the inserter assembly 1000 and interior housing 1008 may be locked together in the fully coupled orientation as rotational displacement of the mating pins 1050 out of the retention shoes 1068 is prevented. Sensor cartridges or lancet cartridges may similarly couple to an inserter assembly 1000.

Referring now to FIG. 111, in certain embodiments, the cartridge 1002 (or other cartridge) may couple to an inserter assembly 1000 in the same manner, but may not have the same cross sectional shape as the inserter assembly 1000. In the example shown in FIG. 111, the inserter assembly 1000 as a roughly obround cross sectional shape while the set cartridge 1002 has a substantially circular shape. In some examples, various different types of a cartridges may have differing cross sectional shapes so that they are readily differentiable upon visual inspection. Different types of cartridges may also be different colors or have different textures, surface finishes, indicia, labeling, etc.

Referring now to FIG. 112, another cross-sectional view of the inserter assembly 1000 of FIGS. 106A-106B is depicted. The inserter assembly 1000 is coupled to an example set cartridge 1002 in FIG. 112. Mating pins 1050 of the set cartridge 1002 may be retained within the retention shoes 1068 of the receptacle body 1060 of the inserter assembly 1000. The deflector members 1078 of the receptacle body 1060 have deflected the cantilevered arms 1076 of the interior housing 1008. This in turn has pushed the housing tabs 1070 out of engagement with the receiving slots 1072 of the exterior housing 1004. Thus, the exterior housing 1004 of the set cartridge 1002 may be disassociated with the set cartridge 1002 to expose the infusion set base 106.

Still referring to FIG. 112, during coupling of a set cartridge 1002 to an inserter assembly 1000, a portion of the set cartridge 1002 may displace the lock members 1112A, B from a projecting state to a retracted state. As shown in FIG. 112, the exterior housing 1004 of the set cartridge 1002 may force the lock members 1112A, B into the retracted state when the set cartridge 1002 is docked against the inserter assembly 1000. When in the retracted state, the lock members 1112A, B may permit rotation of the set cartridge 1002.

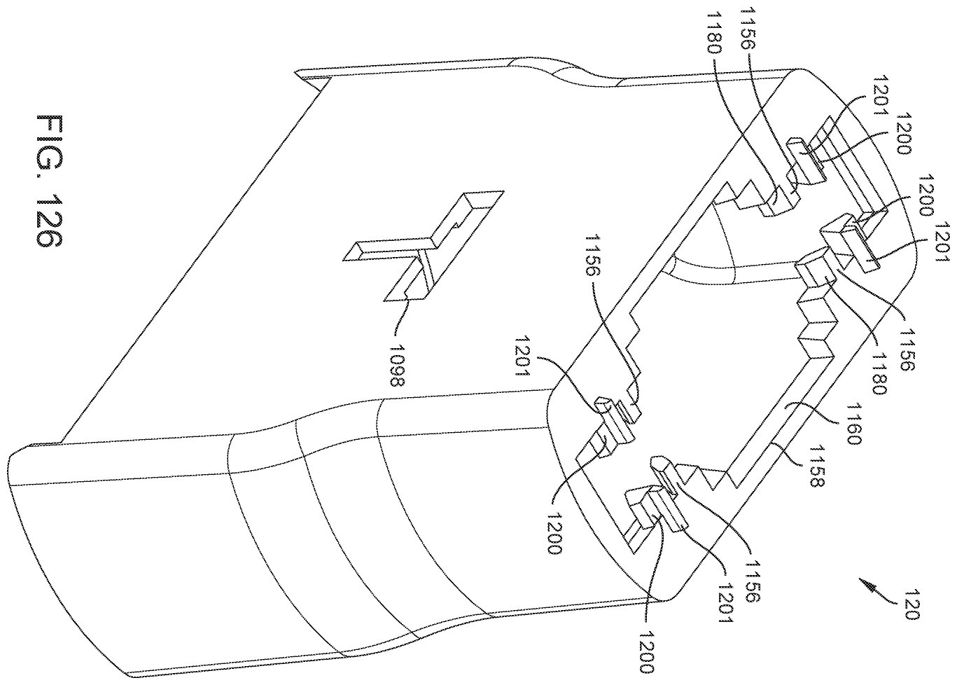

Referring now to FIG. 113, a lock member 1112A of the inserter assembly 1000 of FIG. 112 is depicted in isolation. As shown, the lock member 1112A may include a lock projection 1114 which may be the portion of the lock member 1112A which extends out of the receptacle body 1060. The lock projection 1114 may extend from a first end 1124 of a locating plate 1116 at a substantially perpendicular angle to the locating plate 1116. The locating plate 1116 may include one or more orifices 1118, 1120 which may allow the locating plate 1116 to be slid over portions of another component of the inserter assembly 1000 to hold the locating plate 1116 in a prescribed location within the inserter assembly 1000. The locating plate 1116 may slide over portions of the retraction latch body 1100 as, for example, shown in FIG. 112. A second end 1126 of the locating plate 1116 opposite the attachment point of the lock projection 1114 may be rounded.

As shown, the locating plate 1116 may include ridges 1122 which may be disposed on the sides of the locating plate 1116. Other components of the inserter assembly 1000 may also include ridges along their side edges (see e.g. the exploded views in FIGS. 106A-106B). These ridges 1122 may be provided as stiffeners which add rigidity to the lock members 1112A, B and any other components on which they are included. In some embodiments, the ridges 1122 may also help to guide or locate other components as they are displaced within an inserter assembly 1000.

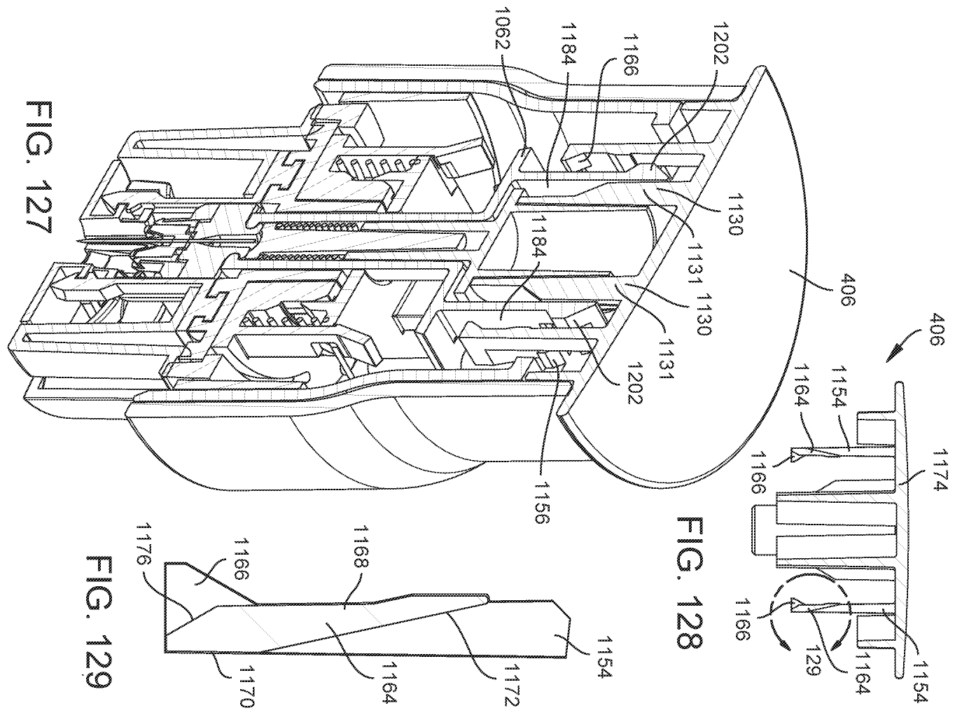

As shown in FIG. 112, the lock members 1112A, B may be biased towards the projecting state. In the example, the lock members 1112A, B are disposed between a respective bias member 1104A, B and a face of the retraction latch body 1100. The rounded end 1126 of each lock member 1112A, B may allow the lock member 1112A, B to pivot as they are actuated to the retracted state. The orifices 1118, 1120 may also be dimensioned to so as to allow such pivoting. When pivoted, the lock members 1112A, B may stress their associated bias member 1104A, B. Thus, when the exterior housing 1004 of the set cartridge 1002 is doffed, the lock members 1112A, B may automatically displace back to the projecting state as the bias members 1104A, B restore to their unstressed state (sec, e.g. FIG. 115). This may, for example, lock an interior housing 1008 of a set cartridge 1002 in place on the inserter assembly 1000.

Referring now to FIG. 114, in certain alternative embodiments (see, e.g. FIGS. 105A-105B) the lock members 1112A, B may be integrated into another component. As shown, the lock members 1112A, B are formed as projections which extend from an arcuate member 1128 which may, for example, be cantilevered to the retraction latch body 1100. When an inserter assembly 1000 including such lock members 1112A, B is assembled, the lock members 1112A, B may extend out of the receptacle body 1060 as shown in FIG. 108. When the lock members 1112A, B are displaced to the retracted state, the cantilevered arcuate members 1128 may resiliently deflect. The cantilevered arcuate members 1128 may resiliently restore to their unstressed state when, for example, the exterior housing 1004 is removed. This may cause the lock members 1112A, B to return to the projecting state and lock the interior housing 1008 in place on the inserter assembly 1000.

Referring now to FIG. 115, after removal of the adhesive backing 111 (see, e.g. FIG. 100A) by pulling on pull tabs 410, the set cartridge 1002 may be placed against the skin 356. The inserter assembly 1000 may then be actuated to a ready state. As the storage state of the example inserter assembly 1000 is a state in which all bias members 1104A, B, 1108 are in a relaxed or unstressed state, the inserter assembly 1000 may be actuated through a setting stage in order to place the inserter assembly 1000 in the ready state. In the setting stage, the bias members 1104A, B, 1108 of the inserter assembly 1000 may be transitioned (e.g. compressed) into a stressed state. To accomplish this, a user may press a first unit (e.g. exterior housing 116 and retainer cap 406) of the inserter assembly 1000 toward the remainder of the inserter assembly 1000 (or second unit of the inserter assembly 1000) and the skin 356. In addition to actuating the inserter assembly 1000 to the ready state, the pressure exerted by the user may help to ensure that the adhesive 374 on the infusion set base 106 is soundly adhered to the skin 356. Though FIG. 115 depicts the inserter assembly 1000 illustrated in FIGS. 106A-106B, the example inserter assembly 1000 of FIGS. 105A-105B may also be progressed through a setting stage by pressing on its exterior housing 116 and retainer cap 406. The inserter assembly 1000 may be considered to have progressed through the setting stage and into a ready state once the first unit of the inserter assembly 1000 has been displaced at least a threshold distance to a ready position. Once progressed through the setting stage, and as described in greater detail elsewhere herein, a retainer may engage with a latch to hold the bias members 1104A, B, 1108 in a stressed state.

Referring now to FIG. 116, in the example embodiment, as the user displaces the exterior housing 116 toward the skin 356, the bias members 1108, 1104A, B (or bias member 1104 of the example shown in FIGS. 105A-105B) may begin to compress. As shown, the retainer cap 406 may include a set of standoffs 1130. The standoffs 1130 may surround a barrel 1132 of a reset body 1110 of the inserter assembly 1000. As the exterior housing 116 is displaced, the standoffs 1130 may be driven into contact with a flange 1134 of the reset body 1110. In the example embodiment, the flange 1134 is disposed in a central region of the barrel 1132 and extends substantially perpendicularly thereto. Further displacement of the exterior housing 116 may cause the standoffs 1130 to push against the flange 1134 such that the reset body 1110 moves together with the exterior housing 116. This may cause the barrel 1132 to advance along the plunger 1106 such that an increasing amount of the plunger 1106 is disposed within the bore of the barrel 1132. As bias member 1108 is captured between an end of the barrel 1132 and a flange 1136 of the plunger 1106, the advancement of the barrel 1132 over the plunger 1106 may cause the bias member 1108 to be compressed.

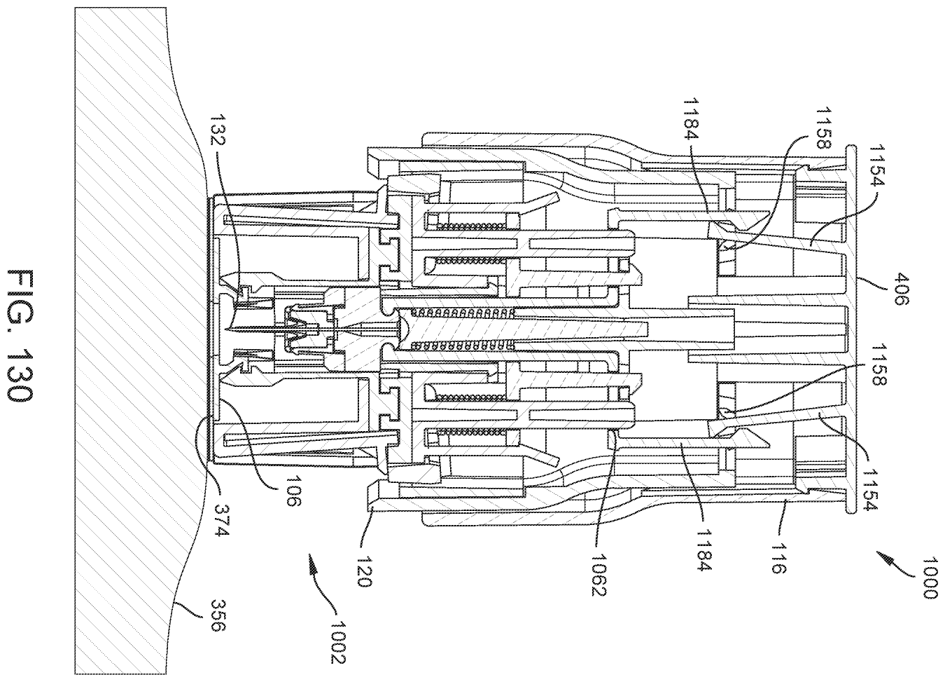

As the reset body 1110 is displaced, bias members 1104A, B (or bias member 1104 of the example shown in FIGS. 105A-105B) may also be compressed. In example embodiments, the retraction spring retainer 1102 may be coupled to the reset body 1110 such that the reset body 1110 and retraction spring retainer 1102 displace together as a unit. To illustrate this, an example reset body 1110 and retraction spring retainer 1102 are depicted coupled together and isolated from a remainder of the inserter assembly 1000 in FIG. 117. Referring to both FIG. 116 and FIG. 117, in the example embodiment, the retraction spring retainer 1102 includes a set of retainer arms 1138. The retainer arms 1138 may be cantilevered to a base 1140 of the retraction spring retainer 1102 which in the example embodiment is depicted as a planar body. Each of the retainer arms 1138 may include a protuberance 1142 disposed at an unsupported or terminal end thereof. A ledge section 1144 may be defined by a portion of each of the protuberances 1142. The flange 1134 of the reset body 1110 may include passages 1146 through which the protuberances 1142 may be displaced to couple the reset body 1110 and retainer arms 1138 via a snap fit engagement. The protuberances 1142 may include ramped portions to facilitate deflection of the retainer arms 1138 as the reset body 1110 and the retraction spring retainer 1102 are coupled to one another. When coupled together, a bottom face of the flange 1134 may rest against at least one step 1148 included on each retainer arm 1138. The retainer arm 1138 widths may change at the step 1148. The portion of the retainer arms 1138 proximal to the base 1140 with respect to the step 1148 may have a width which is greater than the width of the passages 1146. Thus, the flange 1136 may be captured between the ledge 1144 and step 1148 of each retainer arm 1138 and be unable to substantially displace relative to the ledge 1144 and step 1148. With the reset body 1110 and retraction spring retainer 1102 coupled together they may be displaced as a unit.

Referring primarily to FIG. 116, bias members 1104A, B may be captured between the retraction spring retainer 1102 and the retraction latch body 1100. One end of the bias members 1104A, B may contact the retraction spring retainer 1102 and the other may contact the lock members 1112A, B. In the example embodiment, the bias members 1104A, B are disposed surrounding guide projections 1150 included on the retraction latch body 1100. The guide projections 1150 may thus serve as locator projections which hold the bias members 1104A, B in place within the inserter assembly 1000. The retraction spring retainer 1102 and the insertion driver 1062 may include guide apertures 1152 through which the guide projections 1150 may extend. As the user pushes down on the exterior housing 116, the retraction spring retainer 1102 may displace toward the retraction latch body 1100 along the guide projections 1150. Since the bias members 1104A, B are captured between the retraction spring retainer 1102 and the retraction latch body 1100, the advancement of retraction spring retainer 1102 may cause the bias members 1104A, B to be compressed.

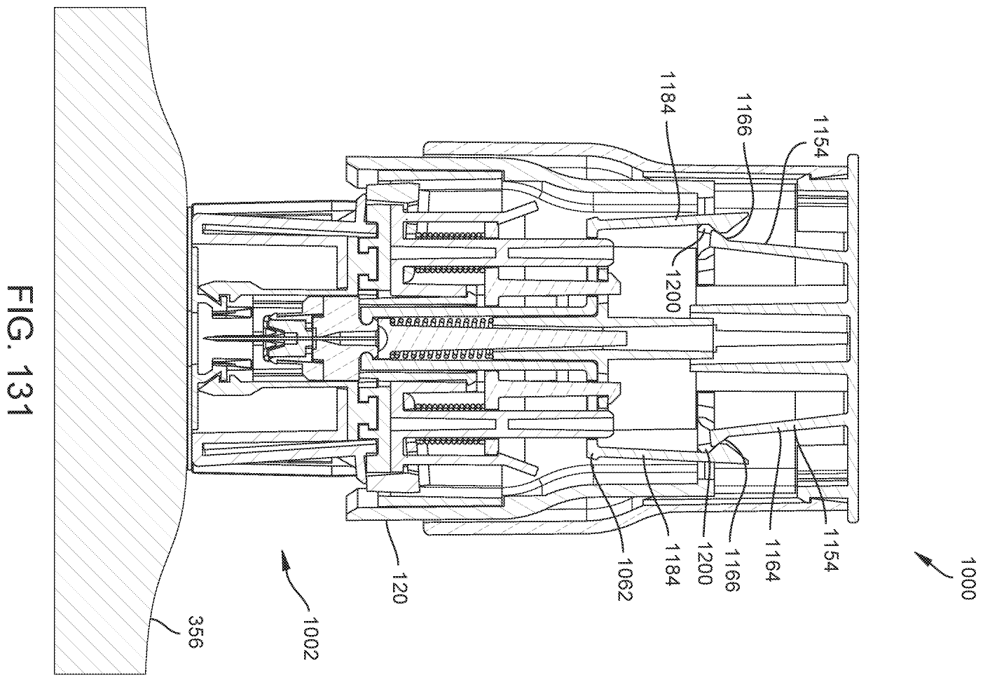

Referring now to FIGS. 118-119, in some embodiments, additional guide projections 1150 may be included, or guide projections 1150 may be included on another component of the inserter assembly 1000. FIG. 119 depicts a three-quarter section view (taken along line 119-119 of FIG. 118) of the example interior housing 120 of the inserter assembly 1000 shown in FIGS. 105A-105B. As shown, the interior housing 120 may include four guide projections 1150 (one is cut away) which extend from the top of the interior housing 120. The guide projections 1150 may be spaced at even angular intervals from one another though the spacing and number of guide projections 1150 may differ in alternative embodiments. The retraction spring retainer 1102 and insertion driver 1062 may have cooperating guide apertures 1152 (see, FIGS. 105A-105B) and may displace along the guide projections 1150.

Referring now to FIG. 120, as the exterior housing 116 of an inserter assembly 1000 continues to displace and the bias members 1104A, B, 1108 continue to compress, the base 1140 of the retraction spring retainer 1102 may contact latch arms 1190 of the retraction latch body 1100. As shown, the latch arms 1190 may be cantilevered from a base 1196 of the retraction latch body 1100. The latch arms 1190 may each include a first portion 1192 which is proximal to the base 1196. The first portions 1192 may, when unstressed, extend substantially perpendicular from the base 1196 and parallel to one another. Each latch arm 1190 may also include a second portion 1194 distal to the base 1196 which may be angled with respect to the first portion 1192. In the example, the second portions 1194 are angled such that distance between the latch arms 1190 increases with distance from the base 1196. A catch 1198 may be included where the second portion 1194 meets the first portion 1192 of each latch arm 1190. As the retraction spring retainer 1102 is driven into contact with the second portion 1194 of each latch arm 1190, the latch arms 1190 may be spread apart from one another as shown in FIG. 120.

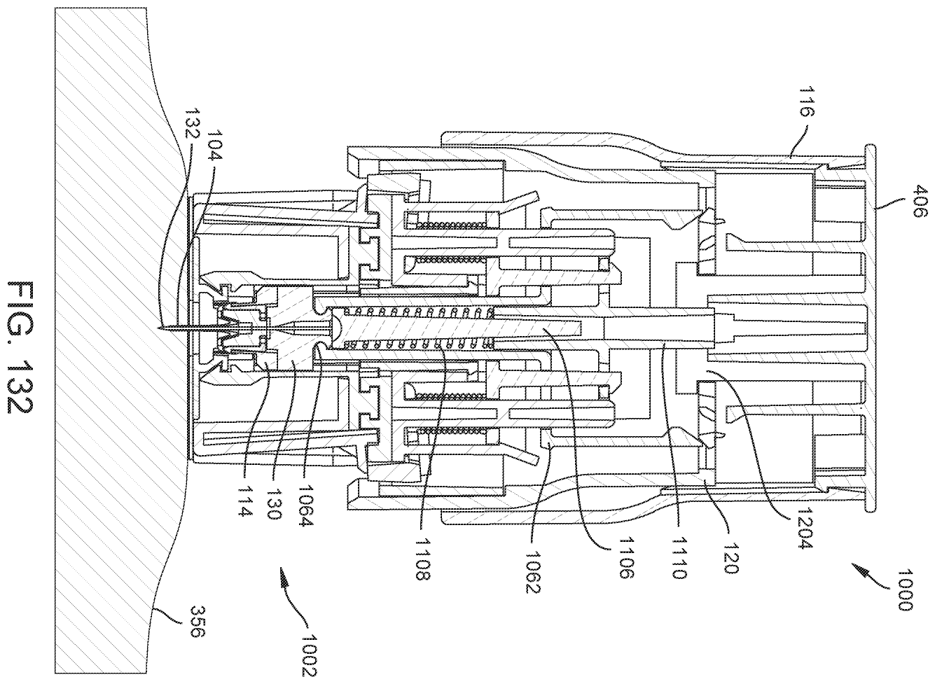

Referring now to FIGS. 121-123, views of the example inserter assemblies 1000 of FIGS. 105A-105B and FIGS. 106A-106B in a ready state are depicted. Once the retraction spring retainer 1102 has been displaced below the second portion 1194 of each latch arm 1190, the latch arms 1190 may resiliently restore from their spread state. When the latch arms 1190 restore to their unstressed state, the catches 1198 may overhang the base 1140 of the retraction spring retainer 1102. Thus, the catches 1198 may hold the retraction spring retainer 1102 in place with the bias members 1104A, B, 1108 compressed. The inserter assembly 1000 may then be ready to be actuated in order to drive installation of the infusion set 102 to the chosen infusion site on the patient. The exterior housing 116 and retainer cap 406 may be considered in a ready position in FIGS. 121-123.

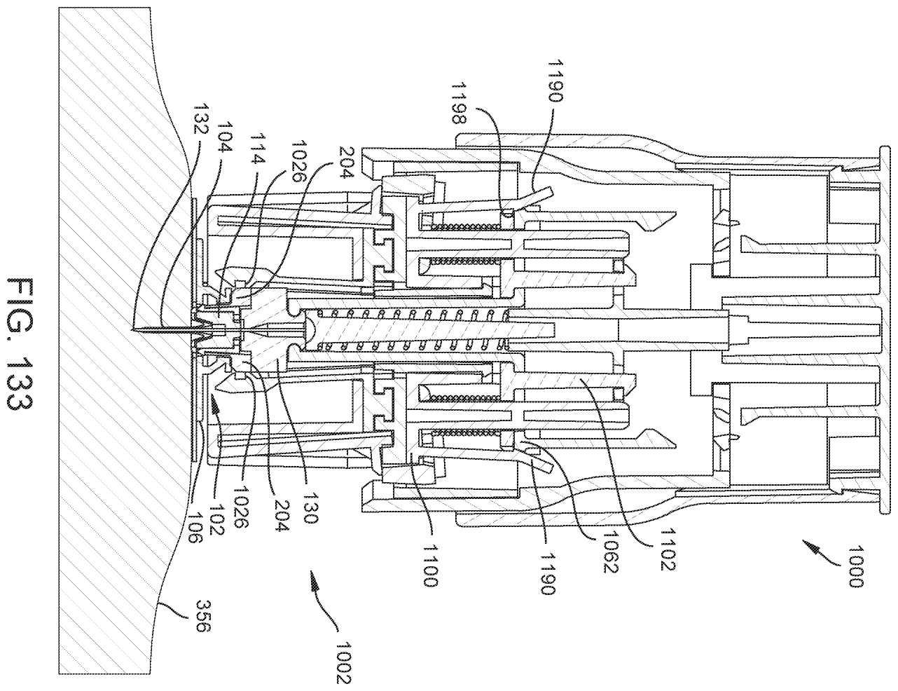

Referring now to FIG. 124 and FIG. 125, a cross-sectional view (FIG. 124) of an example inserter assembly 1000 and a detailed view (FIG. 125) of an indicated region thereof are shown. As the exterior housing 116 of the exemplary inserter assembly 1000 is pressed toward the skin 356 (see, e.g. FIG. 116) during the setting stage, release fingers 1154 extending from the retainer cap 406 may deflect around director wedges 1156 included on the interior housing 120. Each of the release fingers 1154 may be connected to a base portion 1174 of the retainer cap 406 in a cantilevered manner. At an unsupported end 1162 of each release finger 1154 a paddle body 1164 having a projection 1166 may be included. The paddle bodies 1164 may have a width greater than the remainder of the release fingers 1154. In some embodiments, the width may be 2-3 times (e.g. 2.15-2.30 or in some examples 2.25 times) the width of the rest of the release finger 1154. The paddle body 1164 may be disposed such that a center region of the paddle body 1164 is connected to the rest of the associated release finger 1154. Thus, the paddle body 1164 may include two evenly sized portions (only one is visible in the FIG. 124 cross-section) which flank the release finger 1154.

Each paddle body 1164 may include a medial face 1168 proximal to the longitudinal axis or midplane of the inserter assembly 1000 and a lateral face 1170 on a side of the paddle body 1164 opposite the medial face 1168. The projection 1166 may be disposed on the lateral face 1170 of each paddle body 1164. The projection 1166 may be centrally disposed on each paddle body 1164 such that the projection 1166 may be displaced without contacting the director wedges 1156 during actuation. The lateral face 1170 of each paddle body 1164 may include a lateral ramp portion 1172 on the flanking portions of the paddle body 1164. The lateral ramp portions 1172 may be included on a section of the paddle body 1164 most distal to the base portion 1174 of the retainer cap 406. The lateral ramp portions 1172 may slope toward the medial face 1168 as distance from the base portion 1174 of the retainer cap 406 increases. The medial face 1168 of the paddle body 1164 may also include a medial ramp portion 1176 on the flanking portions of the paddle body 1164. The medial ramp portions 1176 may be included on a portion of the paddle body 1164 most proximal to the base portion 1174 of the retainer cap 406. The medial ramp portions 1176 may slope toward the lateral face 1170 as distance to the base portion 1174 of the retainer cap 406 decreases.

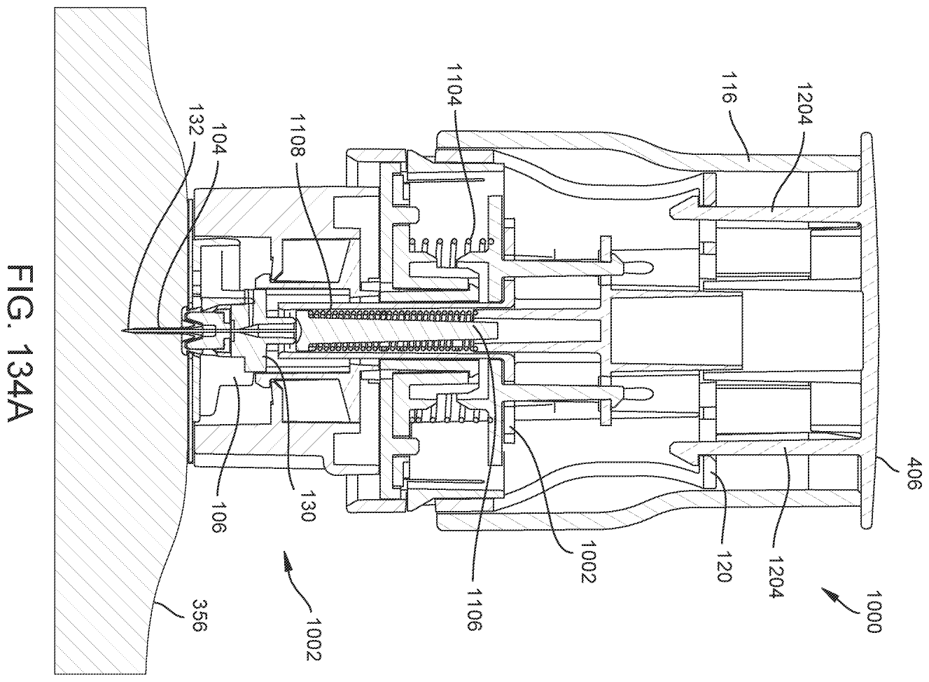

Referring now also to FIG. 126, in the example embodiment, the director wedges 1156 extend from the side wall 1160 of a passage 1158 through the top of the interior housing 120 (shown in isolation in FIG. 126). The director wedges 1156 are included in pairs. The director wedges 1156 of each pair extend from opposing side walls 1160 of the passage toward one another. The director wedges 1156 include a top face 1180 which slopes toward the midplane of the inserter assembly 1000 as distance from the top of the interior housing 120 increases. The director wedges 1156 also include a bottom face 1182 which is parallel to the top face 1180. The ends of the top and bottom faces 1180, 1182 most distal to the top of the interior housing 120 may be rounded or chamfered to form a point (as shown in FIGS. 124-126). As the exterior housing 116 is displaced toward the body during the setting phase, the release fingers 1154 and paddle bodies 1164 may displace along displacement paths into which the director wedges 1156 extend. The lateral ramp portions 1172 of the paddle body 1164 of each release finger 1154 may contact the top faces 1180 of a pair director wedges 1156 during this displacement. Further displacement may cause the lateral ramp portions 1172 to slide along the top faces 1180 of the director wedges 1156. This may generate deflection of the associated release finger 1154 and movement of the projection 1166 of the paddle body 1164 toward the midplane (see, e.g., FIG. 116) of the inserter assembly 1000. The projection 1166 of the paddle body 1164 may pass through the gap between the associated pair of director wedges 1156 as this displacement occurs. Once the paddle body 1164 has been displaced below the director wedges 1156, the release finger 1154 may be free to resiliently restore to its undeflected state (see, e.g., FIG. 120).

As will be described in greater depth later in the specification, the exterior housing 116 may be displaced relative to the interior housing 120 in the opposite direction to fire the inserter assembly 1000. As this occurs, the medial ramp portions 1176 of the paddle body 1164 of each release finger 1154 may contact the bottom face or underside 1182 of the associated director wedges 1156. Further displacement may cause the medial ramp portion 1176 to slide along the underside 1182 of the director wedges 1156. This may generate deflection of the release finger 1154 and projection 1166 of the paddle body 1164 away from the midplane of the inserter assembly 1000. The projection 1166 of the paddle body 1164 may pass through the gap between the associated pair of director wedges 1156 as the exterior housing 116 is displaced.

As the release fingers 1154 are deflected away from the midplane of the inserter assembly 1000, the projections 1166 may deflect toward cantilevered arms 1184 on the insertion driver 1062. The arms 1184 may form resilient projections which may deflect if sufficient force is exerted against them. The unsupported end of the arms 1184 may include a curved or ramped section. A notch or pair of notches 1188 may also be present on each of the cantilevered arms 1184. In the example, each arm 1184 includes a pair of notches 1188 located near the unsupported ends of the arms 1184. The notches 1188 may be disposed similarly to the notches 342 shown in FIG. 71 for example. Each of the one or more notches 1188 may engage with a cooperating projection 1200. The cooperating projection 1200 may be included on the interior housing 120 and may project from the side wall 1160 of the passage 1158 through the top of the interior housing 120. The interaction of the notch(es) 1188 and cooperating projection(s) 1200 may maintain the bias member 1108 under compression and serve as an insertion prevention latch or insertion driver latch.

With the release fingers 1154 deflected toward the arms 1184, further displacement of the exterior housing 116 away from the skin 356 may cause the projections 1166 on the paddle bodies 1164 to collide with protrusions 1202 on the cantilevered arms 1184. This may dislodge the notches 1188 of the cantilevered arms 1184 from cooperating projections 1200 of the interior housing 120. Thus, once the first unit of the inserter assembly 1000 (e.g. exterior housing 116 and retainer cap 406) has been pulled away from the remainder of the inserter assembly 1000 (second unit of the inserter assembly 1000) beyond a threshold distance (which may be measured from the ready position), the insertion driver 1062 may be dislodged from the insertion driver latch. This may free the spring 1108 to transition to its unstressed state and displace the insertion driver 1062 toward the skin 356. As the spring 1108 transitions from its stressed state to a relaxed state, the inserter driver 1062 may displace from its stowed state to an extended position in which at least a portion of the insertion driver 1062 projects out of the inserter assembly 1000 and into the cartridge 1002. As shown in best in FIG. 126, a ridge 1201 which blocks displacement of the cantilevered arms 1184 may be included medial to each of the cooperating projections 1200. This may help to ensure that the cantilevered arms 1184 may only be released from the cooperating projections 1200 when intended by the user.

Referring now to FIG. 127, a cross section of the inserter assembly 1000 shown in FIGS. 105A-105B is depicted. In some embodiments, and as shown in FIG. 127, the release fingers 1154 may be disposed outboard (with respect to the midplane) as opposed to inboard (sec e.g. embodiment of FIGS. 106A-106B) of the cantilevered arms 1184 of the insertion driver 1062. In such embodiments, to be displaced off their respective cooperating projections 1200 of the interior housing 120, they may be displaced toward the midplane of the inserter assembly 1000. In such embodiments, the standoffs 1130 included on the retainer cap 406 may include an enlarged section 1131. The enlarged sections 1131 may block the cantilevered arms 1184 from being dislodged until the inserter assembly 1000 has been withdrawn from the skin 356 more than a certain amount.

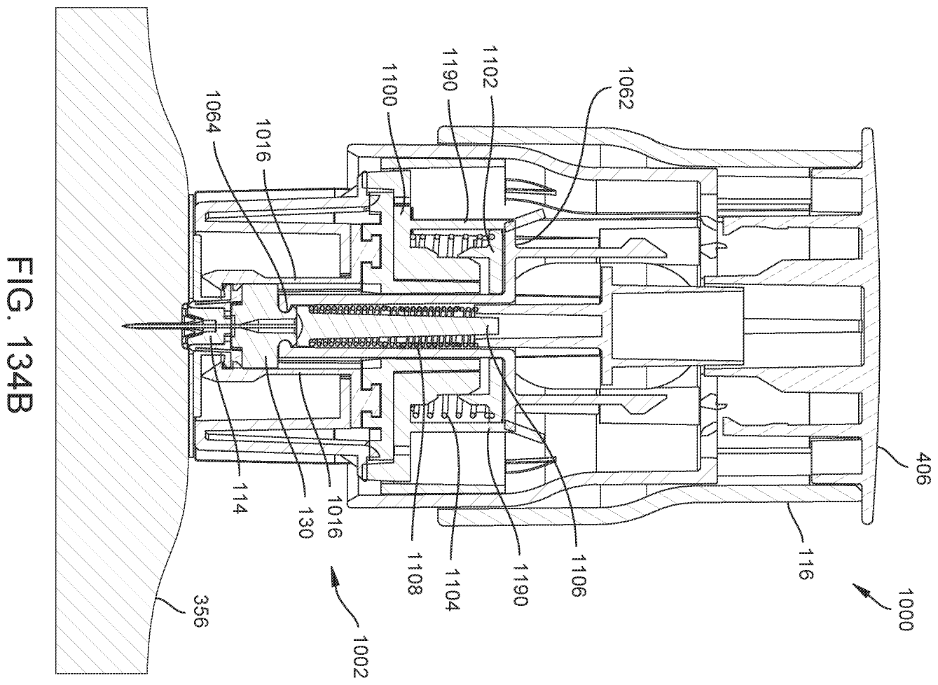

Referring also to FIG. 129, a detailed view of the indicated region of FIG. 128, the release fingers 1154 may have paddle bodies 1164 configured to deflect the release fingers 1154 in opposite directions to those described in relation to FIGS. 124-126. Each paddle body 1164 may include a medial face 1168 proximal to the longitudinal axis or midplane of the inserter assembly 1000 and a lateral face 1170 on a side of the paddle body 1164 opposite the medial face 1168. A projection 1166 may be disposed on the medial face 1170 of each paddle body 1164. The projection 1166 may be centrally disposed on each paddle body 1164 such that the projection 1166 may be displaced without contacting the director wedges 1156 during actuation. The lateral face 1170 of each paddle body 1164 may include a lateral ramp portion 1172 on the flanking portions of the paddle body 1164. The lateral ramp portions 1172 may be included on a section of the paddle body 1164 most proximal to the base portion 1174 (sec, e.g., FIG. 128) of the retainer cap 406. The lateral ramp portions 1172 may slope toward the medial face 1168 as distance from the base portion 1174 of the retainer cap 406 decreases. The medical face 1168 of the paddle body 1164 may also include a medial ramp portion 1176 on the flanking portions of the paddle body 1164. The medial ramp portions 1176 may be included on a portion of the paddle body 1164 most distal from the base portion 1174 of the retainer cap 406. The medial ramp portions 1176 may slope toward the lateral face 1170 as distance to the base portion 1174 of the retainer cap 406 increases.

As the exterior housing 116 is pressed toward the skin 356 during the setting stage, the medial ramped portions 1176 may deflect the paddle bodies 1164 of the release fingers 1154 outwardly around the director wedges 1156. As the inserter assembly 1000 is lifted from the skin 356 to trigger insertion, the lateral ramped portions 1172 may deflect the release fingers 1154 inwardly around the director wedges 1156 and towards the cantilevered arms 1184. With the release fingers 1154 deflected toward the arms 1184, further displacement of the exterior housing 116 away from the skin 356 may cause the projections 1166 on the paddle bodies 1164 to collide with protrusions 1202 on the cantilevered arms 1184. This may dislodge the notches 1188 (described above) of the cantilevered arms 1184 from cooperating projections 1200 (described above) of the interior housing 120. This may free the spring 1108 to transition to its unstressed state and displace the insertion driver 1062 toward the skin 356.

As shown in FIG. 130, actuation of an inserter assembly 1000 may be triggered as the inserter assembly 1000 is lifted up from the skin 356 so as to be removed from the patient. No other depression, twisting, squeezing, etc. of a trigger, button, housing sleeve or other portion of an inserter assembly 100 by a user may be needed to provoke the actuation, however, the actuation may still be under the control of the user. In alternative embodiments, and as discussed elsewhere herein, a discrete manual triggering action may be employed to trigger actuation of an inserter assembly 1000. Where triggering occurs automatically as the inserter assembly 1000 is withdrawn from the skin 356, relative movement of the free component(s) of the inserter assembly 1000 with respect to the restricted component(s) may trigger actuation. As described above, actuation may be triggered by, for example, displacing or dislodging a latch and freeing one or more bias members to begin driving actuation. Thus, one or more trigger internal to the inserter assembly 1000 may be actuated as a result of the removal action of the inserter assembly 1000 from the body. From the perspective of a user, such an inserter assembly 1000 may simply be placed on the skin 356 and then withdrawn to execute placement of the infusion set 102. This may be advantageous for a number of reasons which are detailed elsewhere in the specification.

FIG. 130 depicts the example inserter assembly 1000 of FIGS. 106A-106B coupled set cartridge 1002 and as it is being withdrawn from the skin 356. As shown, the adhesive 374 on the infusion set base 106 may adhere to the skin 356 resulting in the skin 356 being tugged upward as the inserter assembly 1000 and set cartridge 1002 are withdrawn. The exterior housing 116 and retainer cap 406 may displace together as a unit with the hand of the user as the user removes the inserter assembly 1000 from their body. This may also be true of the exterior housing 116 and retainer cap 406 of the example inserter assembly 1000 shown in FIGS. 105A-105B. The other components of the example inserter assemblies 1000 and set cartridges 1002 may not be constrained to displace as a unit with the exterior housing 116 and retainer cap 406. These components may form a second unit which maybe held behind, unable to displace relative to the patch of skin 356 adhered to the adhesive 374.

During removal of the inserter assembly 1000 and set cartridge 1002, the exterior housing 116 and retainer cap 406 may displace away from the skin 356 substantially along the axis of the insertion sharp 132. This displacement relative to the other components may cause the release fingers 1154 to deflect towards the cantilevered arms 1184 of the insertion drive 1062 as described above. The resiliency of release fingers 1154 may cause the entire inserter assembly 1000 and set cartridge 1002 to move together for at least a portion of the inserter assembly 1000 withdrawal motion from the skin 356. During this portion of the removal action of the inserter assembly 1000, the skin 356 may be lifted off underlying body structures. Portions of the inserter assembly 1000 may again displace relative to one another once the force exerted by the elasticity of the skin exceeds a force threshold and the release fingers 1154 fully deflect around the deflector wedges 1158.

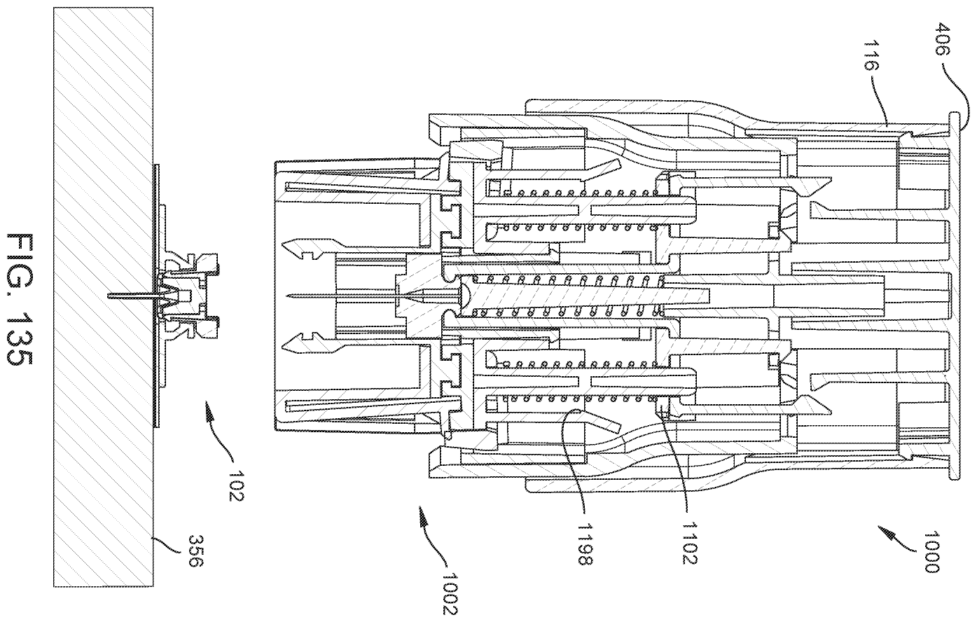

Referring now to FIG. 131, as the exterior housing 116 and retainer cap 406 continue to displace relative to the rest of the inserter assembly 1000 and set cartridge 1002, the cantilevered arms 1184 of the insertion driver 1062 may be dislodged off the associated cooperating projections 1200 of the interior housing 120. As described above with respect to FIGS. 124-126 or FIGS. 127-129, this may be caused by the projections 1166 of the paddle bodies 1164 on the release fingers 1154 colliding with protrusions 1202 at the unsupported ends of the cantilevered arms 1184.

Referring now to FIG. 132, this may allow the bias member 1108 between the reset body 1110 and the plunger 1106 to transition to its uncompressed state. As the bias member 1108 transitions to its relaxed state, the plunger 1106 and insertion driver 1062 may be propelled towards the skin 356. As the sharp holder 130 is coupled into the port 1064 of the insertion driver 1062, the sharp holder 130 and cannula subassembly 114 coupled thereto may also be shot towards the skin 356 along an insertion path. As shown in FIG. 132, which depicts the inserter assembly 1000 of FIGS. 106A-106B for sake of example, the insertion sharp 132 and cannula 104 have just punctured through the skin 356. During puncture, the skin 356 may still be in a state in which it is tugged up away from muscle and other underlying body structures. The cannula subassembly 114 has begun to be advanced toward the infusion set base 106 as well. Also as shown, the retainer cap 406 of the inserter assembly 100 may include one or more stop arms 1204 which prevent relative movement beyond a certain point between the interior housing 120 and the casing formed by the exterior housing 116 and retainer cap 406. The stop arms 1204 may catch on a portion of the top of the interior housing 120 inhibiting further relative displacement. The stop arms 1204 may be engaged as the insertion of the cannula 104 begins or is in progress. Thus, additional lifting of the skin 356 may occur during the cannula 104 insertion movement.

In some embodiments, a bias member may be included such that the stop arms 1204 must compress the bias member prior to the cantilevered arms 1184 of the insertion driver 1062 being dislodged from the cooperating projections 1200. This may aid in further lifting the skin 356 before insertion is triggered. In some embodiments, the portions of the interior housing 120 which the stop arms 1204 contact may be resiliently cantilevered in the path of the stop arms 1204. Thus, as the inserter assembly 1000 is withdrawn, the cantilevered portions of the interior housing 120 may be deflected by the stop arms 1204 before the cantilevered arms 1184 are dislodged from the cooperating projections 1200. The resiliency of these cantilevered portions may be chosen to ensure that the skin is lifted at least a certain amount. In other embodiments, springs (e.g. compression springs, leaf type springs, etc.) may be placed in the path of the stop arms 1204. These springs may need to be brought to a stressed state via displacement of the stop arms 1204 before the cantilevered arms 1184 are released. Again, the springs may be chosen such that at least a desired amount of skin lifting occurs prior to the cantilevered arms 1184 being freed from the cooperating projections 1200.

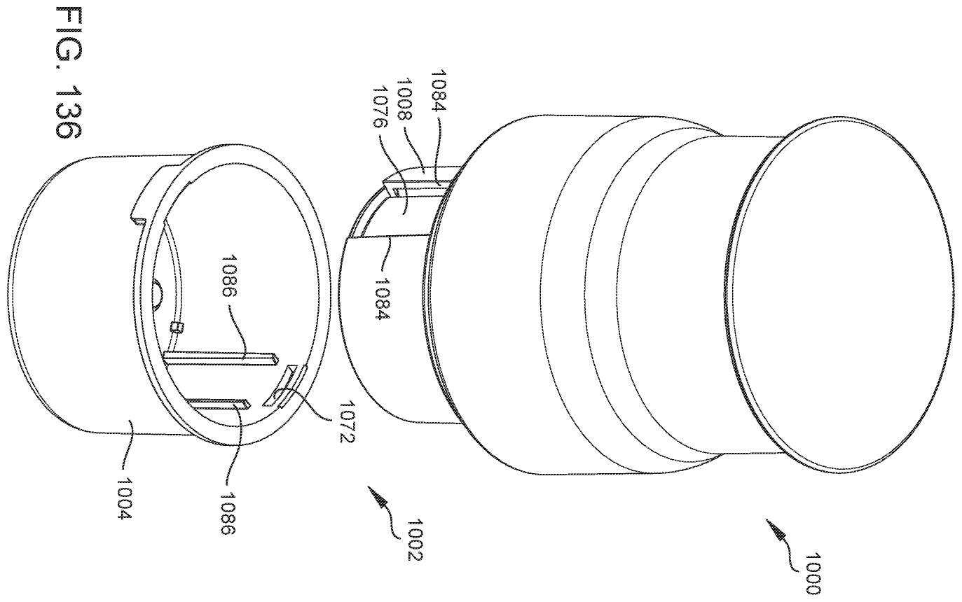

Referring now to FIG. 133, another cross sectional view of an example inserter assembly 1000 and set cartridge 1002 are shown. As shown, the spring 1108 has restored to a relaxed state and completed the insertion movement of the sharp holder 130, insertion sharp 132, and cannula subassembly 114 toward the skin 356. The relaxed state may be a completely relaxed state or a comparatively relaxed state where the spring 1108 is still exerting some pressure against the sharp holder 130 to prevent it from jostling about. Similarly to as shown in FIG. 77B, a notch 184 of the cannula subassembly 114 may be in engagement with a protuberance 182 of the infusion set base 106 locking the cannula subassembly 114 in place and completing assembly of the infusion set 102. As shown, when the cannula subassembly 114 latches into the base 106, the cars 204 on the cannula subassembly 114 may press against the nubs 1026 included on the retainer arms 1016. This may cause the retainer arms 1016 to be splayed apart resulting in disengagement of the retainer arms 1016 from the infusion set base 106. Thus, the assembled infusion set 102 may be installed at the infusion site and released from the set cartridge 1002.

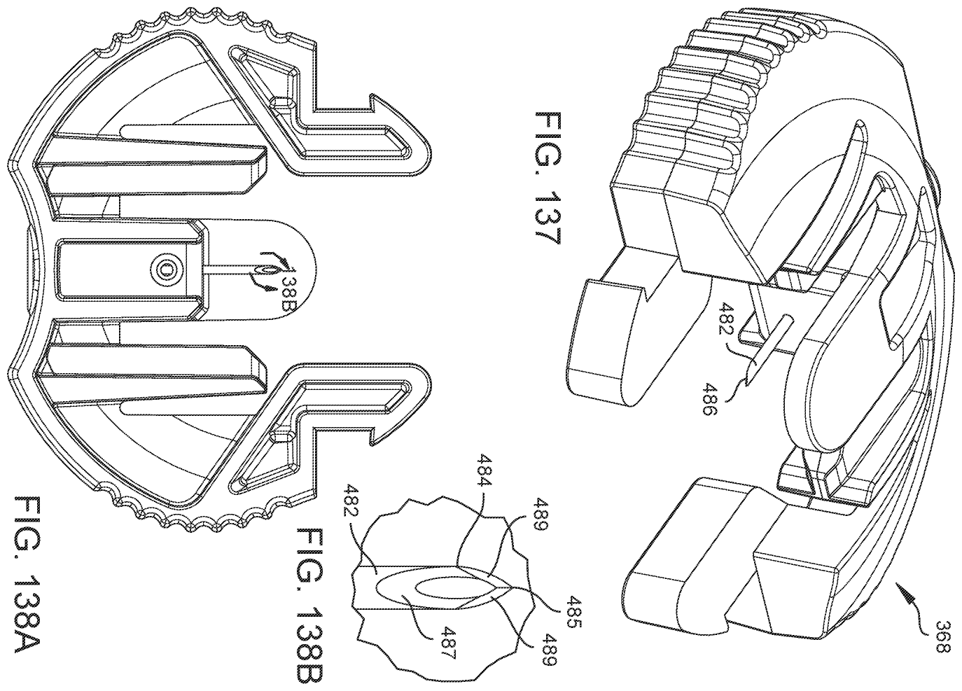

As also shown in FIG. 133, the insertion driver 1062 may collide with the latch arms 1190 of the retraction latch body 1100 as the now assembled infusion set 102 is released. This collision may cause the latch arms 1190 to spread apart freeing the retraction spring retainer 1102 from engagement with the catch 1198 of the latch arms 1190. Thus, FIG. 133 depicts the insertion assembly 1000 at a retraction release state. The example inserter assembly 1000 of FIGS. 105A-105B is shown in the retraction release state in FIGS. 134A-134B.

Referring now to FIG. 135, with the retraction spring retainer 1102 freed from the catches 1198, the bias members 1104A, B (or bias member 1104 for the inserter assembly 1000 of FIGS. 105A-105B) may transition to their unstressed state driving the retraction spring retainer 1102 toward the retainer cap 406 of the exterior housing 116. As a portion of the insertion driver 1062 is between the retraction spring retainer 1102 and the retainer cap 406, the insertion driver 1062 may also be displaced along with the retraction spring retainer 1102 as the bias members 1104A, B relax. Since the sharp holder 130 is coupled to the insertion driver 1062, displacement of the insertion driver 1062 may cause the insertion sharp 132 to be removed from the skin 356 and retracted back into the set cartridge 1002.

In certain alternative embodiments, retraction of the insertion sharp 132 may not be automatic and/or may not be spring biased. For example, the insertion sharp 132 may remain in the advanced position and the removal action of the user may manually pull the insertion sharp 132 out of the cannula 104. In such embodiments, bias members 1104A, B may be omitted. In some embodiments, a button press or similar interaction may be required to trigger a spring biased retraction of the insertion sharp 132.

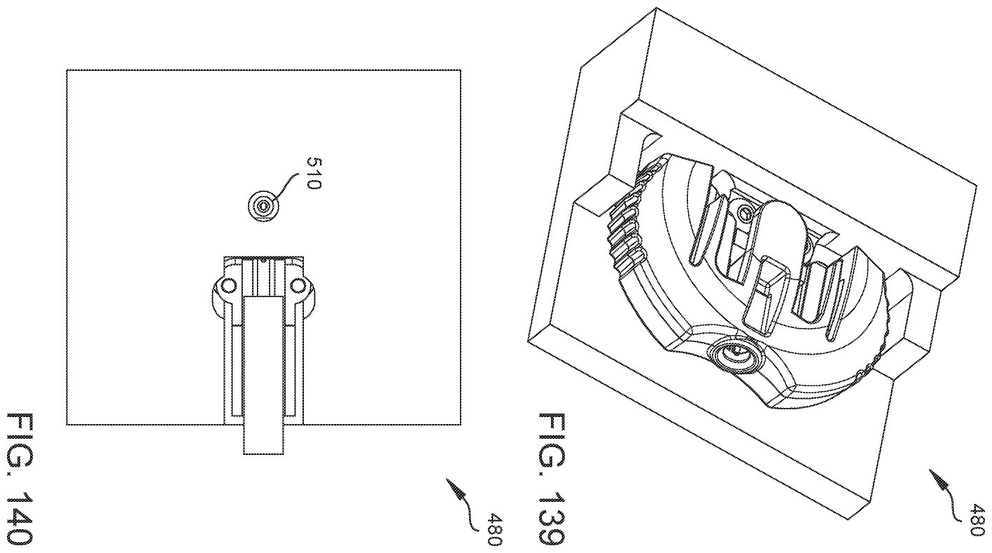

Referring now to FIG. 136, the exterior housing 1004 is shown separated from the remainder of a spent set cartridge 1002 which is installed on an inserter assembly 1000. Once the infusion set 102 is installed at the desired infusion site, the exterior housing 1004 may be used as a removal and containment tool for the spent set cartridge 1002. As shown, the cantilevered arms 1076 of the interior housing 1008 may be recessed with respect to the external face 1082 of the interior housing 1008. A recess edge wall 1084 may be present on each side of the cantilevered arms 1076. The exterior housing 1004 may include a set of stops 1086 which may fit within the recesses formed in the interior housing 1008. After insertion of the infusion set 102, the exterior housing 1004 may be slid over the interior housing 1008 and the spent set cartridge 1002 may be rotated to an uncoupled state. The stops 1086 may catch against the recess edge walls 1084 and ensure that the interior housing 1008 rotates in tandem with the exterior housing 1004. Referring now also to FIG. 108, this may displace the ramped projections 1074 of the housing tabs 1070 out of abutment with the deflector members 1078 of the receptacle body 1060. Due to the resiliency of the cantilevered arms 1076, the housing tabs 1070 may restore back into engagement with the receiver slots 1072 in the exterior housing 1004. The spent set cartridge 1002 may then be removed from the inserter assembly 1000.

After use, the exterior housing 1004 may serve as a containment for the remaining components of the spent set cartridge 1002. This may ensure that the used insertion sharp 132 is enclosed within the set cartridge 1002. Thus, the point of the insertion sharp 132 may be inaccessible to the user. The engagement of the housing tabs 1070 with the receiver slots 1072 may lock the insertion sharp 132 within the spent set cartridge 1002. In certain embodiments, placement of the infusion set 102 on the infusion site via the inserter assembly 1000 may be performed with one hand. Thus, after removing the exterior housing 1004, the user may be encouraged to hold the exterior housing 1004 in their free hand as the infusion set 102 is applied with the inserter assembly 1000. The user may then remove the spent set cartridge 1002 by reattaching the exterior housing 1004 to the interior housing 1008 and separating the set cartridge 1002 from the inserter 1000. Occupying both hands of the user during the process may aid in limiting opportunity for a user to inadvertently come into contact with the insertion sharp 132.

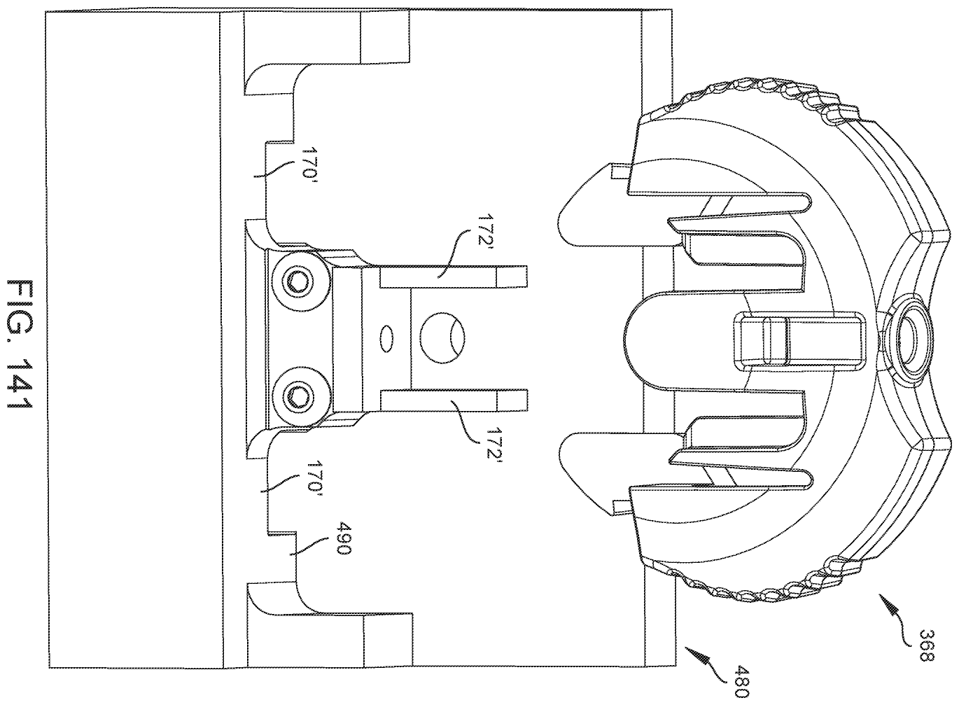

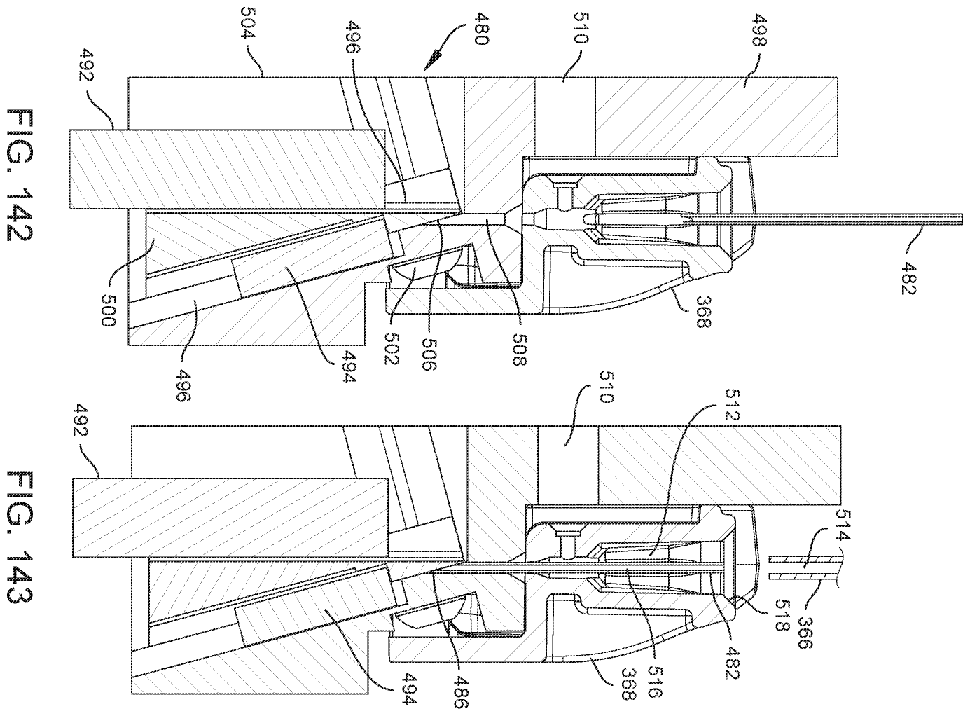

Referring now to FIGS. 137-141, an example tubing connector 368 (FIGS. 137-138B) and a fixture 480 (FIGS. 139-140) for assembling a tubing connector 368 are depicted. The fixture 480 may aid in assembly of a sharp 482 and infusion tubing 366 (see, e.g. FIG. 6) into a tubing connector 368. The fixture 480 may ensure that the sharp 482 is positioned so as to have an exposed portion 484 of a prescribed length. In various embodiments, this may aid in ensuring that the sharp 482 extends a desired distance into a septum 110 (see e.g. FIG. 5B) when the tubing connector 368 is coupled to an infusion set 102 (see, e.g. FIG. 6). For example, the length of the exposed portion 484 may be sized so as to extend into a fluid introduction volume of the cannula subassembly 114 as described elsewhere herein.

In the example, the sharp 482 is depicted with a lancet tip though other types of sharps may be used. For example, a sharp 482 with a back bevel may be used in certain embodiments. The exemplary sharp 482 (see FIG. 138B) includes a primary grind region 487 which is at an angle (e.g. an angle between 10-20° such as 15.5°) with respect to the longitudinal axis of the sharp 482. A set of secondary grinds 489 are also included and form the point 485 of the sharp 482.

Additionally, the fixture 480 may ensure that the sharp 482 is brought into a prescribed rotational orientation. This may be achieved through the use of magnetism. Thus, tips 486 of sharps 482 of tubing connectors 368 may be uniformly oriented across tubing connectors 368. This may be desirable for a variety of reasons. For example, during the typical usage life of an infusion set 102, the tubing connector 368 may be disconnected and reconnected a number of times. As the tip 486 is ramped, there may be a tendency for the sharp 482 to veer from the axis of insertion as the tubing connector 368 is advanced into engagement with the infusion set base 106. This tendency may be exaggerated with repeated connection and disconnection. By ensuring the tip 486 is always oriented in a particular orientation any veering of the sharp 482 may occur in a predictable direction and the septum 110 may be designed to accommodate such veering. This may allow for the septum 110 to be made smaller in portions where veering of the sharp 482 is not expected to occur. The septum 110 may be made smaller in footprint or height and with less material. Additionally, the fluid introduction volume formed by the septum recess 196 (see, e.g. FIG. 5B) may be made smaller. This would minimize any hold up volume in the infusion set 102 and minimize an amount of drug or agent expended filling this volume. Such rotational alignment of the sharp 482 may allow for a wider variety of tip 486 bevels to be used. It may also minimize any potential for occlusion due to the tip 486 being entirely surround by septum 110 material after a connection is made. Alternatively, it may allow for connection between the infusion set 102 and tubing connector 368 to be made in a more forgiving manner. As a result, the range of acceptable tolerances on various features of the infusion set 102 and tubing connector 368 may be greater. Likewise, the amount of material needed to form guides 172 (see, e.g. FIG. 6) or flanking projection 372 of the tubing connector 368 may be lessened.

As best shown in FIG. 141, the fixture 480 may include a dock 490 for a tubing connector 368. The dock 490 may mimic the mating interface of an infusion set base 106 (sec, e.g. FIG. 6) and may, for example, include guides 172′ and connector receivers 170′. Thus, the tubing connector 368 may engage the fixture 480 and be retained in fixture 480. The dock 490 may also position the tubing connector 368 in a desired location and orientation on the fixture 480.

The fixture 480 may include an adhesive port 510 (best shown in FIG. 140) which may allow glue, epoxy, or adhesive to be introduced into the tubing connector 368 such that the sharp 482 may be retained in the tubing connector 368 once properly positioned therein. A UV cure adhesive may be used in certain embodiments.

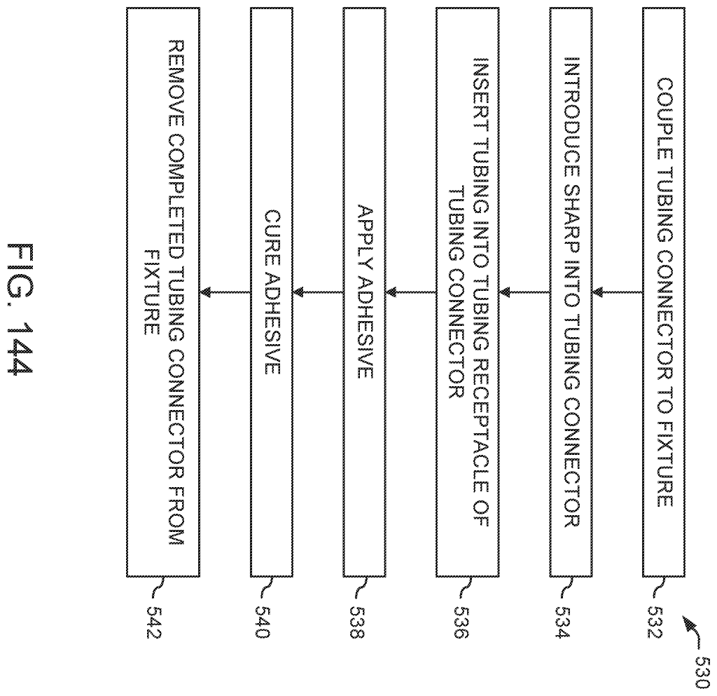

Referring now to FIGS. 142-143, two cross-sectional views of a tubing connector 368 installed on a dock 490 are shown. A sharp 482 is shown being installed into the tubing connector 368 in FIG. 142. The sharp 482 is in place within the tubing connector 368 and rotationally clocked such that the point 485 of the sharp 482 is in a specified position which in the example embodiment is a 12 o'clock position. In the example embodiment, the point 485 of the tip 486 of the sharp 482 is positioned to be substantially in line with and perpendicular to the axis of a lumen 202 of a cannula 104 of the infusion set 102 when the tubing connector 368 is connected thereto. The primary grind 487 and secondary grinds 489 on the tip 486 are also in a downward facing position.

As shown, the fixture 480 includes a first body 498 and a second body 500. The bodies 498, 500 may be coupled together via any suitable method including bonding, welding, adhesive, fasteners, etc. In the example, screws 502 are used to couple the first body 498 and second body 500. The second body 500 may fit within a cavity or slot 504 in the first body 498 which may serve to position the second body 500 with respect to the first body 498. As shown, the second body 500 may include a sloped face 506. The sloped face 506 may have an angle with respect to the axis of the sharp 482 which is substantially equal to the angle of one of the grinds 487, 489 on the sharp 482. In the example, the angle is about 15.5° which is the angle of the primary grind 487 of the sharp 482. The sloped face 506 may include a portion which is in line with a sharp receiving bore 508 of the first body 498. This portion may act as a support surface for a primary grind 487 of a sharp 482. The second body 500 may be constructed of a hard, non-metallic material.

As shown, the fixture 480 may include a first magnet 492 and a second magnet 494. The first magnet 492 may be larger than the second magnet 494. The dimensions of each edge of the second magnet 494 may be half the size of the dimensions of the respective sides of the first magnet 492. In some embodiments, the first magnet 492 may be a ⅛ in×⅛ in×½ in NdFeB magnet. The second magnet 494 may be a 1/16 in× 1/16 in×¼ in NdFeB magnet. In alternative embodiments electromagnets which may, for example, be equivalent to the permanent magnets just described may be used. The first magnet 492 and second magnet 494 may be located in channels 496 included in the fixture 480. The channel 496 for the second magnet 494 may be at an angle with respect to the channel 496 for the first magnet 492. In the example embodiment the angle is about 15.5°. The angle may reflect the angle of one of the grinds 487, 489 of the sharp 482. The angle may be the same as that of the sloped face 506 of the second body 500. The first magnet 492 may be oriented such that each of its poles are respectively most proximal to the opposing pole of the second magnet 494. The ends of the magnets 492, 494 are set back from the support surface portion of the sloped face 506. In the example embodiment, the ends of the magnets 492, 494 are set back about 0.060 inches.

As a sharp 482 is introduced into the tubing connector 368, the magnetic fields generated by the magnets 492, 494 may rotate and guide the sharp 482 into the desired rotational orientation. The magnetic fields may also draw the sharp 482 into contact with the sloped face 506 setting the length of the exposed portion 484 of the sharp 482. During introduction of the sharp 482, the sharp 482 may be displaced into the desired position and orientation with no or minimal contact of the point 485 or secondary grinds 489 with any material of the tubing connector 368 or the fixture 480. Only the primary grind 487 of the sharp 482 may rest against the sloped face 506. The beveled sections formed by the secondary grinds 489 as well as the point 485 may not be in contact with the sloped face 506. This may help to ensure that the tip 486 remains sharp and may help prevent any attenuation of the piercing capabilities of the tip 486.

With the sharp 482 in place, infusion tubing 366 may be coupled to the tubing connector 368. The tubing 366 may be introduced into a tubing receptacle 512 of the tubing connector 368. The lumen 514 of the tubing 336 may be placed into fluid communication with the lumen 516 of the sharp 482. The tubing receptacle 512 of the tubing connector 368 may include at least one tapered region 518 which may aid in funneling the tubing 366 into the tubing receptacle 512. Any force exerted on the sharp 482 as the tubing 366 is slid into place may be transmitted into the sloped face 506 through the primary grind 487 of the sharp 482. This may help to ensure the tip 486 of the sharp 482 is protected as the tubing 366 is assembled onto the tubing connector 368.

Once the tubing 366 is in place, an applicator may be advanced into the port 510. Glue or adhesive may be dispensed into an aperture 520 of the tubing connector 368. In some embodiments a UV curing adhesive may be used. UV light may be emitted toward the tubing connector 368 to cure the adhesive and fixedly retain the sharp 482 and tubing 366 onto the tubing connector 368. In some embodiments, UV emitting LEDS (no shown) may be included in the fixture 480 for this purpose, though an external light source may also be utilized. The tubing connector 368 may then be removed from the fixture 480.

Referring now also to FIG. 144, a flowchart 530 depicting a number of example actions which may be executed to assemble a tubing connector 368 is shown. In block 532, a tubing connector 368 may be coupled to a fixture 480. A sharp 482 may be introduced into the tubing connector 368 in block 534. The fixture 480 may be oriented such that the sharp 482 is introduced into the tubing connector 368 along the direction of acceleration due to gravity to allow gravity to assist in the introduction. Magnets 492, 494 in the fixture 480 may orient the sharp 482 in a desired position as the sharp 482 is introduced. In block 536, infusion tubing 366 may be inserted into a tubing receptacle 512 of the tubing connector 368. Adhesive may be applied into the tubing connector 540 in block 538. The adhesive may be cured in block 540. In block 542, the completed tubing connector 368 may be removed from the fixture 480.

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in drawings are presented only to demonstrate certain examples of the disclosure. The drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g. “a” “an” or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B.

Furthermore, the terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

What is claimed is:

1. A method for assembling a cannula subassembly, the method comprising:
providing a housing;
positioning a molded cannula such that a part of a first portion of the cannula is external to the housing and an enlarged region at an end of the cannula is disposed within the housing an on a seat of the housing;
compressing a septum against a portion of the exterior surface of the enlarged region and forming a fluid tight seal between at least the septum and the enlarged region; and
capturing the septum at least partially within the housing by coupling a retainer to the housing.

2. The method of claim 1, wherein capturing the septum comprises retaining a surface of the septum against an end wall of the housing.

3. The method of claim 2, wherein the end wall includes a passage therethrough and positioning the cannula comprises passing the first portion of the cannula at least partially through the passage.

4. The method of claim 1, wherein positioning the cannula comprises locating the enlarged region at least partially within a depression which at least partially defines the seat.

5. The method of claim 1, wherein positioning the cannula comprises locating a portion of the enlarged region on a raised projection surrounding an aperture in the housing through which the first portion of the cannula extends, the raised projection at least partially defining the seat.

6. The method of claim 1, wherein capturing the septum comprises engaging at least one retaining finger of the retainer into a cooperating receiving feature of the housing.

7. The method of claim 1, wherein capturing the septum comprises receiving a nub portion of the septum at least partially within a channel extending through the retainer and exerting a compressive force on the nub with a sidewall of the channel.

8. The method of claim 1, wherein compressing the septum comprises one of the list consisting of establishing a fluid seal against a straight walled portion of the enlarged region which is raised from a flange of the enlarged region and extends in a direction parallel to an axis of elongation of the cannula and establishing a fluid seal at least against a straight walled portion of the enlarged region that extends parallel to the axis of elongation of the cannula.

9. The method of claim 1, wherein compressing the septum comprises exerting a compressive force against the septum with a number of protuberances defined on the inner wall of the housing.

10. The method of claim 1, wherein the method further comprises coupling the cannula subassembly within a patient care assembly base.

11. A method for assembling a patient care assembly, the method comprising:
providing a septum housing;
positioning a molded cannula such that a part of a first portion of the cannula is external to the housing and an enlarged region at an end of the cannula is disposed on a seat within the housing;
capturing a septum at least partially within the housing and compressed against an exterior surface of the enlarged region so as to form a fluid tight seal;
coupling the septum housing in place within a receptacle of the patient care assembly.

12. The method of claim 11, wherein capturing the septum comprises exerting a compressive force against the septum with a number of protuberances defined on the inner wall of the housing.

13. The method of claim 11, wherein capturing the septum comprises coupling a retainer to the housing and receiving a nub portion of the septum at least partially within a channel extending through the retainer, the channel exerting a compressive force on the nub.

14. The method of claim 11, wherein positioning the cannula comprises one of the list consisting of: locating the enlarged region at least partially within a depression which at least partially defines the seat and locating a portion of the enlarged region on a raised projection surrounding an aperture in the housing through which the first portion of the cannula extends, the raised projection at least partially defining the seat.

15. The method of claim 11, wherein capturing the septum comprises establishing a fluid seal at least against a straight walled portion of the enlarged region that extends parallel to an axis of elongation of the cannula.

16. A method for assembling a patient care assembly, the method comprising:
positioning a molded cannula within a cannula subassembly housing with a part of a first portion of the cannula external to the housing and an enlarged region at an end of the cannula disposed on a seat within the housing;
capturing a septum at least partially within the housing and compressed against an exterior surface of the enlarged region so as to form a fluid tight seal;
coupling the housing in place within a receptacle of the patient care assembly.

17. The method of claim 16, wherein capturing the septum comprises exerting a compressive force against the septum with a number of protuberances defined on the inner wall of the housing.

18. The method of claim 16, wherein capturing the septum comprises coupling a retainer to the housing and receiving a nub portion of the septum at least partially within a channel extending through the retainer, the channel exerting a compressive force on the nub.

19. The method of claim 16, wherein positioning the cannula comprises one of the list consisting of: locating the enlarged region at least partially within a depression which at least partially defines the seat and locating a portion of the enlarged region on a raised projection surrounding an aperture in the housing through which the first portion of the cannula extends, the raised projection at least partially defining the seat.

20. The method of claim 16, wherein capturing the septum comprises one of the list consisting of establishing a fluid seal against a straight walled portion of the enlarged region which is raised from a flange of the enlarged region and extends in a direction parallel to an axis of elongation of the cannula and establishing a fluid seal at least against a straight walled portion of the enlarged region that extends parallel to the axis of elongation of the cannula.
